(19) United States
(12) Patent Application Publication
(10) Pub. No.: US 2024/0277878 A1
(43) Pub. Date: Aug. 22, 2024

(54) RADIOLABELED FIBRONECTIN BASED SCAFFOLDS AND ANTIBODIES AND THERANOSTIC USES THEREOF

(71) Applicant: BRISTOL-MYERS SQUIBB COMPANY, Princeton, NJ (US)

(72) Inventors: Paul E. MORIN, Pennington, NJ (US); David J. DONNELLY, Doylestown, PA (US); Wendy S. HAYES, Princeton, NJ (US); Ralph Adam SMITH, Yardley, PA (US); Samuel J. BONACORSI, Flemington, NJ (US); David K. LEUNG, West Windsor, NJ (US); Wolfgang WEBER, Munich (DE); Angela KRACKHARDT, Munich (DE)

(21) Appl. No.: 17/798,262

(22) PCT Filed: Feb. 26, 2021
(86) PCT No.: PCT/US2021/019972

§ 371 Date: Aug. 8, 2022

Related U.S. Application Data
(60) Provisional application No. 62/983,412, filed on Feb. 28, 2020.

(51) Int. Cl. A61K 51/10; A61K 51/08; A61P 35/00

(52) U.S. Cl. CPC A61K 51/1027; A61K 51/088; A61P 35/00; A61K 2121/00; A61K 2123/00

(57) ABSTRACT

Provided herein are radioimaging and radiotherapeutic agents for use in detecting, treating and monitoring cancer in a subject.

### REFERENCE TO RELATED APPLICATIONS

This application claims priority to U.S. Provisional Application No. 62/983,412, entitled “Radiolabeled Fibronectin Based Scaffolds and Antibodies and Theranostic Uses Thereof” filed Feb. 28, 2020, the entire contents of which are hereby incorporated by reference herein.

### BACKGROUND

Theranostics uses specific biological pathways in the human body, to acquire diagnostic images and also to deliver a therapeutic dose of radiation to the patient. A specific diagnostic test shows a particular molecular target on at a disease site, for example a tumor, allowing a therapeutic agent to specifically bind to the target at the disease site, rather than more broadly targeting other tissues thereby avoiding adverse effects. This combined approach provides a more targeted, efficient pharmacotherapy than previous one-medicine-fits-all approaches.

Accordingly, there remains a need for suitable radiolabeled agents for use in non-invasive in vivo imaging and therapeutic methods to assess target expression and distribution, and to obtain reliable, diagnostic, prognostic and therapeutic information.

### SUMMARY

Provided herein are theranostic combinations of radioimaging agents for use in imaging cancer in a subject, and radiotherapeutic agents for treating the cancer in a subject. The combination of the radioimaging agents and radiotherapeutic agents provided herein are useful in methods of detecting cancer cells, tumor size and location, cancer cell metastasis, and cancer cell migration in a subject, combined with methods of radiotherapy to treat the cancer, as well as monitoring the efficacy of a radiotherapeutic administered to the subject.

In one aspect, provided herein is a combination for use in diagnosis, monitoring and treatment of cancer in a subject, the combination comprising (a) a radioimaging agent comprising a fibronectin based scaffold (FBS) polypeptide which binds to a target expressed by the cancer and a radionuclide; and (b) a radiotherapeutic agent comprising the FBS polypeptide and a radionuclide, wherein the FBS polypeptide of the radioimaging agent and the radiotherapeutic agent bind to the target.

In some embodiments, the radionuclide of the radioimaging and/or radionuclide is linked to the FBS polypeptide by a chelating agent, e.g., a bifunctional chelating agent (BFC), which comprises a reactive group that forms a covalent bond with an amine, carboxyl, carbonyl or thiol functional group on the targeting protein or peptide. In some embodiments, the chelator is covalently attached to the FBS polypeptide via a linker, e.g., a peptide linker.

In some embodiments, the FBS polypeptide comprises a human ¹⁰Fn3 domain. In certain embodiments the ¹⁰Fn3 domain binds to human PD-L1 (i.e., is an anti-PD-L1 Adnectin). In particular embodiments, the BC, DE, and FG loops of the anti-PD-L1 Adnectin comprise the amino acid sequences of: (a) SEQ ID NOs: 6, 7, and 8, respectively; (b) SEQ ID NOs: 21, 22, and 23, respectively; (c) SEQ ID NOs: 36, 37, and 38, respectively; (d) SEQ ID NOs: 51, 52, and 53, respectively; (e) SEQ ID NOs: 66, 67, and 68, respectively; (f) SEQ ID NOs: 81, 82, and 83, respectively; or (g) SEQ ID NOs: 97, 98, and 99, respectively. In one embodiment, the anti-PD-L1 Adnectin comprises SEQ ID NO: 96 or SEQ ID NO: 80.

In another aspect, provided herein is a radiotherapeutic agent comprising an anti-PD-L1 antibody or antigen binding fragment thereof and a radionuclide. In some embodiments, the radiotherapeutic agent is an anti-PD-L1 antibody or antigen binding fragment thereof comprising the three VH CDR of antibody 12A4. In some embodiments, the radiotherapeutic agent is an antibody or antigen binding fragment comprising the three CDRs of the VH of antibody 12A4. In some embodiments, the radiotherapeutic agent is an anti-PD-L1 antibody or antigen binding fragment thereof that comprises the CDRs of the VH and the CDRs of the VL of antibody 12A4. In some embodiments, the radiotherapeutic agent is an anti-PD-L1 antibody or antigen binding fragment thereof that comprises the VH and VL of antibody 12A4. In one embodiment, the radiotherapeutic agent comprises 12A4 or an antigen binding fragment thereof.

In a related aspect, provided herein is a method of treating cancer comprising administering to a subject in need thereof a radiolabeled anti-PD-L1 antibody or antigen binding fragment thereof.

In another aspect, provided herein is a combination for use in detecting and treating cancer in a subject, comprising (a) a radioimaging agent comprising a PD-L1 antibody or antigen binding fragment thereof and a radionuclide; and (b) a radiotherapeutic agent comprising a PD-L1 antibody or antigen binding fragment thereof and a radionuclide, wherein the PD-L1 antibody or antigen binding fragment thereof of the radioimaging agent and the radiotherapeutic agent have the same antigen binding specificity.

In some embodiments, the radionuclide of the radioimaging agent and/or radiotherapeutic agent is linked to the anti-PD-L1 antibody or antigen binding fragment thereof by a chelating agent, e.g., a bifunctional chelating agent (BFC), which comprises a reactive group that forms a covalent bond with an amine, carboxyl, carbonyl or thiol functional group on the targeting protein or peptide. In some embodiments, the chelator is covalently attached to the anti-PD-L1 antibody or antigen binding fragment thereof via a linker, e.g., a peptide linker.

In some embodiments, the radioimaging agent and/or radiotherapeutic agent is an anti-PD-L1 antibody or antigen binding fragment thereof comprising the three VH CDR of antibody 12A4. In some embodiments, the radioimaging and/or radiotherapeutic agent is an antibody or antigen binding fragment comprising the three CDRs of the VH of antibody 12A4. In some embodiments, the radioimaging and/or radiotherapeutic agent is an anti-PD-L1 antibody or antigen binding fragment thereof that comprises the CDRS of the VH and the CDRs of the VL of antibody 12A4. In some embodiments, the radioimaging and/or radiotherapeutic agent is an anti-PD-L1 antibody or antigen binding fragment thereof that comprises the VH and VL of antibody 12A4. In one embodiment, the radioimaging and/or radiotherapeutic agent is 12A4.

In another aspect, provided herein are methods of using the combination of radioimaging agents and radiotherapeutic agents provided herein for diagnosing, monitoring and treating cancer in a subject. In some embodiments, the method comprises: (a) administering to the subject an radioimaging agent comprising a fibronectin based scaffold (FBS) polypeptide which binds to a target expressed by cancer cells and a radionuclide suitable for radioimaging; (b) obtaining a radioimage of all or a portion of the subject to determine the presence of the target in the subject; (c) administering a radiotherapeutic agent comprising an FBS polypeptide and a radionuclide suitable for radiotherapy wherein the radioimaging agent and radiotherapeutic agent bind to the same target.

In some embodiments, the method comprises (a) administering to the subject a radioimaging agent comprising a fibronectin based scaffold (FBS) polypeptide which binds to a target expressed by cancer cells and a radionuclide suitable for radioimaging; (b) obtaining a radioimage of all or a portion of the subject to determine the presence of the target in the subject; (c) administering a radiotherapeutic agent comprising an FBS polypeptide and a radionuclide suitable for radiotherapy wherein the radioimaging agent and radiotherapeutic agent bind to the same target.

In some embodiments, the radioimaging agent is also administered after the radiotherapeutic agent to monitor target levels in the subject, and further administration of the radiotherapeutic agent is determined based on the target levels identified with the radioimaging agent.

In some embodiments, the cancer is a PD-L1 expressing cancer and the radioimaging agent and radiotherapeutic agent are an anti-PD-L1 Adnectin. In some embodiments, the radioimaging agent and therapeutic agent are an anti-PD-L1 antibody or antigen binding fragment thereof. In other embodiments, the radioimaging agent is an anti-PD-L1 Adnectin and the radiotherapeutic agent is an anti-PD-L1 antibody or fragment thereof.

Also provided herein are pharmaceutical compositions comprising the radioimaging and/or radiotherapeutic agents provided herein. Also provided herein are kits comprising the radioimaging and radiotherapeutic agents provided herein, and instructions for use.

### BRIEF DESCRIPTION OF THE DRAWINGS

FIG. 1 depicts the characterization of PD-L1 transduced U698M cells (left). Representative flow cytometric evaluation of PD-L1 expression for transduced U698M (bottom) and non-transduced U698M (top) analyzed by a PD-L1 staining (right). Quantification of PD-L1 molecules per cell for both non-transduced and transduced U698M cells.

FIG. 2 depicts maximum intensity projections (MIP) of static pPET scans of ⁶⁸Ga-Adnectin 1 h p.i. (upper panel) and 2 h p.i. (lower panel) in PD-L1 positive U-698-M (white arrows) and U-698-M wild-type (clear arrows) xenograft bearing NSG mice (0%-9% ID/g). Mouse #481: Coinjection of 68Ga-Adnectin+Blocking with 9 mg/kg unlabeled Adnectin.

FIG. 3A-C depicts axial slices: CT (FIG. 3A), PET (FIG. 3B), PET/CT (FIG. 3C) of ⁶⁸Ga-Adnectin in U698M-PDL1+ (white arrows) and U698M wild-type (clear arrows) tumor-bearing NSG mice at 1 h p.i. Data are expressed as 0-9% ID/g.

FIG. 4 depicts ROI Quantification of static PET scans of ⁶⁸Ga-Adnectin 1 h p.i. and 2 h p.i. in PD-L1 positive U-698-M and U-698-M wild-type xenograft bearing mice. One mouse was coinjected with ⁶⁸Ga-Adnectin+9 mg/kg unlabeled Adnectin (Blocking).

FIG. 5A depicts maximum intensity projections (MIP) of a dynamic PET scan. Summation images of different time-frames (0-9% ID/g) of ⁶⁸Ga-Adnectin in PD-L1 positive U-698-M and U-698-M wild-type xenograft bearing mice over an acquisition time of 90 min.

FIG. 5B. Time-activity curves in % ID/g for blood pool (heart), kidneys, muscle, liver and PD-L1 positive and negative tumor derived from dynamic pPET/CT data.

FIG. 6 depicts biodistribution data (in % ID/g) of ⁶⁸Ga-Adnectin in PD-L1 positive U-698-M and U-698-M wild-type xenograft bearing mice (1 h and 2 h p.i.).

FIG. 7 depicts representative ex vivo FACS analysis from two mice, assessing the GFP (top) and PD-L1 (bottom) expression on wildtype (cross-hatched) and PD-L1 transduced (shaded) tumors.

FIG. 8 depicts ex vivo analysis of PD-L1 positive cells within U-698-M tumors. Corresponding immunohistochemical HE-staining and PD-L1-staining of wildtype and PD-L1 transduced tumors.

FIG. 9A and FIG. 9B depict SPECT/CT imaging of ¹⁷⁷Lu-anti-PD-L1 Adnectin in U698M-PDL1+ and U698M wild-type (WT) tumor-bearing NSG mice at 1 h, 24 h, 72 h and 7 d p.i. (Injected Dose: 37 MBq; 100 μg Adnectin. n=1) and FIG. 9C at 1 h, 24 h, 48 h and 72 h p.i. (Injected Dose: 33 MBq; 69 μg Adnectin. n=1).

FIG. 10A depicts SPECT/CT imaging and ex vivo biodistribution of ¹⁷⁷Lu-anti-PD-L1 Adnectin in U698M-PDL1+ and U698M WT tumor-bearing NSG mice at 72 h p.i. (Injected Dose: 33 MBq; 69 μg Adnectin. n=1) and FIG. 10B at 7 d p.i. (Injected Dose: 37 MBq; 100 μg Adnectin. n=1).

FIG. 11A depicts SPECT/CT imaging of ¹⁷⁷Lu-anti-PD-L1 mAb in U698M-PDL1+ and U698M WT tumor-bearing NSG mice at 5 h, 24 h, 48 h, 72 h and 7 d p.i. (Injected Dose: 25 MBq; 100 μg mAb. n=1) and FIG. 11B at 5 h, 24 h, 48 h and 72 h p.i. and 96 h p.i. (Injected Dose: 34 MBq; 102 μg mAb. n=1).

FIG. 12A depicts SPECT/CT imaging and ex vivo biodistribution of ¹⁷⁷Lu-anti-PD-L1 mAb in U698M-PDL1+ and U698M WT tumor-bearing NSG mice at 96 h p.i. (Injected Dose: 34 MBq; 102 μg mAb. n=1) and FIG. 12B at 7 d p.i. (Injected Dose: 25 MBq; 102 μg mAb. n=1).

FIG. 13 depicts biodistribution of ¹⁷⁷Lu-anti-PD-L1 mAb in U698M-PDL1+ and U698M tumor-bearing NSG mice at 5 h (n=1), 24 h p.i. (n=1) and 72 h p.i. (n=1). Injected Dose: 1.5 MBq, app. 13 μg mAb.

FIG. 14 depicts the in vitro stability of ⁶⁸Ga-Adnectin up to 4 h as determined by Radio-TLC and Radio-HPLC.

FIG. 15A-B depict Radiochemical Yields (RCYs) of ⁶⁸Ga-labeling of NOTA- versus DOTA-Adnectin as a function of the amount of starting activity, precursor and reaction time.

FIG. 16 depicts the maximum intensity projections (MIP) of comparative dynamic pPET scans of ⁶⁸Ga-labeled DOTA- and NOTA-Adnectin. Summation images of different time-frames (0-9% ID/g) of ⁶⁸Ga-DOTA- and ⁶⁸Ga-NOTA-Adnectin in PD-L1 positive U-698-M (white arrows) and U-698-M wild-type (clear arrows) xenograft bearing mice over an acquisition time of 90 min. (B=Bladder, K=Kidneys).

FIG. 17 depicts the maximum intensity projections (MIP) of static pPET scans (5-6 MBq, app. 10 μg) of ⁶⁸Ga-DOTA-Adnectin (upper panel) and ⁶⁸Ga-NOTA-Adnectin (lower panel) 1 h p.i. in PD-L1 positive U-698-M (white arrows) and U-698-M wild-type (clear arrows) xenograft bearing NSG mice (0%-9% ID/g). Blocking: Coinjection of ⁶⁸Ga-DOTA/NOTA-Adnectin+Blocking with 9 mg/kg unlabeled Adnectin.

FIG. 18 depicts the biodistribution of ⁶⁸Ga-DOTA- and ⁶⁸Ga-NOTA-Adnectin in PD-L1 positive U-698-M and U-698-M wild-type tumor-bearing NSG mice at 1 h p.i (5-6 MBq, app. 10 μg). Data are expressed as % ID/g (mean±SD).

FIG. 19 depicts SPECT/CT imaging and ex vivo autoradiography, HE- and IHC staining of ¹⁷⁷Lu-anti-PD-L1 Adnectin in a U698M-PDL1+ and U698M WT tumor-bearing NSG mouse at 7 d p.i (Injected Dose: 37 MBq; 100 μg Adnectin.

FIG. 20 depicts the binding affinity and specificity of ¹⁷⁷Lu-mAb towards PD-L1 determined in competitive radioligand binding assays. (Left) Cell bound activity of ¹⁷⁷Lu-mAb in the presence of increasing concentrations of cold ligand (DOTA-mAb). (Right) Cell bound activity of ¹⁷⁷Lu-mAb in the presence of cold ligand (0.1 nM) on PD-L1 positive and wild-type U-689-M cells.

### DETAILED DESCRIPTION

### I. Definitions

Unless defined otherwise, all technical and scientific terms used herein have the same meaning as commonly understood by the skilled artisan. Although any methods and compositions similar or equivalent to those described herein can be used in practice or testing of the present invention, the preferred methods and compositions are described herein.

As used herein, the singular forms “a”, “an” and “the” include plural referents unless the context clearly dictates otherwise. The use of “or” or “and” means “and/or” unless stated otherwise. Furthermore, use of the term “including” as well as other forms, such as “include”, “includes”, and “included”, is not limiting.

As used herein, “about” means within plus or minus ten percent of a number. For example, “about 100” would refer to any number between 90 and 110.

As used herein, the term “theranostic” refers to a combination of a radiolabeled diagnostic agent and a radiotherapeutic. Generally, the radiolabeled agents are administered separately, e.g., the radiolabeled diagnostic agent contains a polypeptide which bind to a targets associated with disease (e.g., cancer) is used to assess the location, extent and target density of a disease conditions (e.g., cancer) through imaging, such as positron emission topography (PET) imaging, followed by radioimmunotherapy (RIT) with the radiotherapeutic agent which binds to the same target associated with diseased cells.

As used herein, “target” as a general reference to a “biological target” refers to a cell, tissue (e.g., cancer or tumor), a pathogenic microorganism (e.g., bacteria, virus, fungus, plant, prion, protozoa or portion thereof), protein (e.g., PD-L1) or other molecule

The term “targeting ligand”, “targeting agent” or “targeting molecule” are used interchangeably to refer to a molecule, such as peptide, protein, glycoprotein, etc., FBS polypeptide (e.g., Adnectin, e.g., PD-L1 Adnectin), antibody, or antigen binding protein, e.g., antigen binding fragment of an antibody, that binds to another molecule. In certain embodiments, a targeting agent is bound to the radiolabel in order to “target” pathway the radiolabel to other molecule to which the targeting agent binds.

“Polypeptide” as used herein refers to any sequence of two or more amino acids, regardless of length, post-translation modification, or function. “Polypeptide,” “peptide,” and “protein” are used interchangeably herein. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D).

“A “polypeptide chain,” as used herein, refers to a polypeptide wherein each of the domains thereof is joined to other domain(s) by peptide bond(s), as opposed to non-covalent interactions or disulfide bonds.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In preferred embodiments, the polypeptide will be purified (1) to greater than 95% by weight of polypeptide as determined by the Lowry method, and most preferably more than 99% by weight, (2) to a degree sufficient to obtain at least residues of N-terminal or internal amino acid sequence by use of a spinning cup sequenator, or (3) to homogeneity by SDS-PAGE under reducing or nonreducing condition using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR™) software. Those skilled in the art can readily determine appropriate parameters for measuring alignment, including any algorithms needed to achieve maximal alignment over the full-length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Antigen binding protein” refers to a protein that binds an antigen, and includes FBS polypeptides (e.g., Adnectins), antibodies and antigen binding fragments (or portions) of antibodies.

As used herein, the terms “FBS binding site,” “Adnectin binding site” and “antibody binding site” refers to the site or portion of a protein (e.g., PD-L1) that interacts or binds to a particular FBS polypeptide (e.g., ¹⁰Fn3 domain of the polypeptide), Adnectin or antibody, respectively. Binding sites can be formed from contiguous amino acids or noncontiguous amino acids juxtaposed by tertiary folding of a protein. Binding sites formed by contiguous amino acids are typically retained on exposure to denaturing solvents, whereas Binding sites formed by tertiary folding are typically lost on treatment of denaturing solvents.

As used herein in the context of FBS polypeptides (e.g., Adnectins) or antigen binding proteins, e.g., antibodies provided herein, the term “cross-reactivity” refers to an FBS polypeptide or antigen binding protein, respectively which binds to more than one distinct protein having identical or very similar binding sites.

“Antigen binding specificity” refers to the portions of an antibody that provides it the ability to bind specifically to a specific region of an antigen, and can be, e.g., the variable regions of the antibody or the CDRs thereof. In cases in which a certain CDR, e.g., VL CDR1, is not important for antigen binding, such CDR is not included in defining the “antigen binding specificity.” Two or more antibodies may have the same antigen binding specificity even if specific amino acids in one or more CDRs are substituted, deleted or added. Whether two antibodies have the same antigen binding specificity can be determined by crystallography, where the antigen binding specificity of two antibodies are the same if they have the same interactions with the target protein, as determined by the crystal structure.

The term “antibody” as used herein (abbreviated “Ab”) refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term also encompasses molecules with full length heavy and/or light chains. The term antibody also includes, but is not limited to, chimeric antibodies, humanized antibodies, and antibodies of various species such as mouse, human, cynomolgus monkey, etc.

In some embodiments, an antibody comprises a heavy chain variable region and a light chain variable region. In some embodiments, an antibody comprises at least one heavy chain comprising a heavy chain variable region and at least a portion of a heavy chain constant region, and at least one light chain comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

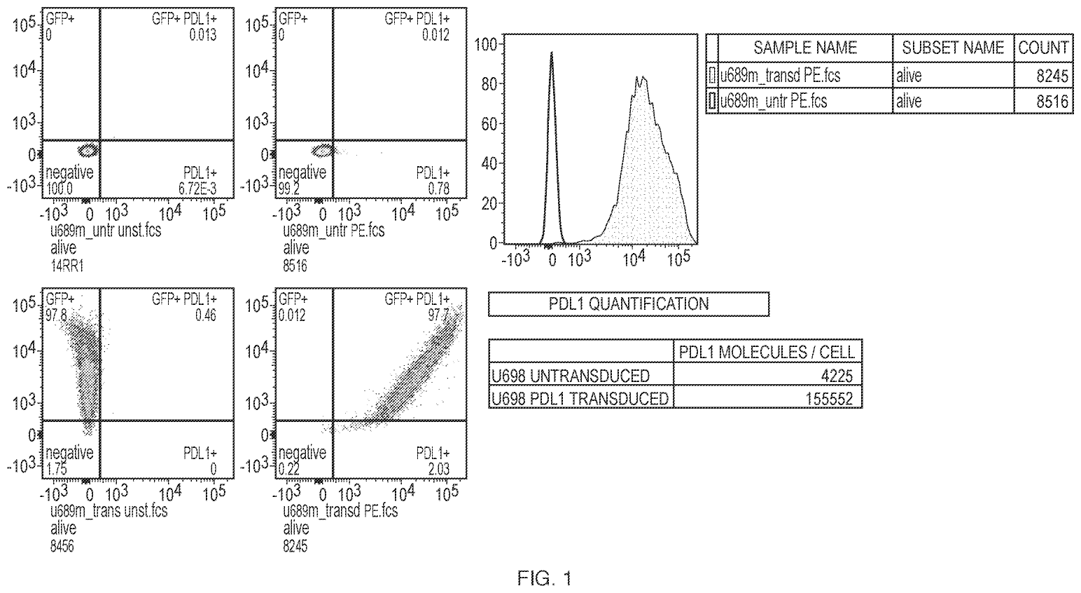

The term “heavy chain variable region” as used herein refers to a region comprising heavy chain CDR1, framework (FR) 2, CDR2, FR3, and CDR3. In some embodiments, a heavy chain variable region also comprises at least a portion of an FR1 and/or at least a portion of an FR4. In some embodiments, a heavy chain CDR1 corresponds to Kabat residues 26 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.); and FIG. 1. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See id.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ3 constant region), and IgG4 (comprising a γ4 constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

In some embodiments, a heavy chain constant region comprises one or more mutations (or substitutions), additions, or deletions that confer a desired characteristic on the antibody. A nonlimiting exemplary mutation is the S241P mutation in the IgG4 hinge region (between constant domains C_{H}1 and C_{H}2), which alters the IgG4 motif CPSCP to CPPCP, which is similar to the corresponding motif in IgG1. That mutation, in some embodiments, results in a more stable IgG4 antibody. See, e.g., Angal et al., Mol. Immunol. 30: 105-108 (1993); Bloom et al., Prot. Sci. 6: 407-415 (1997); Schuurman et al., Mol. Immunol. 38: 1-8 (2001).

The term “heavy chain” (abbreviated HC) as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising light chain CDR1, framework (FR) 2, CDR2, FR3, and CDR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

The term “light chain constant region” as used herein refers to a region comprising a light chain constant domain, C_{L}. Nonlimiting exemplary light chain constant regions include λ and κ.

The term “light chain” (abbreviate LC) as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

A “chimeric antibody” as used herein refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, a chimeric antibody comprises at least one rat variable region and at least one mouse constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is a Fab, an scFv, a (Fab′)2, etc.

A “CDR-grafted antibody” as used herein refers to a humanized antibody in which the complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The terms “specifically binds,” “specific binding,” “selective binding, and “selectively binds,” as used interchangeably herein refers to an FBS polypeptide (e.g., Adnectin) or antigen binding protein that exhibits affinity for a target (e.g., PD-L1), but does not significantly bind (e.g., less than about 10% binding) to different polypeptides as measured by a technique available in the art such as, but not limited to, Scatchard analysis and/or competitive binding assays (e.g., competition ELISA, BIACORE assay). The term is also applicable where, e.g., a binding domain of an FBS polypeptide or antibody described herein is specific for a target (e.g., PD-L1).

The term “preferentially binds” as used herein refers to the situation in which an FBS polypeptide (e.g., an Adnectin) or antigen binding protein described herein binds to a target (e.g., PD-L1) at least about 20% greater than it binds a different polypeptide as measured by a technique available in the art such as, but not limited to, Scatchard analysis and/or competitive binding assays (e.g., competition ELISA, BIACORE assay).

The term “K_{D},” as used herein, is intended to refer to the dissociation equilibrium constant of a particular target (e.g., PD-L1) interaction or the affinity of an FBS polypeptide (e.g., Adnectin) or antigen binding protein for a protein (e.g., PD-L1), as measured using a surface plasmon resonance assay or a cell binding assay. A “desired K_{D},” as used herein, refers to a K_{D} of an Adnectin that is sufficient for the purposes contemplated. For example, a desired K_{D} may refer to the K_{D} of an Adnectin required to elicit a functional effect in an in vitro assay, e.g., a cell-based luciferase assay.

The term “kₐ”, as used herein is intended to refer to the association rate constant for the binding of an FBS polypeptide or antigen binding protein into a complex with the target.

The term “k_{d}”, as used herein, is intended to refer to the dissociation rate constant for the dissociation of an FBS polypeptide or antigen binding protein from a complex with the target.

The term “IC₅₀”, as used herein, refers to the concentration of an FBS polypeptide (e.g., Adnectin) or antibody that inhibits a response, either in an in vitro or an in vivo assay, to a level that is 50% of the maximal inhibitory response, i.e., halfway between the maximal inhibitory response and the untreated response.

The term “PK” is an acronym for “pharmacokinetic” and encompasses properties of a compound including, by way of example, absorption, distribution, metabolism, and elimination by a subject. A “PK modulation protein” or “PK moiety” as used herein refers to any protein, peptide, or moiety that affects the pharmacokinetic properties of a biologically active molecule when fused to or administered together with the biologically active molecule. Examples of a PK modulation protein or PK moiety include PEG, human serum albumin (HSA) binders (as disclosed in U.S. Publication Nos. 2005/0287153 and 2007/0003549, PCT Publication Nos. WO 2009/083804 and WO 2009/133208), human serum albumin and variants thereof, transferrin and variants thereof, Fc or Fc fragments and variants thereof, and sugars (e.g., sialic acid).

The “serum half-life” of a protein or compound is the time taken for the serum concentration of the polypeptide to be reduced by 50%, in vivo, for example due to degradation of the sequence or compound and/or clearance or sequestration of the sequence or compound by natural mechanisms. The half-life can be determined in any manner known per se, such as by pharmacokinetic analysis. Suitable techniques will be clear to the person skilled in the art, and may for example generally involve the steps of suitably administering to a subject a suitable dose of the amino acid sequence or compound described herein; collecting blood samples or other samples from the subject at regular intervals; determining the level or concentration of the amino acid sequence or compound described herein in said blood sample; and calculating, from (a plot of) the data thus obtained, the time until the level or concentration of the amino acid sequence or compound described herein has been reduced by 50% compared to the initial level upon dosing. Reference is, for example, made to the standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

Half-life can be expressed using parameters such as the t_{1/2}-alpha, t_{1/2}-beta, HL_Lambdaz, and the area under the curve (AUC).

In one example, the term “radiochemical” as used herein refers to an organic, inorganic or organometallic compound comprising a covalently-attached or coordinately-attached (ligand) radioactive isotope, particularly including radioactive imaging probes and radiotherapeutic agents intended for administration to a patient, which are also referred to in the art as radiopharmaceuticals, radiotracers or radioligands.

The term “radioactive isotope” or “radioactive element” refers to isotopes exhibiting radioactive decay (for example, emitting positrons, beta particles, gamma radiations etc.) and radiolabeling agents comprising a radioactive isotope. Isotopes or elements are also referred to in the art as radioisotopes or radionuclides.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The terms “diagnosis” or “detection” can be used interchangeably. Whereas diagnosis usually refers to defining a tissue's specific histological status, detection recognizes and locates a tissue, lesion or organism containing a particular detectable target.

The term “detectable” refers to the ability to detect a signal over the background signal. The term “detectable quantity” refers to the amount of the detectable compound that is administered is sufficient to enable detection of binding of the compound to the cancer cells. The term “detectable signal” as used herein in the context of imaging agents and diagnostics, is a signal derived from non-invasive imaging techniques such as, but not limited to, positron emission tomography (PET). The detectable signal is detectable and distinguishable from other background signals that may be generated from the subject. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between the detectable signal and the background. Standards and/or calibration curves can be used to determine the relative intensity of the detectable signal and/or the background.

A “detectably effective amount” or “imaging effective quantity” of a composition comprising an imaging agent described herein is defined as an amount sufficient to yield an acceptable image using equipment that is available for clinical use. A detectably effective amount of an imaging agent provided herein may be administered in more than one injection. The detectably effective amount can vary according to factors such as the degree of susceptibility of the individual, the age, sex, and weight of the individual, idiosyncratic responses of the individual, and the like. Detectably effective amounts of imaging compositions can also vary according to instrument and methodologies used. Optimization of such factors is well within the level of skill in the art.

“PD-L1 positive” as used herein can be interchangeably used with “PD-L1 expression of at least about 5%.” A PD-L1 positive tumor can thus have at least about 5%, at least about 10%, or at least about 20% of tumor cells expressing PD-L1. In certain embodiments, “PD-L1 positive” means that there are at least 100 cells that express PD-L1 on the surface of the cells. PD-L1 expression can be measured by any methods known in the art. In some embodiments, PD-L1 positive tumors express detectable levels of PD-L1 as measured by the radioimaging methods provided herein. In other embodiments, PD-L1 expression is measured by an automated IHC.

“Treatment” or “therapy” of a subject refers to any type of intervention or process performed on, or the administration of an active agent to, the subject with the objective of reversing, alleviating, ameliorating, inhibiting, slowing down, progression, development, severity or recurrence of a symptom, complication, condition or biochemical indicia associated with a disease.

“The term “therapeutically effective amount” refers to at least the minimal dose, but less than a toxic dose, of an agent which is necessary to impart a therapeutic benefit to a subject.

As used herein, an “effective amount” refers to at least an amount effective, at dosages and for periods of time necessary, to achieve the desired result.

As used herein, a “sufficient amount” refers to an amount sufficient to achieve the desired result.

As used herein, “administering,” as used in the context of the imaging agents and therapeutic agents provided herein refers to the physical introduction of a composition comprising an imaging agent or therapeutic agent to a subject, using any of the various methods and delivery systems known to those skilled in the art. Preferred routes of administration for the imaging agents described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an imaging agent described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “co-administration” or the like, as used herein, are meant to encompass administration of the selected pharmaceutical agents to a single patient, and are intended to include regimens in which the agents are administered by the same or different route of administration or at the same or different time.

The terms “patient” and “subject” and “individual” are used interchangeable herein to refer to a human, e.g., a human that receives a composition comprising an imaging agent and a therapeutic agent in accordance with the methods provided herein. For in vitro applications, such as in vitro diagnostic and research applications, body fluids and cell samples of the above subjects will be suitable for use, such as blood, urine, or tissue samples.

The term “sample” can refer to a tissue sample, cell sample, a fluid sample, and the like. The sample may be taken from a subject. The tissue sample can include hair (including roots), buccal swabs, blood, saliva, semen, muscle, or from any internal organs. The fluid may be, but is not limited to, urine, blood, ascites, pleural fluid, spinal fluid, and the like. The body tissue can include, but is not limited to, skin, muscle, endometrial, uterine, and cervical tissue.

The term “isotopically pure” means that the element, compound, or composition contains a greater proportion of one isotope in relation to other isotopes. In certain embodiments, the element, compound, or composition is greater than about 40%, 50%, or 60% isotopically pure.

As used herein, a labeled molecule is “purified” when the labeled molecule is partially or wholly separated from unlabeled molecules, so that the fraction of labeled molecules is enriched compared to the starting mixture. A “purified” labeled molecule may comprise a mixture of labeled and unlabeled molecules in almost any ratio, including but not limited to about 5:95; 10:90; 15:85; 20:80; 25:75; 30:70; 40:60; 50:50; 60:40; 70:30; 75:25; 80:20; 85:15; 90:10; 95:5; 97:3; 98:2; 99:1 or 100:0.

The term “bioorthogonal chemistry” refers to any chemical reaction that can occur inside of living systems without interfering with native biochemical processes. The term includes chemical reactions that are chemical reactions that occur in vitro at physiological pH in, or in the presence of water. To be considered bioorthogonol, the reactions are selective and avoid side-reactions with other functional groups found in the starting compounds. In addition, the resulting covalent bond between the reaction partners should be strong and chemically inert to biological reactions and should not affect the biological activity of the desired molecule.

The term “click chemistry” refers to a set of reliable and selective bioorthogonal reactions for the rapid synthesis of new compounds and combinatorial libraries. Properties of for click reactions include modularity, wideness in scope, high yielding, stereospecificity and simple product isolation (separation from inert by-products by non-chromatographic methods) to produce compounds that are stable under physiological conditions. In radiochemistry and radiopharmacy, click chemistry is a generic term for a set of labeling reactions which make use of selective and modular building blocks and enable chemoselective ligations to radiolabel biologically relevant compounds in the absence of catalysts. A “click reaction” can be with copper, or it can be a copper-free click reaction.

The term “prosthetic group” or “bifunctional labeling agent” refers to a small organic molecule containing a radionuclide that is capable of being linked to peptides or proteins.

The terms “chelator” and “chelator ligand” as used herein with respect to radiopharmaceutical chemistry refers to a molecule that is joined to a polypeptide for the purpose of labeling the polypeptide with a radionuclide by loading the chelator with the radionuclide, and include bifunctional chelators (BFC), which contains reactive functional groups that can be covalently coupled (conjugated) to a targeting molecule (e.g., peptide, protein, nucleotide, nanoparticle). BFCs utilize functional groups such as carboxylic acids or activated esters (e.g., N-hydroxy-succinimide NHS-ester, tetrafluorophenyl TFP-ester) for amide couplings, isothiocyanates for thiourea couplings and maleimides for thiol couplings.

A “cancer” refers a broad group of various diseases characterized by the uncontrolled growth of abnormal cells in the body. Unregulated cell division and growth divide and grow results in the formation of malignant tumors that invade neighbouring tissues and may also metastasize to distant parts of the body through the lymphatic system or bloodstream.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

An “immunoregulator” refers to a substance, an agent, a signaling pathway or a component thereof that regulates an immune response. “Regulating,” “modifying” or “modulating” an immune response refers to any alteration in a cell of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function in the cancer microenvironment.

The term “immunotherapy” refers to the treatment of a subject afflicted with, or at risk of contracting or suffering a recurrence of, a disease by a method comprising inducing, enhancing, suppressing or otherwise modifying an immune response.

As used herein, “positron emission tomography” or “PET” refers to a non-invasive, nuclear medicine technique that produces a three-dimensional image of tracer location in the body. The method detects pairs of gamma rays emitted indirectly by a positron-emitting radionuclide (tracer), which is introduced into the body on a biologically active molecule. PET imaging tools have a wide variety of uses and aid in drug development both preclinically and clinically. Exemplary applications include direct visualization of in vivo saturation of targets; monitoring uptake in normal tissues to anticipate toxicity or patient to patient variation; quantifying diseased tissue; tumor metastasis; and monitoring drug efficacy over time, or resistance over time.

Various aspects described herein are described in further detail in the following subsections.

### II. Fibronectin-Based Scaffolds (FBS)

In one aspect, the targeting molecule used in the radiolabeled imaging and radiolabeled therapeutic compounds described herein is an FBS protein.

As used herein, a “fibronectin based scaffold” or “FBS” protein or moiety refers to proteins or moieties that are based on a fibronectin type III (“Fn3”) repeat. Fn3 is a small (about 10 kDa) domain that has the structure of an immunoglobulin (Ig) fold (i.e., an Ig-like β-sandwich structure, consisting of seven β-strands and six loops). Fibronectin has 18 Fn3 repeats, and while the sequence homology between the repeats is low, they all share a high similarity in tertiary structure. Fn3 domains are also present in many proteins other than fibronectin, such as adhesion molecules, cell surface molecules, e.g., cytokine receptors, and carbohydrate binding domains. For reviews see Bork et al., Proc. Natl. Acad. Sci. USA, 89(19):8990-8994 (1992); Bork et al., J. Mol. Biol., 242(4):309-320 (1994); Campbell et al., Structure, 2(5):333-337 (1994); Harpez et al., J. Mol. Biol., 238(4):528-539 (1994)). The term “FBS” protein or moiety is intended to include scaffolds based on Fn3 domains from these other proteins (i.e., non fibronectin molecules).

An Fn3 domain is small, monomeric, soluble, and stable. It lacks disulfide bonds and, therefore, is stable under reducing conditions. Fn3 domains comprise, in order from N-terminus to C-terminus, a beta or beta-like strand, A; a loop, AB; a beta or beta-like strand, B; a loop, BC; a beta or beta-like strand, C; a loop, CD; a beta or beta-like strand, D; a loop, DE; a beta or beta-like strand, E; a loop, EF; a beta or beta-like strand, F; a loop, FG; and a beta or beta-like strand, G. The seven antiparallel β-strands are arranged as two beta sheets that form a stable core, while creating two “faces” composed of the loops that connect the beta or beta-like strands. Loops AB, CD, and EF are located at one face (“the south pole”) and loops BC, DE, and FG are located on the opposing face (“the north pole”). There are at least 15 different Fn3 modules in human Fibronectin, and while the sequence homology between the modules is low, they all share a high similarity in tertiary structure.

The loops in Fn3 molecules are structurally similar to complementary determining regions (CDRs) of antibodies, and when altered, may be involved in binding of the Fn3 molecule to a target, e.g., a target protein. Other regions of Fn3 molecules, such as the beta or beta-like strands and N-terminal or C-terminal regions, when altered, may also be involved in binding to a target. Any or all of loops AB, BC, CD, DE, EF and FG may participate in binding to a target. Any of the beta or beta-like strands may be involved in binding to a target. Fn3 domains may also bind to a target through one or more loops and one or more beta or beta-like strands. Binding may also require the N-terminal or C-terminal regions. An FBS domain for use in a protein may comprise all loops, all beta or beta-like strands, or only a portion of them, wherein certain loops and/or beta or beta-like strands and/or N- or C-terminal regions are modified (or altered), provided that the FBS domain preferably binds specifically to a target. For example, an FBS domain may comprise 1, 2, 3, 4, 5 or 6 loops, 1, 2, 3, 4, 5, 6, 7, or 8 beta strands, and optionally an N-terminal and/or C-terminal region, wherein one or more loops, one or more beta strands, the N-terminal region and/or the C-terminal regions are modified relative to the wild-type FBS domain.

An example of FBS proteins that are based on human ¹⁰Fn3 domains are adnectins (Adnexus, a wholly owned subsidiary of Bristol-Myers Squibb). Adnectins are ¹⁰Fn3 molecules in which CDR-like loop regions, β-strands, N-terminal and/or C-terminal regions of a ¹⁰Fn3 domain has been modified to evolve a protein capable of binding to a compound of interest. For example, U.S. Pat. No. 7,115,396 describes ¹⁰Fn3 domain proteins wherein alterations to the BC, DE, and FG loops result in high affinity TNFα binders. U.S. Pat. No. 7,858,739 describes Fn3 domain proteins wherein alterations to the BC, DE, and FG loops result in high affinity VEGFR2 binders.

A “region” of a ¹⁰Fn3 domain as used herein refers to either a loop (AB, BC, CD, DE, EF and FG), a β-strand (A, B, C, D, E, F and G), the N-terminus (corresponding to amino acid residues 1-7 of SEQ ID NO: 1), or the C-terminus (corresponding to amino acid residues 93-94 of SEQ ID NO: 1) of the human ¹⁰Fn3 domain.

A “scaffold region” refers to any non-loop region of a human ¹⁰Fn3 domain. The scaffold region includes the A, B, C, D, E, F and G β-strands as well as the N-terminal region (amino acids corresponding to residues 1-7 of SEQ ID NO: 1) and the C-terminal region (amino acids corresponding to residues 93-94 of SEQ ID NO: 1 and optionally comprising the 7 amino acids constituting the natural linker between the 10^{th} and the 11^{th} repeat of the Fn3 domain in human fibronectin).

In certain embodiments, an FBS moiety is based on an Fn3 repeat other than the 10^{th} repeat of the type III domain of fibronectin, e.g., human fibronectin. For example, an FBS moiety may be similar to any of the other fibronectin type III repeats, e.g., the 1^{st}, 2^{nd}, 3^{rd}, 4^{th}, 5^{th}, 6^{th}, 7^{th}, 8^{th}, 9^{th}, 11^{th}, 12^{th}, 13^{th}, 14^{th}, 15^{th}, 16^{th}, 17^{th}, and 18^{th} Fn3 repeats. In yet other embodiments, an FBS moiety may be from a molecule other than fibronectin. Exemplary FBS moieties may be derived from tenascin, a protein that is composed of 15 Fn3 domains with similar sequence similarities to one another as found in fibronectin. These repeats are described, e.g., in Jacobs et al., Protein Engineering, Design & Selection, 25:107 (2012). Based on the homology of the repeats in the fibronectin molecule and those in the tenascin molecule, artificial molecules based on these homologies have been created. Proteins comprising a consensus amino acid sequence based on the homology of the domains in the fibronectin molecule are referred to as Fibcon and FibconB (WO 2010/093627 and Jacobs et al. (2012) supra.) and those based on the homology of the domains in the tenascin molecule are referred to as Tencon (WO 2010/051274, WO 2010/051310 and WO 2011/137319, which are specifically incorporated by reference herein). A Fibcon, FibconB or Tencon moiety, or target binding variants thereof, whether by itself or linked to a heterologous moiety may be fused as described herein. Fn3 domains from other proteins, e.g., cell surface hormone and cytokine receptors, chaperonins, and carbohydrate-binding domains, may be conjugated as described herein.

FBS proteins specific for any desired target molecule can be generated and tested using art recognized methods. Methods for testing the binding properties of FBS proteins are also well-known. For example, one way to rapidly make and test Fn3 domains with specific binding properties is the nucleic acid-protein fusion technology of Adnexus, a Bristol-Myers Squibb R&D Company. This disclosure utilizes the in vitro expression and tagging technology, termed ‘PROfusion’ which exploits nucleic acid-protein fusions (RNA- and DNA-protein fusions) to identify novel polypeptides and amino acid motifs that are important for binding to proteins. Nucleic acid-protein fusion technology is a technology that covalently couples a protein to its encoding genetic information. For a detailed description of the RNA-protein fusion technology and fibronectin-based scaffold protein library screening methods see Szostak et al., U.S. Pat. Nos. 6,258,558, 6,261,804, 6,214,553, 6,281,344, 6,207,446, 6,518,018 and 6,818,418; Roberts et al., Proc. Natl. Acad. Sci., 1997; 94:12297-12302; and Kurz et al., Molecules, 2000; 5:1259-64, all of which are herein incorporated by reference.

As described herein, FBS polypeptides suitable for use in the methods provided herein comprise an Fn3 domain in which one or more of the solvent accessible loops has been randomized or mutated. In certain embodiments, the Fn3 domain is an Fn3 domain derived from the wild-type tenth module of the human fibronectin type III domain (¹⁰Fn3): VSDVPRDLEVVAATPTSLLISWDAPAVTVRYYRITYGETGGNSPVQEFTVPGSKSTA TISGLKPGVDYTITVYAVTGRGDSPASSKPISINYRT (SEQ ID NO: 1) (94 amino acids; AB, CD, and EF loops are underlined; the core ¹⁰Fn3 domain begins with amino acid 9 (“E”) and ends with amino acid 94 (“T”) and corresponds to an 86 amino acid polypeptide). The core wild-type human ¹⁰Fn3 domain is set forth in SEQ ID NO: 2.

Both variant and wild-type ¹⁰Fn3 proteins are characterized by the same structure, namely seven beta-strand domain sequences designated A through G and six loop regions (AB loop, BC loop, CD loop, DE loop, EF loop, and FG loop) which connect the seven beta-strand domain sequences. The beta strands positioned closest to the N- and C-termini may adopt a beta-like conformation in solution. In SEQ ID NO: 1, the AB loop corresponds to residues 14-17, the BC loop corresponds to residues 23-31, the CD loop corresponds to residues 37-47, the DE loop corresponds to residues 51-56, the EF loop corresponds to residues 63-67, and the FG loop corresponds to residues 75-87.

Accordingly, in certain embodiments, an FBS polypeptide used in the methods provided herein is an ¹⁰Fn3 polypeptide that is at least 40%, 50%, 60%, 65%, 70%, 75%, 80%, 85%, or 90% identical to the human ¹⁰Fn3 domain, shown in SEQ ID NO: 1, or its core sequence, as shown in SEQ ID NO: 2. Much of the variability will generally occur in one or more of the loops or one or more of the beta strands or N- or C-terminal regions. Each of the beta or beta-like strands of a ¹⁰Fn3 polypeptide may consist essentially of an amino acid sequence that is at least 80%, 85%, 90%, 95% or 100% identical to the sequence of a corresponding beta or beta-like strand of SEQ ID NO: 1 or 2, provided that such variation does not disrupt the stability of the polypeptide in physiological conditions.

In certain embodiments, one or more loops selected from BC, DE, and FG may be extended or shortened in length relative to the corresponding human fibronectin loop. In some embodiments, the length of the loop may be extended by 2-25 amino acids. In some embodiments, the length of the loop may be decreased by 1-11 amino acids. To optimize antigen binding, therefore, the length of a loop of ¹⁰Fn3 may be altered in length as well as in sequence to obtain the greatest possible flexibility and affinity in antigen binding.

In certain embodiments, the FBS polypeptide comprises a Fn3 domain that comprises an amino acid sequence of the non-loop regions that is at least 80, 85, 90, 95, 98, 99, or 100% identical to the non-loop regions of SEQ ID NO: 1 or 2, wherein at least one loop selected from BC, DE, and FG is altered. In some embodiments, the altered BC loop has up to 1, 2, 3, 4, 5, 6, 7, 8, 9, or 10 amino acid substitutions, up to 1, 2, 3, or 4 amino acid deletions, up to 1, 2, 3, 4, 5, 6, 7, 8, 9, or 10 amino acid insertions, or a combination thereof.

In some embodiments, one or more residues of the integrin-binding motif “arginine-glycine-aspartic acid” (RGD) (amino acids 78-80 of SEQ ID NO: 1) may be substituted so as to disrupt integrin binding. In some embodiments, the FG loop of the polypeptides provided herein does not contain an RGD integrin binding site. In one embodiment, the RGD sequence is replaced by a polar amino acid-neutral amino acid-acidic amino acid sequence (in the N-terminal to C-terminal direction). In some embodiments, the RGD sequence is replaced with SGE. In one embodiment, the RGD sequence is replaced with RGE.

In certain embodiments, the FBS polypeptide comprises a ¹⁰Fn3 domain that is defined generally by following the sequence:

EVVAA(Z)ₐLLISW(Z)ₓYRITY(Z)_{b}FTV(Z)_{y}ATISGL(Z)_{c}YTITVYA(Z)_{z}ISINYRT (SEQ ID NO: 3)
wherein the AB loop is represented by (Z)ₐ, the CD loop is represented by (Z)_{b}, the EF loop is represented by (Z)_{c}, the BC loop is represented by (Z)ₓ, the DE loop is represented by (Z)_{y}, and the FG loop is represented by (Z)_{z}. Z represents any amino acid and the subscript following the Z represents an integer of the number of amino acids. In particular, a may be anywhere from 1-15, 2-15, 1-10, 2-10, 1-8, 2-8, 1-5, 2-5, 1-4, 2-4, 1-3, 2-3, or 1-2 amino acids; and b, c, x, y and z may each independently be anywhere from 2-20, 2-15, 2-10, 2-8, 5-20, 5-15, 5-10, 5-8, 6-20, 6-15, 6-10, 6-8, 2-7, 5-7, or 6-7 amino acids. The sequences of the beta strands may have anywhere from 0 to 10, from 0 to 8, from 0 to 6, from 0 to 5, from 0 to 4, from 0 to 3, from 0 to 2, or from 0 to 1 substitutions, deletions or additions across all 7 scaffold regions relative to the corresponding amino acids shown in SEQ ID NO: 1 or 2. In certain embodiments, the sequences of the beta strands may have anywhere from 0 to 10, from 0 to 8, from 0 to 6, from 0 to 5, from 0 to 4, from 0 to 3, from 0 to 2, or from 0 to 1 conservative substitutions across all 7 scaffold regions relative to the corresponding amino acids shown in SEQ ID NO: 1 or 2. In certain embodiments, the core amino acid residues are fixed and any substitutions, conservative substitutions, deletions or additions occur at residues other than the core amino acid residues.
A. Modifications in N-Terminal and/or C-Terminal Regions

In certain embodiments, the amino acid sequences of the N-terminal and/or C-terminal regions of the polypeptides provided herein may be modified by deletion, substitution or insertion relative to the amino acid sequences of the corresponding regions of the wild-type human ¹⁰Fn3 domain (SEQ ID NO: 1 or 2). The ¹⁰Fn3 domains generally begin with amino acid number 1 of SEQ ID NO: 1. However, domains with amino acid deletions are also encompassed by the invention. Additional sequences may also be added to the N- or C-terminus of a ¹⁰Fn3 domain having the amino acid sequence of SEQ ID NO: 1 or 2. For example, in some embodiments, the N-terminal extension consists of an amino acid sequence selected from the group consisting of: M, MG, and G. In certain embodiments, an MG sequence may be placed at the N-terminus of the ¹⁰Fn3 defined by SEQ ID NO: 1. The M will usually be cleaved off, leaving a G at the N-terminus. In addition, an M, G or MG may also be placed N-terminal to any of the N-terminal extensions shown in Table 3.

In exemplary embodiments, an alternative N-terminal region having from 1-20, 1-15, 1-10, 1-8, 1-5, 1-4, 1-3, 1-2, or 1 amino acids in length can be added to the N-terminal region of SEQ ID NO: 1 or 2 or any adnectin set forth in Table 3. Exemplary alternative N-terminal regions include (represented by the single letter amino acid code) M, MG, G, MGVSDVPRDL (SEQ ID NO: 574) and GVSDVPRDL (SEQ ID NO: 575). Other suitable alternative N-terminal regions, which may be linked, e.g., to the N-terminus of an adnectin core sequence, include, for example, XₙSDVPRDL (SEQ ID NO: 576), XₙDVPRDL (SEQ ID NO: 577), XₙVPRDL (SEQ ID NO: 578), XₙPRDL (SEQ ID NO: 579) XₙRDL (SEQ ID NO: 580), XₙDL (SEQ ID NO: 581), or XₙL, wherein n=0, 1 or 2 amino acids, wherein when n=1, X is Met or Gly, and when n=2, X is Met-Gly. When a Met-Gly sequence is added to the N-terminus of a ¹⁰Fn3 domain, the M will usually be cleaved off, leaving a G at the N-terminus. In some embodiments, the alternative N-terminal region comprises the amino acid sequence MASTSG (SEQ ID NO: 582).

In exemplary embodiments, an alternative C-terminal region having from 1-20, 1-15, 1-10, 1-8, 1-5, 1-4, 1-3, 1-2, or 1 amino acids in length can be added to the C-terminal region of SEQ ID NO: 1 or 2 or any FBS polypeptide set forth in Table 3. Specific examples of alternative C-terminal region sequences include, for example, polypeptides comprising, consisting essentially of, or consisting of, EIEK (SEQ ID NO: 584), EGSGC (SEQ ID NO: 585), EIEKPCQ (SEQ ID NO: 586), EIEKPSQ (SEQ ID NO: 587), EIEKP (SEQ ID NO: 588), EIEKPS (SEQ ID NO: 589), or EIEKPC (SEQ ID NO: 590). In some embodiments, the alternative C-terminal region comprises EIDK (SEQ ID NO: 591), and in particular embodiments, the alternative C-terminal region is either EIDKPCQ (SEQ ID NO: 592) or EIDKPSQ (SEQ ID NO: 593). Additional suitable alternative C-terminal regions are set forth in SEQ ID NOs: 594-618.

In certain embodiments, an FBS polypeptide is linked to a C-terminal extension sequence that comprises E and D residues, and may be between 8 and 50, 10 and 30, 10 and 20, 5 and 10, and 2 and 4 amino acids in length. In some embodiments, tail sequences include ED-based linkers in which the sequence comprises tandem repeats of ED. In exemplary embodiments, the tail sequence comprises 2-10, 2-7, 2-5, 3-10, 3-7, 3-5, 3, 4 or 5 ED repeats.

In certain embodiments, the ED-based tail sequences may also include additional amino acid residues, such as, for example: EI, EID, ES, EC, EGS, and EGC. Such sequences are based, in part, on known Adnectin tail sequences, such as EIDKPSQ (SEQ ID NO: 593), in which residues D and K have been removed. In exemplary embodiments, the ED-based tail comprises an E, I or EI residues before the ED repeats.

In certain embodiments, the N- or C-terminal extension sequences are linked to the FBS polypeptide with known linker sequences (e.g., SEQ ID NOs: 629-678 in Table 3). In some embodiments, sequences may be placed at the C-terminus of the ¹⁰Fn3 domain to facilitate attachment of a pharmacokinetic moiety. For example, a cysteine containing linker such as GSGC (SEQ ID NO: 638) may be added to the C-terminus to facilitate site directed PEGylation on the cysteine residue.

In certain embodiments, an alternative C-terminal moiety, which can be linked to the C-terminal amino acids RT (i.e., amino acid 94) comprises the amino acids PₘXₙ, wherein P is proline, X is any amino acid, m is an integer that is at least 1 and n is 0 or an integer that is at least 1. In certain embodiments, the alternative C-terminal moiety comprises the amino acids PC. In certain embodiments, the alternative C-terminal moiety comprises the amino acids PI, PC, PID, PIE, PIDK (SEQ ID NO: 605), PIEK (SEQ ID NO: 606), PIDKP (SEQ ID NO: 607), PIEKP (SEQ ID NO: 608), PIDKPS (SEQ ID NO: 609), PIEKPS (SEQ ID NO: 610), PIDKPC (SEQ ID NO: 611), PIEKPC (SEQ ID NO: 612), PIDKPSQ (SEQ ID NO: 613), PIEKPSQ (SEQ ID NO: 614), PIDKPCQ (SEQ ID NO: 615), PIEKPCQ (SEQ ID NO: 616), PHHHHHH (SEQ ID NO: 617), and PCHHHHHH (SEQ ID NO: 618). Exemplary anti-PD-L1 Adnectins having PC at their C-terminus are provided in the Examples and Table 4.

In certain embodiments, the FBS polypeptides described herein have a 6× his tail (SEQ ID NO: 619).

In certain embodiments, the FBS polypeptides comprise a ¹⁰Fn3 domain having both an alternative N-terminal region sequence and an alternative C-terminal region sequence, and optionally a 6× his tail.

### B. Fusions, Including Pharmacokinetic Moieties

In certain embodiments, an imaging agent, e.g., comprising an FBS protein (or generally, any antigen binding protein), is linked to a moiety that modulates, e.g., increases, its blood PK by small increments to enhance the imaging contrast or increase avidity of the radiolabeled imaging and/or therapeutic agent. In some embodiments, the clearance rate of the polypeptide in a mammal (e.g., mouse, rat, or human) is, or is increased by greater than two-fold, greater than three-fold, greater than four-fold or greater than five-fold relative to the unmodified FBS protein. Moieties that slow clearance of a protein from the blood, herein referred to as “PK moieties”, include polyoxyalkylene moieties (e.g., polyethylene glycol), sugars (e.g., sialic acid), and well-tolerated protein moieties (e.g., Fc and fragments and variants thereof, transferrin, or serum albumin). The FBS protein may also be fused to albumin or a fragment (portion) or variant of albumin as described in U.S. Publication No. 2007/0048282, or may be fused to one or more serum albumin binding FBS proteins, as described herein.

Other PK moieties that can be used in the invention include those described in Kontermann et al., (Current Opinion in Biotechnology 2011; 22:868-76), herein incorporated by reference. Such PK moieties include, but are not limited to, human serum albumin fusions, human serum albumin conjugates, human serum albumin binders (e.g., Adnectin PKE, AlbudAb, ABD), XTEN fusions, PAS fusions (i.e., recombinant PEG mimetics based on the three amino acids proline, alanine, and serine), carbohydrate conjugates (e.g., hydroxyethyl starch (HES)), glycosylation, polysialic acid conjugates, and fatty acid conjugates.

In some embodiments, the invention provides radio-labeled FBS proteins fused to a PK moiety that is a polymeric sugar. In some embodiments, the PK moiety is a polyethylene glycol moiety. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161).

The term “PEG” is used broadly to encompass any polyethylene glycol molecule, without regard to size or to modification at an end of the PEG, and can be represented by the formula: X—O(CH₂CH₂O)ₙ-1CH₂CH₂OH, where n is 2 or more, e.g., 20 to 2300 and X is H or a terminal modification, e.g., a C₁₋₄ alkyl. PEG can contain further chemical groups which are necessary for binding reactions, which result from the chemical synthesis of the molecule; or which act as a spacer for optimal distance of parts of the molecule. In addition, such a PEG can consist of one or more PEG side-chains which are linked together. PEGs with more than one PEG chain are called multiarmed or branched PEGs. Branched PEGs are described in, for example, European Published Application No. 473084A and U.S. Pat. No. 5,932,462.

One or more PEG molecules may be attached at different positions on the protein, and such attachment may be achieved by reaction with amines, thiols or other suitable reactive groups. The amine moiety may be, for example, a primary amine found at the N-terminus of a polypeptide or an amine group present in an amino acid, such as lysine or arginine. In some embodiments, the PEG moiety is attached at a position on the polypeptide selected from the group consisting of: a) the N-terminus; b) between the N-terminus and the most N-terminal beta strand or beta-like strand; c) a loop positioned on a face of the polypeptide opposite the target-binding site; d) between the C-terminus and the most C-terminal beta strand or beta-like strand; and e) at the C-terminus.

PEGylation may be achieved by site-directed PEGylation, wherein a suitable reactive group is introduced into the protein to create a site where PEGylation preferentially occurs. In some embodiments, the protein is modified to introduce a cysteine residue at a desired position, permitting site-directed PEGylation on the cysteine. Mutations may be introduced into a protein coding sequence to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein. Alternatively, surface residues may be predicted by comparing the amino acid sequences of binding polypeptides, given that the crystal structure of the framework, based on which binding polypeptides are designed and evolved, has been solved (see Himanen et al., Nature 2001; 414:933-8) and thus the surface-exposed residues identified. PEGylation of cysteine residues may be carried out using, for example, PEG-maleimide, PEG-vinylsulfone, PEG-iodoacetamide, or PEG-orthopyridyl disulfide.

The PEG is typically activated with a suitable activating group appropriate for coupling to a desired site on the polypeptide. PEGylation methods are well-known in the art and further described in Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16: 157-182.

PEG may vary widely in molecular weight and may be branched or linear. Typically, the weight-average molecular weight of PEG is from about 100 Daltons to about 150,000 Daltons. Exemplary weight-average molecular weights for PEG include about 1,000, Daltons, about 2,000 Daltons, about 5,000 Daltons, about 10,000, Daltons, about 20,000 Daltons, about 40,000 Daltons, about 60,000 Daltons and about 80,000 Daltons. In certain embodiments, the molecular weight of PEG is about 5,000 Daltons. Branched versions of PEG having a total molecular weight of any of the foregoing can also be used. In some embodiments, the PEG has two branches. In other embodiments, the PEG has four branches. In one embodiment, the PEG is a bis-PEG (NOF Corporation, DE-200MA).

Similar to antibodies, selective PEGylation of FBS polypeptides can be used to fine-tune (increase in increments) the half-life of the FBS polypeptides if necessary.

Conventional separation and purification techniques known in the art can be used to purify PEGylated FBS proteins, such as size exclusion (e.g., gel filtration) and ion exchange chromatography. Products may also be separated using SDS-PAGE. Products that may be separated include mono-, di-, tri-, poly- and un-PEGylated FBS polypeptides, as well as free PEG. The percentage of mono-PEG conjugates can be controlled by pooling broader fractions around the elution peak to increase the percentage of mono-PEG in the composition. About 90% mono-PEG conjugates represent a good balance of yield and activity.

In certain embodiments, the FBS polypeptides desirably have a short half-life, for example, when used in PET imaging. In certain embodiments, an FBS polypeptide has a half-life in blood or serum of 30 minutes to 3 hours, 30 minutes to 120 minutes, 60 minutes to 120 minutes, or 80 minutes to 100 minutes. In certain embodiments, the half-life of and FBS polypeptide is similar to that of the radiolabel that is attached to it.

### C. Targets

Exemplary in vivo target molecules which bind the radiolabeled FBS polypeptide imaging and therapeutic agents described herein are those associated with various diseases or conditions, such as a malignant disease, a cardiovascular disease, an infectious disease, an inflammatory disease, an autoimmune disease, or a neurological disease, in which it is desirable to kill certain cells. Provided herein are radiolabeled imaging agents and radiotherapeutic agents comprising an FBS polypeptide or antigen binding protein which binds specifically to a target molecule, such as a target protein on the surface of human cells.

In certain embodiments, the FBS polypeptide comprises a human ¹⁰Fn3 domain. In certain embodiments, the FBS polypeptide or antigen binding protein binds to a cell surface molecule, e.g., a cell surface molecule on a tumor cell

For treating cancer, any antigen located on a tumor cell and preferably not generally present on healthy cells, can be used as a target for the radioimaging and radiotherapeutic agents described herein. One such antigen is PD-L1. Other antigens include any tumor antigen, such as those against which antibody drug conjugates are made. Exemplary targets include: MUC1, MUC16, EGFR, EphB2, EphA, Eph-A4 and PMSA, AXL kinase antigen 66, CD20, CD22, CD30, CD33, PTK7, CD123, 5T4, Her2, and CD56.

FBS polypeptides which bind to a specific target may be identified by using standard procedures known to those skilled in the art. One way to rapidly make and test Fn3 domains with specific binding properties is the nucleic acid-protein fusion technology of Adnexus, a Bristol-Myers Squibb R&D Company. This disclosure utilizes the in vitro expression and tagging technology, termed ‘PROfusion’ which exploits nucleic acid-protein fusions (RNA- and DNA-protein fusions) to identify novel polypeptides and amino acid motifs that are important for binding to proteins. Nucleic acid-protein fusion technology is a technology that covalently couples a protein to its encoding genetic information. For a detailed description of the RNA-protein fusion technology and fibronectin-based scaffold protein library screening methods see Szostak et al., U.S. Pat. Nos. 6,258,558, 6,261,804, 6,214,553, 6,281,344, 6,207,446, 6,518,018 and 6,818,418; Roberts et al., Proc. Natl. Acad. Sci., 1997; 94:12297-12302; and Kurz et al., Molecules, 2000; 5:1259-64, all of which are herein incorporated by reference.

Exemplary FBS proteins or moieties included, but are not limited to those which bind to mesothelian, glypican, TL1A, CD8, myostatin, LPA1 receptors, TNF-alpha, VEGFR2, PCSK9, IL-23, EGFR or IGF1R and those which are described, e.g., in WO 2010/093627, WO 2011/130324, WO 2009/083804, WO 2009/133208, WO 02/04523, WO 2012/016245, WO 2009/023184, WO 2010/051310, WO 2011/020033, WO 2011/051333, WO 2011/051466, WO 2011/092233, WO 2011/100700, WO 2011/130324, WO 2011/130328, WO 2011/137319, WO 2010/051274, WO 2009/086116, WO 09/058379, WO2013/067029 and WO2012/016245 (all of which are specifically incorporated by reference herein): any of the FBS proteins or moieties described in these publications may be used as described herein.

### D. PD-L1 FBS Polypeptides

In certain embodiments, the FBS polypeptide used in the methods provided herein binds to PD-L1. PD-L1 overexpression is associated with a poorer prognosis in a variety of cancers, particularly, breast, gastric, renal cell, ovarian, non-small lung, hematological cancers and melanoma. Bladder cancer, triple-negative breast cancer.

In certain embodiments, an anti-PD-L1 FBS polypeptide comprises a human ¹⁰Fn3 domain. In some embodiments, the human ¹⁰Fn3 domain may comprise the sequence as set forth in SEQ ID NO: 3, wherein at least one of BC, DE, and FG loops as represented by (Z)ₓ, (Z)_{y}, and (Z)_{z}, respectively, are altered. As described above, amino acid residues corresponding to residues 23-31, 51-56, and 75-87 of SEQ ID NO: 1 define the BC, DE, and FG loops, respectively. However, it should be understood that not every residue within the loop region needs to be modified in order to achieve a ¹⁰Fn3 binder having strong affinity for a desired target (e.g., PD-L1).

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, have amino acid sequences at least 75%, 80%, 85%, 90%, 95%, 97%, 98%, or 99% identical to the BC, DE or FG loop sequences set forth in SEQ ID NOs: 21, 22, and 23, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, comprise BC, DE, and FG loops having the amino acid sequences of SEQ ID NOs: 21, 22, and 23, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, have amino acid sequences at least 75%, 80%, 85%, 90%, 95%, 97%, 98%, or 99% identical to the BC, DE or FG loop sequences set forth in SEQ ID NOs: 36, 37, and 38, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, comprise BC, DE, and FG loops having the amino acid sequences of SEQ ID NOs: 36, 37, and 38, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, have amino acid sequences at least 75%, 80%, 85%, 90%, 95%, 97%, 98%, or 99% identical to the BC, DE or FG loop sequences set forth in SEQ ID NOs: 51, 52, and 53, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, comprise BC, DE, and FG loops having the amino acid sequences of SEQ ID NOs: 51, 52, and 53, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, have amino acid sequences at least 75%, 80%, 85%, 90%, 95%, 97%, 98%, or 99% identical to the BC, DE or FG loop sequences set forth in SEQ ID NOs: 66, 67, and 68, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, comprise BC, DE, and FG loops having the amino acid sequences of SEQ ID NOs: 66, 67, and 68, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, have amino acid sequences at least 75%, 80%, 85%, 90%, 95%, 97%, 98%, or 99% identical to the BC, DE or FG loop sequences set forth in SEQ ID NOs: 6, 7, and 8, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, comprise BC, DE, and FG loops having the amino acid sequences of SEQ ID NOs: 6, 7, and 8, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, have amino acid sequences at least 75%, 80%, 85%, 90%, 95%, 97%, 98%, or 99% identical to the BC, DE or FG loop sequences set forth in SEQ ID NOs: 81, 82, and 83, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, comprise BC, DE, and FG loops having the amino acid sequences of SEQ ID NOs: 81, 82, and 83, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, have amino acid sequences at least 75%, 80%, 85%, 90%, 95%, 97%, 98%, or 99% identical to the BC, DE or FG loop sequences set forth in SEQ ID NOs: 97, 98, and 99, respectively. In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, comprise BC, DE, and FG loops having the amino acid sequences of SEQ ID NOs: 97, 98, and 99, respectively.

In certain embodiments, an anti-PD-L1 FBS polypeptide comprises the sequence set forth in SEQ ID NO: 3, wherein BC, DE and FG loops as represented by (Z)ₓ, (Z)_{y}, and (Z)_{z}, respectively, comprise BC, DE, and FG loops having the amino acid sequences of SEQ ID NOs: 113, 114, and 115, respectively; SEQ ID NOs: 124, 125 and 126, respectively; SEQ ID NOs: 135, 136 and 137, respectively; SEQ ID NOs: 146, 147 and 148, respectively; SEQ ID NOs: 157, 158 and 159, respectively; SEQ ID NOs: 168, 169 and 170, respectively; SEQ ID NOs: 179, 180 and 181, respectively; SEQ ID NOs: 190, 191 and 192, respectively; SEQ ID NOs: 201, 202 and 203, respectively; SEQ ID NOs: 212, 213 and 214, respectively; SEQ ID NOs: 223, 224 and 225, respectively; SEQ ID NOs: 234, 235, and 236, respectively; SEQ ID NOs: 245, 246 and 247, respectively; SEQ ID NOs: 256, 257 and 258, respectively; SEQ ID NOs: 267, 268 and 269, respectively; SEQ ID NOs: 278, 279 and 280, respectively; SEQ ID NOs: 289, 290 and 291, respectively; SEQ ID NOs: 300, 301 and 302, respectively; SEQ ID NOs: 311, 312 and 313, respectively; SEQ ID NOs: 322, 323 and 324, respectively; SEQ ID NOs: 333, 334 and 335, respectively; SEQ ID NOs: 344, 345 and 346, respectively; SEQ ID NOs: 355, 356 and 357, respectively; SEQ ID NOs: 366, 367 and 368, respectively; SEQ ID NOs: 377, 378 and 379, respectively; SEQ ID NOs: 388, 389 and 390 respectively; SEQ ID NOs: 399, 400 and 401, respectively; SEQ ID NOs: 410, 411 and 412, respectively; SEQ ID NOs: 421, 422 and 423, respectively; SEQ ID NOs: 432, 433 and 434 respectively; SEQ ID NOs: 443, 444 and 445, respectively; SEQ ID NOs: 454, 455 and 456, respectively; SEQ ID NOs: 465, 466 and 467, respectively; SEQ ID NOs: 476, 477 and 478, respectively; SEQ ID NOs: 487, 488 and 489, respectively; SEQ ID NOs: 498, 499 and 500, respectively; SEQ ID NOs: 509, 510 and 511, respectively; SEQ ID NOs: 520, 521 and 522, respectively; SEQ ID NOs: 531, 530 and 531, respectively; SEQ ID NOs: 542, 543 and 544, respectively; SEQ ID NOs: 553, 554 and 555, respectively; or SEQ ID NOs: 564, 565 and 566, respectively. The scaffold regions of such anti-PD-L1 FBS polypeptides may comprise anywhere from 0 to 20, from 0 to 15, from 0 to 10, from 0 to 8, from 0 to 6, from 0 to 5, from 0 to 4, from 0 to 3, from 0 to 2, or from 0 to 1 substitutions, conservative substitutions, deletions or additions relative to the scaffold amino acids residues of SEQ ID NO: 3. Such scaffold modifications may be made, so long as the anti-PD-L1 FBS polypeptide is capable of binding PD-L1 with a desired K_{D}.

In certain embodiments, the BC loop of the anti-PD-L1 FBS polypeptide comprises an amino acid sequence selected from the group consisting of: SEQ ID NOs:6, 21, 36, 51, 66, 81, and 97.

In certain embodiments, the DE loop of the anti-PD-L1 FBS polypeptide comprises an amino acid sequence selected from the group consisting of: SEQ ID NOs: 7, 22, 37, 52, 67, 82, and 98.

In certain embodiments, the FG loop of the anti-PD-L1 FBS polypeptide comprises an amino acid sequence selected from the group consisting of: SEQ ID NOs: 8, 23, 38, 53, 68, 83, and 99.

In certain embodiments, the anti-PD-L1 FBS polypeptide comprises a BC, DE and FG loop amino acid sequence at least 70%, 75%, 80%, 85%, 90%, 95%, 98%, 99% or 100% identical to any one of SEQ ID NOs: 6, 21, 36, 51, 66, 81, and 97; 7, 22, 37, 52, 67, 82, and 98; and 8, 23, 38, 53, 68, 83, and 99, respectively.

In certain embodiments, the anti-PD-L1 FBS polypeptide comprises an amino acid sequence at least 70%, 75%, 80%, 85%, 90%, 95%, 98%, 99%, or 100% identical to any one of SEQ ID NOs: 5, 20, 35, 50, 65, 80, 96, 112, 123, 134, 145, 156, 167, 178, 189, 200, 211, 222, 233, 244, 255, 266, 277, 288, 299, 310, 321, 332, 343, 354, 365, 376, 387, 398, 409, 420, 431, 442, 453, 464, 475, 486, 497, 508, 519, 530, 541, 552 and 563.

In certain embodiments, the anti-PD-L1 FBS polypeptides described herein comprise an amino acid sequence at least 80%, 85%, 90%, 95%, 98%, 99% or 100% identical to the non-BC, DE, and FG loop regions of SEQ ID NOs: 5, 20, 35, 50, 65, 80, or 96.

In certain embodiments, the anti-PD-L1 FBS polypeptide comprises an amino acid sequence at least 70%, 75%, 80%, 85%, 90%, 95%, 98%, 99% or 100% identical to any one of SEQ ID NOs: 9-15, 24-30, 39-45, 54-60, 6975, 84-91, 100-107, 116-122, 127-133, 138-144, 150-155, 160-166, 171-177, 182-188, 193-199, 204-210, 215-221, 227-232, 237-243, 248-254, 259-265, 271-276, 291-287, 292-298, 303-309, 314-320, 325-331, 337-342, 347-353, 358-364, 369-375, 380-386, 391-397, 402-408, 413-419, 424-430, 435-441, 446-452, 457-463, 468-474, 479-485, 490-496, 501-507, 512-518, 523-529, 534-540, 545-551, and 556-562. In certain embodiments, the anti-PD-L1 FBS polypeptides described herein comprise an amino acid sequence at least 80%, 85%, 90%, 95%, 98%, 99% or 100% identical to the non-BC, DE, and FG loop regions of any one of SEQ ID NOs: 9-15, 24-30, 39-45, 54-60, 6975, 84-91, and 100-107.

In certain embodiments, the anti-PD-L1 FBS polypeptide comprises BC, DE, and FG loops as set forth in SEQ ID NOs: 6, 7, and 8, respectively.

In certain embodiments, the anti-PD-L1 FBS polypeptide comprises BC, DE, and FG loops as set forth in SEQ ID NOs: 21, 22, and 23, respectively.

In certain embodiments, the anti-PD-L1 FBS polypeptide comprises BC, DE, and FG loops as set forth in SEQ ID NOs: 36, 37, and 38, respectively.

In certain embodiments, the anti-PD-L1 FBS polypeptide comprises BC, DE, and FG loops as set forth in SEQ ID NOs: 51, 52, and 53, respectively.

In certain embodiments, the anti-PD-L1 FBS polypeptide comprises BC, DE, and FG loops as set forth in SEQ ID NOs: 66, 67, and 68, respectively.

In certain embodiments, the anti-PD-L1 FBS polypeptide comprises BC, DE, and FG loops as set forth in SEQ ID NOs: 81, 82, and 83, respectively.

In certain embodiments, the anti-PD-L1 FBS polypeptide comprises BC, DE, and FG loops as set forth in SEQ ID NOs: 97, 98, and 99, respectively.

In certain embodiments, BC, DE and/or FG loop amino acid sequences described herein (e.g., SEQ ID NOs: 6, 21, 36, 51, 66, 81, and 97; 7, 22, 37, 52, 67, 82, and 98; and 8, 23, 38, 53, 68, 83, and 99, respectively) are grafted into non-¹⁰Fn3 domain protein scaffolds. For instance, one or more loop amino acid sequences is exchanged for or inserted into one or more CDR loops of an antibody heavy or light chain or fragment thereof. In some embodiments, the protein domain into which one or more amino acid loop sequences are exchanged or inserted includes, but is not limited to, consensus Fn3 domains (Centocor, US), ankyrin repeat proteins (Molecular Partners AG, Zurich Switzerland), domain antibodies (Domantis, Ltd, Cambridge, MA), single domain camelid nanobodies (Ablynx, Belgium), lipocalins (e.g., anticalins; Pieris Proteolab AG, Freising, Germany), Avimers (Amgen, CA), affibodies (Affibody AG, Sweden), ubiquitin (e.g., affilins; Scil Proteins GmbH, Halle, Germany), protein epitope mimetics (Polyphor Ltd, Allschwil, Switzerland), helical bundle scaffolds (e.g. alphabodies, Complix, Belgium), Fyn SH3 domains (Covagen AG, Switzerland), or atrimers (Anaphor, Inc., CA).

In some embodiments, the FBS polypeptides bind to human PD-L1 with a KD of 10 nM, 1 nM, 0.5 nM, 0.1 nM or less, as determined, e.g., by SPR (Biacore) and exhibit one or more of the following properties:
1. Inhibition of the interaction between human PD-L1 and human PD-1 by at least 50%, 70%, 80%, 90% or more, as determined, e.g., by flow cytometry, e.g., using a human PD-1Fc protein and human PD-L1 positive cells, such as L2987 cells;
2. Inhibition of the binding of human CD80 (B7-1) to human PD-L1 by at least 50%, 70%, 80%, 90% or more, as determined, e.g., in an ELISA assay or by SPR (Biacore);
3. Inhibition of the binding of the anti-PD-L1 antibody 12A4 (described, e.g., in U.S. Pat. No. 7,943,743) to human PD-L1 by at least 50%, 70%, 80%, 90% or more, as determined, e.g., in an ELISA assay or by SPR (Biacore); and
4. Inhibit cell proliferation in a mixed lymphocyte reaction (MLR).

In certain embodiments, an anti-PD-L1 FBS polypeptide binds to human PD-L1 with a KD of 1 nM or less and exhibits each one of properties 1-4. In certain embodiments, an anti-PD-L1 FBS polypeptide binds to human PD-L1 with a KD of 0.1 nM or less and exhibits each one of properties 1-4.

Provided herein are FBS polypeptides that comprise an amino acid sequence that is at least 70%, 80%, 90%, 95%, 97%, 98% or 99% identical to an anti-PD-L1 FBS polypeptide described herein or a portion thereof (e.g., the BC, DE and FG loops), bind to human PD-L1 with a KD of 10 nM, 1 nM, 0.5 nM, 0.1 nM or less.

In certain embodiments, an anti-PD-L1 FBS polypeptide comprises an amino acid sequence that is at least 70%, 80%, 90%, 95%, 97%, 98% or 99% identical to an anti-PD-L1 FBS polypeptide described herein or a portion thereof (e.g., the BC, DE and FG loops), binds to human PD-L1 with a KD of 1 nM or less and exhibits each one of properties 1-4. In certain embodiments, an anti-PD-L1 FBS polypeptide comprises an amino acid sequence that is at least 70%, 80%, 90%, 95%, 97%, 98% or 99% identical to an anti-PD-L1 FBS polypeptide described herein or a portion thereof (e.g., the BC, DE and FG loops), binds to human PD-L1 with a KD of 0.1 nM or less and exhibits each one of properties 1-4.

In certain embodiments, the anti-PD-L1 FBS polypeptides compete (e.g., cross-compete) for binding to PD-L1 with the particular anti-PD-L1 FBS polypeptides described herein. Such competing FBS polypeptides can be identified based on their ability to competitively inhibit binding to PD-L1 of FBS polypeptides described herein in standard PD-L1 binding assays. For example, standard ELISA assays can be used in which a recombinant PD-L1 protein is immobilized on the plate, one of the FBS polypeptides is fluorescently labeled and the ability of non-labeled FBS polypeptides to compete off the binding of the labeled FBS polypeptide is evaluated.

In certain embodiments, a competitive ELISA format can be performed to determine whether two anti-PD-L1 FBS polypeptides bind overlapping FBS polypeptide binding sites on PD-L1. In one format, FBS polypeptide #1 is coated on a plate, which is then blocked and washed. To this plate is added either PD-L1 alone, or PD-L1 pre-incubated with a saturating concentration of FBS polypeptide #2. After a suitable incubation period, the plate is washed and probed with a polyclonal anti-PD-L1 antibody, such as a biotinylated anti-PD-L1 polyclonal antibody, followed by detection with streptavidin-HRP conjugate and standard tetramethylbenzidine development procedures. If the OD signal is the same with or without preincubation with FBS polypeptide #2, then the two FBS polypeptides bind independently of one another, and their FBS polypeptide binding sites do not overlap. If, however, the OD signal for wells that received PD-L1/FBS polypeptide #2 mixtures is lower than for those that received PD-L1 alone, then binding of FBS polypeptide #2 is confirmed to block binding of FBS polypeptide #1 to PD-L1.

Alternatively, a similar experiment is conducted by surface plasmon resonance (SPR, e.g., BIAcore). FBS polypeptide #1 is immobilized on an SPR chip surface, followed by injections of either PD-L1 alone or PD-L1 pre-incubated with a saturating concentration of FBS polypeptide #2. If the binding signal for PD-L1/FBS polypeptide #2 mixtures is the same or higher than that of PD-L1 alone, then the two FBS polypeptides bind independently of one another, and their FBS polypeptide binding sites do not overlap. If, however, the binding signal for PD-L1/FBS polypeptide #2 mixtures is lower than the binding signal for PD-L1 alone, then binding of FBS polypeptide #2 is confirmed to block binding of FBS polypeptide #1 to PD-L1. A feature of these experiments is the use of saturating concentrations of FBS polypeptide #2. If PD-L1 is not saturated with FBS polypeptide #2, then the conclusions above do not hold. Similar experiments can be used to determine if any two PD-L1 binding proteins bind to overlapping FBS polypeptide binding sites.

Both assays exemplified above may also be performed in the reverse order where FBS polypeptide #2 is immobilized and PD-L1-FBS polypeptide #1 are added to the plate. Alternatively, FBS polypeptide #1 and/or #2 can be replaced with a monoclonal antibody and/or soluble receptor-Fc fusion protein.

In certain embodiments, competition can be determined using a HTRF sandwich assay.

In certain embodiments, the competing FBS polypeptide is an FBS polypeptide that binds to the same FBS polypeptide binding site on PD-L1 as a particular anti-PD-L1 FBS polypeptide described herein. Standard mapping techniques, such as protease mapping, mutational analysis, HDX-MS, x-ray crystallography and 2-dimensional nuclear magnetic resonance, can be used to determine whether an FBS polypeptide binds to the same FBS polypeptide binding site or epitope as a reference FBS polypeptide (see, e.g., Epitope Mapping Protocols in Methods in Molecular Biology, Vol. 66, G. E. Morris, Ed. (1996)). An epitope is defined by the method used to locate it. For example, in certain embodiments, a PD-L1 FBS polypeptide or antibody binds to the same epitope as that of one of the PD-L1 FBS polypeptides described herein, as determined by HDX-MS or as determined by X-ray crystallography.

Candidate competing anti-PD-L1 FBS polypeptides can inhibit the binding of anti-PD-L1 FBS polypeptides described herein to PD-L1 by at least 50%, at least 55%, at least 60%, at least 65%, at least 70%, at least 75%, at least 80%, at least 85%, at least 90%, at least 95%, at least 97%, at least 98%, or at least 99% and/or their binding is inhibited by at least 50%, at least 55%, at least 60%, at least 65%, at least 70%, at least 75%, at least 80%, at least 85%, at least 90%, at least 95%, at least 97%, at least 98%, or at least 99% by anti-PD-L1 FBS polypeptides. The % competition can be determined using one of the methods described above. “% competition” is defined in the context of one specific assay.

### E. Production of Fibronectin Scaffolds

### Recombinant Protein Expression—Vectors and Polynucleotides

Also included in the present disclosure are nucleic acid sequences encoding any of the proteins described herein. As appreciated by those skilled in the art, because of third base degeneracy, almost every amino acid can be represented by more than one triplet codon in a coding nucleotide sequence. In addition, minor base pair changes may result in a conservative substitution in the amino acid sequence encoded but are not expected to substantially alter the biological activity of the gene product. Therefore, a nucleic acid sequence encoding a protein described herein may be modified slightly in sequence and yet still encode its respective gene product. Certain exemplary nucleic acids encoding the anti-PD-L1 Adnectins and their fusions described herein include nucleic acids having the sequences set forth in SEQ ID NOs: 16-19, 31-34, 46-49, 61-64, 76-79, 92-95, and 108-111.

Also contemplated are nucleic acid sequences that are at least 50%, such as at least 55%, at least 60%, at least 65%, at least 70%, at least 75%, at least 80%, at least 85%, at least 90%, at least 95%, at least 96%, at least 97%, at least 98%, or at least 99% identical to SEQ ID NOs: 16-19, 31-34, 46-49, 61-64, 76-79, 92-95, and 108-111, and encode a protein that binds to PD-L1. In some embodiments, nucleotide substitutions are introduced so as not to alter the resulting translated amino acid sequence.

Nucleic acids encoding any of the various proteins or polypeptides described herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA, 100(2):438-442 (Jan. 21, 2003); Sinclair et al., Protein Expr Purif., 26(I):96-105 (October 2002); Connell, N. D., Curr. Opin. Biotechnol., 12(5):446-449 (October 2001); Makrides et al., Microbiol. Rev., 60(3):512-538 (September 1996); and Sharp et al., Yeast, 7(7):657-678 (October 1991).

General techniques for nucleic acid manipulation are described in, for example, Sambrook et al., Molecular Cloning: A Laboratory Manual, 2nd Edition, Vols. 1-3, Cold Spring Harbor Laboratory Press (1989), or Ausubel, F. et al., Current Protocols in Molecular Biology, Green Publishing and Wiley-Interscience, New York (1987) and periodic updates, herein incorporated by reference. Generally, the DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding site, and sequences that control the termination of transcription and translation. The ability to replicate in a host, usually conferred by an origin of replication, and a selection gene to facilitate recognition of transformants is additionally incorporated.

The proteins described herein may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. An exemplary N-terminal leader sequence for production of polypeptides in a mammalian system is: METDTLLLWVLLLWVPGSTG (SEQ ID NO: 583), which is removed by the host cell following expression.

For prokaryotic host cells that do not recognize and process a native signal sequence, the signal sequence is substituted by a prokaryotic signal sequence selected, for example, from the group of the alkaline phosphatase, penicillinase, 1 pp, or heat-stable enterotoxin II leaders.

For yeast secretion the native signal sequence may be substituted by, e.g., a yeast invertase leader, a factor leader (including Saccharomyces and Kluyveromyces alpha-factor leaders), or acid phosphatase leader, the C. albicans glucoamylase leader, or the signal sequence described in U.S. Pat. No. 5,631,144. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tracycline, (b) complement auxotrophic deficiencies, or (c) supply critical nutrients not available from complex media, e.g., the gene encoding D-alanine racemase for Bacilli.

Expression and cloning vectors usually contain a promoter that is recognized by the host organism and is operably linked to the nucleic acid encoding the protein described herein, e.g., a fibronectin-based scaffold protein. Promoters suitable for use with prokaryotic hosts include the phoA promoter, beta-lactamase and lactose promoter systems, alkaline phosphatase, a tryptophan (trp) promoter system, and hybrid promoters such as the tan promoter. However, other known bacterial promoters are suitable. Promoters for use in bacterial systems also will contain a Shine-Dalgarno (S.D.) sequence operably linked to the DNA encoding the protein described herein. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding protein described herein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the peptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of mRNA encoding the protein described herein. One useful transcription termination component is the bovine growth hormone polyadenylation region. See WO 94/11026 and the expression vector disclosed therein.

The recombinant DNA can also include any type of protein tag sequence that may be useful for purifying the protein. Examples of protein tags include, but are not limited to, a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, New York (1985)), the relevant disclosure of which is hereby incorporated by reference.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (Bio/Technology, 6:47 (1988)). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides described herein would make expression in E. coli as the preferred method for expression. The protein is then purified from culture media or cell extracts.

### F. Protein Production

Also described herein are cell lines that express an anti-PD-L1 Adnectin or fusion polypeptide thereof. Creation and isolation of cell lines producing an anti-PD-L1 Adnectin can be accomplished using standard techniques known in the art, such as those described herein.

Host cells are transformed with the herein-described expression or cloning vectors for protein production and cultured in conventional nutrient media modified as appropriate for inducing promoters, selecting transformants, or amplifying the genes encoding the desired sequences.

Adnectins of the present invention can also be obtained in aglycosylated form by producing the Adnectins in, e.g., prokaryotic cells (e.g., E. coli). Notably, aglycosylated forms of the Adnectins described herein exhibit the same affinity, potency, and mechanism of action as glycosylated Adnectins when tested in vitro.

The host cells used to produce the proteins of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma)) are suitable for culturing the host cells. In addition, many of the media described in Ham et al., Meth. Enzymol., 58:44 (1979), Barites et al., Anal. Biochem., 102:255 (1980), U.S. Pat. Nos. 4,767,704, 4,657,866, 4,927,762, 4,560,655, 5,122,469, 6,048,728, 5,672,502, or U.S. Pat. No. RE 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins described herein can also be produced using cell-free translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Proteins described herein can also be produced by chemical synthesis (e.g., by the methods described in Solid Phase Peptide Synthesis, 2nd Edition, The Pierce Chemical Co., Rockford, Ill. (1984)). Modifications to the protein can also be produced by chemical synthesis.

The proteins of the present invention can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, get filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, or preferably at least 95% pure, and most preferably at least 98% pure. Regardless of the exact numerical value of the purity, the polypeptide is sufficiently pure for use as a pharmaceutical product.

### (i) High Throughput Protein Production (HTPP)

Selected binders cloned into the PET9d vector upstream of a HIS₆tag and are transformed into E. coli BL21 DE3 plysS cells and inoculated in 5 ml LB medium containing 50 μg/mL kanamycin in a 24-well format and grown at 37° C. overnight. Fresh 5 ml LB medium (50 μg/mL kanamycin) cultures are prepared for inducible expression by aspiration of 200 μl from the overnight culture and dispensing it into the appropriate well. The cultures are grown at 37° C. until A₆₀₀ 0.6-0.9. After induction with 1 mM isopropyl-$-thiogalactoside (IPTG), the culture is expressed for 6 hours at 30° C. and harvested by centrifugation for 10 minutes at 2750 g at 4° C.

Cell pellets (in 24-well format) are lysed by resuspension in 450 μl of Lysis buffer (50 mM NaH₂PO₄, 0.5 M NaCl, 1× Complete™ Protease Inhibitor Cocktail-EDTA free (Roche), 1 mM PMSF, 10 mM CHAPS, 40 mM imidazole, 1 mg/ml lysozyme, 30 μg/ml DNAse, 2 μg/ml aprotonin, pH 8.0) and shaken at room temperature for 1-3 hours. Lysates are cleared and re-racked into a 96-well format by transfer into a 96-well Whatman GF/D Unifilter fitted with a 96-well, 1.2 ml catch plate and filtered by positive pressure. The cleared lysates are transferred to a 96-well Nickel or Cobalt-Chelating Plate that had been equilibrated with equilibration buffer (50 mM NaH₂PO₄, 0.5 M NaCl, 40 mM imidazole, pH 8.0) and are incubated for 5 min. Unbound material is removed by positive pressure. The resin is washed twice with 0.3 ml/well with Wash buffer #1 (50 mM NaH₂PO₄, 0.5 M NaCl, 5 mM CHAPS, 40 mM imidazole, pH 8.0). Each wash is removed by positive pressure. Prior to elution, each well is washed with 50 μl Elution buffer (PBS+20 mM EDTA), incubated for 5 min, and this wash is discarded by positive pressure. Protein is eluted by applying an additional 100 μl of Elution buffer to each well. After a 30 minute incubation at room temperature, the plate(s) are centrifuged for 5 minutes at 200 g and eluted protein collected in 96-well catch plates containing 5 μl of 0.5 M MgCl₂ added to the bottom of elution catch plate prior to elution. Eluted protein is quantified using a total protein assay with wild-type ¹⁰Fn3 domain as the protein standard.

### (ii) Midscale Expression and Purification of Insoluble Fibronectin-Based Scaffold Protein Binders

For expression of insoluble clones, the clone(s), followed by the HIS₆tag, are cloned into a pET9d (EMD Bioscience, San Diego, CA) vector and are expressed in E. coli HMS174 cells. Twenty ml of an inoculum culture (generated from a single plated colony) is used to inoculate 1 liter of LB medium containing 50 μg/ml carbenicillin and 34 μg/ml chloramphenicol. The culture is grown at 37° C. until A₆₀₀ 0.6-1.0. After induction with 1 mM isopropyl-β-thiogalactoside (IPTG) the culture is grown for 4 hours at 30° C. and is harvested by centrifugation for 30 minutes at >10,000 g at 4° C. Cell pellets are frozen at −80° C. The cell pellet is resuspended in 25 ml of lysis buffer (20 mM aH2P0₄, 0.5 M NaCl, 1× Complete Protease Inhibitor Cocktail-EDTA free (Roche), ImM PMSF, pH 7.4) using an ULTRA-TURRAX® homogenizer (IKA works) on ice. Cell lysis is achieved by high pressure homogenization (>18,000 psi) using a Model M-1 10S MICROFLUIDIZER® (Microfluidics). The insoluble fraction is separated by centrifugation for 30 minutes at 23,300 g at 4° C. The insoluble pellet recovered from centrifugation of the lysate is washed with 20 mM sodiumphosphate/500 mM NaCl, pH7.4. The pellet is resolubilized in 6.0M guanidine hydrochloride in 20 mM sodium phosphate/500M NaCl pH 7.4 with sonication followed by incubation at 37 degrees for 1-2 hours. The resolubilized pellet is filtered to 0.45 μm and loaded onto a Histrap column equilibrated with the 20 mM sodium phosphate/500 M NaCl/6.0 M guanidine pH 7.4 buffer. After loading, the column is washed for an additional 25 CV with the same buffer. Bound protein is eluted with 50 mM Imidazole in 20 mM sodium phosphate/500 mM NaCl/6.0 M guan-HCl pH7.4. The purified protein is refolded by dialysis against 50 mM sodium acetate/150 mM NaCl pH 4.5.

(iii) Midscale Expression and Purification of Soluble Fibronectin-Base Scaffold Protein Binders

For expression of soluble clones, the clone(s), followed by the HIS₆tag, are cloned into a pET9d (EMD Bioscience, San Diego, CA) vector and expressed in E. coli HMS174 cells. Twenty ml of an inoculum culture (generated from a single plated colony) is used to inoculate 1 liter of LB medium containing 50 μg/ml carbenicillin and 34 μg/ml chloramphenicol. The culture is grown at 37° C. until A₆₀₀ 0.6-1.0. After induction with 1 mM isopropyl-β-thiogalactoside (IPTG), the culture is grown for 4 hours at 30° C. and harvested by centrifugation for 30 minutes at >10,000 g at 4° C. Cell pellets are frozen at −80° C. The cell pellet is resuspended in 25 ml of lysis buffer (20 mM NaH₂PO₄, 0.5 M NaCl, 1× Complete Protease Inhibitor Cocktail-EDTA free (Roche), ImM PMSF, pH 7.4) using an ULTRA-TURRAX® homogenizer (IKA works) on ice. Cell lysis is achieved by high pressure homogenization (>18,000 psi) using a Model M-1 10S MICROFLUIDIZER® (Microfluidics). The soluble fraction is separated by centrifugation for 30 minutes at 23,300 g at 4° C. The supernatant is clarified via 0.45 μm filter. The clarified lysate is loaded onto a Histrap column (GE) pre-equilibrated with the 20 mM sodium phosphate/500M NaCl pH 7.4. The column is then washed with 25 column volumes of the same buffer, followed by 20 column volumes of 20 mM sodium phosphate/500 M NaCl/25 mM Imidazole, pH 7.4 and then 35 column volumes of 20 mM sodium phosphate/500 M NaCl/40 mM Imidazole, pH 7.4. Protein is eluted with 15 column volumes of 20 mM sodium phosphate/500 M NaCl/500 mM Imidazole, pH 7.4, fractions are pooled based on absorbance at A2so and dialyzed against 1×PBS, 50 mM Tris, 150 mM NaCl; pH 8.5 or 50 mM NaOAc; 150 mM NaCl; pH4.5. Any precipitate is removed by filtering at 0.22 μm.

### G. Biochemical/Biological Characterization

Binding of the protein targeting molecules described herein to a target molecule may be assessed in terms of equilibrium constants (e.g., dissociation, Kr) and in terms of kinetic constants (e.g., on-rate constant, kₒₙ and off-rate constant, k_{off}). A protein targeting molecule will generally bind to a target molecule with a K_{D} of less than 500 nM, 100 nM, 10 nM, 1 nM, 500 pM, 200 pM, or 100 pM, although higher K_{D} values may be tolerated where the k_{off} is sufficiently low or the kₒₙ, is sufficiently high.

Exemplary assays for determining the binding affinity of a protein targeting molecule include, but are not limited to, solution phase methods such as the kinetic exclusion assay (KinExA) (Blake et al., JBC 1996; 271:27677-85; Drake et al., Anal Biochem 2004; 328:35-43), surface plasmon resonance (SPR) with the Biacore system (Uppsala, Sweden) (Welford et al., Opt. Quant. Elect 1991; 23:1; Morton and Myszka, Methods in Enzymology 1998; 295:268) and homogeneous time resolved fluorescence (HTRF) assays (Newton et al., J Biomol Screen 2008; 13:674-82; Patel et al., Assay Drug Dev Technol 2008; 6:55-68).

In certain embodiments, biomolecular interactions can be monitored in real time with the Biacore system, which uses SPR to detect changes in the resonance angle of light at the surface of a thin gold film on a glass support due to changes in the refractive index of the surface up to 300 nm away. Biacore analysis generates association rate constants, dissociation rate constants, equilibrium dissociation constants, and affinity constants. Binding affinity is obtained by assessing the association and dissociation rate constants using a Biacore surface plasmon resonance system (Biacore, Inc.). A biosensor chip is activated for covalent coupling of the target. The target is then diluted and injected over the chip to obtain a signal in response units of immobilized material. Since the signal in resonance units (RU) is proportional to the mass of immobilized material, this represents a range of immobilized target densities on the matrix. Association and dissociation data are fit simultaneously in a global analysis to solve the net rate expression for a 1:1 bimolecular interaction, yielding best fit values for kₒₙ, k_{off} and Rₘₐₓ (maximal response at saturation). Equilibrium dissociation constants for binding, K_{D}'s are calculated from SPR measurements as k_{off}/kₒₙ.

In some embodiments, the protein targeting molecules described herein exhibit a K_{D} in the SPR affinity assay of 500 nM or less, 400 nM or less, 300 nM or less, 200 nM or less, 150 nM or less, 100 nM or less, 90 nM or less, 80 nM or less, 70 nM or less, 60 nM or less, 50 nM or less, 40 nM or less, 30 nM or less, 20 nM or less, 15 nM or less, 10 nM or less, 5 nM or less, or 1 nM or less.

It should be understood that the assays described herein above are exemplary, and that any method known in the art for determining the binding affinity between proteins (e.g., fluorescence based-transfer (FRET), enzyme-linked immunosorbent assay, and competitive binding assays (e.g., radioimmunoassays)) can be used to assess the binding affinities of the protein targeting molecules described herein.

### H. In Vitro Assays for Binding Affinity

An Anti-PD-L1 Adnectin that binds to and antagonizes PD-L1 can be identified using various in vitro assays. In certain embodiments, the assays are high-throughput assays that allow for screening multiple candidate Adnectins simultaneously.

Exemplary assays for determining the binding affinity of an anti-PD-L1 Adnectin includes, but is not limited to, solution phase methods such as the kinetic exclusion assay (KinExA) (Blake et al., JBC 1996; 271:27677-85; Drake et al., Anal Biochem 2004; 328:35-43), surface plasmon resonance (SPR) with the Biacore system (Uppsala, Sweden) (Welford et al., Opt. Quant. Elect 1991; 23:1; Morton and Myszka, Methods in Enzymology 1998; 295:268) and homogeneous time resolved fluorescence (HTRF) assays (Newton et al., J Biomol Screen 2008; 13:674-82; Patel et al., Assay Drug Dev Technol 2008; 6:55-68).

In certain embodiments, biomolecular interactions can be monitored in real time with the Biacore system, which uses SPR to detect changes in the resonance angle of light at the surface of a thin gold film on a glass support due to changes in the refractive index of the surface up to 300 nm away. Biacore analysis generates association rate constants, dissociation rate constants, equilibrium dissociation constants, and affinity constants. Binding affinity is obtained by assessing the association and dissociation rate constants using a Biacore surface plasmon resonance system (Biacore, Inc.). A biosensor chip is activated for covalent coupling of the target. The target is then diluted and injected over the chip to obtain a signal in response units of immobilized material. Since the signal in resonance units (R U) is proportional to the mass of immobilized material, this represents a range of immobilized target densities on the matrix. Association and dissociation data are fit simultaneously in a global analysis to solve the net rate expression for a 1:1 bimolecular interaction, yielding best fit values for kₒₙ, k_{off} and Rₘₐₓ, (maximal response at saturation). Equilibrium dissociation constants for binding, K_{D}'s are calculated from SPR measurements as k_{off}/kₒₙ.

In some embodiments, the anti-PD-L1 Adnectins described herein exhibit a Ku of binding to human PD-L1 in the SPR affinity assay described in Example 2 of 500 nM or less, 400 nM or less, 300 nM or less, 200 nM or less, 150 nM or less, 100 nM or less, 90 nM or less, 80 nM or less, 70 nM or less, 60 nM or less, 50 nM or less, 40 nM or less, 30 nM or less, 20 nM or less, 15 nM or less, 10 nM or less, 5 nM or less, or 1 nM or less.

It should be understood that the assays described herein above are exemplary, and that any method known in the art for determining the binding affinity between proteins (e.g., fluorescence based-transfer (FRET), enzyme-linked immunosorbent assay, and competitive binding assays (e.g., radioimmunoassays)) can be used to assess the binding affinities of the anti-PD-L1 Adnectins described herein.

### III. PD-L1 Antibodies

In another aspect, provided herein are radiolabeled anti-PD-L1 antibodies and antigen binding fragments thereof for use in therapeutic and/or diagnostic methods. Also provided herein are theranostics using anti-PD-L1 antibodies and antigen binding fragments thereof.

A “PD-L1 antibody” refers to an antibody that binds specifically to human PD-L1. PD-L1 antibodies for use as described herein include those described in the literature, such as those described in U.S. Pat. No. 7,943,743 and WO 2013/173223. Other PD-L1 antibodies that can be used as described herein include: atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), and CK-301 (Checkpoint Therapeutics; see Gorelik et al., AACR:Abstract 4606 (April 2016)).

In some embodiments, the radiolabeled anti-PD-L1 antibody or antigen binding fragment thereof comprises the three VH CDR of antibody 12A4. In some embodiments, the radiotherapeutic agent is an antibody or antigen binding fragment comprising the three CDRs of the VH of antibody 12A4. In some embodiments, the radiotherapeutic agent is an anti-PD-L1 antibody or antigen binding fragment thereof that comprises the CDRS of the VH and the CDRs of the VL of antibody 12A4. In some embodiments, the radiotherapeutic agent is an anti-PD-L1 antibody or antigen binding fragment thereof that comprises the VH and VL of antibody 12A4. In one embodiment, the radiotherapeutic agent is 12A4.

In some embodiments, provided herein is method for treating cancer comprising administering to a subject having cancer a radiotherapeutic agent that comprises a radiolabeled anti-PD-L1 antibody or antigen binding fragment thereof as disclosed herein. In some embodiments, the radiotherapeutic agent is an anti-PD-L1 antibody or antigen binding fragment thereof that comprises the CDRS of the VH and the CDRs of the VL of antibody 12A4. In some embodiments, the radiotherapeutic agent is an anti-PD-L1 antibody or antigen binding fragment thereof that comprises the VH and VL of antibody 12A4. In some embodiment, the radiotherapeutic agent is 12A4. In some embodiments, the radiolabeled antibody 12A4, or an antigen binding fragment thereof, is labeled with mu.

In some embodiments, a provided herein is a theranostic method which comprises administering to a subject having cancer, a radioimaging agent that comprises an anti-PD-L1 antibody that is labeled with a radionuclide effective for imaging purposes, such as ⁶⁸Ga, and a radiotherapeutic agent that comprises an anti-PD-L1 antibody that is labeled with a radionuclide effective for that therapeutic purposes, wherein the PD-L1 antibody of the radioimaging agent and that of the radiotherapeutic agent have the same antigen binding specificity. Specific embodiments are set forth in the claims.

In one embodiment, the PD-L1 antibody that is used in the radioimaging agent and in the radiotherapeutic agent is the antibody 12A4, which was used in the Examples, or an antigen binding fragment thereof.

In one embodiment, a method for treating cancer comprises administering to a subject having cancer a radioimaging agent that comprises the antibody 12A4, or an antigen binding fragment thereof, labeled with ⁶⁸Ga, and a radiotherapeutic agent that comprises the antibody 12A4, or an antigen binding fragment thereof, labeled with ¹⁷⁷Lu.

The antibody 12A4 is an IgG4/S228P antibody comprising the following amino acid sequences:

| SEQ ID | | Sequence |
|---|---|---|
| 679 | VH | QVQLVQSGAEVKKPGSSVKVSCKTSGDTFSTYAISWVRQAPGQGLEWMGGIIPIFGKAHY |
| | | AQKFQGRVTITADESTSTAYMELSSLRSEDTAVYFCARKFHFVSGSPFGMDVWGQGTTVT |
| | | VSS |
| 680 | VL | EIVLTQSPATLSLSPGERATLSCRASQSVSSYLAWYQQKPGQAPRLLIYDASNRATGIPA |
| | | RFSGSGSGTDFTLTISSLEPEDFAVYYCQQRSNWPTFGQGTKVEIK |
| 681 | VH CDR1 | TYAIS |
| 682 | VH CDR2 | GIIPIFGKAHYAQKFQ |
| 683 | VH CDR3 | KFHFVSGSPFGMDV |
| 684 | VL CDR1 | RASQSVSSYLA |
| 685 | VL CDR2 | DASNRAT |
| 686 | VL CDR3 | QQRSNWPT |

Various methods are known in the art for labeling proteins, such as antibodies, including antibody fragments, with radionuclides. An exemplary method is described in the Examples. Other methods include the following. For example, proteins, e.g., antibodies, can be labeled with a radionuclide, e.g., ⁶⁸Ga and 17Lu, using NODAGA as chelator, as described, e.g., in Wangler et al. (2011) J. Nuclear Med. 52(4); 586. Proteins, e.g., antibodies, can be labeled with a radionuclide, e.g., ⁶⁸Ga and ¹⁷⁷Lu, using NOTA as chelator, as described, e.g., in Bing et al. (2015) Sci. Rep. 5:8626. Antibodies can also be labeled with ¹⁷⁷Lu using DOTA, as described, e.g., in Rasaneh et al. (2009) 36:363 or using DTPA, as described, e.g., in Dho et al. Scientific Reports 8 (2018), Article number: 8960. Proteins, e.g., antibodies, can also be labeled directly with a radionuclide, e.g., ⁶⁸Ga and ¹⁷⁷Lu, without using a chelator, as described, e.g., in Migliari et al. (2017) Med Clin Arch 1:DOI: 10.15761/MCA.1000116.

In some embodiments, the radiotherapeutic agent is [¹⁷⁷Lu]-anti-PD-L1 antibody or antigen binding fragment thereof that is produced using NODAGA as a chelator. In some embodiments, the radiotherapeutic agent is [¹⁷⁷Lu]-anti-PD-L1 antibody or antigen binding fragment thereof that is produced using NOTA as a chelator. In some embodiments, the radiotherapeutic agent is [¹⁷⁷Lu]-anti-PD-L1 antibody or antigen binding fragment thereof that is produced using DOTA as a chelator.

### IV. Radiolabeling

### A. Radioimaging Agents

Radionuclides which can be used for labelling the imaging agents provided herein (e.g., FBS polypeptides and antigen binding proteins) include any radionuclide which is suitable for use in radioimaging techniques such as PET or SPECT. For example, radionuclides which are used for labeling the radioimaging agents provided herein typically have a half-life which is long enough to allow synthesis and analysis of the radiotracer molecule, injection into the patient, in vivo localization, clearance from non-target tissues and the production of a clear image.

In some embodiments, the radionuclide is a β+ emitter or a γ-emitter. Suitable radionuclides for the imaging agents provided herein include, but are not limited to, ⁶⁸Ga, ¹⁸F, ⁶⁴Cu, ¹²³I, ¹³¹I, ¹²⁵I, ¹¹C, ⁷⁵Br, ¹²⁴I, ¹³N, ³²P, ³⁵C, ^{99m}Tc, ¹⁵³Gd, ¹¹¹In, ⁶⁷Ga, ²⁰¹Tl, ⁹⁰Y, ¹⁸⁸Rh, ¹⁵³Sm, ⁸⁹Sr and ²¹¹At. In some embodiments, the radionuclide is selected from ⁶⁸Ga, ¹⁸F, and ⁶⁴Cu. In one embodiment, the radionuclide is ⁶⁸Ga. In some embodiments, ¹⁷⁷Lu is used as the radionuclide for imaging, although the images may not be as sharp relative to those using ⁶⁸Ga.

### B. Radiotherapeutic Agents

Radionuclides which can be used for labelling the radiotherapeutic agents provided herein (e.g., FBS polypeptides and antigen binding proteins) include any radionuclide which is suitable for use in the radiotherapy, e.g., is cytotoxic. In the context of cancers, radiotherapeutic treatments can decrease the number of cancer cells, decrease the number of metastases, decrease tumor volume, increase life expectancy, induce chemo- or radiosensitivity in cancer cells, inhibit angiogenesis near cancer cells, inhibit cancer cell proliferation, inhibit tumor growth, prevent or reduce metastases, prolong a subject's life, reduce cancer-associated pain, and/or reduce relapse or re-occurrence of cancer following treatment. In particular embodiments, therapeutic treatments reduce, delay, or prevent further metastasis from occurring.

Suitable cytotoxic radionuclides include, but are not limited to β-emitters, α-emitters metals, Auger emitters, and those which emit a combination of radiation types. In some embodiments, the radionuclide selected from ⁹⁰Y, ⁶⁷Cu, ²¹³Bi, ²¹²Bi, ¹⁸⁶Re, ⁶⁷Cu, ⁹⁰Y, ²¹³Bi, ¹⁷⁷Lu, ⁶⁷G, ²²⁵Ac and ²²⁷Th. In certain embodiments, the radiotherapeutic FBS polypeptide (e.g., anti-PD-L1 Adnectin) or antibody comprises ¹⁷⁷Lu.

### C. Chelators

In certain embodiments, the radioactive agent is conjugated to the targeting agent, e.g., imaging agent or therapeutic agent, at one or more amino acid residues. In certain embodiments, one or more, such as two or more, three or more, four or more, or a greater number of radionuclides can be present in the labelled polypeptide (e.g., FBS polypeptide or antigen binding protein).

In some embodiments, the radionuclide is attached to the targeting agent by a chelating agent (e.g., see U.S. Pat. No. 8,808,665). In some embodiments, the chelating agent is a bifunctional chelating (BFC) agent. Suitable combinations of chelating agents and radionuclides have been extensively reviewed (e.g., Price et al., Chem. Soc. Rev. 43:260-290, 2014). Art-recognized methods for labelling polypeptides with radionuclides include those described in, for example, US2014/0271467; Gill et al., Nature Protocols 2011; 6:1718-25; Berndt et al. Nuclear Medicine and Biology 2007; 34:5-15, Inkster et al., Bioorganic & Medicinal Chemistry Letters 2013; 23:3920-6, the contents of which are herein incorporated by reference in their entirety.

In some embodiments, the chelating agent is NOTA or its derivatives; methylhydroxamates derived from triaza- and tetraazamacrocycles (NOTHA 2 and DOTHA 2); 1,4,7-triazacyclononane-1-glutaric acid-4,7-diacetic acid (NODAGA) or its derivatives; diethylenetriaminepentaacetate (DTPA) or its derivatives; 1,4,7,10-tetraazadodecanetetraacetate (DOTA) and its derivatives; 1,4,7,10-tetraazadodecane-1,4,7-triacetate (D03A) and its derivatives; 3,6,9,15-tetraazabicyclo[9.3. 1]pentadeca-1 (15),11,13-triene-3,6,9-triacetic acid) (PCTA) or its derivatives; 1,4,7,10-tetraazacyclotridecanetetraacetic acid (TRITA) and its derivatives; 1,4,8,11-tetraazacyclotetradecane-1,4,8,11-tetraacetic acid (TETA) and its derivatives; 1,4,7,10-tetraazadodecanetetramethylacetate (DOTMA) and its derivatives; 1,4,7,10-tetraazadodecane-1,4,7-trimethylacetate (D03MA) and its derivatives; N,N′,N″,N′″-tetraphosphonatomethyl-1,4,7,10-tetraazacyclododecane (DOTP) and its derivatives; 1,4,7,10-tetraazacyclododecane-1,4,7,10-tetrakis(methylene methylphosphonic acid) (DOTMP) and its derivatives; 1,4,7,10-tetraazacyclododecane-1,4,7,10-tetrakis(methylene phenylphosphonic acid) (DOTPP) and its derivatives; or N,N′-ethylenedi-L-cysteine or its derivatives.

In certain embodiments, the radionuclide is attached to the targeting agent via a bifunctional chelating (BFC) moiety. Bifunctional chelators which can be used in the radiolabeled compositions disclosed herein are commercially available (e.g., Sigma Aldrich; Click Chemistry Tools), or may be synthesized according to well-known chemical reactions.

In certain embodiments, the BFC is a cyclooctyne comprising a reactive group that forms a covalent bond with an amine, carboxyl, carbonyl or thiol functional group on the targeting protein or peptide. Reactive groups on the cyclooctyne include esters, acids, hydroxyl groups, aminooxy groups, maliemides, α-halogenketones and α-halogenacetamides.

In certain embodiments, the BFC is selected from cyclooctyne based agents including but not limited to, DOTA and its derivatives (CB-DO2A, 3p-C-DEPA, TCMC, Oxo-DO3A), NODAGA, NOTA, TE2A, CB-TE2A, CB-TE1A1P, CB-TE2P, MM-TE2A, DM-TE2A, diamsar and derivatives, NODASA, NETA, TACN-TM, DTPA, 1B4M-DTPA, CHX-A″-DTPA, TRAP (PRP9), NOPO, AAZTA and derivatives (DATA), DBCO, DIBO, DFO, H₂dedpa, H₄octapa, H₂azapa, H₅decapa, H₆phospa, HBED, SHBED, BPCA, CP256, PCTA, HEHA, PEPA, EDTA, TETA, and TRITA based chelating agents, and close analogs and derivatives thereof.

In certain embodiments, the chelating agent is DOTA. In some embodiments, the chelating agent is NODOGA. In other embodiments, the chelating agent is NOTA.

In certain embodiments, the cyclooctyne comprises a hydrophilic polyethylene glycol (PEG)_{y} spacer arm, wherein y is an integer from 1 to 8. In certain embodiments, y is an integer from 2 to 6. In certain embodiments, y is 4 or 5.

In some embodiments, the chelator is a BFC containing maleimide. In some embodiments, the BFC is maleimide-DOTA, maleimide-NODGA or maleimide-NOTA, which can be attached covalently to the targeting moiety (e.g., FBS polypeptide or antibody) via cysteine residues near the C-terminus of the polypeptide.

In certain embodiments, the chelating agent is NODAGA and the radionuclide is ⁶⁴Cu. In certain embodiments, the targeting agent, e.g., imaging agent or therapeutic agent, comprises an FBS polypeptide or an anti-PD-L1 polypeptide (e.g., an anti-PD-L1 Adnectin or anti-PD-L1 antibody described herein). In certain embodiments, the targeting agent is an anti-PD-L1 Adnectin comprising the amino acid sequence set forth in SEQ ID NOs: 80, 88, 96 or 104, the chelating agent is NODAGA, and the radionuclide is ⁶⁴Cu. In certain embodiments, the targeting agent is an anti-PD-L1 antibody comprising (i) (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ⁶⁴Cu.

In certain embodiments, the chelating agent is NODAGA and the radionuclide is ⁶⁸Ga. In certain embodiments, the targeting agent, e.g., imaging agent or therapeutic agent, comprises an FBS polypeptide or an anti-PD-L1 polypeptide (e.g., an anti-PD-L1 Adnectin or antibody described herein. In certain embodiments, the targeting agent is an anti-PD-L1 Adnectin comprising the amino acid sequence set forth in SEQ ID NO: 80, 88, 96 or 104, the chelating agent NODAGA, and the radionuclide is ⁶⁸Ga. In certain embodiments, the targeting agent is an anti-PD-L1 antibody (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ⁶⁸Ga.

In certain embodiments, the chelating agent is NODAGA and the radionuclide is ¹⁷⁷Lu. In certain embodiments, the targeting agent, e.g., imaging agent or therapeutic agent, comprises an FBS polypeptide or an anti-PD-L1 polypeptide (e.g., an anti-PD-L1 Adnectin or anti-PD-L1 antibody described herein). In certain embodiments, the targeting agent is an anti-PD-L1 Adnectin comprising the amino acid sequence set forth in SEQ ID NO: 80, 88, 96 or 104, the chelating agent is NODAGA, and the radionuclide is ¹⁷⁷Lu. In certain embodiments, the targeting agent is an anti-PD-L1 antibody comprising (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ¹⁷⁷Lu.

In certain embodiments, the chelating agent is DOTA and the radionuclide is ⁶⁴Cu. In certain embodiments, the targeting agent, e.g., imaging agent or therapeutic agent, comprises an FBS polypeptide or an anti-PD-L1 polypeptide (e.g., an anti-PD-L1 Adnectin or anti-PD-L1 antibody described herein). In certain embodiments, the targeting agent is an anti-PD-L1 Adnectin comprising the amino acid sequence set forth in SEQ ID NO: 80, 88, 96 or 104, the chelating agent is DOTA, and the radionuclide is ⁶⁴Cu. In certain embodiments, the targeting agent is an anti-PD-L1 antibody comprising (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ⁶⁴Cu.

In certain embodiments, the chelating agent is DOTA and the radionuclide is ⁶⁸Ga. In certain embodiments, the targeting agent, e.g., imaging agent or therapeutic agent, comprises an FBS polypeptide or an anti-PD-L1 polypeptide (e.g., an anti-PD-L1 Adnectin or anti-PD-L1 antibody described herein). In certain embodiments, the targeting agent is an anti-PD-L1 Adnectin comprising the amino acid sequence set forth in SEQ ID NO: 80, 88, 96 or 104), the chelating agent NODAGA, and the radionuclide ⁶⁸Ga. In certain embodiments, the targeting agent is an anti-PD-L1 antibody comprising (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ⁶⁸Ga.

In certain embodiments, the chelating agent is DOTA and the radionuclide is ¹⁷⁷Lu. In certain embodiments the targeting agent, e.g., imaging agent or therapeutic agent, comprises an FBS polypeptide or an anti-PD-L1 polypeptide (e.g., an anti-PD-L1 Adnectin or anti-PD-L1 antibody described herein). In certain embodiments, the targeting agent is an anti-PD-L1 Adnectin comprising the amino acid sequence set forth in SEQ ID NO: 80, 88, 96 or 104), the chelating agent DOTA, and the radionuclide ¹⁷⁷Lu. In certain embodiments, the targeting agent is an anti-PD-L1 antibody comprising (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ¹⁷⁷Lu.

In certain embodiments, the chelating agent is NOTA and the radionuclide is ⁶⁴Cu. In certain embodiments, the targeting agent, e.g., imaging agent or therapeutic agent, comprises an FBS polypeptide or an anti-PD-L1 polypeptide (e.g., an anti-PD-L1 Adnectin or anti-PD-L1 antibody described herein). In certain embodiments, the targeting agent is an anti-PD-L1 Adnectin comprising the amino acid sequence set forth in SEQ ID NO: 80, 88, 96 or 104), the chelating agent NODAGA, and the radionuclide ⁶⁴Cu. In certain embodiments, the targeting agent is an anti-PD-L1 antibody comprising (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ⁶⁴Cu.

In certain embodiments, the chelating agent is NOTA and the radionuclide is ⁶⁸Ga. In certain embodiments, the targeting agent, e.g., imaging agent or therapeutic agent, comprises an FBS polypeptide or an anti-PD-L1 polypeptide (e.g., an anti-PD-L1 Adnectin or anti-PD-L1 antibody described herein). In certain embodiments, the targeting agent is an anti-PD-L1 Adnectin comprising the amino acid sequence set forth in SEQ ID NO: 80, 88, 96 or 104), the chelating agent NODAGA, and the radionuclide ⁶⁸Ga. In certain embodiments, the targeting agent is an anti-PD-L1 antibody comprising (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ⁶⁸Ga.

In certain embodiments, the chelating agent is NOTA and the radionuclide is ¹⁷⁷Lu. In certain embodiments, the targeting agent, e.g., imaging agent or therapeutic agent, comprises FBS polypeptide or an anti-PD-L1 polypeptide (e.g., an anti-PD-L1 Adnectin or anti-PD-L1 antibody described herein). In certain embodiments, the targeting agent is an anti-PD-L1 Adnectin comprising the amino acid sequence set forth in SEQ ID NO: 80, 88, 96 or 104), the chelating agent NODAGA, and the radionuclide ¹⁷⁷Lu. In certain embodiments, the targeting agent is an anti-PD-L1 antibody comprising (i) VH CDR1, CDR2 and CDR3 comprising SEQ ID Nos: 681, 682 and 683, respectively, and VL CDR1, CDR2 and CDR3 comprising SEQ ID Nos: 684, 685 and 686, respectively (i.e., the CDRs of 12A4) or (ii) a VH comprising SEQ ID NO: 679 and a VL comprising SEQ ID NO: 680 (i.e., the VH and VL of 12A4), the chelating agent is NODAGA, and the radionuclide is ¹⁷⁷Lu.

In certain embodiments, the radioimaging agent is ⁶⁸Ga-NODAGA-FBS. In certain embodiments, the radioimaging agent is ⁶⁸Ga-NODAGA-anti-PD-L1 Adnectin. In certain embodiments, the radioimaging agent is ⁶⁸Ga-DOTA-FBS. In other embodiments, the radioimaging agent is ⁶⁸Ga-DOTA-anti-PD-L1 Adnectin. In other embodiments, the radioimaging agent is 68Ga-NOTA-FBS. In still other embodiments, the radioimaging agent is ⁶⁸Ga-NOTA-anti-PD-L1 Adnectin. In some embodiments, the targeting agent is an anti-PD-L1 antibody. In certain embodiments, the anti-PD-L1 antibody comprises the VH and VL of 12A4.

In certain embodiments, the radiotherapeutic agent is ¹⁷⁷Lu-DOTA-FBS. In certain embodiments, the radiotherapeutic agent is ¹⁷⁷Lu-DOTA-anti-PD-L1 Adnectin. In certain embodiments, the radiotherapeutic agent is ¹⁷⁷Lu-NOTA-FBS. In certain embodiments, the radiotherapeutic agent is ¹⁷⁷Lu-NOTA-anti-PD-L1 Adnectin.

In particular embodiments, the theranostic combination provided herein comprises a radioimaging agent comprising 68Ga-DOTA-FBS, and a radiotherapeutic agent comprising ¹⁷⁷Lu-DOTA-FBS. In one embodiment, the theranostic combination comprises ⁶⁸Ga-DOTA-anti-PD-L1 Adnectin, and a radiotherapeutic agent comprising ¹⁷⁷Lu-DOTA-anti-PD-L1 Adnectin.

### D. Linkers

In some embodiments, the targeting FBS polypeptide of a radioimaging and/or radiotherapeutic agent described herein can be directly linked to the chelator. In alternative embodiments, the FBS polypeptide is linked to the chelator via a linking molecule. For example, additional residues may also be added at either terminus of the FBS polypeptide for the purpose of providing a “linker” by which the FBS polypeptide is covalently linked to the chelator via a conjugating moiety, e.g., a chelator.

Peptide linkers are usually at least one residue and can be 40 or more residues, more often 1 to 10 residues. In some embodiments, the peptide linkers contain glycine, tyrosine, cysteine, lysine, glutamic and aspartic acid. In some embodiments, the linker can be a flexible peptide linker. A flexible peptide linker can be about 20 or fewer amino acids in length. For example, a peptide linker can contain about 12 or fewer amino acid residues, e.g., 1, 2, 3, 4, 5, 6, 7, 8, 9, 10, 11, and 12. In some cases, a peptide linker comprises two or more of the following amino acids: glycine, serine, lysine, alanine, and threonine.

Exemplary linkers include the N- and C-terminal tails provided in Section IIA. In some embodiments, the FBS polypeptide comprises a peptide linker at the C-terminus. In some embodiments, the FBS polypeptide includes peptide linker comprising a cysteine residue or a lysine residue.

In some embodiments, FBS polypeptide includes a peptide linker comprising, consisting essentially of, or consisting of, EGSGC (SEQ ID NO: 585), EIEKPCQ (SEQ ID NO: 586), EIEKPC (SEQ ID NO: 590), GSGC (SEQ ID NO: 638), PC, PIDKPC (SEQ ID NO: 611), PIEKPC (SEQ ID NO: 612), PIDKPCQ (SEQ ID NO: 615), or PIEKPCQ (SEQ ID NO: 616).

In particular embodiments, the FBS polypeptide comprises a C-terminal linker comprising, consisting essentially of, or consisting of EIDKPCQ (SEQ ID NO: 592) or PC.

Alternatively, a linking molecule can comprise a non-amino acid moiety. Such moieties comprise a biocompatible polymer including two or more repeating units linked to each other. Examples of the non-peptide polymer include but are not limited to: polyethylene glycol (PEG), polypropylene glycol (PPG), co-poly (ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, and heparin. Typically, such linkers will have a range of molecular weight of from about 1 kDa to 50 kDa, depending upon a particular linker. For example, a typical PEG has a molecular weight of about 1 to 5 kDa, and polyethylene glycol has a molecular weight of about 5 kDa to 50 kDa, and more preferably about 10 kDa to 40 kDa.

### V. Formulations

Further provided are compositions, e.g., a pharmaceutical compositions, containing one or a combination of targeting agents, e.g., the radioimaging and/or radiotherapeutic agents, described herein, formulated separately or together with a pharmaceutically acceptable carrier. Such compositions may include one or a combination of (e.g., two or more different) the targeting agents described herein.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, ¹⁸F-labeled targeting agent may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. Supplementary active compounds can also be incorporated into the compositions.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the ¹⁸F-labeled targeting agent in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of targeting agent which can be combined with a carrier material to produce a single dosage form will vary depending upon the subject being treated, and the particular mode of administration. The amount of targeting agent which can be combined with a carrier material to produce a single dosage form will generally be that amount of the composition which produces a detectable effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

### VI. Methods of Use

### A. Combination Detection and Treatment Methods

Provided herein is a method of detecting and treating target positive cells (e.g., cancer) in a subject comprising administering to the subject a radioimaging agent provided herein (e.g., an anti-PD-L1 Adnectin or an anti-PD-L1 antibody or fragment thereof), and detecting the imaging agent, the detected radioimaging agent defining the location of the target positive cells in the subject, and then administering a radiotherapeutic which is cytotoxic to cells expressing the target.

The methods of detecting and treating target positive cells provided herein allow localization of the site and extent of the disease as well as the biodistribution of target expression. The radioimaging agent also aids in determining the optimal therapeutic dosage or activity to be administered, e.g., based on anticipated tumoricidal doses measured in the tumor site, and for monitor response to treatment.

In certain embodiments, the radiolabeled imaging agents provided herein can be used to image cells or tissues which express a desired target, e.g., PD-1 expressing tumors. For example, the radiolabeled imaging agent is administered to a subject in an amount sufficient to uptake the radiolabeled imaging agent into the tissue of interest (e.g., the PD-L1-expressing tumor). The subject is then imaged using an imaging system such as PET for an amount of time appropriate for the particular radionuclide being used. The radiolabeled imaging agent bound target expressing cells or tissues, e.g., PD-L1-expressing tumors, are then detected by the imaging system.

PET imaging with the imaging agent may be used to qualitatively or quantitatively detect cells expressing a target, e.g., PD-L1. In certain embodiments, the radioimaging agent is used as a biomarker, and the presence or absence of a positive signal in a subject is indicative that, e.g., the subject would be responsive to a corresponding radiotherapeutic agent provided herein.

In certain embodiments, the progression or regression of disease (e.g., tumor) can be imaged as a function of time or treatment. For instance, the size of the tumor can be monitored in a subject undergoing treatment with a radiotherapeutic provided herein and the extent of regression of the tumor can be monitored in real-time based on detection of the radiolabeled imaging agent. The distribution of the radioimaging agent within one or more tumors or healthy cells may also be visualized, and monitored prior and/or during a treatment and/or a disease.

The amount effective to result in uptake of the radioimaging agent provided herein (e.g., FBS polypeptide, anti-PD-L1 Adnectin, anti-PDL-antibody or fragment thereof) into the cells or tissue of interest (e.g., tumors) may depend upon a variety of factors, including for example, the age, body weight, general health, sex, and diet of the host; the time of administration; the route of administration; the rate of excretion of the specific probe employed; the duration of the treatment; the existence of other drugs used in combination or coincidental with the specific composition employed; and other factors.

In certain embodiments, radioimaging of tissues expressing the target (e.g., PD-L1) with the radioimaging agent is conducted before administration of the radiotherapeutic. In certain embodiments, the radioimaging agent is administered after administration of the radiotherapeutic agent to a subject, e.g., to monitor the efficacy of the radiotherapy.

In certain embodiments, the radioimaging agents provide a contrast of at least 50%, 75% or more.

In certain embodiments, the radioimaging agents described herein are used to detect target positive cells in a subject by administering to the subject a radioimaging agent provided herein (e.g., an anti-PD-L1 radioimaging agent) disclosed herein, and detecting the radioimaging agent, the detected radioimaging agent defining the location of the target positive cells in the subject. In certain embodiments, the imaging agent is detected by positron emission tomography.

Typically, for PET imaging purposes it is desirable to provide the recipient with a dosage of radioimaging agent that is in the range of from about 0.1 mg to 200 mg as a single intravenous infusion, although a lower or higher dosage also may be administered as circumstances dictate. It may be desirable to provide the recipient with a dosage that is in the range of from about 0.1 mg to 10 mg per square meter of body surface area of the protein or peptide for the typical adult, although a lower or higher dosage also may be administered as circumstances dictate. Examples of dosages of proteins or peptides that may be administered to a human subject for imaging purposes are 10 μg to 1000 μg, 100 μg to 1000 μg, 100 μg to 500 μg, 200 μg to 500 μg, and 300 μg to 400 μg, although higher or lower doses may be used. For example, ⁶⁸Ga labeled anti-PD-L1 Adnectin, (e.g., [⁶⁸Ga]-DOTA-A02) imaging agents may be administered in an amount, e.g., as a bolus injection, to a human ranging from 10 μg to 1000 μg, 100 μg to 1000 μg, 100 μg to 500 μg, 200 μg to 500 μg, and 300 μg to 400 μg.

In certain embodiments, administration occurs in an amount of radioimaging agent (e.g., ⁶⁸Ga-anti-PD-L1 Adnectin), of between 0.005 μg/kg of body weight to 50 μg/kg of body weight per day, e.g., between 0.02 μg/kg of body weight to 10 μg/kg, e.g., per day, between 0.1 μg/kg of body weight to 10 μg/kg of body weight, e.g., per day, between 1 μg/kg of body weight to 10 μg/kg of body weight, e.g., per day, between 2 μg/kg of body weight to 6 μg/kg of body weight, e.g., per day or between 4 μg/kg of body weight to 5 μg/kg of body weight, e.g., per day.

Dosage regimens are adjusted to provide the optimum detectable amount for obtaining a clear image of the tissue or cells which uptake the radioimaging agent. It is especially advantageous to formulate parenteral compositions in dosage unit form for ease of administration and uniformity of dosage. Dosage unit form as used herein refers to physically discrete units suited as unitary dosages for the subjects to which the radiolabeled targeting agent is to be administered. The specification for the dosage unit forms described herein are dictated by and directly dependent on (a) the unique characteristics of the targeting portion of the radiolabeled imaging agent; (b) the tissue or cells to be targeted; (c) the limitations inherent in the imaging technology used.

In certain embodiments, the radiolabeled imaging agent described herein can be formulated to ensure proper distribution in vivo. For example, the blood-brain barrier (BBB) excludes many highly hydrophilic compounds. Agents may cross the BBB by formulating them, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994).

Two types of PET procedures may be used. One type involves obtaining single time point estimates of tracer uptake or static imaging that provides a spatial map of regional tracer concentration. With static imaging, only an average value is measured (e.g. Standardized Uptake Value, SUV). The second type is referred to as dynamic tracer imaging, which can provide considerably more information about in vivo biology by delineating both the temporal and spatial pattern of tracer uptake. See, e.g., Muzi et al. Magn Reson Imaging. 2012 30(9): 1203-1215.

For quantification of tracer uptake, the clinician may visually identify tumor lesions on a PET scan and determine a region-of-interest (ROI) around these lesions. uptake of the radioimaging agent in these ROI's may be corrected for body weight and injected dose and quantified as standardized uptake value (SUVmax and SUVmean).

Tomographic images are obtained through image reconstruction. For determining the distribution of radiotracer, ROIs may be drawn on the reconstructed image including, but not limited to, the lungs, liver, heart, kidney, skin, or other organs and tissue (e.g., cancer tissue). Radiotracer uptakes over time in these regions are used to generate time activity curves (TAC) obtained in the absence of any intervention or in the presence of the unlabeled targeting agent at the various dosing paradigms examined. Data may be expressed as radioactivity per unit time per unit volume (μci/cc/mCi injected dose).

PET may be accompanied by a low-dose or diagnostic CT-scan for anatomic reference purposes.

Alternatively, a first scan, prior to treatment, may indicate that the subject does not express the target (e.g., PD-L1) in a majority of tumors, and that a treatment with a radiotherapeutic as disclosed herein, would not be successful.

In certain embodiments, provided herein are methods comprising comparing a PET scan conducted at a first time point after the radioimaging agent with a PET scan conducted at a second time point, and/or later time point, e.g., after treatment with a radiotherapeutic provided herein. Such comparison may inform on a patient's evolution of the disease, a patient's response to a treatment, a patient's potential adverse reaction or other.

In certain embodiments, the subject is suspected of having a PD-L1 expressing cancer and the method comprises (a) administering to the subject a PD-L1 imaging agent, e.g., an ⁶⁸Ga labeled PD-L1 Adnectin imaging agent, at a dose of about 3-10 mCi (100-333 MBq); and (b) conducting a PET scan of the subject about 1-120 minutes (such as 30-120, 30-60 or 60-120 minutes) after step (a), wherein steps (a) and (b) are conducted at least 1, 2, 3, 4 or 5 time points and if the subject has a level of PD-L1 in one tumor or across several tumors that is equal to or above that required for treatment, the subject is then administered an anti-PD-L1 radiotherapeutic as provided herein.

Provided herein is a method of treating a subject having a PD-L1 cancer, comprising (a) administering to the subject an anti-PD-L1 radioimaging agent; and (b) conducting a PET scan of the subject about 1-120 minutes (such as 30-120, 30-60 or 60-120 minutes) after step (a), to determine the level of PD-L1 in one tumor or across several tumors and (c) administering to the subject a radiotherapeutic agent as provided herein.

Also provided herein are methods of monitoring the progress of radiotherapy against PD-L1-expressing tumors in a subject, the method comprising
(a) administering to a subject in need thereof an anti-PD-L1 radioimaging agent described herein at a first time point and obtaining an image of at least a portion of the subject to determine the size of the tumor;
(b) administering a radiotherapeutic agent (e.g., a radiolabeled anti-PD-L1 Adnectin or anti-PD-L1 antibody or antigen binding fragment thereof) described herein to the subject;
(c) administering to the subject the radioimaging agent at one or more subsequent time points and obtaining an image of at least a portion of the subject at each time point;
wherein the dimension and location of the tumor at each time point is indicative of the progress of the disease.

Provided herein is a method of treating a subject having cancer, comprising
(a) administering to a subject in need thereof a radioimaging agent provided herein, and obtaining an image of at least a portion of the subject to determine the presence of PD-L1 in one or more tumors; and, if PD-L1 is detected in one or more tumors, then,
(b) administering a radiotherapeutic provided herein to the subject.

Provided herein is a method of monitoring the progress of an anti-tumor therapy against PD-L1-expressing tumors in a subject, the method comprising
(a) administering to a subject in need thereof a radioimaging agent comprising an anti-PD-L1 Adnectin or an anti-PD-L1 antibody or antigen binding fragment thereof at a first time point and obtaining an image of at least a portion of the subject to determine the size of the tumor;
(b) administering a radiotherapeutic comprising an anti-PD-L1 Adnectin or an anti-PD-L1 antibody or antigen binding fragment thereof to the subject;
(c) administering to the subject the radioimaging agent at one or more subsequent time points and obtaining an image of at least a portion of the subject at each time point; wherein the dimension and location of the tumor at each time point is indicative of the subject's response to the radiotherapeutic agent.

### B. Radiotherapeutic Methods

Also provided herein, is a method of treating cancer comprising administering to a subject in need thereof a radiolabeled anti-PD-L1 antibody or antigen binding fragment thereof. In some embodiments, the radiolabeled anti-PD-L1 antibody (or antigen binding fragment thereof) is administered in combination with an anti-PD-L1 antibody (or antigen binding fragment thereof) which is not radiolabeled.

Non-limiting examples of cancers that may be detected and/or treated using the radiolabeled PD-L1 Adnectins and/or anti-PD-L1 antibodies (or antigen binding fragment thereof) provided herein, are those that are PD-L1 positive, and based on the indications of very broad applicability of anti-PD-L1 immunotherapy disclosed in WO2013/173223, include bone cancer, skin cancer, cancer of the head or neck, breast cancer, lung cancer, cutaneous or intraocular malignant melanoma, renal cancer, uterine cancer, castration-resistant prostate cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, carcinomas of the ovary, gastrointestinal tract and breast, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, multiple myeloma, environmentally induced cancers including those induced by asbestos, metastatic cancers, and any combinations of said cancers. The PD-L1 Adnectins are also applicable to the treatment of metastatic cancers.

Exemplary cancers that may be treated using the radiolabeled anti-PD-L1 Adnectins and/or anti-PD-L1 antibodies (and antigen binding fragments thereof) described herein include MEL (e.g., metastatic malignant melanoma), RCC, squamous NSCLC, non-squamous NSCLC, CRC, ovarian cancer (OV), gastric cancer (GC), breast cancer (BC), pancreatic carcinoma (PC), and carcinoma of the esophagus. Additionally, the radiolabeled PD-L1 Adnectins and anti-PD-L1 antibodies ((and antigen binding fragments thereof) described herein also are suitable for use in treating refractory or recurrent malignancies.

### C. Combination Therapies

In some embodiments, this disclosure relates to methods of administering the radiotherapeutic agents provided herein with additional cancer treatments, including chemotherapeutic regimes, surgery, hormone deprivation and angiogenesis inhibitors, for the treatment of various cancers. In some embodiments, the radiotherapeutic agent is combined with an immunogenic agent, for example, a preparation of cancerous cells, purified tumor antigens (including recombinant proteins, peptides, and carbohydrate molecules), antigen-presenting cells such as dendritic cells bearing tumor-associated antigens, cells transfected with genes encoding immune stimulating cytokines (He et ah, 2004), and/or another immunotherapeutic Ab (e.g., an anti-CTLA-4, anti-PD-L1 and/or anti-LAG-3 Ab). Non-limiting examples of tumor vaccines that can be used include peptides of melanoma antigens, such as peptides of gplOO, MAGE antigens, Trp-2, MARTI and/or tyrosinase, or tumor cells transfected to express the cytokine GM-CSF.

### VII. Kits and Articles of Manufacture

The radioimaging and/or radiotherapeutic agents described herein can be provided in a kit, e.g., a packaged combination of radioimaging and radiotherapeutic agents (e.g., in separate containers) in predetermined amounts with instructions for use in the methods described herein.

For example, an article of manufacture containing materials useful for the detection and/or treatment of the disorders or conditions described herein, or for use in the methods of detection and/or treatment described herein, are provided. The article of manufacture comprises one or more containers and a label. Suitable containers include, for example, bottles, vials, syringes, and test tubes. The containers may be formed from a variety of materials such as glass or plastic.

In some embodiments, the article of manufacture comprises the components for producing a radiotherapeutic agent as provided herein, for example, an anti-PD-L1 antibody (or antigen binding fragment thereof), or an anti-PD-Lf Adnectin. In some embodiments, the kit comprises a radiolabeled anti-PD-L1 antibody (or antigen binding fragment thereof), or a radiolabeled anti-PD-L1 Adnectin. In some embodiments, the article of manufacture further comprises an anti-PD-L1 antibody (or antigen binding fragment thereof), or an anti-PD-L1 Adnectin which is not radiolabeled.

In other embodiments, the kit comprises two or more components for use in the detection and treatment. In some embodiments, the kit may hold a composition described herein for in vivo imaging, and may have a sterile access port (for example the container may be an intravenous solution bag or a vial having a stopper pierceable by a hypodermic injection needle), and a second container may hold a composition described herein for radiotherapy. The article of manufacture may further comprise a container comprising a pharmaceutically-acceptable buffer, such as phosphate-buffered saline, Ringer's solution and dextrose solution. In some embodiments, the article of manufacture may also comprise compositions which are not radiolabeled for use in combination with the radiotherapeutic agent. It may further include other materials desirable from a commercial and user standpoint, including other buffers, diluents, filters, needles, syringes, and package inserts with instructions for use.

In certain embodiments, a kit comprises one or more reagents necessary for forming an ⁶⁸Ga labelled anti-PD-L1 Adnectin in vivo imaging agent, such as a [⁶⁸Ga]-DOTA-PD-L1 Adnectin, as further described herein and one or more reagents necessary for forming a ¹⁷⁷Lu-labelled anti-PD-L1 Adnectin radiotherapeutic agent. The kits may comprise a radioimaging agent and a radiotherapeutic agent, but without their radionuclide, or at least without the radionuclide of the radioimaging agent.

The kits may further comprise vials, solutions and optionally additional reagents necessary for the manufacture of the radiolabeled imaging and radiolabeled therapeutic agents, and may contain instructions to complete the synthesis of the agents, e.g., per the methods described in the Examples.

In some embodiments, the kit can further contain at least one additional reagent (e.g., pharmaceutically acceptable carrier). In some embodiments, the kit includes the reaction precursors to be used to generate the labeled probe according to the methods disclosed herein. The components of the kit can be tailored to the particular biological condition to be monitored as described herein. The kit can further include appropriate buffers and reagents known in the art for administering various combinations of the components listed above to the host cell or host organism. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like. Suitable containers include, for example, bottles, vials, syringes, and test tubes. The containers may be formed from a variety of materials such as glass or plastic. It may further include other materials desirable from a commercial and user standpoint, including other buffers, diluents, filters, needles, syringes, and package inserts with instructions for use

### INCORPORATION BY REFERENCE

All documents and references, including patent documents, e.g., PCT/US15/62485 and PCT/US15/62502 and websites, described herein are individually and specifically incorporated by reference herein into this document to the same extent as if there were written in this document in full or in part. The contents of WO2016086021, WO 2017/210302, WO2016086036 and WO/2017/210335 are also specifically incorporated by reference herein, particularly the sections pertaining to imaging agents.

### VARIOUS EMBODIMENTS

Provided below are non-limiting embodiments of the invention.

1. A combination for use in diagnosis, monitoring and treatment of cancer in a subject, the combination comprising (a) a radioimaging agent comprising a fibronectin based scaffold (FBS) polypeptide which binds to a target expressed by the cancer and a radionuclide; and (b) a radiotherapeutic agent comprising the FBS polypeptide and a radionuclide, wherein the FBS polypeptide of the radioimaging agent and the radiotherapeutic agent bind to the target.

2. The combination of embodiment 1, wherein the FBS polypeptide of the radioimaging agent and the radiotherapeutic agent are the same.

3. The combination of embodiment 1 or embodiment 2, wherein the imaging agent comprises a radionuclide that is a β+ emitter or a γ-emitter.

4. The combination of any one of the preceding embodiments, wherein the imaging agent comprises a radionuclide selected from the group consisting of ⁶⁸Ga, ¹⁸F, ⁶⁴Cu, ¹²³I, ¹³¹I, ¹²⁵I, ¹¹C, ⁷⁵Br, ¹²⁴I, ¹³N, ³²P, ³⁵C, ^{99m}Tc, ¹⁵³Gd, ¹¹¹In, ⁶⁷Ga, ²⁰¹Tl, ⁹⁰Y, ¹⁸⁸Rh, ¹⁵³Sm, ⁸⁹Sr and ²¹¹At.

5. The combination of embodiment 4, wherein the imaging agent comprises a radionuclide selected from the group consisting of ⁶⁸Ga, ⁶⁴Cu, ⁸⁶Y, ⁴⁴Sc or ¹⁸F.

6. The combination of embodiment 1, wherein the imaging agent comprises ⁶⁸Ga.

7. The combination of any one of the preceding embodiments, wherein the radiotherapeutic agent comprises a radionuclide that is a β− emitter, α-emitter, Auger emitters or combination thereof.

8. The combination of embodiment 7, wherein the radiotherapeutic agent comprises a radionuclide selected from the group consisting of ⁹⁰Y, ⁶⁷Cu, ²¹³Bi, ²¹Bi, ¹⁸⁶Re, ⁶⁷Cu, ⁹⁰Y, ²¹³Bi, ¹⁷⁷Lu, ⁶⁷G, ²²⁵Ac and ²²⁷Th.

9. The combination of any one of the preceding embodiments, wherein the radiotherapeutic agent comprises ¹⁷⁷Lu.

10. The combination of any one of the preceding embodiments, wherein the radionuclide is linked to the FBS polypeptide by a chelating agent.

11. The combination of a embodiment 10, wherein the chelating agent is the same for the radioimaging agent as the radiotherapeutic agent

12. The combination of embodiment 10 or embodiment 11, wherein the chelating agent is a cyclooctyne derivative.

13. The combination of any one of embodiments 10-12, wherein the chelating agent is a bifunctional chelating agent (BFC).

14. The combination of any one of embodiments 10-13, wherein the chelating agent comprises a reactive group that forms a covalent bond with an amine, carboxyl, carbonyl or thiol functional group on the targeting protein or peptide.

15. The combination of embodiment 14, wherein the chelator is covalently linked to the FBS polypeptide via a cysteine residue near the C-terminus of the polypeptide.

16. The combination of any one of embodiments 10-15, wherein the chelating agent is NODAGA or a derivative thereof.

17. The combination of embodiment any one of embodiments 10-15, wherein the chelating agent is DOTA or a derivative thereof.

18. The combination of any one of embodiments 10-15, wherein the chelating agent is NOTA or a derivative thereof.

19. The combination of any one of embodiments 10-18, wherein the chelator is covalently attached to the FBS polypeptide by a linker.

20. The combination of embodiment 19, wherein the linker is attached to the C-terminus of the FBS polypeptide.

21. The combination of embodiment 19 or embodiment 20, the linker is a peptide linker selected from the group consisting of EGSGC (SEQ ID NO: 585), EIEKPCQ (SEQ ID NO: 586), EIDKPCQ (SEQ ID NO: 592), EIEKPC (SEQ ID NO: 590), GSGC (SEQ ID NO: 638), PC, PIDKPC (SEQ ID NO: 611), PIEKPC (SEQ ID NO: 612), PIDKPCQ (SEQ ID NO: 615), or PIEKPCQ (SEQ ID NO: 616).

22. The combination of embodiment 21, wherein the peptide linker is PC.

23. The combination of any one of the preceding embodiments, wherein the radioimaging agent comprises an FBS polypeptide bound to a ⁶⁸Ga by DOTA.

24. The combination of any one embodiments 1-22, wherein the radioimaging agent comprises an FBS polypeptide bound to a ⁶⁸Ga by NODAGA.

25. The combination of any one embodiments 1-22, wherein the radioimaging agent comprises an FBS polypeptide bound to a ⁶⁸Ga by NOTA.

26. The combination of any one of embodiments 1-22, wherein the radioimaging agent comprises an FBS polypeptide bound to a ⁶⁴Cu by DOTA.

27. The combination of any one of embodiments 1-22, wherein the radioimaging agent comprises an FBS polypeptide bound to a ⁶⁴Cu by NODOGA.

28. The combination of any one of embodiments 1-22, wherein the radioimaging agent comprises an FBS polypeptide bound to a ⁶⁴Cu by NOTA.

29. The combination of any one of the preceding embodiments, wherein the radiotherapeutic agent comprises an FBS polypeptide bound to ¹⁷⁷Lu by DOTA.

30. The combination of any one of embodiments 1-28, wherein the radiotherapeutic agent comprises an FBS polypeptide complexed to ¹⁷⁷Lu by NODAGA or NOTA.

31. The combination of any one of the preceding embodiments, wherein the FBS polypeptide comprises a human ¹⁰Fn3 domain which binds to the target molecule.

32. The combination of embodiment 31, wherein the ¹⁰Fn3 domain binds to human PD-L1.

33. The combination of embodiment 31, wherein the ¹⁰Fn3 domain binding to human PD-L1 comprises AB, BC, CD, DE, EF, and FG loops, (b) the ¹⁰Fn3 has at least one loop selected from loop BC, DE, and FG with an altered amino acid sequence relative to the sequence of the corresponding loop of the human ¹⁰Fn3 domain (SEQ ID NO: 1), and (c) the polypeptide specifically binds to human PD-L1.

34. The combination of embodiment 34, wherein the ¹⁰Fn3 domain binds to human PD-L1 with a KD of less than 500 nM, 100 nM, 10 nM, 1 nM, 500 pM, 200 pM, or 100 pM.

35. The combination of any one of embodiments 32-34, wherein the BC, DE, and FG loops of the ¹⁰Fn3 domain comprise the amino acid sequences of: (a) SEQ ID NOs: 6, 7, and 8, respectively; (b) SEQ ID NOs: 21, 22, and 23, respectively; (c) SEQ ID NOs: 36, 37, and 38, respectively; (d) SEQ ID NOs: 51, 52, and 53, respectively; (e) SEQ ID NOs: 66, 67, and 68, respectively; (f) SEQ ID NOs: 81, 82, and 83, respectively; or (g) SEQ ID NOs: 97, 98, and 99, respectively.

36. The combination of any one of embodiments 32-35, wherein the 10Fn3 domain comprises an amino acid sequence at least 80%, 85%, 90%, 95%, 98%, 99% or 100% identical to SEQ ID NO: 5, 20, 35, 50, 65, 80, or 96.

37. The combination of embodiment 36, wherein the ¹⁰Fn3 domain comprises an amino acid sequence at least 80%, 85%, 90%, 95%, 98%, 99% or 100% identical to SEQ ID NO: 80, 88, 96 or 104.

38. The combination of embodiment 36, wherein the ¹⁰Fn3 domain comprises an amino acid sequence at least 80%, 85%, 90%, 95%, 98%, 99% or 100% identical to SEQ ID NO: 80 or 88.

39. The combination of any one of embodiments 32-37, wherein the ¹⁰Fn3 domain comprises SEQ ID NO: 96 or 104.

40. The combination of any of the preceding embodiments for diagnosis, monitoring and treating a PD-L1 expressing cancer, wherein the combination comprises (a) a radioimaging agent comprising an FBS polypeptide comprising a human ¹⁰Fn3 domain comprising SEQ ID NO: 80, 88, 96 or 104 (A02 or E01 Adnectins), wherein the C-terminus of the ¹⁰Fn3 domain is covalently bound to a linker comprising the amino acid sequence PC; and the radionuclide is ⁶⁸Ga complexed to the cysteine residue of the linker by a chelating agent; and (b) a radiotherapeutic agent comprising an FBS polypeptide comprising a human ¹⁰Fn3 domain comprising SEQ ID NO: 80, 88, 96 or 104 (A02 or E01 Adnectins), wherein the C-terminus of the ¹⁰Fn3 domain is covalently bound to a linker comprising the amino acid sequence PC; and the radionuclide is ¹⁷⁷Lu complexed to the cysteine residue of the linker by a chelating agent.

41. The combination of embodiment 40, wherein the radioimaging agent and radiotherapeutic agent comprise the same human ¹⁰Fn3 domain.

42. The combination of embodiment 40 or embodiment 41, wherein the chelating agent of the radioimaging agent and the radioimaging agent is the same.

43. The combination of embodiment 42, wherein the chelating agent is DOTA.

44. The combination of embodiment 42, wherein the chelating agent is NODAGA.

45. The combination of embodiment 42, wherein the chelating agent is NOTA.

46. A pharmaceutical composition comprising the radioimaging agent of any one of embodiments 1-45.

47. A pharmaceutical composition comprising the radiotherapeutic agent of any one of embodiments 1-45.

48. A kit for use in radioimaging and radiotherapy, comprising (a) a fibronectin based scaffold (FBS) polypeptide which binds to a target and a chelator for a radionuclide suitable for radioimaging; and (b) an FBS polypeptide which binds to a target and a chelator for a radionuclide suitable for radiotherapy, wherein the FBS polypeptide is the same in (a) and (b), and wherein the kit contains instructions for chelation of the FBS polypeptide to the radionuclides.

49. The kit of embodiment 48, wherein the radionuclide for radioimaging is selected from the group consisting of ⁶⁸Ga, ¹⁸F, ⁶⁴Cu, ¹²³I, ¹³¹I, ¹²⁵I, ¹¹C, ⁷⁵Br, ¹²⁴I, ¹³N, ³²P, ³⁵C, ^{99m}Tc, ¹⁵³Gd, ¹¹¹In, ⁶⁷Ga, ²⁰¹Tl, ⁹⁰Y, ¹⁸⁸Rh, ¹⁵³Sm, ⁸⁹Sr and ²¹¹At.

50. The kit of embodiment 49, wherein the radionuclide for radioimaging is selected from the group consisting of ⁶⁸Ga, ⁶⁴Cu, ⁸⁶Y, ⁴⁴Sc or ¹⁸F.

50, The kit of embodiment 49, wherein the radionuclide for radioimaging is ⁶⁸Ga.

51. The kit of any one of embodiments 48-50, wherein the radionuclide for radiotherapy is selected from the group consisting of ⁹⁰Y, ⁶⁷Cu, ²¹³Bi, ²¹²Bi, ¹⁸⁶Re, ⁶⁷Cu, ⁹⁰Y, ²¹³Bi, ¹⁷⁷Lu, ⁶⁷G, ²²⁵Ac and ²³⁷Th.

52. The kit of any one of embodiments 48-51, wherein the radionuclide for radiotherapy is ¹⁷⁷Lu.

53. The kit of any one of embodiments 48-52, wherein the radionuclide for radioimaging is ⁶⁸Ga, and the radionuclide for radiotherapy is ¹⁷⁷Lu.

54. The kit of any one of embodiments 48-53, wherein the chelator for (a) and/or (b) is a cyclooctyne derivative.

55. The kit of any one of embodiments 48-53, wherein the chelator for (a) and/or (b) is NODAGA or a derivative thereof.

56. The kit of any one of embodiments 48-53, wherein the chelator for (a) and/or (b) is, DOTA or a derivative thereof.

57. The kit of any one of embodiments 48-53, wherein the chelator for (a) and/or (b) is NOTA or a derivative thereof.

58. The kit of any one of embodiments 48-57, wherein the chelator for (a) and (b) is the same.

59. The kit of any one of embodiments 48-58, wherein the FBS polypeptide comprises a human ¹⁰Fn3 domain.

60. The kit of embodiment 59, comprising the combination of any one of embodiments the human ¹⁰Fn3 domain binds to human PD-L1.

61. The kit of embodiment 59, wherein the ¹⁰Fn3 domain binding to human PD-L1 comprises AB, BC, CD, DE, EF, and FG loops, (b) the ¹⁰Fn3 has at least one loop selected from loop BC, DE, and FG with an altered amino acid sequence relative to the sequence of the corresponding loop of the human ¹⁰Fn3 domain (SEQ ID NO: 1), and (c) the polypeptide specifically binds to human PD-L1.

62. The kit of embodiment 61, wherein the BC, DE, and FG loops of the ¹⁰Fn3 domain comprise the amino acid sequences of: (a) SEQ ID NOs: 6, 7, and 8, respectively; (b) SEQ ID NOs: 21, 22, and 23, respectively; (c) SEQ ID NOs: 36, 37, and 38, respectively; (d) SEQ ID NOs: 51, 52, and 53, respectively; (e) SEQ ID NOs: 66, 67, and 68, respectively; (f) SEQ ID NOs: 81, 82, and 83, respectively; or (g) SEQ ID NOs: 97, 98, and 99, respectively.

63. The combination of embodiment 62, wherein the ¹⁰Fn3 domain comprises an amino acid sequence at least 80%, 85%, 90%, 95%, 98%, 99% or 100% identical to SEQ ID NO: 104.

64. The combination of embodiment 62, wherein the ¹⁰Fn3 domain comprises an amino acid sequence at least 80%, 85%, 90%, 95%, 98%, 99% or 100% identical to SEQ ID NO: 88.

65. The combination of any one of embodiments 32-37, wherein the ¹⁰Fn3 domain comprises SEQ ID NO: 88.

66. The kit of any one of embodiments 48-65, wherein the kit comprises one or more radionuclides.

67. The kit of embodiment 66, wherein the one or more radionuclides are 68Ga and ¹⁷⁷Lu.

68. A method of diagnosing and treating cancer in a subject comprising: (a) administering to the subject an radioimaging agent comprising a fibronectin based scaffold (FBS) polypeptide which binds to a target expressed by cancer cells and a radionuclide suitable for radioimaging; (b) obtaining a radioimage of all or a portion of the subject to determine the presence of the target in the subject; (c) administering a radiotherapeutic agent comprising an FBS polypeptide and a radionuclide suitable for radiotherapy wherein the radioimaging agent and radiotherapeutic agent bind to the same target.

69. The method of embodiment 68, wherein the radioimaging agent and radiotherapeutic agent, respectively, are as defined in any one of embodiments 1-47.

70. The method of clam 68 or 69, wherein the radioimaging agent is also administered after the radiotherapeutic agent to monitor target levels in the subject, and further administration of the radiotherapeutic agent is determined based on the target levels identified with the radioimaging agent.

71. A method of treating a subject having a PD-L1 expressing cancer, comprising a. determining the presence of PD-L1 in a subject having cancer, comprising a. administering to the subject a radioimaging agent comprising an FBS polypeptide comprising a human ¹⁰Fn3 domain which binds to human PD-L1 linked to a chelator and ⁶⁸Ga, and if PD-L1 is found to be present in one or more tumors of the subject, b. administering to the subject an FBS polypeptide comprising a human ¹⁰Fn3 domain which binds to human PD-L1 linked to a chelator and ¹⁷⁷Lu.

72. A combination for use in detecting and treating cancer in a subject, comprising (a) a radioimaging agent comprising a PD-L1 antibody and a radionuclide; and (b) a radiotherapeutic agent comprising a PD-L1 antibody and a radionuclide, wherein the PD-L1 antibody of the radioimaging agent and the radiotherapeutic agent have the same antigen binding specificity.

73. The combination of embodiment 72, wherein the radionuclide of the radioimaging agent is ⁶⁸Ga, ¹⁸F, ⁶⁴Cu, ¹²³I, ¹³¹I, ¹²⁵I, ¹¹C, ⁷⁵Br, ¹²⁴I, ¹³N, ³²P, ³⁵C, ^{99m}TC, ¹⁵³Gd, ¹¹¹In, ⁶⁷Ga, ²⁰¹Tl, ⁹⁰Y, ¹⁸⁸Rh, ¹⁵³Sm, ⁸⁹Sr and ²¹¹At.

74. The combination of embodiment 73, wherein the radionuclide of the radioimaging agent is 68Ga.

75. The combination of any one of embodiments 72-74, wherein the radionuclide of the radiotherapeutic agent is ⁹⁰Y, ⁶¹Cu, ²¹³Bi, ²¹²Bi, ¹⁸⁶Re, ⁶⁷Cu, ⁹⁰Y, ²³Bi, ¹⁷⁷Lu, ⁶⁷G, ²²⁵Ac and ²²⁷Th.

76. The combination of embodiment 75, wherein the radionuclide of the radiotherapeutic agent is ¹⁷⁷Lu.

77. The combination of any one of embodiments 72-76, wherein the radionuclide of the radioimaging agent is ⁶⁸Ga and the radionuclide of the radiotherapeutic agent is ¹⁷⁷Lu.

78. The combination of any one of embodiments 72-77, wherein the radionuclide of the radioimaging agent and/or that of the radiotherapeutic agent is linked directly to the PD-L1 antibody.

79. The combination of any one of embodiments 72-78, wherein the radionuclide of the radioimaging agent and that of the radiotherapeutic agent is linked directly to the PD-L1 antibody.

80. The combination of any one of embodiments 72-77, wherein the radionuclide of the radioimaging agent and/or that of the radiotherapeutic agent is linked to the PD-L1 antibody through a chelator.

81. The combination of any one of embodiments 72-77, wherein the radionuclide of the radioimaging agent and that of the radiotherapeutic agent is linked to the PD-L1 antibody through a chelator.

82. The combination of embodiment 80 or 81, wherein the chelator is NODAGA, DOTA, NOTA or DTPA.

83. The combination of any one of embodiments 80-82, wherein the chelator of the radiotherapeutic agent is the same as the chelator of the radiotherapeutic agent.

84. The combination of embodiment 83, wherein the chelator is NODAGA.

85. The combination of embodiment 83, wherein the chelator is DOTA.

86. The combination of embodiment 83, wherein the chelator is NOTA.

87. The combination of embodiment 83, wherein the chelator is DTPA.

88. The combination of any one of embodiments 83-87, wherein the chelator is NODAGA and the radionuclide of the radioimaging agent is ⁶⁸Ga and the radionuclide of the radiotherapeutic agent is ¹⁷⁷Lu.

89. The combination of any one of embodiments 83-87, wherein the chelator is DOTA and the radionuclide of the radioimaging agent is ⁶⁸Ga and the radionuclide of the radiotherapeutic agent is ¹⁷⁷Lu.

90. The combination of any one of embodiments 83-87, wherein the chelator is NOTA and the radionuclide of the radioimaging agent is ⁶⁸Ga and the radionuclide of the radiotherapeutic agent is ¹⁷⁷Lu.

91. The combination of any one of embodiments 83-87, wherein the chelator is DTPA and the radionuclide of the radioimaging agent is ⁶⁸Ga and the radionuclide of the radiotherapeutic agent is ¹⁷⁷Lu.

92. The combination of any one of embodiments 72-91, wherein the antibody binds to human PD-L1 with a KD of 10⁻⁷ M, 10⁻⁸ M, 10⁻⁹ M or less.

93. The combination of any one of embodiments 72-92, wherein the antibody of the radioimaging agent and/or that of the radiotherapeutic agent is a full length antibody comprising a full length heavy chain, with or without the C-terminal lysine, and a full length light chain.

94. The combination of embodiment 93, wherein the antibody of the radioimaging agent and that of the radiotherapeutic agent is a full length antibody comprising a full length heavy chain with or without the C-terminal lysine, and a full length light chain.

95. The combination of any one of embodiments 72-94, wherein the antibody of the radioimaging agent and/or that of the radiotherapeutic agent is an antigen binding fragment.

96. The combination of embodiment 95, wherein the antibody of the radioimaging agent is an antigen binding fragment of an antibody and that of the radiotherapeutic agent is a full length antibody comprising a full length heavy chain with or without the C-terminal lysine, and a full length light chain.

97. The combination of embodiment 96, wherein the antibody of the radioimaging agent and that of the radiotherapeutic agent is an antigen binding fragment of an antibody.

98. The combination of any one of embodiments 72-97, wherein the antigen-binding fragment comprises the variable heavy chain (VH) and the variable light chain (VL) of the antibody.

99. The combination of any one of embodiments 72-98, wherein the antibody of the radioimaging agent and that of the radiotherapeutic agent comprise an amino acid sequence that is at least 95% identical.

100. The combination of embodiment 99, wherein the antibody of the radioimaging agent and that of the radiotherapeutic agent comprise an amino acid sequence that is at least 97% identical.

101. The combination of embodiment 100, wherein the antibody of the radioimaging agent and that of the radiotherapeutic agent comprise an amino acid sequence that is at least 98% identical.

102. The combination of any of embodiment 101, wherein the antibody of the radioimaging agent and that of the radiotherapeutic agent comprise an amino acid sequence that is 99% identical.

103. The combination of any of embodiments 72-102, wherein the antibody of the radioimaging agent and that of the radiotherapeutic agent comprise the same VH CDR1, CDR2 and CDR3.

104. The combination of embodiment 103, wherein the antibody of the radioimaging agent and that of the radiotherapeutic agent comprise the same VH CDR1, CDR2 and CDR3 and the same VL CDR1, CDR2 and CDR3.

105. The combination of embodiment 104, wherein the antibody of the radioimaging agent and that of the radiotherapeutic agent comprise the same VH and VL.

106. The combination of any of embodiment 105, wherein the antibody or antigen-binding fragment of the radioimaging agent and that of the radiotherapeutic agent are identical, except wherein the heavy chain of one antibody may comprise a C-terminal cysteine.

107. The combination of embodiment 106, wherein the antigen-binding fragment does not comprise CH2 or CH3 regions.

108. The combination of any one of embodiments 72-107, wherein the antibody comprises the VH CDRs and VL CDRs of antibody 12A4.

109. The combination of any one of embodiments 72-108, wherein the antibody comprises the VH and VL of antibody 12A4.

110. The combination of any one embodiments 72-94, 99-106 and 108-109, wherein the antibody comprises the heavy and light chains of antibody 12A4.

111. The combination of embodiment 72, wherein (a) the radionuclide of the radioimaging agent is 68Ga; (b) the radionuclide of the radiotherapeutic agent is 177Lu; and wherein the PD-L1 antibody of the radioimaging agent and that of the radiotherapeutic agent comprise VH CDR1, CDR2, CDR3 and the VL CDR2, CDR2 and CDR3 of 12A4.

112. The combination of embodiment 111, wherein the PD-L1 antibody of the radioimaging agent and that of the radiotherapeutic agent comprise the VH and VL of 12A4.

113. The combination of embodiment 112, wherein the PD-L1 antibody of the radioimaging agent and that of the radiotherapeutic agent comprise the heavy and light chains of 12A4.

114. A kit comprising the combination of any one of embodiments 72-113, and instructions for use.

115. A method for treating cancer in a subject, comprising to the subject (a) at a first time, a radioimaging agent comprising a PD-L1 antibody and a radionuclide; and (b) at a second time, a radiotherapeutic agent comprising a PD-L1 antibody and a radionuclide, wherein the PD-L1 antibody of the radioimaging agent and the radiotherapeutic agent have the same antigen binding specificity.

116. The method of embodiment 115, wherein the radioimaging agent and the radiotherapeutic agents are the radioimaging agent and the radiotherapeutic agent, respectively, defined in any one of embodiments 72-113.

117. The method of embodiment 115 or 116, wherein the first time and the second time are different times.

118. The method of embodiment 115 or 116, wherein at least once, the first time is the same as the second time.

119. The method of any one of embodiments 115-118, wherein the first time is before the second time.

120. The method of any one of embodiments 115-119, wherein in the order of (i) to (iii): (i) the radioimaging agent is administered to the subject; (ii) the radioimaging agent is detected in the subject; and (iii) the radiotherapeutic agent is administered to the subject.

121. The method of any one of embodiments 115-119, wherein in the order of (i) to (iii), (i) the radioimaging agent is administered to the subject; (ii) the presence of the radioimaging agent is determined in the subject; and (iii) if the radioimaging agent is detected in the subject, then the radiotherapeutic agent is administered to the subject.

122. The method of any one of embodiments 115-121, wherein the radioimaging agent is also administered after the radiotherapeutic agent, e.g., to monitor PD-L1 levels in the subject, and further administration of the radiotherapeutic agent is determined based on the PD-L1 levels identified with the radioimaging agent.

123. A combination for use in detecting and treating cancer in a subject, comprising (a) a radioimaging agent comprising a PD-L1 Adnectin and a radionuclide; and (b) a radiotherapeutic agent comprising a PD-L1 antibody or antigen binding fragment thereof and a radionuclide.

124. The combination of embodiment 123, wherein the radioimaging agent comprises a radionuclide selected from the group consisting of ⁶⁸Ga, ¹⁸F, ⁶⁴Cu, ¹²³I, ¹³¹I, ¹²⁵I, ¹¹C, ⁷⁵Br, ¹²⁴I, ¹³N, ³²P, ³⁵C, ^{99m}Tc, ¹⁵³Gd, ¹¹¹In, ⁶⁷Ga, ²⁰¹Tl, ⁹⁰Y, ¹⁸⁸Rh, ¹⁵³Sm, ⁸⁹Sr and ²¹At.

125. The combination of embodiment 123, wherein the radioimaging agent comprises 68Ga.

126. The combination of any one of embodiments 123-125, wherein the radionuclide is linked to the anti-PD-L1 Adnectin by a chelating agent selected from NODAGA, DOTA or NOTA.

127. The combination of any one of embodiments 123-126, wherein the anti-PD-L1 Adnectin comprises SEQ ID NO: 80, 88, 96 or 104.

128. The combination of any one of embodiments 123-127, wherein the radiotherapeutic agent comprises a radionuclide selected from the group consisting of ⁹⁰Y, ⁶⁷Cu, ²¹Bi, ²¹²Bi, ¹⁸⁶Re, ⁶⁷Cu, ⁹⁰Y, ²¹³Bi, ¹⁷⁷Lu, ⁶⁷G, ²²⁵Ac and ²²⁷Th.

129. The combination of any one of embodiments 123-128, wherein the radiotherapeutic agent comprises ¹⁷⁷Lu.

130. The combination of any one of embodiments 123-129, wherein the radionuclide is linked to the anti-PD-L1 antibody or antigen binding fragment thereof by a chelating agent selected from NODAGA, DOTA, NOTA or DPTA.

131. The combination of any one of embodiments 123-130, wherein the chelator is linked to random lysine residues, e.g, 2-5 lysine residues, in the antibody

132. The combination of any one of embodiments 123-131, anti-PD-L1 antibody or antigen binding fragment thereof comprises the three CDRs of the VH of antibody 12A4.

133. The combination of any one of embodiments 123-132, anti-PD-L1 antibody or antigen binding fragment thereof comprises the VH CDRs and VL CDRs of antibody 12A4.

134. The combination of any one of embodiments 123-133, anti-PD-L1 antibody or antigen binding fragment thereof comprises the VH and VL of antibody 12A4.

135. The combination of any one of embodiments 123-134, anti-PD-L1 antibody or antigen binding fragment thereof comprises the heavy and light chains of antibody 12A4.

136. A method of detecting and treating a subject having a PD-L1 expressing cancer, comprising a. determining the presence of PD-L1 in a subject having cancer, comprising administering to radioimaging agent of any one of embodiments 124-127, and if PD-L1 is found to be present in one or more tumors of the subject, b. administering to the subject the radiotherapeutic agent of any one of embodiments 128-135.

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### EXAMPLES

### Example 1: Linking of Chelator to Anti-hPD-L1 Adnectin

This Example describes the linking of the anti-PD-L1 Adnectins to the chelators NODAGA and DOTA. As maleimide chemistry is used to link the Adnectins to NODAGA (CheMatech), both Adnectins were modified to include a peptide linker containing proline followed by a cysteine at the C-terminus. The amino acid sequences of the modified E01 and A02 Adnectins are provided in SEQ ID NOs: 104 and 88, respectively. The cysteine is used for linking the Adnectins to the chelating agent as previously described (WO 2017/210302).

A 10-fold molar excess of maleimide-NODAGA (CheMatech) or DOTA was dissolved in PBS pH 7.4 and added to the purified Adnectins in the presence of 1 mM TCEP. Final DMSO concentrations did not exceed 5% in the conjugation mixtures. Conjugation mixtures were left at room temperature for one hour before mass spec analysis. After MS confirmation of conjugation, the samples were purified by size-exclusion chromatography using a HiLoad 26/60 Superdex 75 column (GE Healthcare) equilibrated in PBS pH 7.2.

### Example 2: Preclinical Evaluation of ⁶⁸Ga-Labeled Anti-hPDL-1 Adnectin as a PET Agent for Imaging PD-L1 Expression

### Summary:

Aim: Tumor cells exploit checkpoint pathways by expressing coinhibitory proteins, like PD-L1 to evade antitumor immune response. As recently demonstrated in first patients, ¹⁸F-BMS-986192 (¹⁸F-Adnectin) provides a promising means for in vivo imaging and quantification of PD-L1 expression in tumors. The high uptake of PD-L1 ligands in tumors suggests that PD-L1 may also be used as a theranostic target. As a first step for theranostic applications of radiolabeled PD-L1 ligands we evaluated biodistribution and tumor uptake of a ⁶⁸Ga labeled BMS-986192 analogue.

Methods: ⁶⁸Ga-labeling of Adnectin was carried out in NaOAc-buffer at pH 5.5 (50° C., 15 min). In vitro stability was determined in human serum at 37° C. for 4 hours. PD-L1 binding assays were performed using the transduced PD-L1 expressing lymphoma cell line U-698M and wild-type U-698M cells as negative control. PD-L1 competitive binding assays were performed using the transduced PD-L1 expressing lymphoma cell line U-698M and wild-type U-698M cells as negative control. Biodistribution and small animal PET studies of ⁶⁸Ga-Adnectin were carried out using PD-L1-positive and negative U-698M-bearing NSG mice.

Results: ⁶⁸Ga-Adnectin was obtained with quantitative RCYs (>97%) and high RCP within 15 min. In vitro stability of ⁶⁸Ga-labeled Adnectin in human serum was (≥95%) at 4 h, and revealed monomeric elution profiles by size exclusion chromatography. High and specific binding of ⁶⁸Ga-Adnectin to human PD-L1 expressing cancer cells was confirmed, which closely correlates with the respective PD-L1 expression level determined by flow cytometry and IHC staining. In vivo, ⁶⁸Ga-Adnectin uptake was high in PD-L1+tumors (9.0±2.1% ID/g at 1 h p.i.) and kidneys (56.9±9.2% ID/g at 1 h p.i.) with negligible uptake in other tissues. PD-L1-negative tumors demonstrated only background uptake of radioactivity (0.6±0.1.%). Co-injection of an excess of unlabelled Adnectin reduced tumor uptake of PD-L1 by more than 80%.

Conclusions: ⁶⁸Ga-Adnectin enables easy radiosynthesis and shows excellent in vitro and in vivo PD-L1 targeting characteristics. The high tumor uptake combined with low background accumulation at early imaging time points demonstrate the feasibility of ⁶⁸Ga-Adnectin for imaging of PD-L1 expression in tumors and is encouraging for theranostic applications of PD-L1 ligands.

The details of these experiments are described below.

### Material and Methods

All reagents were obtained from Sigma Aldrich (Munich, Germany) unless otherwise stated. Maleimido-mono-amide-DOTA (1,4,7,10-Tetraazacyclododecane-1,4,7-tris-acetic acid-10-maleimido-ethylacetamide) was purchased from Macrocyclics (Plano, USA). ⁶⁸Ga was obtained from a ⁶⁸Ge/⁶⁸Ga generator (Galliapharm, Eckert & Ziegler AG, Berlin, Germany). Analytical radio-size exclusion chromatography (radio-SEC) of the ⁶⁸Ga-labeled Adnectin was carried out using a bioZen SEC-2 (300×4.6 mm) column (Phenomenex LTD, Aschaffen-burg, Germany) on a Shimadzu HPLC system equipped with a NaI(TI) scintillation detector (2″×2″) and a SPD M20A diode array UV/Vis detector. The tracer was eluted with 0.1 M phosphate buffer (pH 6.8) with a constant flow of 0.35 ml/min. Radio-TLC was performed using Varian silica impregnated ITLC chromatography paper (Varian Inc., CA, USA) and 0.1 M aq. sodium citrate buffer (pH 5.5) as mobile phase. TLC stripes were analyzed on a B-FC-3600 TLC Scanner (Bioscan, Washington, USA).

### ⁶⁸Ga Labeling

Proteins BMS-936559 ((also known as MDX 1105 and 12A4 (U.S. Pat. No. 7,943,743); “mAb”) and A02 (“Adnectin”) with a C-terminal PC tail (SEQ ID NO: 88) were formulated in PBS buffer (pH 7.4) with concentrations of 0.9 mg/mL for the Adnectin and 2.6 mg/mL for the mAb.

A ⁶⁸Ge/⁶⁸Ga generator with TiO₂ matrix (Eckert & Ziegler Radiopharma, Germany) was eluted with 0.05 M aq. HCl (4 ml). To a fraction of 1 ml ⁶⁸Ga-eluate, containing the highest activity (170-240 MBq), 100 μl of 1 M NaOAc (pH 5.5) and 222 μl Adnectin (200 μg in PBS) were added, resulting in 1.32 ml labeling solution at pH 5.5. The solution was mixed briefly and incubated for 15 min at 50° C. The ⁶⁸Ga-labeled Adnectin was purified by gel filtration on a PD-10 column (GE Healthcare, Buckinghamshire, UK).

Radiochemical yield and Radiochemical purity was analyzed by radio-TLC using Varian silica impregnated ITLC chromatography paper (Varian Inc., CA, USA) and a 1:1 (v/v) mixture of 0.1 M_{aq}. sodium citrate buffer (pH 5.5) as mobile phase, where the ⁶⁸Ga-labeled protein stays at the origin (R_{F}=0) and free ⁶⁸Ga^{III} is eluted with the solvent front (R_{F}=0.8−1). TLC stripes were analyzed on a B-FC-3600 TLC Scanner (Bioscan, Washington, USA).

### In Vitro Stability of ⁶⁸Ga-Adnectin in Human Serum

⁶⁸Ga-labeling of DOTA-Adnectin was performed according to the optimized protocol as described above. ⁶⁸Ga-labeled Ad was purified by gel filtration on a PD-10 column (GE Healthcare, Buckinghamshire, UK) before analysis. In vitro stability study was carried out by adding 200 μl of ⁶⁸Ga-Adnectin (18 MBq in 0.9% NaCl) to 800 μl freshly prepared human serum (Seronorm™ Human, IGZ Instruments AG, Zurich), followed by incubation at 37° C. for up to 4 hours. To investigate the stability of ⁶⁸Ga-Adnectin in human serum, radio-HPLC and radio-TLC were performed at 0, 1, 2, 3, and 4 hours.

Radio-TLC was performed as described above. Analytical radio-size exclusion chromatography (radio-SEC) of the ⁶⁸Ga-labeled protein was carried out using a bioZen SEC-2 (300×4.6 mm) column (Phenomenex LTD, Aschaffen-burg, Germany) on a Shimadzu HPLC system equipped with a NaI(TI) scintillation detector (2″×2″) and a SPD M20A diode array UV/Vis detector. The protein was eluted with 0.1 M phosphate buffer pH 6.8 with a constant flow of 0.35 μl/min.

### Cultivation of Cell Lines

B-cell lymphoma cells U-698-M were purchased from ATTC (Manassas, Virginia, USA). Cultures were maintained in either RPMI medium supplemented with 10% FBS and penicilline/streptomycine (100 IU/ml) (U-698-M). Cells were grown at 37° C. in a humidified atmosphere of 5% CO₂.

### Transfection of Cell Lines

RD114 cells (Ward et al. (2003) Mol Ther. 8:804) were used as a viral packaging cell line to produce retroviral supernatant. Twenty-four (24) hours before transfection, 0.3×10⁶ RD114 cells per well were seeded in 3 ml/well cDMEM medium in a tissue-culture treated 6-well plate. The next day, a transfection mixture for each construct consisting of 9 μl TransIT-293 transfection reagent (Mirus, Madison, USA) in 200 μl serum-free DMEM (Invitrogen, Carlsbad, USA) was prepared and incubated for 20 min at room temperature. Three (3) μg of the retroviral vector containing genes for PD-L1 and GFP was added to the transfection mixture, mixed carefully and incubated for 30 min at room temperature. The transfection mixture was then added dropwise to the cells with gentle panning followed by an incubation step for 48 h at 37° C. After two days at 37° C., retrovirus-containing supernatant was used for transduction of cell lines.

### Transduction of Cell Lines

Tissue culture 24 well plates were coated with 400 μl/well RetroNectin (Takara, Japan) solution and incubated overnight at 4° C. The next day, the RetroNectin solution was replaced with 500 μl/well of a 2% BSA solution in PBS followed by an incubation period of 30 min at 37° C. The wells were then washed with 2 ml PBS followed by seeding 1×10⁶ human B cell lymphoma U-698-M cells (DSZM, Braunschweig, Germany) in 1 ml medium together with protamine sulfate (cEND=4 μg/ml; MP Biomedicals, Illkirch, France) and HEPES (cEND=5 mM; Invitrogen, Carlsbad, USA). 1 ml/well of retroviral supernatant of the transfected RD114 cells was harvested and then filtered through a 0.45 μm filter followed by adding it to the corresponding well of the 24-well plate containing the cell suspension. The plates were centrifuged at 820 g and 37° C. for 90 min without a brake and incubated for 24 h at 37° C. After 24 h, transduced cells were harvested and seeded in a new RetroNectin coated 24-well plate with new medium. Again, filtered virus supernatant was added together with protamine sulfate and HEPES as described before. The same centrifugation step as above was performed before incubating the triplicates for 24 h at 37° C. After 24 h, transduced cells were harvested, washed with RPMI-Media and resuspended in corresponding culture media (3 ml).

### Competitive Binding Assay

The affinity of Adnectin towards human PD-L1 was determined in a competitive binding experiment, using stable transduced U698M-PDL1 cells with elevated PD-L1 expression. Specific binding was confirmed using non-transfected U-689-M cells as a negative control. On the day of the experiments, the cells were separated from their normal medium by centrifugation, washed with PBS and adjusted to a concentration of 2×10⁶ cells/ml in RPMI (Seromed, Berlin, Germany) supplemented with 5% bovine serum albumin (BSA). 400.000 cells (200 μl of the cell suspension) were transferred into vials and left to equilibrate at 37° C. for a minimum of 15 min. Then, 50 μl/vial of a solution containing a mixture of ⁶⁸Ga-labeled (25 μL) and unlabeled protein (25 μL, competitor) with increasing concentrations (10⁻¹⁰-10⁻⁶ M) was added (triplicates of each concentration).

After 1 h incubation at 37° C., the incubation was terminated by centrifugation at 600×g (1,200 rpm, Biofuge 15) for 5 min, the supernatant of each vial was removed and cells were thoroughly washed 2 times with 250 μl PBS. After centrifugation at 600×g (1,200 rpm, Biofuge 15) for 5 min, the washing media was combined with the previously removed supernatant, representing the amount of free radioligand. The amount of cell bound activity (cell pellet) as well as the amount of free radioligand was measured in a 2470 Wizard² γ-counter (PerkinElmer, MA, USA). The proportions of mean cell bound activities were plotted against the concentration of the unlabeled ligand. Each data point is the average of at least three determinations. Half maximal inhibitory concentration (IC₅₀) values were determined by non-linear regression analysis according to the following equation:

$$Y = Nonspecific + \frac{( Total - Nonspecific )}{1 + \frac{[ Drug ]}{{IC}_{50}}}$$

Due to the high structural similarity of ⁶⁸Ga-labeled and unlabeled ligand a nearly identical affinity for PD-L1 is assumed, resulting in homologous competitive binding.

### U-698-M Tumor Model

The transduced U-698-M PDL-1 positive and U-698-M wild-type cell line were detached from the surface of the culture flask using Trypsin/EDTA (0.05% and 0.02%) in PBS, centrifuged, and resuspended in PBS and approximately 1×10⁷ cells/200 μL of the U-698-M PDL-1⁺ cells were inoculated subcutaneously on the right shoulder and U-698-M wild-type cells on the left shoulder of 6 to 8 weeks old NSG mice (male, Charles River WIGA GmbH, Sulzfeld, Germany). Tumors were grown for 2 to 4 weeks to reach 0.6-1 cm in diameter.

### Small-Animal PET-Imaging

Mice were intravenously injected via tail vein with approximately 5-7 MBq (˜10-13 μg) of ⁶⁸Ga-labeled Adnectin. In vivo imaging studies were performed using a Siemens Inveon small-animal PET/CT scanner. Static images were recorded at 1 h and 2 h p.i. with an acquisition time of 20 min. For blocking studies, unlabeled Adnectin (9 mg/kg) was coinjected with ⁶⁸Ga-Adnectin. Dynamic imaging was performed after on-bed injection for 1.5 h under isoflurane anesthesia. Reconstruction of the images was carried out using 3-dimensional ordered-subsets expectation maximum (OSEM3D) algorithm with scanner and attenuation correction. Data analysis was carried out using Inveon Workplace software (Siemens).

### FACS Analysis

Flow cytometric analysis was performed on an LSRII (BD Bioscience), and results were analyzed using FlowJo 7.6.5 software. Centrifugation steps were carried out at 500 g and 4° C. for 5 min. Per staining, 0.5-1 Mio of the cells which had to be characterized were washed with FACS-buffer. Cells were incubated with 50 μl human serum for 10 min at 4° C. to prevent non-specific binding of the antibody. After a further washing step with FACS-Buffer, 2 μl of PE-labelled Mouse Anti-Human CD274, Clone MIH1, (BD Bioscience, Franklin Lakes, USA) and 1.5 μl 7AAD was added followed by incubating the mixture for 30 min at 4° C. in the dark. The mixture was again washed with FACS buffer after antibody staining to remove excess antibody before taking up the cells in 200 μl FACS buffer. The cells were stored at 4° C. in the dark until measurement.

For ex vivo flow cytometric analysis, tumor and organs were singularized using 40 μm cell strainer. After washing with FACS buffer, erythrocytes were lysed using ACK lysis buffer for 5 min at room temperature and used for flow cytometric analysis.

### Histology and Immunohistochemistry

Tumor tissues were fixed in 10% neutral-buffered formalin solution for at least 48 hrs, dehydrated under standard conditions (Leica ASP300S, Wetzlar, Germany) and embedded in paraffin. Serial 2 μm-thin sections prepared with a rotary microtome (HM355S, Thermo Fisher Scientific, Waltham, USA) were collected and subjected to histological and immunohistochemical analysis. Hematoxylin-Eosin (H.-E.) staining was performed on deparaffinized sections with Eosin and Mayer's Haemalaun according to a standard protocol.

Immunohistochemistry of tumor tissues was performed using a Bond RXm system (Leica, Wetzlar, Germany, all reagents from Leica) with primary antibodies against PD-L1 antibody (clone 28-8, ab205921). Briefly, slides were deparaffinized using deparaffinization solution, pretreated with Epitope retrieval solution 2 (EDTA buffer pH9). The primary antibodies were diluted (1:500) and applied for 15 min. Antibody binding was detected with a polymer refine detection kit without post primary agent and visualized with DAB as a dark brown precipitate. Counterstaining was done with hematoxylin. Slides were then dehydrated manually by alcohol washes of increasing concentration (70%, 96%, 100%) and xylene and cover slipped using Pertex® mounting medium (Histolab, Goeteborg, Sweden, 00801). A positive control was included in each run.

The stained slides were scanned with an automated slide scanner (Leica Biosystems, Wetzlar, Germany, AT-2) and the Aperio Imagescope software (version 12.3, Leica Biosystems, Wetzlar, Germany) was used for taking representative images.

### Biodistribution

About 5-7 MBq of the ⁶⁸Ga-labeled Adnectin (˜10 μg) were injected into the tail vein of the U-698-M-PD-L1+ and U-698-M wild-type tumor bearing NSG mice under isoflurane anesthesia. Animals were sacrificed at 1 h p.i. (n=4) and 2 h p.i. (n=4), the organs of interest were dissected, and the activity in the weighed tissues samples was quantified using a γ-counter.

### Results and Discussion

### ⁶⁸Ga Labeling

⁶⁸Ga-labeling of the PD-L1 binding DOTA-Adnectin was performed following the optimized labeling procedure regarding buffer, pH, temperature and protein amount (170-240 MBq, 1M NaOAc, pH 5.5, 200 μg Adnectin, 50° C.; see first report). High labeling efficiencies with quantitative RCYs of >78% were obtained after 15 min. After purification specific activities (SA) of 4.7-7.3 GBq/μmol and RCP >98% were achieved. Due to the ease of tracer preparation, the synthesis of ⁶⁸Ga-Adnectin should be fully compatible with everyday clinical workflow and should be well suited for automated radiosynthesis in clinical routine.

### In Vitro Stability of ⁶⁸Ga-Adnectin in Human Serum

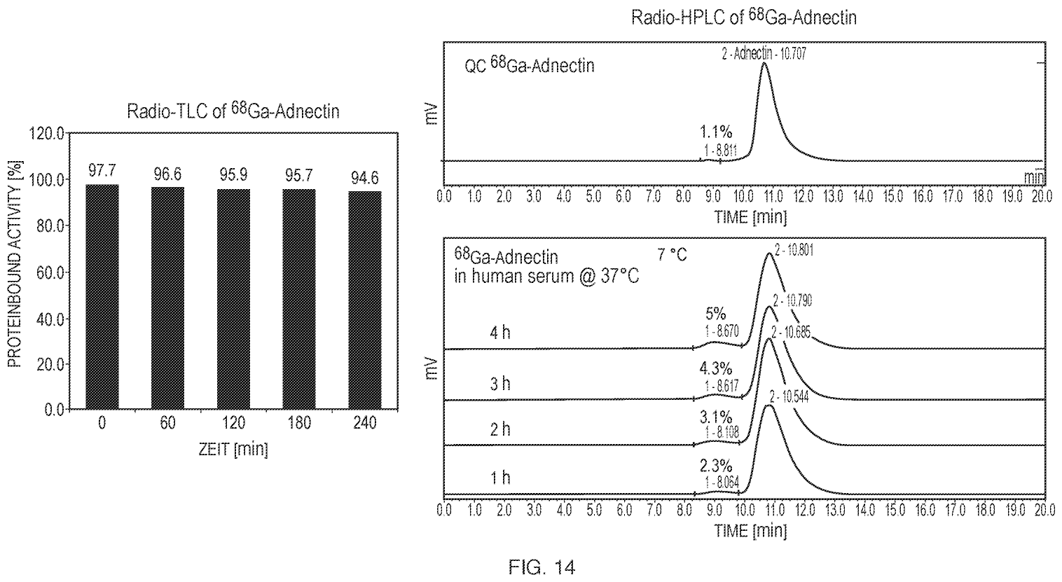

In vitro stability of ⁶⁸Ga-labeled Adnectin in human serum at 37° C. was determined by Radio-TLC and Radio-HPLC up to 4 h (FIG. 1). Radio-chromatograms of ⁶⁸Ga-Adnectin revealed monomeric elution profiles with a radioactive impurity of higher molecular weight (8.97 min), which slightly increases up to 5% after 4 h of incubation at 37° C. in human sera. Radio-TLC analysis revealed moderate transmetallation (5%) for ⁶⁸Ga-Adnectin within 4 h. Therefore, a reasonable in vitro stability of ⁶⁸Ga-labeled Adnectin (≥95% of intact tracer) in human serum was confirmed for up to 4 h post synthesis, which ensures the prolonged availability of intact tracer in the circulation within the scheduled imaging time for ⁶⁸Ga-labeled tracers (FIG. 14).

### Transduction of Cell Lines

The B cell lymphoma cell line U698M was retrovirally transduced with PD-L1 linked to GFP and a stable cell line was generated by PD-L1-based sorting. Non-transduced U698M cells revealed only a low expression of PD-L1 of approximately 4.000 molecules per cell. PD-L1 expression could be considerably increased by stable transduction, revealing ˜155.000 PD-L1 molecules per cell for U-698-M. The stable transduced U-698-M cells were therefore suitable for the further evaluation of PD-L1 binding radioligands in vitro and in vivo. (FIG. 1)

### Small-Animal PET-Imaging

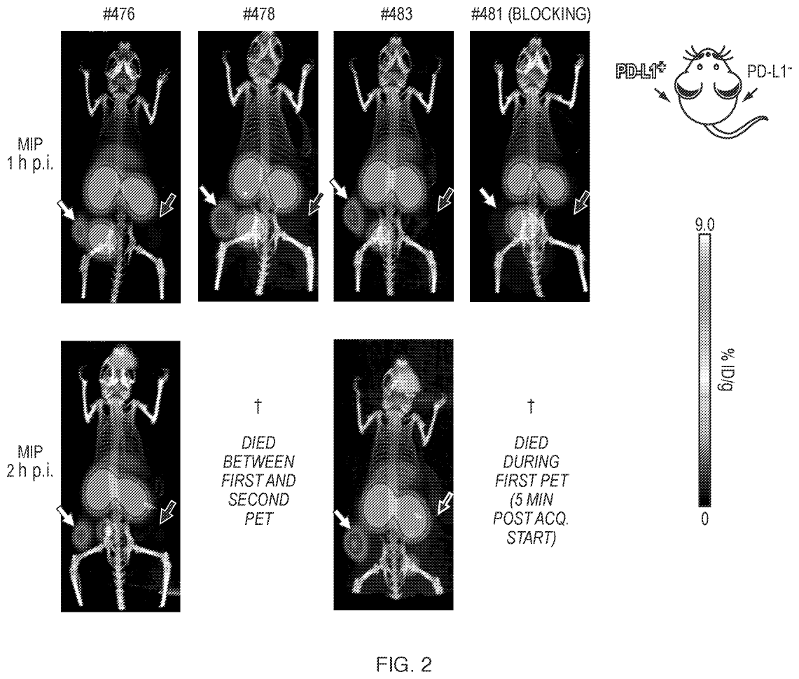

Comparative static pPET scans were performed with ⁶⁸Ga-Adnectin in PD-L1 positive and PD-L1 wild-type tumor-bearing NSG mice at 1 h p.i and 2 h p.i. Approximately 5-7 MBq (10-13 μg; 0.9-1.2 nmol) of the respective tracer were injected in mice (FIG. 2).

⁶⁸Ga-Adnectin displayed a rapid blood clearance with low unspecific whole-body uptake and predominant renal clearance with a slight shift from renal to hepatobiliary excretion, shown by a slightly enhanced tracer uptake in the liver. ⁶⁸Ga-Adnectin showed comparable high tumor uptake in PDL-1 positive tumors after 1 h and 2 h p.i.

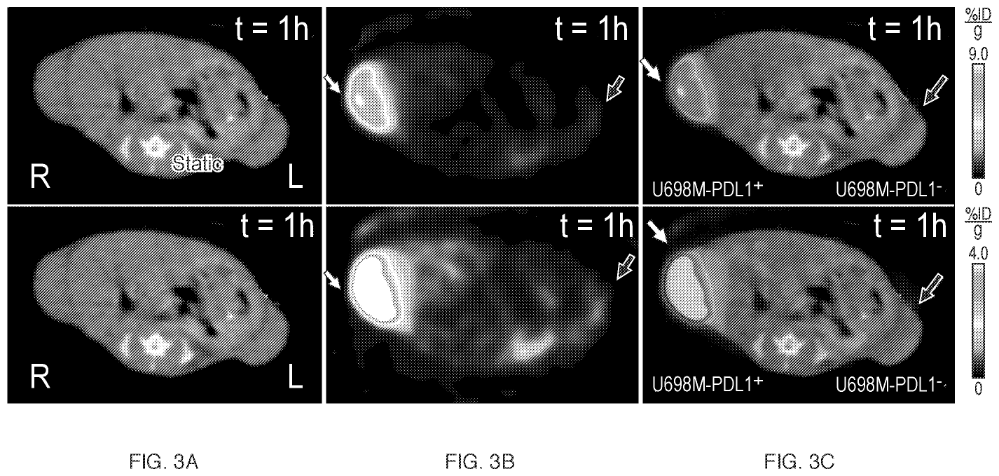

As shown in FIG. 3, the direct comparison of ⁶⁸Ga-Adnectin accumulation in PD-L1 positive U-698-M and PD-L1 U-698-M wild-type tumors confirmed PD-L1 specific binding for ⁶⁸Ga-Adnectin. Additionally, blocking experiments with unlabeled Adnectin (9 mg/kg) demonstrated that ⁶⁸Ga-Adnectin uptake is specific and PD-L1 mediated (FIGS. 2 and 3).

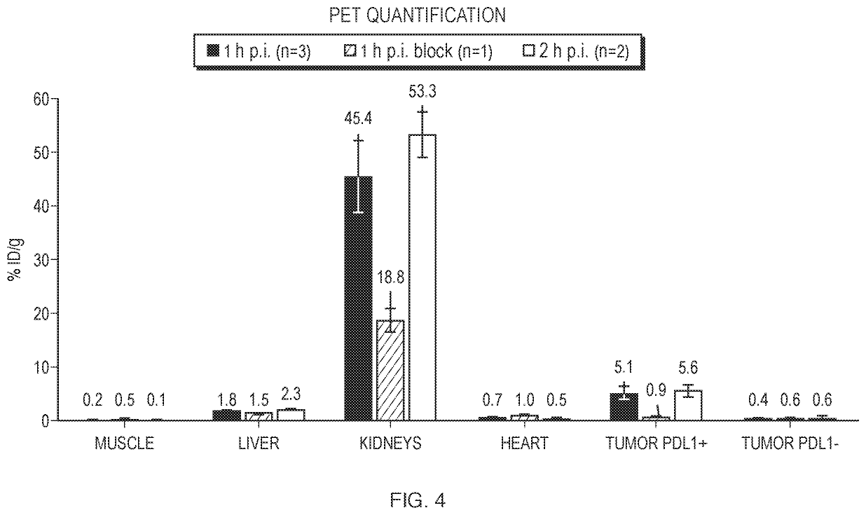

ROI Quantification of the static pPET images of ⁶⁸Ga-Adnectin revealed favorable pharmacokinetics with predominant renal clearance (FIG. 4). Whereas, kidney and liver uptake slightly increased within 2 h p.i. tumor uptake of ⁶⁸Ga-Adnectin remains equally high, resulting in higher tumor-to-background ratios at 1 h p.i. Therefore, high-contrast PET imaging after 1 h p.i seems to be more preferable. As seen in PET images ⁶⁸Ga-Adnectin is PD-L1 specific and blockable. Interestingly, blocking experiments with an excess of unlabeled Adnectin revealed significant reduction of kidney accumulation of the tracer (FIG. 4).

### Dynamic μPET/CT Imaging—Time-Activity Curves

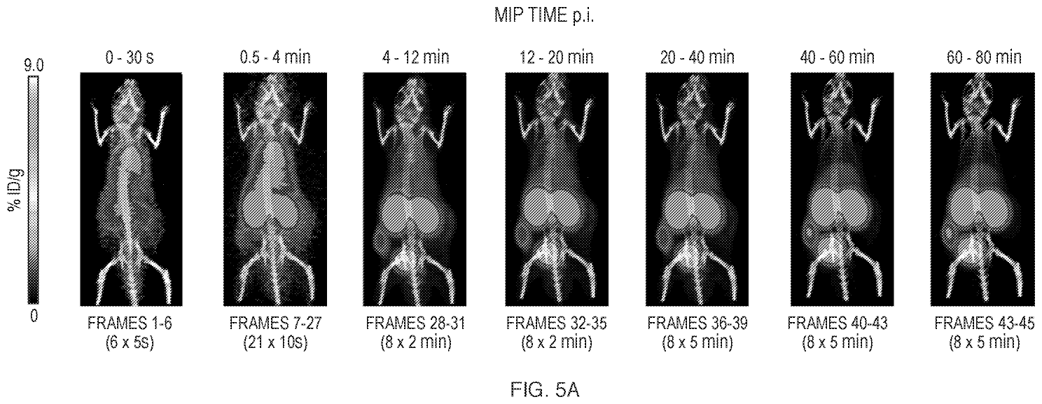

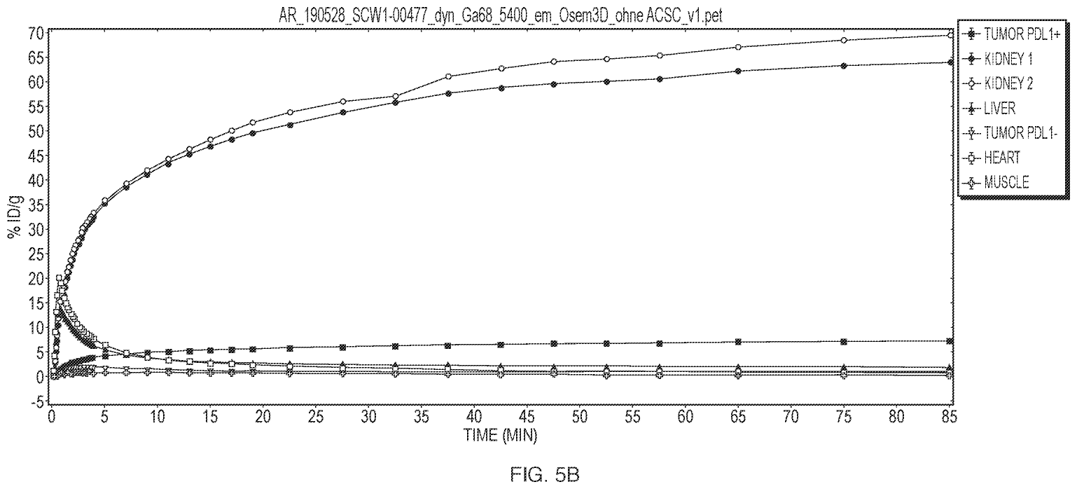

The pharmacokinetics of ⁶⁸Ga-Adnectin were investigated by carrying out dynamic pPET/CT scans over a period of 1.5 h in PD-L1 positive U-698-M and U-698-M wild-type xenograft bearing mice (FIG. 5). To achieve comparable results the injected amount of activity and protein were adjusted to similar values as for static PET imaging of ⁶⁸Ga-Adnectin.

The summation images at 40-90 min p.i. of the dynamic pPET/CT scan of ⁶⁸Ga-Adnectin are comparable to those obtained for static pPET imaging 1 h and 2 h p.i (FIG. 2). ⁶⁸Ga-Adnectin showed a rapid clearance from the blood pool and from non-target tissues (e.g., liver) with low non-specific whole-body uptake, and predominant renal clearance demonstrated by continuously increasing accumulation in the kidneys over time with a slight shift from renal to hepatobiliary excretion, shown by only a slightly enhanced uptake in the liver. ⁶⁸Ga-Adnectin uptake in PD-L1 positive tumors was fast, within 4 min. p.i., and showed increasing PD-L1⁺ tumor uptake within 60 min p.i. with high retention over 1.5 h.

No accumulation was observed in U-698-M wild-type xenografts, confirming PD-L1 specific binding of ⁶⁸Ga-Adnectin. Additionally, blocking experiments with excess of unlabeled Adnectin (9 mg/kg) demonstrated that ⁶⁸Ga-Adnectin uptake is specific and PD-L1 mediated.

### Biodistribution

Comparative static pPET scans were performed with ⁶⁸Ga-Adnectin in PD-L1 positive and PD-L1 wild-type tumor-bearing NSG mice at 1 h p.i and 2 h p.i. To achieve comparable results the injected amount and activity of ⁶⁸Ga-Adnectin (app. 5-6 MBq, 10 μg) was equal as for dynamic PET imaging.

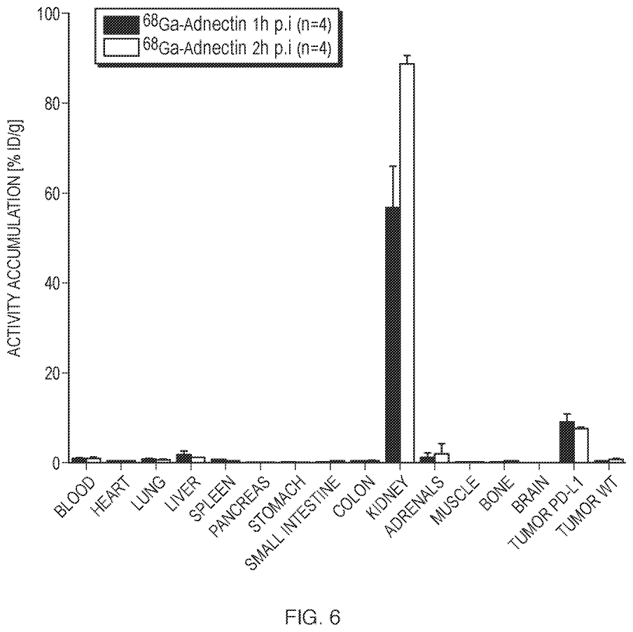

The static PET/CT images of ⁶⁸Ga-Adnectin at 1 h and 2 h p.i. (FIG. 5) are comparable to those summation images at 40-90 min p.i. obtained by the dynamic pPET/CT scan The biodistribution data of ⁶⁸Ga-Adnectin in PD-L1 positive-U-698-M and U-698-M wild-type tumor-bearing mice (1 h and 2 h p.i.) are shown in FIG. 6 and summarized in Table 1. The data fully mirror the results of dynamic pPET/CT imaging.

TABLE 1
Biodistribution of ⁶⁸Ga-Adnectin in PD-L1 positive U-698-M and U-698-M wild-
type tumor-bearing NSG mice at 1 h and 2 h p.i. Data are expressed as % ID/g (mean ±
SD). Right panel: Tumor-to-organ ratios of ⁶⁸Ga-Adnectin 1 h and 2 h p.i.

| | ⁶⁸Ga-Adnectin | | | ⁶⁸Ga-Adnectin | |
|---|---|---|---|---|---|
| | Biodistribution [% ID/g] | | | Tumor-to-Organ Ratios | |
| | 1 h p.i | 2 h p.i. | | 1 h p.i | 2 h p.i. |
| Blood | 1.0 ± 0.3 | 1.1 ± 0.3 | T/Blood | 8.9 ± 3.3 | 7.1 ± 2.1 |
| Heart | 0.5 ± 0.1 | 0.4 ± 0.1 | T/Heart | 19.4 ± 6.3 | 17.4 ± 4.4 |
| Lung | 0.8 ± 0.2 | 0.7 ± 0.2 | T/Lung | 10.8 ± 3.4 | 11.4 ± 3.0 |
| Liver | 1.9 ± 0.7 | 1.2 ± 0.2 | T/Liver | 4.7 ± 1.9 | 6.1 ± 1.1 |
| Spleen | 0.7 ± 0.2 | 0.5 ± 0.2 | T/Spleen | 12.2 ± 5.0 | 16.4 ± 5.8 |
| Pancreas | 0.2 ± 0.1 | 0.2 ± 0.04 | T/Pancreas | 41.1 ± 23.2 | 36.2 ± 6.9 |
| Stomach | 0.3 ± 0.04 | 0.3 ± 0.1 | T/Stomach | 33.2 ± 9.0 | 27.2 ± 6.9 |
| Small intestine | 0.3 ± 0.1 | 0.5 ± 0.1 | T/Small | 27.3 ± 9.4 | 15.2 ± 3.7 |
| Colon | 0.3 ± 0.03 | 0.4 ± 0.2 | intestine | | |
| Kidney | 56.9 ± 9.2 | 88.9 ± 1.8 | T/Colon | 32.7 ± 8.1 | 18.4 ± 7.4 |
| Adrenals | 1.2 ± 1.0 | 2.0 ± 2.2 | T/Kidney | 0.2 ± 0.01 | 0.1 ± 0.0 |
| Muscle | 0.2 ± 0.1 | 0.2 ± 0.1 | T/Adrenals | 7.3 ± 5.9 | 3.6 ± 3.8 |
| Bone | 0.3 ± 0.1 | 0.5 ± 0.1 | T/Muscle | 45.4 ± 20.4 | 32.9 ± 14.2 |
| Brain | 0.04 ± 0.01 | 0.1 ± 0.04 | T/Bone | 27.6 ± 10.5 | 15.0 ± 3.6 |
| Tumor PD-L1+ | 9.0 ± 2.1 | 7.6 ± 0.4 | T/Brain | 216.8 ± 76.7 | 130.8 ± 82.7 |
| Tumor wild- | 0.6 ± 0.1 | 0.7 ± 0.4 | | | |
| type | | | | | |

ROI Quantification of the static μPET images of ⁶⁸Ga-Adnectin revealed that the predominant renal clearance with only slightly increased uptake in the liver and non-targeted tissues at 2 h p.i. At 1 h p.i., tumor uptake of ⁶⁸Ga-Adnectin remains equally high, resulting in higher tumor-to-background ratios at 1 h p.i. The significant uptake of ⁶⁸Ga-Adnectin in PD-L1 positive tumor xenografts is highly specific, as no accumulation in PD-L1 negative tumors was observed. Due to the higher kidney uptake 2 h p.i. with similar high tumor uptake as for 1 h p.i., earlier imaging time points (1 h p.i.) seem to be more recommendable, due to the higher tumor-to-organ ratios. Interestingly, blocking experiments with an excess of unlabeled Adnectin revealed significant reduction of kidney accumulation of the tracer.

### FACS Analysis

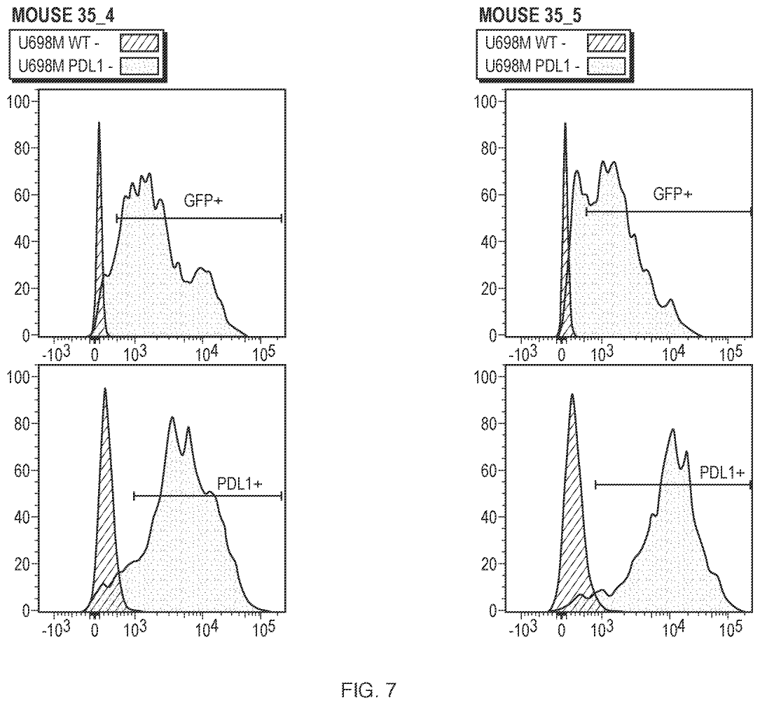

Ex vivo flow cytometry (FACS) analysis of the dissected tumors from 2 mice confirmed the imaging results showing higher ⁶⁸Ga-Adnectin uptake in the PD-L1 transduced tumors compared to U-698-M wild-type tumor tissues. In both mice, PD-L1 expression levels in transduced tumors was highly increased to non-transduced, confirming the generation of a stable PD-L1 tumor cell line in vivo and PD-L1 mediated uptake of ⁶⁸Ga-Adnectin (FIG. 7).

### Ex Vivo Histology and Immunohistochemistry

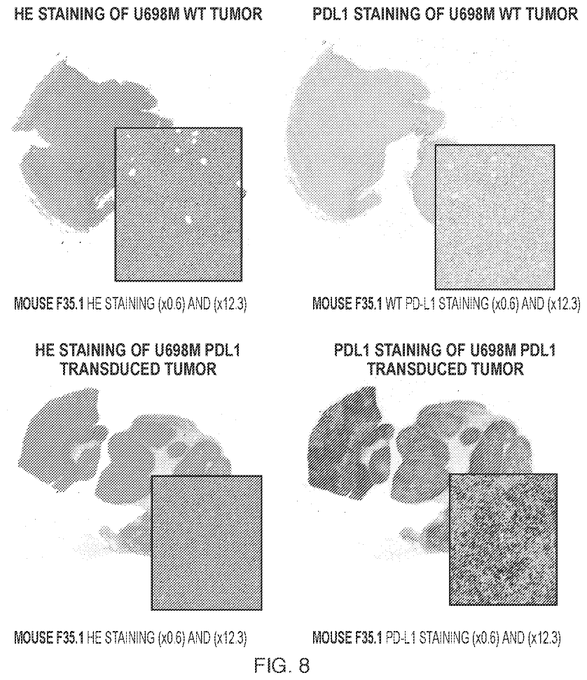

To further confirm the results of FACS analysis we performed ex vivo immunohistochemistry analysis of the wildtype and PD-L1⁺ tumors. Strong PD-L1 expression was found in all U-698-M PD-L⁺ tumors (FIG. 8, lower panel) while no PD-L1 expression was found in all U-698-M wildtype tumors (FIG. 8, upper panel). These results confirm the in vivo imaging results as well as the ex vivo flow cytometry analysis.

### Conclusions

⁶⁸Ga-Adnectin was obtained with quantitative RCYs with high RCP (>97%). High and specific binding of ⁶⁸Ga-Adnectin to human PD-L1-expressing cancer cells was confirmed, which closely correlates with the respective PD-L1 expression level determined by flow cytometry and IHC staining. In vivo, ⁶⁸Ga-Adnectin uptake was high in PD-L1⁺ tumors (9.0±2.1% ID/g at 1 h p.i.) and kidneys (56.9±9.2% ID/g at 1 h p.i.) with negligible uptake in other tissues. PD-L1 negative tumors demonstrated only background uptake of radioactivity (0.6±0.1% ID/g). Co-injection of an excess of unlabeled Adnectin reduced tumor uptake of PD-L1 by more than 80%.

⁶⁸Ga-Adnectin enables easy, fast and efficient radiosynthesis and shows excellent in vitro and in vivo PD-L1 targeting characteristics. The high tumor uptake combined with low background accumulation at early imaging time points demonstrate the feasibility of ⁶⁸Ga-Adnectin for imaging of PD-L1 expression in tumors and is encouraging for theranostic applications of PD-L1 ligands.

### Example 3: Comparative Preclinical Evaluation of ⁶⁸Ga-Labeled NOTA-Adnectin Vs. DOTA-Adnectin

All materials and methods used in this study are as described in Examples 1-2.

⁶⁸Ga-labeling of the PD-L1 binding DOTA- or NOTA-conjugated Adnectin was performed following the optimized labeling procedure regarding buffer, pH, temperature and protein amount (130-250 MBq, 1M NaOAc, pH 5.5, 100 or 200 μg DOTA/NOTA-Adnectin, 50° C.), as previously described herein.

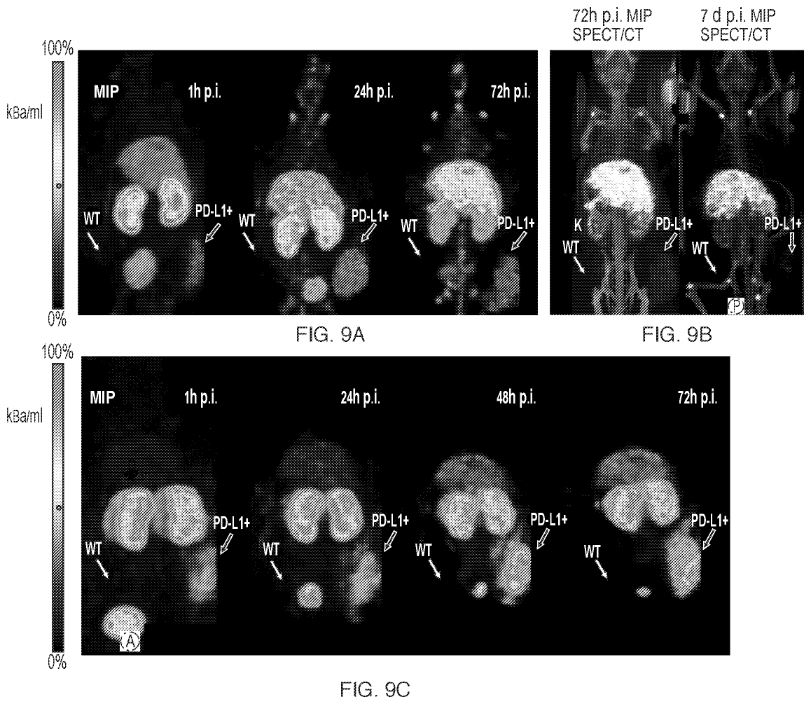

The radiochemical yields (RCYs) were assessed as a function of the amount of radioactivity, precursor and reaction time (FIG. 9). ⁶⁸Ga-labeling using app. 130 MBq ⁶⁸GaCl₃ and 200 μg of the respective precursor (DOTA- vs. NOTA-Adnectin) revealed significantly higher RCYs (≥95%) for NOTA-Adnectin even after 5 min in comparison to ⁶⁸Ga-labeling of DOTA-Adnectin at 5 and 15 min reaction time (FIG. 9A). A 2-fold reduction of NOTA-Adnectin amount (100 μg) showed comparable high and quantitative RCYs ≥95% as for 200 μg precursor.

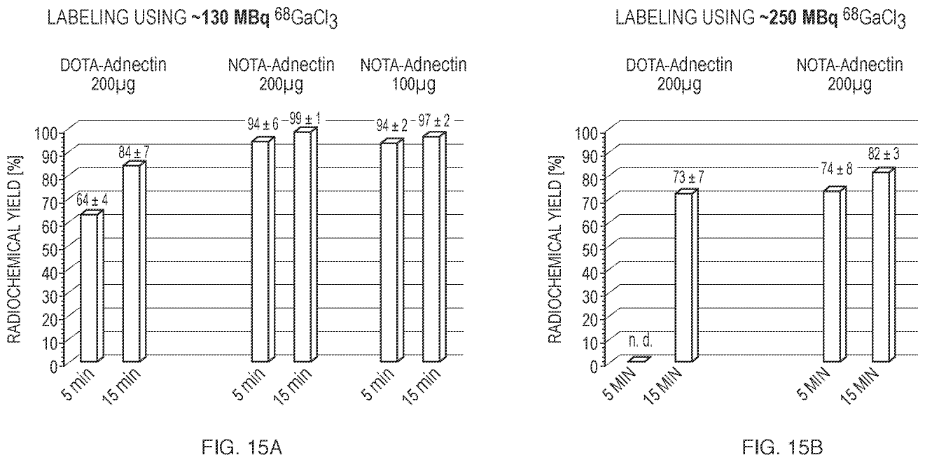

Therefore, while both DOTA-Adnectin and NOTA-Adnectin are suitable for use in the methods provided herein, NOTA-Adnectin allows the use of much lower precursor concentrations for labeling than required for DOTA-Adnectin. This allows ⁶⁸Ga-NOTA-Adnectin to be prepared with much higher specific activities. The use of higher starting activities (app. 250 MBq of ⁶⁸GaCl₃) obtained higher RCYs for NOTA-Adnectin even after 5 min of labelling compared to DOTA-functionalized Adnectin. As shown in FIG. 15, NOTA is a more effective chelating agent for ⁶⁸Ga (stability constant, K_{ML}=31.1), compared to DOTA (K_{ML}=21.3). In addition, ⁶⁸Ga-labeling of NOTA is compatible with heat sensitive proteins due to its short radiolabelling time and ability to radiolabel at room temperature.

### Dynamic μPET/CT Imaging

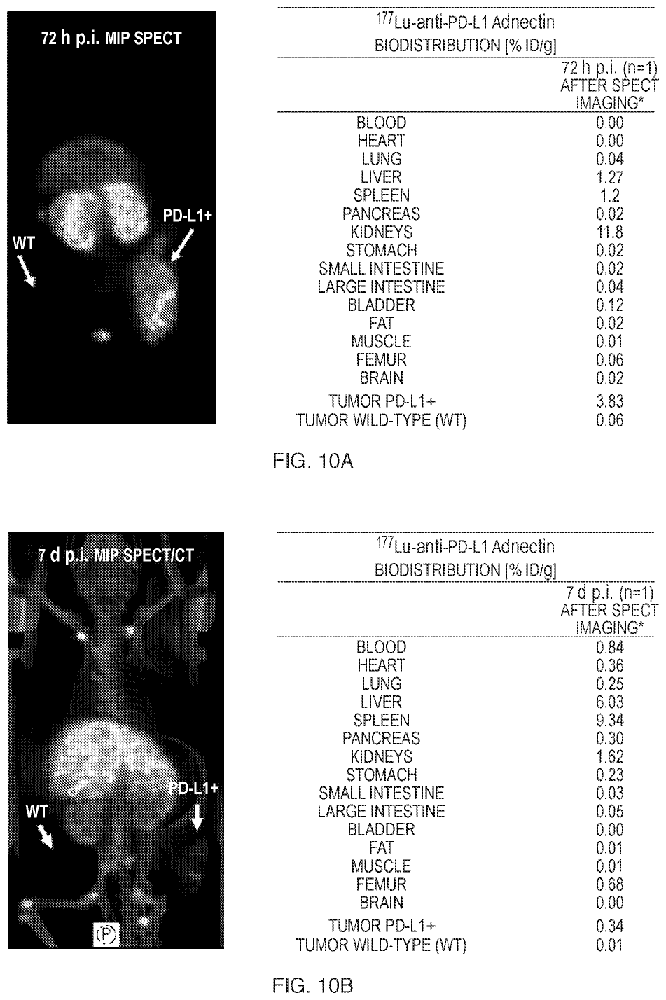

The pharmacokinetic of ⁶⁸Ga-NOTA-Adnectin was investigated by carrying out a dynamic pPET/CT scan over a period of 1.5 h in PD-L1 positive U-698-M and U-698-M wild-type xenograft bearing mice. A comparison of the dynamic pPET scans of ⁶⁸Ga-DOTA- versus ⁶⁸Ga-NOTA-Adnectin is shown in FIG. 10. The amount of injected activity and cold mass was identical for both PET scans of ⁶⁸Ga-DOTA- and ⁶⁸Ga-NOTA-Adnectin. Both tracers exhibited PD-L1-dependent binding in PD-L1+ tumor xenografts with no uptake in PD-L1 negative tumors. ⁶⁸Ga-NOTA-Adnectin showed faster accumulation in PD-L1+ tumors and higher uptake after 90 min p.i.

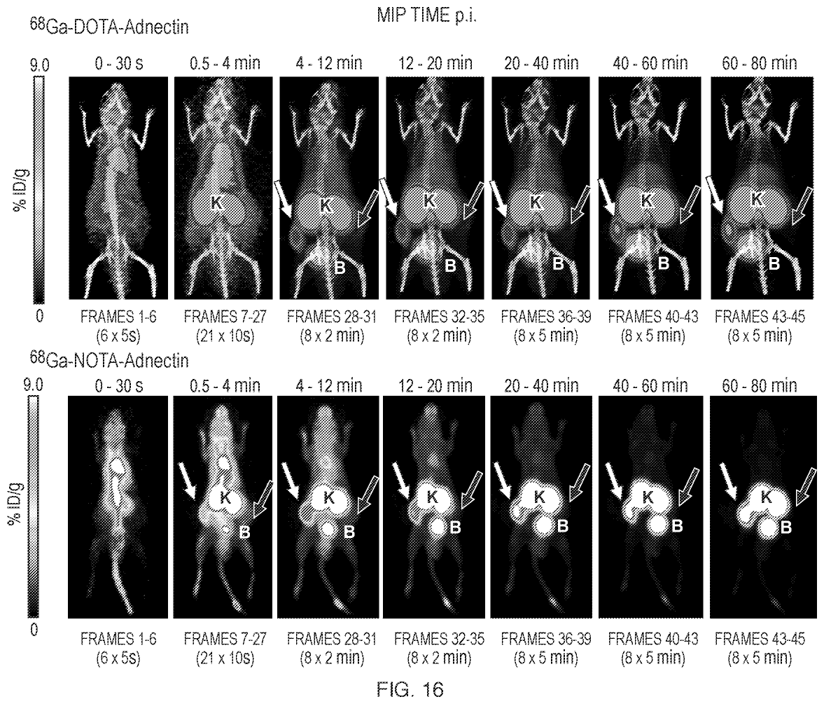

The distribution of both tracers within normal organs and tissues was favorable, with low blood and normal tissue uptake and rapid clearance. The highest accumulation of both tracers was observed in the kidneys. Beside to the predominant renal excretion of both tracers, for ⁶⁸Ga-DOTA-Adnectin a slightly enhanced uptake in the liver was observed. (FIG. 16)

### Static μPET/CT Imaging

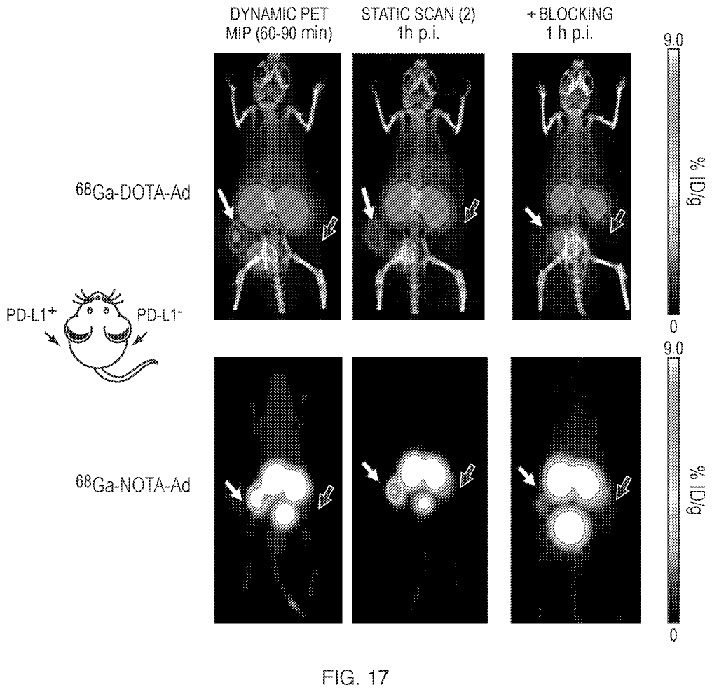

Comparative static pPET scans were performed with ⁶⁸Ga-NOTA-Adnectin in PD-L1 positive and PD-L1 wild-type tumor-bearing NSG mice at 1 h p.i and 2 h p.i. To achieve comparable results the injected amount and activity of ⁶⁸Ga-DOTA- and ⁶⁸Ga-NOTA-Adnectin (app. 5-6 MBq, 10 μg) was equal as for dynamic PET imaging (FIG. 16). The static PET/CT images of ⁶⁸Ga-NOTA- and ⁶⁸Ga-DOTA-Adnectin at 1 h (FIG. 17) are comparable to those summation images at 40-90 min p.i. obtained by the dynamic pPET/CT scans for both tracers.

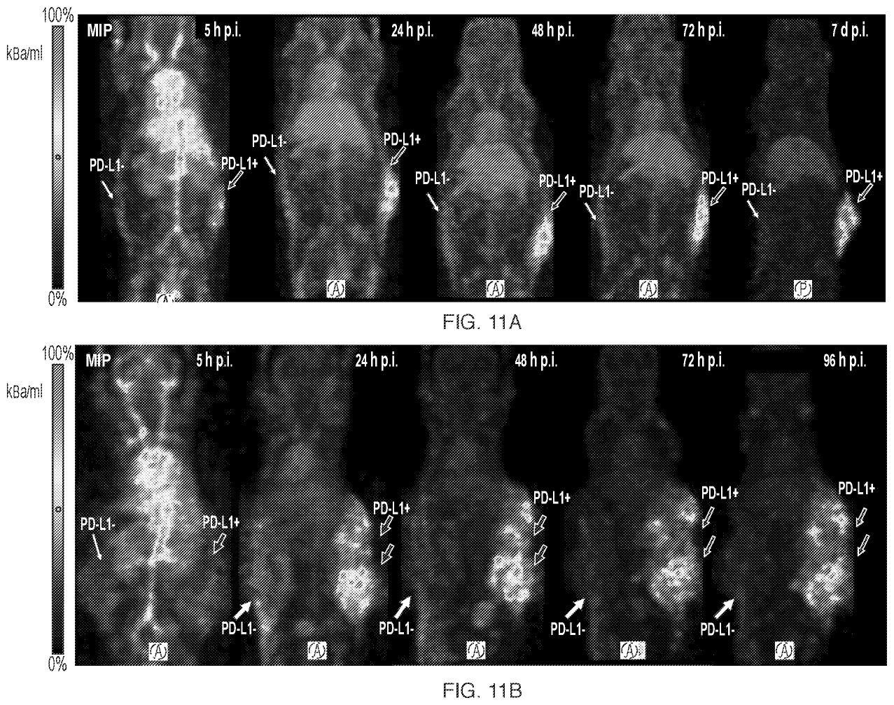

Both radiotracers enable specific visualization of PD-L1+ tumors and no accumulation was observed in U-698-M wild-type xenografts, confirming PD-L1 specific binding of ⁶⁸Ga-DOTA- and ⁶⁸Ga-NOTA-Adnectin. Additionally, blocking experiments with excess of unlabeled Adnectin (9 mg/kg) demonstrated that ⁶⁸Ga-labeled Adnectin uptake is for both tracers specific and PD-L1 mediated (FIG. 11). As anticipated from dynamic PET imaging, both radioligands show high renal uptake with significant bladder activity, indicating rapid renal clearance of ⁶⁸Ga-DOTA- and ⁶⁸Ga-NOTA-Adnectin. Whereas ⁶⁸Ga-NOTA-Adnectin revealed exclusively renal excretion, a slight shift from renal to hepatobiliary excretion was observed for ⁶⁸Ga-DOTA-Adnectin, shown by a slightly enhanced tracer uptake in the liver.

### Biodistribution

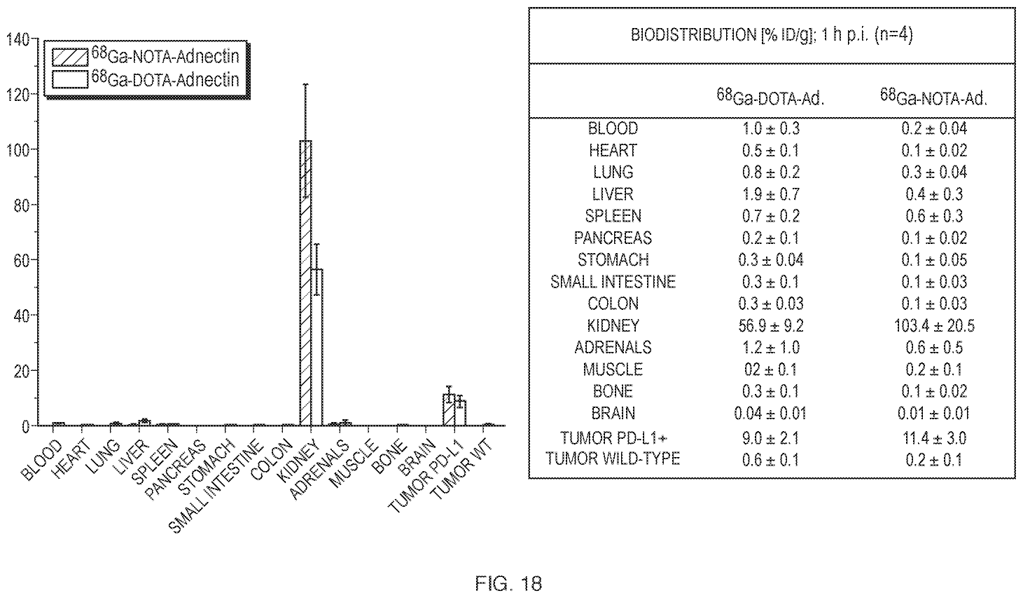

The biodistribution data of ⁶⁸Ga-DOTA- and ⁶⁸Ga-NOTA-Adnectin in PD-L1 positive-U-698-M and U-698-M wild-type tumor-bearing mice (1 h p.i.) are summarized in FIG. 18. The data fully mirror the results of pPET/CT imaging. Both tracers exhibit comparable organ distribution with low accumulation in non-target tissues. The kidney uptake of ⁶⁸Ga-NOTA-Adnectin was 2-fold higher with 103±21% ID/g than for ⁶⁸Ga-DOTA-Adnectin (57±9% ID/g) at 1 h p.i. In addition, a slightly enhanced uptake (>2% ID/g) of ⁶⁸Ga-NOTA-Adnectin in PD-L1+ tumor was observed in comparison to ⁶⁸Ga-DOTA-Adnectin. Tumor uptake of both tracers was specific and PD-L1 mediated. As seen in PET images ⁶⁸Ga-DOTA-Adnectin showed a low liver uptake with 2% ID/g, whereas no uptake was observed for ⁶⁸Ga-NOTA-Adnectin.

### Conclusion

The comparison of the radiolabelling efficiencies for ⁶⁸Ga-labeled DOTA- versus NOTA-Adnectin revealed higher RCYs within shorter synthesis time for the NOTA-conjugated compound. In addition, higher specific activities can be achieved for ⁶⁸Ga-NOTA-Adnectin due to the lower required amounts of NOTA-Adnectin for quantitative ⁶⁸Ga-labelling. Therefore, due to the mild labelling conditions and the quantitative radiochemical yields with high radiochemical purity, preparations of ⁶⁸Ga-NOTA-Adnectin for application in clinical routine are particularly suitable for use in kit-like preparations.

In comparative preclinical studies both tracers showed comparable in vivo PD-L1-targeting characteristics with selective and specific accumulation in PD-L1 positive tumors. Therefore, an exchange of the chelator unit within the protein seems to have no influence on the human PD-L1 binding affinities of Adnectin.

In addition, both tracers revealed comparable and favourable pharmacokinetics with fast renal excretion and low uptake in non-target tissues. A slight difference was observed for both ligands regarding the uptake in excretion organs like the kidneys and liver. Whereas ⁶⁸Ga-NOTA-Adnectin was exclusively excreted via the urinary pathway resulting in 2-fold higher kidney uptake compared to ⁶⁸Ga-DOTA-Adnectin, the DOTA-conjugated protein showed lower kidney accumulation with increased liver uptake, due to a slight shift from renal to hepatobiliary excretion. Since NOTA is a hexadentate N₃O₃ macrocyclic chelator, ⁶⁸Ga-labeling of NOTA-Adnectin was expected to produce a neutral compound, while the ⁶⁸Ga-DOTA complex is negatively charged, which may have influence on the pharmacokinetic behaviour of both radiotracers. The lower liver uptake of ⁶⁸Ga-NOTA-Adnectin might also offer an advantage for imaging tumors with higher contrast.

### Example 4: Preclinical Evaluation of ¹⁷⁷Lu-Labeled Anti-hPDL-1 Adnectin and ¹⁷⁷Lu-Labeled Anti-hPDL-1 mAb

Molecular imaging of the immune checkpoint ligand PD-L1 is increasingly investigated as a strategy to guide patient selection and monitor PD-1:PD-L1-targeted immunotherapy. In addition, PD-L1 targeted imaging can be expanded towards theranostic approaches, providing a versatile molecular platform for specific molecular targeting, both for diagnostics and therapy. The primary goals of the project are (1) to investigate whether two types of PD-L1 ligands developed by BMS are suitable for imaging (⁶⁸Ga/¹⁷⁷Lu) of PD-L1 expression on tumors and radionuclide-based treatment (¹⁷⁷Lu); and (2) to obtain preliminary data on the effectiveness and toxicity of PD-L1 targeted radionuclide therapy.

### Material and Methods

### ¹⁷⁷Lu Labeling

¹⁷⁷LuCl₃ was purchased from IDB Holland (LuMark®; Baarle-Nassau, The Netherlands) or ITG (EndolucinBeta®; Garching, Germany). The initial radioactivity used for the ¹⁷⁷Lu-labeling of the proteins were measured using a dose calibrator (Capintec INC., New Jersey, US).

Proteins BMS-936559 (labeled with 2-5 DOTA per antibody molecule, as described in Example 2; “mAb”) and A02 adnectin with PC tail (SEQ ID NO: 88) (“Adnectin”) were formulated in PBS buffer (pH 7.4) with concentrations of 0.9 mg/mL for Ad and 2.6 mg/mL for mAb.

Radio-TLC was performed using Varian silica impregnated ITLC chromatography paper (Varian Inc., CA, USA) and 0.1 M_{aq}. sodium citrate buffer (pH 5.5) as mobile phase, where the ¹⁷⁷Lu-labeled protein stays at the origin (R_{F}=0) and free ¹⁷⁷Lu^{III} is eluted with the solvent front (R_{F}=0.8−1). TLC stripes were analyzed on a B-FC-3600 TLC Scanner (Bioscan, Washington, USA).

For ¹⁷⁷Lu-labeling, 100 μL of 1 M NaOAc (pH 5.5) were added to 60 μL ¹⁷⁷LuCl₃ (app. 100-130 MBq) in 0.04 M HCl. To this solution 79 μL mAb (200 μg) or 222 μL Adnectin (200 μg) in PBS (pH 7.4) were added and mixed by shaking, resulting in a reaction solution with a final pH of 5.5. Heating to 42° C. was performed by placing the closed eppendorf vials into a heated Eppendorf mixer. After 1-2 h radioactivity incorporation was determined by radio-TLC. The labeled proteins were purified by gel filtration on a PD-10 column (GE Healthcare, Buckinghamshire, UK).

### Cell Culture and Animal Model

PD-L1 positive U-698 M and U-698-M wild-type cells were maintained as described in Examples 1-2. Male NSG mice (6 to 8 weeks, Charles River WIGA GmbH, Sulzfeld, Germany) were inoculated subcutaneously with 1×10⁷ cells/200 μL of the U-698-M PDL-1 positive (right flank) and U-698-M wild-type (left flank) cell line. Tumors were grown for 2 to 3 weeks to reach 0.6-1 cm in diameter.

### Small-Animal SPECT-Imaging

Mice were intravenously injected via tail vein with. approximately 34-37 MBq (˜70-100 μg) of ¹⁷⁷Lu-labeled Adnectin or 25-34 MBq (˜100-102 μg) of ¹⁷⁷Lu-mAb. In vivo imaging studies were performed using a Mediso Inveon nanoScan SPECT/CT scanner equipped with the NSP-106 multipinhole mouse collimator and with energy windows of 20% centered over the 56-, 113- and 208 keV energy peaks of ¹⁷⁷Lu. Static images were recorded at 1 h, 5 h, 24 h, 48 h, 72 h, 96 h and 7d p.i. with an acquisition time of 40 min. CT imaging was done before each whole body-SPECT. Reconstruction of the images and data analysis was carried out using an VivoQuant™ and InterView™ FUSION software.

### Ex Vivo Autoradiography, Histology and Immunohistochemistry

The animals were sacrificed and PD-L1 positive and wild-type U-698-M tumors were dissected. Tumor tissues were fixed in 10% neutral-buffered formalin solution for at least 48 h, dehydrated under standard conditions (Leica ASP300S, Wetzlar, Germany) and embedded in paraffin. Serial 2 μm-thin sections prepared with a rotary microtome (HM355S, Thermo Fisher Scientific, Waltham, USA) were collected and subjected to histological and immunohistochemical analysis. Hematoxylin-Eosin (H.-E.) staining was performed on deparaffinized sections with Eosin and Mayer's Haemalaun according to a standard protocol.

Immunohistochemistry of tumor tissues was performed using a Bond RXm system (Leica, Wetzlar, Germany, all reagents from Leica) with primary antibodies against PD-L1 antibody (clone 28-8, ab205921). Briefly, slides were deparaffinized using deparaffinization solution, pretreated with Epitope retrieval solution 2 (EDTA buffer pH9). The primary antibodies were diluted (1:500) and applied for 15 min. Antibody binding was detected with a polymer refine detection kit without post primary agent and visualized with DAB as a dark brown precipitate. Counterstaining was done with hematoxylin. Slides were then dehydrated manually by alcohol washes of increasing concentration (70%, 96%, 100%) and xylene and cover slipped using Pertex® mounting medium (Histolab, Goeteborg, Sweden, 00801). A positive control was included in each run. The stained slides were scanned with an automated slide scanner (Leica Biosystems, Wetzlar, Germany, AT-2) and the Aperio Imagescope software (version 12.3, Leica Biosystems, Wetzlar, Germany) was used for taking representative images.

Biodistribution of ¹⁷⁷Lu-Labeled Adnectin and ¹⁷⁷Lu-Labeled mAb

About 1 MBq of ¹⁷⁷Lu-labeled Adnectin (˜2.5 μg) or 0.5-1.5 MBq ¹⁷⁷Lu-mAb (13-15 μg) were injected into the tail vein of the U-698-M-PD-L1⁺ and U-698-M wild-type tumor bearing mice under isoflurane anesthesia. Animals were sacrificed at 1 h (Adnectin), 5 h (mAb), 24 h, 72 h, 5 d and 7 d p.i. the organs of interest were dissected. The activity in the weighed tissues samples was quantified using a γ-counter (1480 Wizard, Wallac, Perkin Elmer) and compared to a radioactive standard. The count data were decay corrected and uptake was expressed as injected activity (dose) per gram of tissue (% ID/g).

Radiation Dose Calculations of ¹⁷⁷Lu-Labeled Adnectin and ¹⁷⁷Lu-Labeled mAb

Using the biodistribution data of the ¹⁷⁷Lu-labeled proteins at different time points, we performed an extrapolation of the absorbed doses to humans. First for both ligands ¹⁷⁷Lu-labeled Adnectin and mAb, organ-activity curves for tumor and normal organs in mice were calculated. The organ-time activity concentrations (=Area under the curve (AUC)) obtained from the biodistribution data ([% IA/organ×hour]ₘₒᵤₛₑ) were translated to human whole-organ percent of injected dose ([[% IA/organ×hour]ₕᵤₘₐₙ) based on the principle that the organ to whole-body time activity concentration ratio of a radiopharmaceuticals in mice would equal to that in humans. This extrapolation to humans is implemented in the following expression:

$$( \frac{\% IA}{Organ \times hour} ) human = ( \frac{\% IA}{Organ \times hour} ) mouse \times [ ( \frac{TBw mouse}{TBw human} ) \times ( \frac{OW human}{OW mouse} ) ]$$

where OWₕᵤₘₐₙ is the human organ weight, OW_{Mouse} is the mouse organ weight, TBW_{Mouse} is the average total body weight of the mice (TBW_{M}=25 g), and TBW_{H} the average total body weight for an adult male (TBW_{H}=73.7 kg). The activity concentration in human heart content was estimated using the AUC value of the mouse blood calculated from the % IA/g values of the mouse blood at each time point obtained from the biodistribution data. The AUC value of the human whole-heart content percent of injected dose ([[% IA/heart content×hour]human) was calculated using the following expression:

$$( \frac{\% IA}{Heart Cont \times hour} ) human = ( \frac{\% IA}{g \times hour} ) mouse \times [ ( \frac{TBw mouse}{TBw human} ) \times ( OW human ) ]$$

where OWₕᵤₘₐₙ is assumed as diastolic heart volume of 200 mL.

For ¹⁷⁷Lu-Adnectin a renal clearance of 100% was assumed within 7 days. For ¹⁷⁷Lu-mAb an excretion of 40% of injected activity via the bowel was calculated after 7 d p.i using the summation of the AUC calculations of the whole mouse at each time point. Clearance of radioactivity after the last studied time point are assumed to occur by physical decay only. From the integrated time-activity curves dose calculation was performed for a selected group of organs using OLINDA. Tumor doses are calculated by assuming complete absorption of the emitted beta particles in the tumor tissue.

### Therapy Study of ¹⁷⁷Lu-Labeled mAb

80 male NSG were injected with either PD-L1 expressing U-698-M (n=40) or wild-type U-698-M cells (n=40). On day 4 post engraftment, mice were randomly divided into 4 groups of each 10 mice injected with PD-L1 positive and 10 mice injected with wild-type U-698-M cells. The groups were injected with either saline, unlabeled mAb or 2 activity doses of ¹⁷⁷Lu-mAb (same amount of cold mAb). The therapeutic regimen in the different groups was concluded in Table 2.

TABLE 2
Therapeutic regimen of ¹⁷⁷Lu-mAb in PD-L1 positive U-698-M or U-698-M wild-
type NSG mice (n = 80).

| Groups | Therapeutic regimen |
|---|---|
| 1) Saline | 200 μL Saline was intravenously injected. |
| 10 mice PD-L1 positive xenografts | |
| 10 mice PD-L1 negative xenografts | |
| 2) Unlabeled mAb | 200 μL of app. 76 μg mAb was intravenously injected. |
| 10 mice PD-L1 positive xenografts | |
| 10 mice PD-L1 negative xenografts | |
| 3) ¹⁷⁷Lu-mAb (high dose) | 200 μL ¹⁷⁷Lu-mAb was intravenously injected: |
| 10 mice PD-L1 positive xenografts | 14.9 ± 0.8 MBq (77 ± 5 μg mAb) (PD-L1+ mice) |
| 10 mice PD-L1 negative xenografts | 14.6 ± 0.9 MBq (68 ± 3 μg mAb) (wild-type mice) |
| 4) ¹⁷⁷Lu-mAb (low dose) | 200 μL ¹⁷⁷Lu-mAb was intravenously injected: |
| 10 mice PD-L1 positive xenografts | 7.2 ± 0.3 MBq (74 ± 3 μg mAb) (PD-L1+ mice) |
| 10 mice PD-L1 negative xenografts | 7.3 ± 0.3 MBq (76 ± 3 μg mAb) (wild-type mice) |

Mice were monitored daily for their general health. Tumor growth was measured every 2 days to assess tumor response. Tumor volumes were calculated using the following formula:

$$Tumor volume = \frac{length \times ( {width}^{2} )}{2}$$

Body weight and blood counts were assessed for side effects. When tumor size was greater than >1 cm, tumor ulceration occurred or bodyweight decreased by more than 10%, mice were sacrificed, and organs collected for HE (and IHC) staining. Monitoring of tumor growth was performed for at least 40 days. Tumor growth curves were compared between ¹⁷⁷Lu-mAb, unlabeled mAb treated animals and animals injected with saline. In addition, tumor growth curves of PD-L1 positive and wild-type xenografts were compared for each group. Furthermore, we will analyze differences in survival among the different treatment by Kaplan-Meier curves and log-rank tests. At the completion of the experiments kidneys and livers are sampled and evaluated for signs of toxicity by HE staining.

### Results

### ¹⁷⁷Lu Labeling

The labeling yield after 1 h-2 h at 42° C. was for ¹⁷⁷Lu-Adnectin 76±17% (n=6) and for ¹⁷⁷Lu-mAb 72±21% (n=7), respectively. After Purification RCP determined by Radio-TLC was for both tracers >95%.

### Small-Animal SPECT-Imaging of ¹⁷⁷Lu-Adnectin

Representative SPECT/CT images of ¹⁷⁷Lu-Adnectin in 2 different PD-L1 positive U-698-M and U-698-M wild-type tumor bearing mice for 1 h, 24 h, 48 h, 72 h and 7 d p.i. are displayed in FIG. 9.

As already seen for ⁶⁸Ga-Adnectin, ¹⁷⁷Lu-labeled Adnectin displayed a rapid blood clearance with low unspecific whole-body uptake and predominant renal clearance at early imaging time points (1 h p.i.). In addition, a slightly enhanced tracer uptake in the liver indicates a slight shift from renal to hepatobiliary excretion.

The SPECT images of both mice (FIG. 9) revealed high specific uptake in PD-L1 positive tumors even at 1 h p.i. In addition, no uptake in PD-L1 negative U-698-M wild-type cells was observed, confirming the specificity of ¹⁷⁷Lu-Adnectin towards PD-L1. A comparison of ¹⁷⁷Lu-Adnectin in both mice imaged with SPECT revealed differences in the pharmacokinetics of the tracer (FIG. 9, A/B vs. C). FIG. 9A showed higher uptake in the kidneys and in the liver at 1 h p.i. with significantly faster renal clearance. Furthermore, radioactive uptake in the joints/bones was observed at 72 h and 7 d p.i. The increasing bone uptake can be explained by in vivo degradation of ¹⁷⁷Lu-Adnectin over time and therefore increasing uptake of free ¹⁷⁷Lu or ¹⁷⁷Lu-complexes. Please note, that all SPECT images are scaled to 100% kBq/mL per mouse. Therefore, clearance from the kidneys result in higher activity signals in the liver. However, it can be assumed that liver uptake of ¹⁷⁷Lu-Adnectin remains nearly constant in the period considered by SPECT imaging (7 d p.i., FIG. 9A).

The second SPECT scan of ¹⁷⁷Lu-Adnectin (FIG. 9C) showed a similar organ distribution however with lower uptake in the excretion organs (kidneys, liver; 1 h p.i.) and significantly delayed clearance kinetics. The delayed renal excretion within 72 h p.i., may be contributed to a reduced metabolism of the mouse (in comparison to mouse 1 from the first SPECT scan). Another possible explanation for the differences in the pharmacokinetics of ¹⁷⁷Lu-Adnectin in both mice is the injection of different Adnectin amounts. Both mice get injected with comparable radioactivity amounts of ¹⁷⁷Lu-adnectin (approx. 35 MBq). However, due to the differences in specific activities for both synthesis, mouse 1 (FIG. 9A/B) was injected with about 100 μg Adnectin, whereas lower amounts of 69 μg Adnectin was administered to mouse 2 (FIG. 9C). The injection of lower tracer amounts could influence the pharmacokinetics of a tracer. It is well known that a reduction of the SA, e.g., by deliberately increasing the concentration of the unlabeled precursor, which could compete with the tracer for binding to the respective target, can significantly influence the specific binding and thus overall pharmacokinetics of a radiopharmaceutical. Whereas for mouse 1, low SA and higher Adnectin amounts result in faster renal clearance kinetics of ¹⁷⁷Lu-Adnectin and lower tumor uptake (FIG. 9A/B), higher kidney and tumor uptake with high retention over 72 h p.i. was achieved, using lower peptide amount with higher SA (FIG. 9C).

To quantify the tissue uptake of ¹⁷⁷Lu-Adnectin mice were sacrificed after SPECT/CT imaging 72 h p.i. (FIG. 10C) and 7 d p.i. (FIG. 10A/B), the organs of interest were dissected, and the activity in the weighed tissues samples was quantified using a γ-counter.

After 72 h p.i. nearly no uptake of ¹⁷⁷Lu-Adnectin in non-target tissue was observed, except of a low splenic uptake and accumulation in the excretion organs like the kidneys and liver. No tracer uptake in PD-L1 negative tumor tissue was observed, demonstrating tumor uptake in PD-L1 positive tumors (3.83% ID/g) is highly specific. In comparison, ex vivo biodistribution of ¹⁷⁷Lu-Adnectin (2. Mouse, 7 d p.i.) still revealed differences in the uptake in PD-L1 positive and PD-L1 negative tumor tissue, however ¹⁷⁷Lu-Adnectin uptake in PD-L1 positive tumors decreased significantly to 0.3% ID/g after 7 d p.i. In addition, nearly complete renal clearance of the tracer was observed within 7 d. p.i, whereas uptake in the spleen (9.3% ID/g) and in the liver (6.0% ID/g) is still apparent. As seen in SPECT images of mouse 1 the liver uptake of ¹⁷⁷Lu-Adnectin was higher than for mouse 2 (FIG. 9C), probably due to the differences in specific activity of ¹⁷⁷Lu-Adnectin for both synthesis.

### Ex Vivo Autoradiography, HE-Staining and Immunohistochemistry

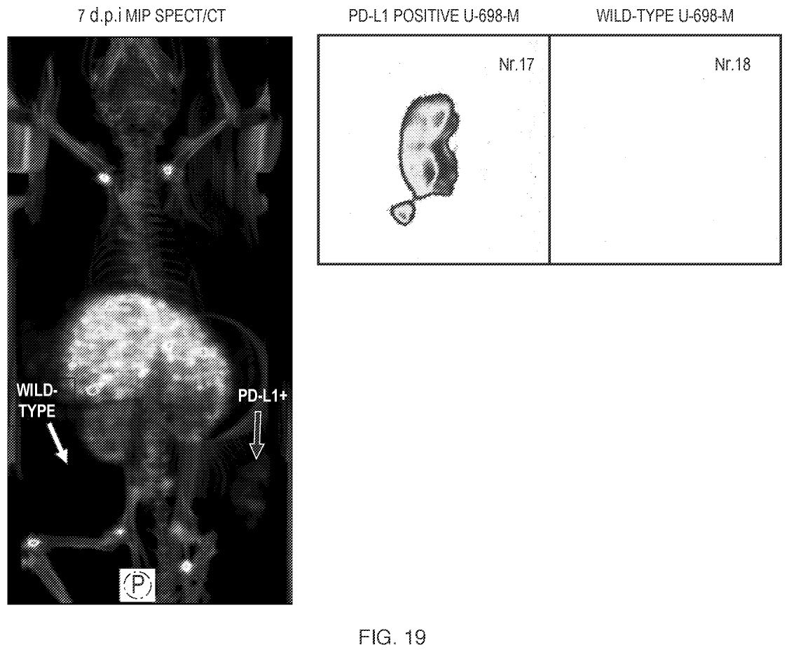

After SPECT imaging the animal was sacrificed and PD-L1 positive and wild-type U-698-M tumors were dissected and prepared for autoradiographic analysis and HE, IHC-staining (FIG. 19).

### Biodistribution of ¹⁷⁷Lu-Adnectin

The biodistribution data of ¹⁷⁷Lu-Adnectin in PD-L1 positive U-698-M and U-698-M wild-type tumor-bearing mice at 1 h, 24 h, 72 h, 5 d and 7 d p.i. is summarized in Table 3. The biodistribution data correlate well with the results obtained for SPECT imaging of ¹⁷⁷Lu-Adnectin. The tracer exhibits low blood activity levels at 1 h p.i. accompanied by low overall background accumulation. ¹⁷⁷Lu-Adnectin shows high initial uptake in the kidney and slightly increased uptake in the liver and spleen. However, while activity is rapidly cleared from the kidneys, hepatic activity levels remain persistently high up to 7 d p.i., suggesting tracer retention in the liver rather than slow and continuous hepatobiliary excretion of the tracer.

TABLE 3
Biodistribution of ¹⁷⁷Lu-Adnectin in U698M-PDL1+ and U698M tumor-bearing
NSG mice at 1 h, 24 h, 72 h, 5 d and 7 d p.i. Injected Dose: 1 MBq; ~2.5 μg
Adnectin/mouse. Data are expressed as % ID/g (mean ± SD).
¹⁷⁷Lu-Adnectin Biodistribution [% ID/g]

| | 1 h p.i | 24 h p.i. | 72 h p.i. | 5 d p.i | 7 d p.i. |
|---|---|---|---|---|---|
| Blood | 0.38 ± 0.02 | 0.03 ± 0.02 | 0.01 ± 0.00 | 0.02 ± 0.01 | 0.01 ± 0.00 |
| Heart | 0.20 ± 0.03 | 0.06 ± 0.01 | 0.12 ± 0.00 | 0.03 ± 0.01 | 0.02 ± 0.01 |
| Lung | 0.54 ± 0.11 | 0.19 ± 0.04 | 0.06 ± 0.01 | 0.08 ± 0.02 | 0.10 ± 0.01 |
| Liver | 7.19 ± 1.62 | 6.54 ± 1.52 | 5.49 ± 0.36 | 5.42 ± 0.92 | 5.49 ± 1.10 |
| Spleen | 3.21 ± 0.44 | 1.90 ± 1.00 | 2.45 ± 1.47 | 3.68 ± 3.80 | 1.91 ± 1.33 |
| Pancreas | 0.12 ± 0.03 | 0.46 ± 0.68 | 0.15 ± 0.21 | 0.02 ± 0.00 | 0.03 ± 0.02 |
| Kidneys | 128.19 ± 6.67 | 59.87 ± 2.53 | 14.45 ± 1.96 | 7.78 ± 2.35 | 4.12 ± 0.90 |
| Stomach | 0.20 ± 0.06 | 0.06 ± 0.01 | 0.08 ± 0.09 | 0.03 ± 0.01 | 0.02 ± 0.00 |
| Small intestine | 0.26 ± 0.03 | 0.07 ± 0.01 | 0.03 ± 0.02 | 0.02 ± 0.01 | 0.01 ± 0.01 |
| Large intestine | 0.19 ± 0.04 | 0.19 ± 0.09 | 0.02 ± 0.01 | 0.03 ± 0.02 | 0.02 ± 0.01 |
| Bladder | 3.98 ± 3.69 | 1.14 ± 1.37 | 0.96 ± 1.49 | 0.19 ± 0.04 | 0.15 ± 0.04 |
| Fat | 1.03 ± 1.14 | 0.12 ± 0.06 | 0.00 ± 0.00 | 0.02 ± 0.00 | 0.01 ± 0.00 |
| Muscle | 0.12 ± 0.04 | 0.08 ± 0.07 | 0.00 ± 0.00 | 0.01 ± 0.00 | 0.01 ± 0.00 |
| Bone | 1.00 ± 1.10 | 0.38 ± 0.19 | 0.50 ± 0.24 | 0.72 ± 0.36 | 0.49 ± 0.15 |
| Brain | 0.01 ± 0.01 | 0.01 ± 0.01 | 0.00 ± 0.00 | 0.01 ± 0.20 | 0.00 ± 0.00 |
| Tumor wild-type | 0.32 ± 0.06 | 0.18 ± 0.03 | 0.08 ± 0.01 | 0.06 ± 0.01 | 0.04 ± 0.02 |
| Tumor PD-L1+ | 7.46 ± 4.70 | 8.82 ± 2.57 | 2.84 ± 0.32 | 1.28 ± 0.27 | 0.49 ± 0.09 |

¹⁷⁷Lu-Adnectin showed high PD-L1 specific uptake after 1 h p.i. in PD-L1 positive tumor xenografts with prolonged retention up to 24 h p.i, while the tracer was effectively cleared from the background, except from the liver and spleen, which leads to increasing tumor-to-background ratios. After 24 h p.i. PD-L1 positive tumor accumulation significantly decreased from 8.8% ID/g to 0.5% ID/g within 7 d p.i. Due to the high PD-L1 expressing tumor uptake combined with low background accumulation at early imaging time points and the, however fast wash-out from PD-L1 positive tumor tissue within 5 d p.i., ¹⁷⁷Lu-Adnectin seems to be more feasible for imaging of PD-L1 expression in tumors than for therapeutic applications of PD-L1 ligands.

### Dosimetry of ¹⁷⁷Lu-Adnectin

TABLE 4
Absorbed doses in different organs [mGy/Mbq] after
application of ¹⁷⁷Lu-Adnectin. Extrapolated from
mouse biodistribution data in U698M-PDL1+ and
U698M tumor-bearing NSG mice at 1 h, 24 h,
72 h, 5 d and 7 d p.i. (see Table 3)
Organ Doses after Application of ¹⁷⁷Lu-Adnectin [mGy/MBq]

| | | | |
|---|---|---|---|
| Adrenals | 1.03E−02 | Ovaries | 5.34E−03 |
| Brain | 3.87E−04 | Pancreas | 1.33E−02 |
| Breasts | 4.54E−03 | Red marrow | 4.62E−03 |
| Gallbladder wall | 1.13E−02 | Osteogenic cells | 1.47E−02 |
| Lower large | 5.16E−03 | Skin | 4.37E−03 |
| intestine wall | | Spleen | 9.76E−02 |
| Small intestine | 6.22E−03 | Testes | 4.47E−03 |
| Stomach wall | 6.43E−03 | Thymus | 4.56E−03 |
| Upper large | 6.54E−03 | Thyroid | 4.18E−03 |
| intestine wall | | Urinary bladder | 2.04E−01 |
| Heart wall | 3.34E−03 | Uterus | 5.97E−03 |
| Kidneys | 1.37E+00 | Total Body | 1.84E−02 |
| Liver | 2.80E−01 | Effective Dose | 6.35E−02 |
| Lungs | 7.43E−03 | | |
| Muscle | 1.40E−03 | | |

The calculated mean specific absorbed 3-doses of ¹⁷⁷Lu-Adnectin to the PD-L1 positive and to the wild-type U-698-M tumors determined values of 0.26 Gy/MBq and 0.017 Gy/MBq.

### Small-Animal SPECT-Imaging of ¹⁷⁷Lu-mAb

To validate the in vivo specificity of ¹⁷⁷Lu-mAb towards PD-L1, SPECT/CT imaging was performed in 2 NSG mice bearing PDL1 positive U-689-M and U-698-M wild-type tumors (FIG. 11). The SPECT images of ¹⁷⁷Lu-mAb revealed in both mice a typical distribution profile known for monoclonal mAbs with high blood-pool activity and accumulation in non-target tissue at early imaging time points (5 h p.i.). After 72 h p.i. significant clearance of background activity was observed. However, hepatic activity levels remain persistently high up to 7 d p.i. After 24 h p.i. high accumulation of ¹⁷⁷Lu-mAb in PD-L1 positive tumors with no uptake in the control tumors was observed, demonstrating specific binding of ¹⁷⁷Lu-mAb to PD-L1 (FIG. 11). Interestingly, for mouse 1 (FIG. 11A) a decrease in tumor volume within a week was observed, probably due to the therapeutic absorbed doses delivered by the amount of ¹⁷⁷Lu-mAb used for SPECT imaging.

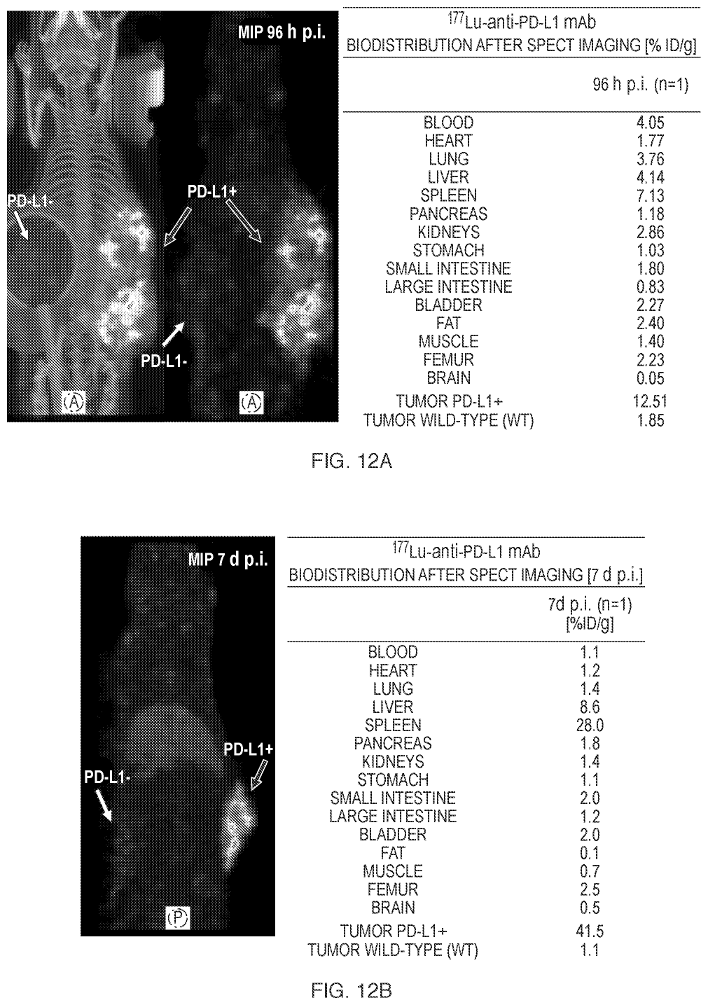

Biodistribution of the SPECT mice displayed 96 h p.i. low blood pool activity, resulting in low unspecific whole-body uptake with significantly elevated uptake of ¹⁷⁷Lu-mAb in PD-L1 positive xenografts (12.5% ID/g at 96 h p.i.; 41% ID/g at 7 d p.i.) in comparison to the U-698-M wild-type tumors (1.9% ID/g at 96 h p.i.; 1.1% ID/g at 7 d p.i), demonstrating successful PD-L1 targeting (FIG. 12).

The biodistribution data 7 d p.i. showed increased blood pool clearance with low background accumulation of the tracer (FIG. 12B) observed in comparison to 96 h p.i., except for the liver and splenic uptake of ¹⁷⁷Lu-mAb, due to the hepatobiliary excretion of ¹⁷⁷Lu-mAb. ¹⁷⁷Lu-mAb specifically binds to human PD-L1 with no binding towards murine PD-L1. Therefore, the high splenic uptake of ¹⁷⁷Lu-mAb in immunodeficient NSG mice is explainable by interactions between the Fc portion of the antibody with murine cells expressing Fc-receptors in non-target organs like the spleen (Sharma et al., Cancer Res. 78(7):1820-1832, 2018). In addition, sequestration of antibody-based agents within the spleen in highly immunodeficient mice, like NSG have been reported (Lyon et al., Nat. Biotechnol. 32:1059-1062, 2014).

¹⁷⁷Lu-mAb showed significantly high and PD-L1 specific uptake (41.5% ID/g) in PDL-1 positive tumor tissue up to 7 d p.i. The significantly higher uptake of ¹⁷⁷Lu-mAb in PD-L1 positive xenografts within 7 d p.i (41.5% ID/g) indicates continuous delivery of the tracer to targeted tissue over time (FIG. 12). In addition, a shrinkage of the PD-L1 positive tumor within 7 d p.i. was observed, indicating a therapeutic effect due to the administration of 177Lu-mAb.

### Biodistribution of ¹⁷⁷Lu-mAb

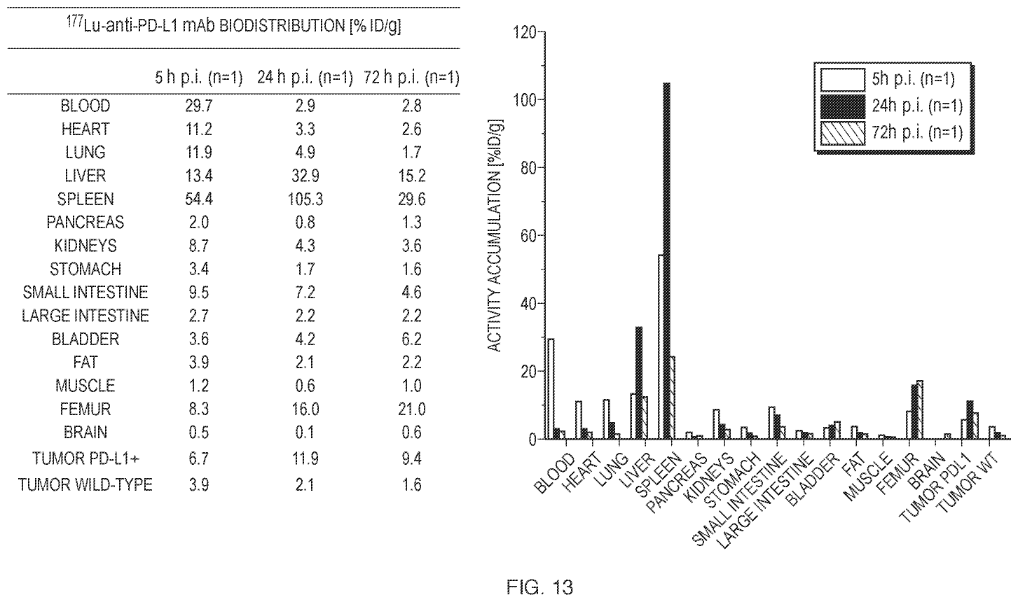

A preliminary biodistribution of ¹⁷⁷Lu-mAb in 3 mice at 5 h, 24 h and 72 h p.i. is shown in FIG. 13. High blood pool and background activity at 5 h p.i. with continuous clearance from non-target tissue within 72 h p.i. was observed. In organs known for degradation of full-sized antibodies, like the liver and spleen, peak uptake was noted 24 h p.i. with significant reduction after 72 h p.i. Whereas clearance of ¹⁷⁷Lu-mAb in all organs was observed within 72 h p.i., bone uptake increased over time. This effect is possibly caused by interactions of the antibody Fc-domain with Fc-γ-receptor expressing innate immune effector cells such as immature macrophages and dendritic cells within the bone marrow of NSG mice (Jonnalagadda et al., Mol. Ther. 2015; 23: 757-768; Guilliams et al., Nat Rev Immunol. 2014; 14:94-108). ¹⁷⁷Lu-mAb showed PD-L1 mediated uptake in PD-L1 expressing tumors over time which was confirmed to be specific due to negligible uptake in PD-L1 negative U-698-M wild-type tumors.

In order to complete the biodistribution data of ¹⁷⁷Lu-mAb (3 mice per time point), 12 additional mice were sacrificed at 5 h (n=2), 24 h (n=2), 72 h (n=2), 5 d (n=3) and 7 d p.i (n=3). The data are summarized in Table 5.

TABLE 5
Biodistribution of ¹⁷⁷Lu-anti PD-L1 mAb in U698M-PDL1+ and U698M
tumor-bearing NSG mice at 5 h (n = 2), 24 h p.i. (n = 2), 72 h p.i. (n = 2),
5 d p.i. (n = 3) and 7 d p.i. (n = 3). Injected Dose: 0.5-0.6 MBq;
15 μg mAb/mouse. Data are expressed as % ID/g (mean ± SD).
¹⁷⁷Lu-anti-PD-L1 mAb-Biodistribution [% ID/g]

| | 5 h p.i | 24 h p.i. | 72 h p.i | 5 d p.i | 7 d p.i |
|---|---|---|---|---|---|
| | (n = 2) | (n = 2) | (n = 2) | (n = 3) | (n = 3) |
| Blood | 11.7 ± 1.5 | 4.8 ± 0.5 | 1.4 ± 0.6 | 0.4 ± 0.1 | 0.2 ± 0.0 |
| Heart | 3.2 ± 1.4 | 4.1 ± 0.2 | 1.4 ± 0.2 | 0.7 ± 0.0 | 0.4 ± 0.1 |
| Lung | 12.8 ± 0.8 | 5.4 ± 0.5 | 1.4 ± 0.2 | 0.6 ± 0.0 | 0.5 ± 0.2 |
| Liver | 18.3 ± 1.5 | 21.8 ± 0.9 | 10.7 ± 0.8 | 7.4 ± 0.4 | 6.7 ± 0.7 |
| Spleen | 3.9 ± 1.9 | 93.5 ± 6.0 | 64.3 ± 12.2 | 50.2 ± 13.4 | 62.6 ± 22.3 |
| Pancreas | 8.1 ± 7.0 | 1.0 ± 0.2 | 0.3 ± 0.2 | 0.2 ± 0.0 | 0.1 ± 0.0 |
| Kidneys | 9.3 ± 3.3 | 4.5 ± 0.6 | 1.7 ± 0.2 | 1.0 ± 0.1 | 0.8 ± 0.1 |
| Stomach | 1.5 ± 0.3 | 1.8 ± 0.3 | 0.3 ± 0.0 | 0.2 ± 0.0 | 0.1 ± 0.0 |
| Small Intestine | 6.1 ± 0.1 | 4.3 ± 1.0 | 1.5 ± 0.1 | 0.8 ± 0.1 | 0.5 ± 0.1 |
| Large Intestine | 2.2 ± 0.0 | 2.6 ± 0.0 | 0.7 ± 0.1 | 0.5 ± 0.0 | 0.5 ± 0.2 |
| Bladder | 3.1 ± 1.7 | 3.5 ± 0.2 | 1.3 ± 0.7 | 1.1 ± 0.1 | 0.8 ± 0.3 |
| Fat | 1.4 ± 0.1 | 0.6 ± 0.1 | 0.5 ± 0.0 | 0.4 ± 0.2 | 0.5 ± 0.2 |
| Muscle | 0.8 ± 0.1 | 0.6 ± 0.0 | 0.2 ± 0.0 | 0.2 ± 0.2 | 0.1 ± 0. |
| Femur | 11.9 ± 2.3 | 11.0 ± 2.0 | 13.6 ± 5.2 | 8.6 ± 3.5 | 6.9 ± 3.2 |
| Brain | 0.2 ± 0.1 | 0.2 ± 0.0 | 1.3 ± 0.1 | 0.0 ± 0.0 | 0.0 ± 0.0 |
| Tumor PD-L1+ | 5.3 ± 1.2 | 6.6 ± 1.0 | 4.5 ± 0.3 | 1.7 ± 0.1 | 1.0 ± 0.2 |
| Tumor wild- | 4.1 ± 0.7 | 5.2 ± 0.2 | 2.2 ± 0.2 | 1.5 ± 0.0 | 0.7 ± 0.6 |
| type | | | | | |

Surprisingly, biodistribution of ¹⁷⁷Lu-anti-PD-L1 mAb in U698M-PDL1+ and U698M tumor-bearing NSG mice revealed only slight differences in the ¹⁷⁷Lu-mAb uptake in U698M-PDL1+ and U698M wild-type tumor xenografts, indicating high unspecific binding in both tumor types. A moderate/low uptake of ¹⁷⁷Lu-mAb in wild-type U-698-M xenografts was to be expected, due to enhanced permeability and retention (EPR) effect of tumor tissue, which favors non-specific tumor accumulation of larger molecules. In addition, low unspecific uptake of different PD-L1-targeting radiolabeled antibodies was observed preclinically in PD-L1 negative tumor tissues. However, the low specific uptake of ¹⁷⁷Lu-mAb in PD-L1 positive tissues might be explained by too low amounts of radiolabeled antibody injected in the mice (0.5-0.6 MBq 177Lu-mAb, 15 μg mAb). Together with the low ¹⁷⁷Lu-labeling efficiency of mAb (˜10% RCY), these results indicate that probably only a small amount of intact ¹⁷⁷Lu-mAb containing non-intact mAb fractions were injected in mice.

The biodistribution of ¹⁷⁷Lu-mAb was repeated in PD-L1 positive and U-698-M wild-type tumor-bearing NSG mice at 5 h, 24 h, 72 h, 5 d and 7 d p.i (n=12). The data are summarized in Table 6. DOTA-conjugated anti-hPD-L1 mAb was formulated in PBS buffer (2.0 mg/mL, pH 7.4) and tested to be intact. In addition, due to the fact that the spleen is a significant sink for PD-L1 antibodies and to increase the amount of available 177Lu-mAb in the tumor environment, higher amounts of ¹⁷⁷Lu-mAb were injected in the mice as for the previous biodistribution where nearly similar uptake in PD-L1 positive and wild-type xenografts was observed (6-7 MBq, ˜30 μg mAb/mouse (new batch) vs. 0.5-0.6 MBq; 15 μg mAb/mouse previous biodistribution; see Table 5).

A comparable organ distribution of ¹⁷⁷Lu-mAb was observed as seen in the preliminary biodistribution in 3 mice (FIG. 9). A high blood pool and background activity at 5 h p.i. with continuous clearance from non-target tissue within 24 h p.i. was observed, except of the liver and the spleen, where uptake remains nearly constant within 7 d p.i., due to the slow hepatobiliary excretion and the degradation of full-sized antibodies in this organs. In addition, the high splenic uptake and the increasing accumulation of radioactivity in the bone over time might be explainable by interactions between the Fc portion of the antibody with murine cells expressing Fc-receptors in non-target organs like the spleen and innate immune effector cells such as immature macrophages and dendritic cells within the bone marrow of NSG mice [1, 3, 4]. In contrast to the previous biodistribution showing nearly no differences in the 177Lu-mAb uptake in U-698-M-PDL1+ and U-698-M wild-type tumor xenografts (Table 5) the biodistribution data obtained with the new batch of 177Lu-mAb showed PD-L1 mediated uptake in PD-L1 expressing tumors over time which was confirmed to be specific due to negligible and 2-3 fold lower uptake in PD-L1 negative U-698-M wild-type tumors. Peak tumor uptake of 177Lu-mAb in PD-L1 positive xenografts was noted 72 h p.i. (13.1±7.2 ID/g), with a reduction to 2.9±1.2 ID/g within 7 d p.i, respectively. In addition, the wild-type U-698-M tumors were significantly larger than PD-L1 positive xenografts, which could have an effect on enhanced permeability of tumor tissue, resulting in higher unspecific antibody uptake in PD-L1 negative tumors.

TABLE 6
Second Biodistribution of ¹⁷⁷Lu-anti PD-L1 mAb (new batch)
in U698M-PDL1+ and U698M tumor-bearing
NSG mice at 5 h (n = 3), 24 h p.i. (n = 3),
72 h p.i. (n = 3), 5 d p.i. (n = 3) and 7 d p.i.
(n = 3). Injected Dose: 6-7 MBq; app.
30 μg mAb/mouse. Data are expressed as % ID/g (mean ± SD).
¹⁷⁷Lu-anti-PD-L1 mAb (new batch)-Biodistribution [% ID/g]

| | 5 h p.i | 24 h p.i. | 72 h p.i | 5 d p.i | 7 d p.i |
|---|---|---|---|---|---|
| | (n = 3) | (n = 3) | (n = 3) | (n = 3) | (n = 3) |
| Blood | 17.7 ± 1.5 | 2.6 ± 0.8 | 2.1 ± 1.0 | 0.6 ± 0.2 | 0.3 ± 0.01 |
| Heart | 6.8 ± 1.5 | 2.2 ± 0.3 | 1.6 ± 0.8 | 0.6 ± 0.02 | 0.3 ± 0.1 |
| Lung | 10.3 ± 2.6 | 2.8 ± 0.1 | 2.0 ± 0.3 | 0.8 ± 0.3 | 0.4 ± 0.2 |
| Liver | 8.9 ± 1.4 | 11.7 ± 6.5 | 10.6 ± 4.0 | 6.5 ± 1.2 | 4.1 ± 0.5 |
| Spleen | 34.6 ± 7.5 | 50.1 ± 13.6 | 35.4 ± 5.1 | 38.3 ± 5.2 | 48.0 ± 6.0 |
| Pancreas | 1.4 ± 0.2 | 1.2 ± 0.7 | 1.5 ± 0.3 | 0.4 ± 0.2 | 0.3 ± 0.2 |
| Kidneys | 5.8 ± 0.2 | 2.5 ± 0.07 | 2.8 ± 0.7 | 1.5 ± 0.2 | 1.0 ± 0.1 |
| Stomach | 2.4 ± 0.3 | 1.3 ± 0.3 | 0.8 ± 0.4 | 0.4 ± 0.1 | 0.2 ± 0.04 |
| Small | 4.8 ± 1.2 | 3.2 ± 0.8 | 2.8 ± 1.8 | 1.0 ± 0.3 | 0.5 ± 0.04 |
| Intestine | | | | | |
| Large | 2.2 ± 0.8 | 1.4 ± 0.3 | 1.7 ± 0.6 | 0.8 ± 0.4 | 0.3 ± 0.03 |
| Intestine | | | | | |
| Bladder | 2.7 ± 0.7 | 2.6 ± 0.3 | 3.5 ± 2.4 | 1.5 ± 0.2 | 0.9 ± 0.1 |
| Fat | 2.1 ± 0.6 | 1.3 ± 0.08 | 1.6 ± 0.5 | 1.0 ± 0.4 | 1.0 ± 0.3 |
| Muscle | 0.8 ± 0.02 | 0.6 ± 0.3 | 1.0 ± 0.6 | 0.6 ± 0.1 | 0.4 ± 0.2 |
| Femur | 5.8 ± 0.7 | 7.2 ± 1.8 | 13.8 ± 6.5 | 8.6 ± 1.8 | 6.0 ± 1.7 |
| Brain | 0.3 ± 0.03 | 0.1 ± 0.1 | 0.8 ± 0.1 | 0.1 ± 0.03 | 0.1 ± 0.04 |
| Tumor | 8.0 ± 2.0 | 6.6 ± 0.3 | 13.1 ± 7.2 | 3.3 ± 0.3 | 2.9 ± 1.2 |
| PD-L1+ | | | | | |
| Tumor | 4.2 ± 1.5 | 3.1 ± 1.6 | 3.9 ± 1.9 | 1.4 ± 1.1 | 1.0 ± 0.3 |
| wild-type | | | | | |

### Dosimetry of ¹⁷⁷Lu-mAb.

TABLE 7
Absorbed doses in different organs [mGy/Mbq]
after application of ¹⁷⁷Lu-mAb.
Extrapolated from mouse biodistribution
data in U698M-PDL1+ and U698M tumor-bearing
NSG mice at 5 h, 24 h, 72 h, 5 d and 7 d p.i (Table 6).
Organ Doses after Application of ¹⁷⁷Lu-mAb [mGy/MBq]

| | | | |
|---|---|---|---|
| Adrenals | 6.83E−02 | Ovaries | 7.43E−02 |
| Brain | 8.04E−03 | Pancreas | 1.30E−01 |
| Breasts | 5.75E−02 | Red marrow | 5.17E−02 |
| Gallbladder wall | 7.70E−02 | Osteogenic cells | 2.07E−01 |
| Lower large intestine wall | 2.50E−00 | Skin | 5.57E−02 |
| Small intestine | 2.32E−01 | Spleen | 2.18E−00 |
| Stomach wall | 7.62E−02 | Testes | 5.79E−02 |
| Upper large intestine wall | 8.19E−01 | Thymus | 5.89E−02 |
| Heart wall | 1.45E−01 | Thyroid | 5.74E−02 |
| Kidneys | 1.92E−01 | Urinary bladder | 8.46E−02 |
| Liver | 7.75E−01 | Uterus | 6.69E−02 |
| Lungs | 2.21E−01 | Total Body | 9.97E−02 |
| Muscle | 8.59E−03 | Effective Dose | 4.27E−01 |

The calculated mean specific absorbed 3-doses of ¹⁷⁷Lu-mAb to the PD-L1 positive and to the wild-type U-698-M tumors determined values of 1.26 Gy/MBq and 0.054 Gy/MBq, respectively.

Therapy Study with ¹⁷⁷Lu-mAb

Due to the higher tumor-to-organ radiation dose ratios of ¹⁷⁷Lu-mAb and the higher retention in PD-L1 positive tissues over 7 d p.i. in comparison to ¹⁷⁷Lu-Adnectin, the potential of ¹⁷⁷Lu-mAb for radioimmunotherapy was evaluated to assess the effectiveness and toxicity of PD-L1 targeted radionuclide therapy.

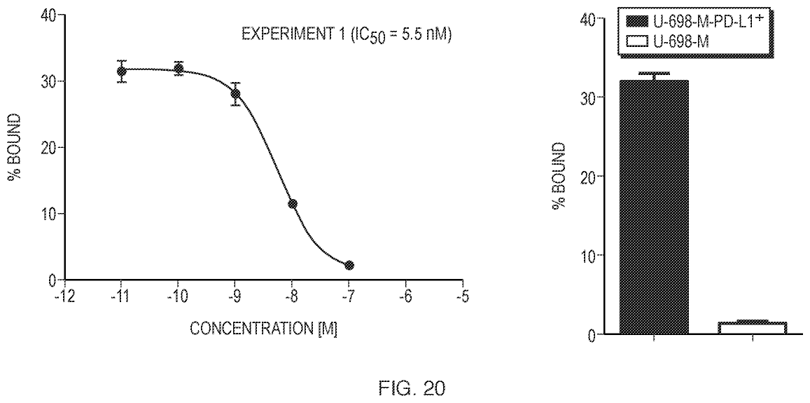

Before starting the therapy study using ¹⁷⁷Lu-mAb, the binding and specificity of ¹⁷⁷Lu-mAb towards human PD-L1 using transduced PD-L1 positive U-698-M cells was again investigated in a competitive binding assay to ensure using intact ¹⁷⁷Lu-mAb for therapeutic studies in mice (FIG. 20). ¹⁷⁷Lu-mAb showed high affinity towards human PD-L1 with an IC₅₀ value of 5.5 nM (FIG. 20, left panel). Specific binding was confirmed using non-transfected U-689-M cells as a negative control, demonstrating in vitro binding of ¹⁷⁷Lu-mAb to tumor cells in a PD-L1 expression-dependent manner (FIG. 20, right panel).

In parallel, additional FACS analysis of the transduced PD-L1 positive and wild-type U-689-M cells were performed to ensure stable PD-L1 expression of the transduced cell line before inoculation of both cell lines in mice for therapeutic investigations using ¹⁷⁷Lu-mAb. Quantification of PD-L1 expression on transduced and wild-type U-698-M cells, determined by FACS analysis revealed a low PD-L1 expression for wild-type U-698-M cells.

In contrast, high PD-L1 expression was observed for stable transduced U-698-M cells. Therefore, both cell lines were suitable for the establishment of PD-L1 positive and PD-L1 negative xenografts for the in vivo evaluation of ¹⁷⁷Lu-mAb radioimmunotherapy.

### Conclusion

¹⁷⁷Lu-Adnectin and ¹⁷⁷Lu-mAb were obtained with quantitative RCYs and high RCP (>95%). SPECT imaging and biodistribution studies of ¹⁷⁷Lu-Adnectin revealed high uptake in PD-L1+ tumors (7.5±4.7% ID/g at 1 h p.i.) and kidneys (129.2±6.7% ID/g at 1 h p.i.) with negligible uptake in PD-L1 negative tumors (0.3±0.1% ID/g at 1 h p.i.) and other tissues at early time points. ¹⁷⁷Lu-Adnectin was effectively cleared from the kidneys over time, however after 24 h p.i. tracer uptake in PD-L1 positive tumors decreased significantly to 0.5% ID/g within 7 d p.i. SPECT imaging of ¹⁷⁷Lu-mAb showed a typical distribution profile known for mAb with high blood pool and background activity at early imaging time points. After 72 h p.i. significant background clearance was observed with high and persistent uptake of ¹⁷⁷Lu-mAb in PD-L1 positive tumors. Specific binding of ¹⁷⁷Lu-mAb to PD-L1 was further confirmed by negligible uptake in PD-L1 negative tumor tissue. Preliminary biodistribution data of ¹⁷⁷Lu-mAb are comparable to those results obtained with SPECT imaging (n=3).

¹⁷⁷Lu-Adnectin and ¹⁷⁷Lu-mAb showed excellent in vivo PD-L1 targeting characteristics. ¹⁷⁷Lu-Adnectin revealed high and specific tumor uptake combined with low background accumulation at early imaging time points. As seen in SPECT and biodistribution studies ¹⁷⁷Lu-Adnectin showed a fast clearance with only low retention in PD-L1 positive tumor tissue within 7 d p.i., resulting in low tumor-to-organ radiation dose ratios together with a high kidney dose. However, due to the fast clearance of ¹⁷⁷Lu-Adnectin from PD-L1 positive tumor tissue within 7 d p.i., Adnectin seems to be more feasible for PD-L1-targeted imaging, for example, using ⁶⁸Ga-labeled Adnectin.

In contrast, ¹⁷⁷Lu-mAb showed prolonged high PD-L1 specific tumor uptake combined with continuous clearance of background activity within 7 d p.i. ¹⁷⁷Lu-mAb SPECT imaging revealed a target specific distribution profile with high blood pool and background activity at early imaging time points. After 24 to 72 h p.i. significant background clearance was observed with high and persistent uptake of ¹⁷⁷Lu-mAb in PD-L1 positive tumors within 7 d p.i. Dosimetry calculations of ¹⁷⁷Lu-mAb revealed acceptable high tumor-to-organ radiation doses indicating the effectiveness of PD-L1 targeted radioimmunotherapy treatment of PD-L1 positive xenografts suggesting a high potential for PD-L1 targeted radionuclide therapy. In addition, ¹⁷⁷Lu-mAb SPECT imaging can be potentially used to perform treatment planning or to determine response to immunotherapy

TABLE 8
SEQUENCE LISTING

| SEQ | | |
|---|---|---|
| ID | DESCRIPTION | SEQUENCE |
| 1 | Full length wild-type | VSDVPRDLEVVAATPTSLLISWDAPAVTVRYYRITYGETGGNS |
| | human ¹⁰Fn3 domain | PVQEFTVPGSKSTATISGLKPGVDYTITVYAVTGRGDSPASSK |
| | | PISINYRT |
| 2 | Core wild-type human | EVVAATPTSLLISWDAPAVTVRYYRITYGETGGNSPVQEFTVP |
| | ¹⁰Fn3 domain | GSKSTATISGLKPGVDYTITVYAVTGRGDSPASSKPISINYRT |
| 3 | Core ¹⁰Fn3-based scaffold | EVVAA(Z)ₐLLISW(Z)ₓYRITY(Z)_{b}FTV(Z)_{y}ATISGI(Z)_{c}Y |
| | with variable AB, BC, CD, | TITVYA(Z)_{z}ISINYRT |
| | DE, EF, and FG loops | |
| 4 | Core ¹⁰Fn3-based scaffold | EVVAATPTSLLISW(Z)ₓYRITYGETGGNSPVQEFTV(Z)_{y}ATISG |
| | with variable BC, DE, and | LKPGVDYTITVYA(Z)_{z}ISINYRT |
| | FG loops | |
| 5 | ATI-968 core (aka | EVVAATPTSLLISWIAPFYNVIYYRITYGETGGNSPVQEFTVPGTG |
| | ADX_1760_C01) | YTATISGLKPGVDYTITVYAVTDGASIASYAFPISINYRT |
| 6 | ATI-968 BC loop | IAPFYNVIY |
| 7 | ATI-968 DE loop | PGTGYT |
| 8 | ATI-968 FG loop | VTDGASIASYAFP |
| 9 | ATI-968 w/ N leader | GVSDVPRDLEVVAATPTSLLISWIAPFYNVIYYRITYGETGGNSPV |
| | | QEFTVPGTGYTATISGLKPGVDYTITVYAVTDGASIASYAFPISIN |
| | | YRT |
| 10 | ATI-968 w/ N leader + his | GVSDVPRDLEVVAATPTSLLISWIAPFYNVIYYRITYGETGGNSPV |
| | tag | QEFTVPGTGYTATISGLKPGVDYTITVYAVTDGASIASYAFPISIN |
| | | YRTHHHHHH |
| 11 | ATI-968 w/ N leader and C | GVSDVPRDLEVVAATPTSLLISWIAPFYNVIYYRITYGETGGNSPV |
| | tail | QEFTVPGTGYTATISGLKPGVDYTITVYAVTDGASIASYAFPISIN |
| | | YRTEIDKPSQ |
| 12 | ATI-968 w/ N leader and C | GVSDVPRDLEVVAATPTSLLISWIAPFYNVIYYRITYGETGGNSPV |
| | tail + his tag | QEFTVPGTGYTATISGLKPGVDYTITVYAVTDGASIASYAFPISIN |
| | | YRTEIDKPSQHHHHHH |
| 13 | ATI-968 w/ N leader and | GVSDVPRDLEVVAATPTSLLISWIAPFYNVIYYRITYGETGGNSPV |
| | modified C-terminus | QEFTVPGTGYTATISGLKPGVDYTITVYAVTDGASIASYAFPISIN |
| | including PC | YRTPC |
| 14 | ATI-968 w/ N leader and | GVSDVPRDLEVVAATPTSLLISWIAPFYNVIYYRITYGETGGNSPV |
| | modified C-terminus | QEFTVPGTGYTATISGLKPGVDYTITVYAVTDGASIASYAFPISIN |
| | including PC + his tag | YRTPCHHHHHH |
| 15 | ATI-968-full length | MGVSDVPRDLEVVAATPTSLLISWIAPFYNVIYYRITYGETGGNSP |
| | | VQEFTVPGTGYTATISGLKPGVDYTITVYAVTDGASIASYAFPSI |
| | | NYRTEIDKPSQHHHHHH |
| 16 | ATI-968-core (nucleotide | GAAGTGGTTGCTGCCACCCCCACCAGCCTGCTGATCAGCTGGATCG |
| | sequence) | CTCCGTTCTACAATGTCATCTATTACCGCATCACTTACGGCGAAAC |
| | | AGGAGGCAATAGCCCTGTCCAGGAGTTCACTGTGCCTGGTACTGGT |
| | | TATACAGCTACAATCAGCGGCCTTAAACCTGGCGTTGATTATACCA |
| | | TCACTGTGTATGCTGTCACTGATGGAGCATCCATTGCTTCATACGC |
| | | GTTTCCAATTTCCATTAATTACCGCACA |
| 17 | ATI-968 w/ N leader | ATGGGAGTTTCTGATGTGCCGCGCGACCTGGAAGTGGTTGCTGCCA |
| | (nucleotide sequence with | CCCCCACCAGCCTGCTGATCAGCTGGATCGCTCCGTTCTACAATGT |
| | N-terminal methionine) | CATCTATTACCGCATCACTTACGGCGAAACAGGAGGCAATAGCCCT |
| | | GTCCAGGAGTTCACTGTGCCTGGTACTGGTTATACAGCTACAATCA |
| | | GCGGCCTTAAACCTGGCGTTGATTATACCATCACTGTGTATGCTGT |
| | | CACTGATGGAGCATCCATTGCTTCATACGCGTTTCCAATTTCCATT |
| | | AATTACCGCACA |
| 18 | ATI-968 w/ N leader and | ATGGGAGTTTCTGATGTGCCGCGCGACCTGGAAGTGGTTGCTGCCA |
| | modified C-terminus | CCCCCACCAGCCTGCTGATCAGCTGGATCGCTCCGTTCTACAATGT |
| | including PC (nucleotide | CATCTATTACCGCATCACTTACGGCGAAACAGGAGGCAATAGCCCT |
| | sequence with N-terminal | GTCCAGGAGTTCACTGTGCCTGGTACTGGTTATACAGCTACAATCA |
| | methionine) | GCGGCCTTAAACCTGGCGTTGATTATACCATCACTGTGTATGCTGT |
| | | CACTGATGGAGCATCCATTGCTTCATACGCGTTTCCAATTTCCATT |
| | | AATTACCGCACACCGTGC |
| 19 | ATI-968 w/ N leader and C | ATGGGAGTTTCTGATGTGCCGCGCGACCTGGAAGTGGTTGCTGCCA |
| | tail + his tag (nucleotide | CCCCCACCAGCCTGCTGATCAGCTGGATCGCTCCGTTCTACAATGT |
| | sequence with N-terminal | CATCTATTACCGCATCACTTACGGCGAAACAGGAGGCAATAGCCCT |
| | methionine) | GTCCAGGAGTTCACTGTGCCTGGTACTGGTTATACAGCTACAATCA |
| | | GCGGCCTTAAACCTGGCGTTGATTATACCATCACTGTGTATGCTGT |
| | | CACTGATGGAGCATCCATTGCTTCATACGCGTTTCCAATTTCCATT |
| | | AATTACCGCACAGAAATTGACAAACCATCCCAGCACCATCACCACC |
| | | ACCACTGA |
| 20 | ATI-964 core (parent of | EVVAATPTSLLISWSYDGSIERYYRITYGETGGNSPVQEFTVPPDQ |
| | ADX_5322_A02) | KTATISGLKPGVDYTITVYAVRLEEAHYYRESPISINYRT |
| 21 | ATI-964 BC loop | SYDGSIERY |
| 22 | ATI-964 DE loop | PPDQKT |
| 23 | ATI-964 FG loop | VRLEEAHYYRESP |
| 24 | ATI-964 w/ N leader | GVSDVPRDLEVVAATPTSLLISWSYDGSIERYYRITYGETGGNSPV |
| | | QEFTVPPDQKTATISGLKPGVDYTITVYAVRLEEAHYYRESPISIN |
| | | YRT |
| 25 | ATI-964 w/ N leader + his | GVSDVPRDLEVVAATPTSLLISWSYDGSIERYYRITYGETGGNSPV |
| | tag | QEFTVPPDQKTATISGLKPGVDYTITVYAVRLEEAHYYRESPISIN |
| | | YRTHHHHHH |
| 26 | ATI-964 w/ N leader and C | GVSDVPRDLEVVAATPTSLLISWSYDGSIERYYRITYGETGGNSPV |
| | tail | QEFTVPPDQKTATISGLKPGVDYTITVYAVRLEEAHYYRESPISIN |
| | | YRTEIDKPSQ |
| 27 | ATI-964 w/ N leader and C | GVSDVPRDLEVVAATPTSLLISWSYDGSIERYYRITYGETGGNSPV |
| | tail + his tag | QEFTVPPDQKTATISGLKPGVDYTITVYAVRLEEAHYYRESPISIN |
| | | YRTEIDKPSQHHHHHH |
| 28 | ATI-964 w/ N leader and | GVSDVPRDLEVVAATPTSLLISWSYDGSIERYYRITYGETGGNSPV |
| | modified C-terminus | QEFTVPPDQKTATISGLKPGVDYTITVYAVRLEEAHYYRESPISIN |
| | including PC | YRTPC |
| 29 | ATI-964 w/ N leader and | GVSDVPRDLEVVAATPTSLLISWSYDGSIERYYRITYGETGGNSPV |
| | modified C-terminus | QEFTVPPDQKTATISGLKPGVDYTITVYAVRLEEAHYYRESPISIN |
| | including PC + his tag | YRTPCHHHHHH |
| 30 | ATI-964-full length | MGVSDVPRDLEVVAATPTSLLISWSYDGSIERYYRITYGETGGNSP |
| | | VQEFTVPPDQKTATISGLKPGVDYTITVYAVRLEEAHYYRESPISI |
| | | NYRTEIDKPSQHHHHHH |
| 31 | ATI-964-core (nucleotide | GAAGTGGTTGCTGCCACCCCCACCAGCCTGCTGATCAGCTGGTCTT |
| | sequence) | ACGACGGTTCGATTGAACGTTATTACCGCATCACTTACGGCGAAAC |
| | | AGGAGGCAATAGCCCTGTCCAGGAGTTCACTGTGCCTCCGGATCAG |
| | | AAGACAGCTACCATCAGCGGCCTTAAACCTGGCGTTGATTATACCA |
| | | TCACTGTGTATGCTGTCAGGCTGGAAGAAGCTCATTACTATCGAGA |
| | | GTCTCCAATTTCCATTAATTACCGCACA |
| 32 | ATI-964 w/ N leader | ATGGGAGTTTCTGATGTGCCGCGCGACCTGGAAGTGGTTGCTGCCA |
| | (nucleotide sequence with | CCCCCACCAGCCTGCTGATCAGCTGGTCTTACGACGGTTCGATTGA |
| | N-terminal methionine) | ACGTTATTACCGCATCACTTACGGCGAAACAGGAGGCAATAGCCCT |
| | | GTCCAGGAGTTCACTGTGCCTCCGGATCAGAAGACAGCTACCATCA |
| | | GCGGCCTTAAACCTGGCGTTGATTATACCATCACTGTGTATGCTGT |
| | | CAGGCTGGAAGAAGCTCATTACTATCGAGAGTCTCCAATTTCCATT |
| | | AATTACCGCACA |
| 33 | ATI-964 w/ N leader and | ATGGGAGTTTCTGATGTGCCGCGCGACCTGGAAGTGGTTGCTGCCA |
| | modified C-terminus | CCCCCACCAGCCTGCTGATCAGCTGGTCTTACGACGGTTCGATTGA |
| | including PC (nucleotide | ACGTTATTACCGCATCACTTACGGCGAAACAGGAGGCAATAGCCCT |
| | sequence with N-terminal | GTCCAGGAGTTCACTGTGCCTCCGGATCAGAAGACAGCTACCATCA |
| | methionine) | GCGGCCTTAAACCTGGCGTTGATTATACCATCACTGTGTATGCTGT |
| | | CAGGCTGGAAGAAGCTCATTACTATCGAGAGTCTCCAATTTCCATT |
| | | AATTACCGCACACCGTGC |
| 34 | ATI-964 w/ N leader and C | ATGGGAGTTTCTGATGTGCCGCGCGACCTGGAAGTGGTTGCTGCCA |
| | tail + his tag (nucleotide | CCCCCACCAGCCTGCTGATCAGCTGGTCTTACGACGGTTCGATTGA |
| | sequence with N-terminal | ACGTTATTACCGCATCACTTACGGCGAAACAGGAGGCAATAGCCCT |
| | methionine) | GTCCAGGAGTTCACTGTGCCTCCGGATCAGAAGACAGCTACCATCA |
| | | GCGGCCTTAAACCTGGCGTTGATTATACCATCACTGTGTATGCTGT |
| | | CAGGCTGGAAGAAGCTCATTACTATCGAGAGTCTCCAATTTCCATT |
| | | AATTACCGCACAGAAATTGACAAACCATCCCAGCACCATCACCACC |
| | | ACCACTGA |
| 35 | ATI-965 core | EVVAATPTSLLISWTAYDSVDKYYRITYGETGGNSPVQEFTVGPRH |
| | | HTATISGLKPGVDYTITVYAVYHTEPGYHAHMPISINYRT |
| 36 | ATI-965 BC loop | TAYDSVDKY |
| 37 | ATI-965 DE loop | GPRHHT |
| 38 | ATI-965 FG loop | VYHTEPGYHAHMP |
| 39 | ATI-965 w/ N leader | GVSDVPRDLEVVAATPTSLLISWTAYDSVDKYYRITYGETGGNSPV |
| | | QEFTVGPRHHTATISGLKPGVDYTITVYAVYHTEPGYHAHMPISIN |
| | | YRT |
| 40 | ATI-965 w/ N leader + his | GVSDVPRDLEVVAATPTSLLISWTAYDSVDKYYRITYGETGGNSPV |
| | tag | QEFTVGPRHHTATISGLKPGVDYTITVYAVYHTEPGYHAHMPISIN |
| | | YRTHHHHHH |
| 41 | ATI-965 w/ N leader and C | GVSDVPRDLEVVAATPTSLLISWTAYDSVDKYYRITYGETGGNSPV |
| | tail | QEFTVGPRHHTATISGLKPGVDYTITVYAVYHTEPGYHAHMPISIN |
| | | YRTEIDKPSQ |
| 42 | ATI-965 w/ N leader and C | GVSDVPRDLEVVAATPTSLLISWTAYDSVDKYYRITYGETGGNSPV |
| | tail + his tag | QEFTVGPRHHTATISGLKPGVDYTITVYAVYHTEPGYHAHMPISIN |
| | | YRTEIDKPSQHHHHHH |
| 43 | ATI-965 w/ N leader and | GVSDVPRDLEVVAATPTSLLISWTAYDSVDKYYRITYGETGGNSPV |
| | modified C-terminus | QEFTVGPRHHTATISGLKPGVDYTITVYAVYHTEPGYHAHMPISIN |
| | including PC | YRTPC |
| 44 | ATI-965 w/ N leader and | GVSDVPRDLEVVAATPTSLLISWTAYDSVDKYYRITYGETGGNSPV |
| | modified C-terminus | QEFTVGPRHHTATISGLKPGVDYTITVYAVYHTEPGYHAHMPISIN |
| | including PC + his tag | YRTPCHHHHHH |
| 45 | ATI-965-full length | MGVSDVPRDLEVVAATPTSLLISWTAYDSVDKYYRITYGE |
| | | TGGNSPVQEFTVGPRHHTATISGLKPGVDYTITVYAVYHTEPGYHA |
| | | HMPISINYRTEIDKPSQHHHHHH |
| 46 | ATI-965-core (nucleotide | GAAGTGGTTGCTGCCACCCCCACCAGCCTGCTGATCAGCTGGACTG |
| | sequence) | CATACGACTCTGTTGACAAATATTACCGCATCACTTACGGCGAAAC |
| | | AGGAGGCAATAGCCCTGTCCAGGAGTTCACTGTGGGCCCTAGACAT |
| | | CACACAGCTACCATCAGCGGCCTTAAACCTGGCGTTGATTATACCA |
| | | TCACTGTGTATGCTGTCTATCACACTGAACCGGGCTATCATGCTCA |
| | | TATGCCAATTTCCATTAATTACCGCACA |
| 47 | ATI-965 w/ N leader | ATGGGAGTTTCTGATGTGCCGCGCGACCTGGAAGTGGTTGCTGCCA |
| | (nucleotide sequence with | CCCCCACCAGCCTGCTGATCAGCTGGACTGCATACGACTCTGTTGA |
| | N-terminal methionine) | CAAATATTACCGCATCACTTACGGCGAAACAGGAGGCAATAGCCCT |
| | | GTCCAGGAGTTCACTGTGGGCCCTAGACATCACACAGCTACCATCA |
| | | GCGGCCTTAAACCTGGCGTTGATTATACCATCACTGTGTATGCTGT |
| | | CTATCACACTGAACCGGGCTATCATGCTCATATGCCAATTTCCATT |
| | | AATTACCGCACA |
| 48 | ATI-965 w/ N leader and | ATGGGAGTTTCTGATGTGCCGCGCGACCTGGAAGTGGTTGCTGCCA |
| | modified C-terminus | CCCCCACCAGCCTGCTGATCAGCTGGACTGCATACGACTCTGTTGA |
| | including PC (nucleotide | CAAATATTACCGCATCACTTACGGCGAAACAGGAGGCAATAGCCCT |
| | sequence with N-terminal | GTCCAGGAGTTCACTGTGGGCCCTAGACATCACACAGCTACCATCA |
| | methionine) | GCGGCCTTAAACCTGGCGTTGATTATACCATCACTGTGTATGCTGT |
| | | CTATCACACTGAACCGGGCTATCATGCTCATATGCCAATTTCCATT |
| | | AATTACCGCACACCGTGC |
| 49 | ATI-965 w/ N leader and C | ATGGGAGTTTCTGATGTGCCGCGCGACCTGGAAGTGGTTGCTGCCA |
| | tail + his tag (nucleotide | CCCCCACCAGCCTGCTGATCAGCTGGACTGCATACGACTCTGTTGA |
| | sequence with N-terminal | CAAATATTACCGCATCACTTACGGCGAAACAGGAGGCAATAGCCCT |
| | methionine) | GTCCAGGAGTTCACTGTGGGCCCTAGACATCACACAGCTACCATCA |
| | | GCGGCCTTAAACCTGGCGTTGATTATACCATCACTGTGTATGCTGT |
| | | CTATCACACTGAACCGGGCTATCATGCTCATATGCCAATTTCCATT |
| | | AATTACCGCACAGAAATTGACAAACCATCCCAGCACCATCACCACC |
| | | ACCACTGA |
| 50 | ATI-966 core | EVVAATPTSLLISWHRFSSIMAYYRITYGETGGNSPVQEFTVAGSV |
| | | NTATISGLKPGVDYTITVYAVTIHNVSFPISINYRT |
| 51 | ATI-966 BC loop | HRFSSIMAY |
| 52 | ATI-966 DE loop | AGSVNT |
| 53 | ATI-966 FG loop | VTIHNVSFP |
| 54 | ATI-966 w/ N leader | GVSDVPRDLEVVAATPTSLLISWHRFSSIMAYYRITYGETGGNSPV |
| | | QEFTVAGSVNTATISGLKPGVDYTITVYAVTIHNVSFPISINYRT |
| 55 | ATI-966 w/ N leader + his | GVSDVPRDLEVVAATPTSLLISWHRFSSIMAYYRITYGETGGNSPV |
| | tag | QEFTVAGSVNTATISGLKPGVDYTITVYAVTIHNVSFPISINYRTH |
| | | HHHHH |
| 56 | ATI-966 w/ N leader and C | GVSDVPRDLEVVAATPTSLLISWHRFSSIMAYYRITYGETGGNSPV |
| | tail | QEFTVAGSVNTATISGLKPGVDYTITVYAVTIHNVSFPISINYRTE |
| | | IDKPSQ |
| 57 | ATI-966 w/ N leader and C | GVSDVPRDLEVVAATPTSLLISWHRFSSIMAYYRITYGETGGNSPV |
| | tail + his tag | QEFTVAGSVNTATISGLKPGVDYTITVYAVTIHNVSFPISINYRTE |
| | | IDKPSQHHHHHH |
| 58 | ATI-966 w/ N leader and | GVSDVPRDLEVVAATPTSLLISWHRFSSIMAYYRITYGETGGNSPV |
| | modified C-terminus | QEFTVAGSVNTATISGLKPGVDYTITVYAVTIHNVSFPISINYRTP |
| | including PC | C |
| 59 | ATI-966 w/ N leader and | GVSDVPRDLEVVAATPTSLLISWHRFSSIMAYYRITYGETGGNSPV |
| | modified C-terminus | QEFTVAGSVNTATISGLKPGVDYTITVYAVTIHNVSFPISINYRTP |
| | including PC + his tag | CHHHHHH |
| 60 | ATI-966-full length | MGVSDVPRDLEVVAATPTSLLISWHRFSSIMAYYRITYGETGGNSP |
| | | VQEFTVAGSVNTATISGLKPGVDYTITVYAVTIHNVSFPISINYRT |
| | | EIDKPSQHHHHHH |
| 61 | ATI-966-core (nucleotide | GAAGTGGTTGCTGCCACCCCCACCAGCCTGCTGATCAGCTGGCATA |
| | sequence) | GGTTCTCTTCTATCATGGCGTATTACCGCATCACTTACGGCGAAAC |
| | | AGGAGGCAATAGCCCTGTCCAGGAGTTCACTGTGGCTGGCTCTGTT |
| | | AACACAGCTACCATCAGCGGCCTTAAACCTGGCGTTGATTATACCA |
| | | TCACTGTGTATGCTGTCACGATCCATAACGTTTCTTTCCCAATTTC |
| | | CATTAATTACCGCACA |
| 62 | ATI-966w/ N leader | ATGGGAGTTTCTGATGTGCCGCGCGACCTGGAAGTGGTTGCTGCCA |
| | (nucleotide sequence with | CCCCCACCAGCCTGCTGATCAGCTGGCATAGGTTCTCTTCTATCAT |
| | N-terminal methionine) | GGCGTATTACCGCATCACTTACGGCGAAACAGGAGGCAATAGCCCT |
| | | GTCCAGGAGTTCACTGTGGCTGGCTCTGTTAACACAGCTACCATCA |
| | | GCGGCCTTAAACCTGGCGTTGATTATACCATCACTGTGTATGCTGT |
| | | CACGATCCATAACGTTTCTTTCCCAATTTCCATTAATTACCGCACA |
| 63 | ATI-966 w/ N leader and | ATGGGAGTTTCTGATGTGCCGCGCGACCTGGAAGTGGTTGCTGCCA |
| | modified C-terminus | CCCCCACCAGCCTGCTGATCAGCTGGCATAGGTTCTCTTCTATCAT |
| | including PC (nucleotide | GGCGTATTACCGCATCACTTACGGCGAAACAGGAGGCAATAGCCCT |
| | sequence with N-terminal | GTCCAGGAGTTCACTGTGGCTGGCTCTGTTAACACAGCTACCATCA |
| | methionine) | GCGGCCTTAAACCTGGCGTTGATTATACCATCACTGTGTATGCTGT |
| | | CACGATCCATAACGTTTCTTTCCCAATTTCCATTAATTACCGCACA |
| | | CCGTGC |
| 64 | ATI-966 w/ N leader and C | ATGGGAGTTTCTGATGTGCCGCGCGACCTGGAAGTGGTTGCTGCCA |
| | tail + his tag (nucleotide | CCCCCACCAGCCTGCTGATCAGCTGGCATAGGTTCTCTTCTATCAT |
| | sequence with N-terminal | GGCGTATTACCGCATCACTTACGGCGAAACAGGAGGCAATAGCCCT |
| | methionine) | GTCCAGGAGTTCACTGTGGCTGGCTCTGTTAACACAGCTACCATCA |
| | | GCGGCCTTAAACCTGGCGTTGATTATACCATCACTGTGTATGCTGT |
| | | CACGATCCATAACGTTTCTTTCCCAATTTCCATTAATTACCGCACA |
| | | GAAATTGACAAACCATCCCAGCACCATCACCACCACCACTGA |
| 65 | ATI-967 core (parent of | EVVAATPTSLLISWQGQLSPSFYYRITYGETGGNSPVQEFTVPVAS |
| | ADX_5417_E01) | GTATISGLKPGVDYTITVYAVTSHGIYFYAPISINYRT |
| 66 | ATI-967 BC loop | QGQLSPSFY |
| 67 | ATI-967 DE loop | PVASGT |
| 68 | ATI-967 FG loop | VTSHGIYFYAP |
| 69 | ATI-967 w/ N leader | GVSDVPRDLEVVAATPTSLLISWQGQLSPSFYYRITYGETGGNSPV |
| | | QEFTVPVASGTATISGLKPGVDYTITVYAVTSHGIYFYAPISINYR |
| | | T |
| 70 | ATI-967 w/ N leader + his | GVSDVPRDLEVVAATPTSLLISWQGQLSPSFYYRITYGETGGNSPV |
| | tag | QEFTVPVASGTATISGLKPGVDYTITVYAVTSHGIYFYAPISINYR |
| | | THHHHHH |
| 71 | ATI-967 w/ N leader and C | GVSDVPRDLEVVAATPTSLLISWQGQLSPSFYYRITYGETGGNSPV |
| | tail | QEFTVPVASGTATISGLKPGVDYTITVYAVTSHGIYFYAPISINYR |
| | | TEIDKPSQ |
| 72 | ATI-967 w/ N leader and C | GVSDVPRDLEVVAATPTSLLISWQGQLSPSFYYRITYGETGGNSPV |
| | tail + his tag | QEFTVPVASGTATISGLKPGVDYTITVYAVTSHGIYFYAPISINYR |
| | | TEIDKPSQHHHHHH |
| 73 | ATI-967 w/ N leader and | GVSDVPRDLEVVAATPTSLLISWQGQLSPSFYYRITYGETGGNSPV |
| | modified C-terminus | QEFTVPVASGTATISGLKPGVDYTITVYAVTSHGIYFYAPISINYR |
| | including PC | TPC |
| 74 | ATI-967 w/ N leader and | GVSDVPRDLEVVAATPTSLLISWQGQLSPSFYYRITYGETGGNSPV |
| | modified C-terminus | QEFTVPVASGTATISGLKPGVDYTITVYAVTSHGIYFYAPISINYR |
| | including PC + his tag | TPCHHHHHH |
| 75 | ATI-967-full length | MGVSDVPRDLEVVAATPTSLLISWQGQLSPSFYYRITYGETGGNSP |
| | | VQEFTVPVASGTATISGLKPGVDYTITVYAVTSHGIYFYAPISINY |
| | | RTEIDKPSQHHHHHH |
| 76 | ATI-967-core (nucleotide | GAAGTGGTTGCTGCCACCCCCACCAGCCTGCTGATCAGCTGGCAGG |
| | sequence) | GACAGCTGTCTCCGTCTTTCTATTACCGAATCACTTACGGCGAAAC |
| | | AGGAGGCAATAGCCCTGTCCAGGAGTTCACTGTGCCTGTTGCTAGT |
| | | GGGACAGCTACCATCAGCGGCCTTAAACCTGGCGTTGATTATACCA |
| | | TCACTGTGTATGCTGTCACTTCTCATGGCATATACTICTACGCTCC |
| | | AATTTCCATTAATTACCGCACA |
| 77 | ATI-967 w/ N leader | ATGGGAGTTTCTGATGTGCCGCGCGACCTGGAAGTGGTTGCTGCCA |
| | (nucleotide sequence with | CCCCCACCAGCCTGCTGATCAGCTGGCAGGGACAGCTGTCTCCGTC |
| | N-terminal methionine) | TTTCTATTACCGAATCACTTACGGCGAAACAGGAGGCAATAGCCCT |
| | | GTCCAGGAGTTCACTGTGCCTGTTGCTAGTGGGACAGCTACCATCA |
| | | GCGGCCTTAAACCTGGCGTTGATTATACCATCACTGTGTATGCTGT |
| | | CACTTCTCATGGCATATACTTCTACGCTCCAATTTCCATTAATTAC |
| | | CGCACA |
| 78 | ATI-967 w/ N leader and | ATGGGAGTTTCTGATGTGCCGCGCGACCTGGAAGTGGTTGCTGCCA |
| | modified C-terminus | CCCCCACCAGCCTGCTGATCAGCTGGCAGGGACAGCTGTCTCCGTC |
| | including PC (nucleotide | TTTCTATTACCGAATCACTTACGGCGAAACAGGAGGCAATAGCCCT |
| | sequence with N-terminal | GTCCAGGAGTTCACTGTGCCTGTTGCTAGTGGGACAGCTACCATCA |
| | methionine) | GCGGCCTTAAACCTGGCGTTGATTATACCATCACTGTGTATGCTGT |
| | | CACTTCTCATGGCATATACTTCTACGCTCCAATTTCCATTAATTAC |
| | | CGCACACCGTGC |
| 79 | ATI-967 w/ N leader and C | ATGGGAGTTTCTGATGTGCCGCGCGACCTGGAAGTGGTTGCTGCCA |
| | tail + his tag (nucleotide | CCCCCACCAGCCTGCTGATCAGCTGGCAGGGACAGCTGTCTCCGTC |
| | sequence with N-terminal | TTTCTATTACCGAATCACTTACGGCGAAACAGGAGGCAATAGCCCT |
| | methionine) | GTCCAGGAGTTCACTGTGCCTGTTGCTAGTGGGACAGCTACCATCA |
| | | GCGGCCTTAAACCTGGCGTTGATTATACCATCACTGTGTATGCTGT |
| | | CACTTCTCATGGCATATACTTCTACGCTCCAATTTCCATTAATTAC |
| | | CGCACAGAAATTGACAAACCATCCCAGCACCATCACCACCACCACT |
| | | GAT |
| 80 | ADX_5322_A02 core | EVVAATPTSLLISWSYDGPIDRYYRITYGETGGNSPVQEFTVPPDQ |
| | | KTATISGLKPGVDYTITVYAVRLEEAHYNREFPISINYRT |
| 81 | ADX_5322_A02 BC loop | SYDGPIDRY |
| 82 | ADX_5322_A02 DE loop | PPDQKT |
| 83 | ADX_5322_A02 FG loop | VRLEEAHYNREFP |
| 84 | ADX_5322_A02 w/ N | GVSDVPRDLEVVAATPTSLLISWSYDGPIDRYYRITYGETGGNSPV |
| | leader | QEFTVPPDQKTATISGLKPGVDYTITVYAVRLEEAHYNREFPISIN |
| | | YRT |
| 85 | ADX_5322_A02 w/ N | GVSDVPRDLEVVAATPTSLLISWSYDGPIDRYYRITYGETGGNSPV |
| | leader + his tag | QEFTVPPDQKTATISGLKPGVDYTITVYAVRLEEAHYNREFPISIN |
| | | YRTHHHHHH |
| 86 | ADX_5322_A02 w/ N | GVSDVPRDLEVVAATPTSLLISWSYDGPIDRYYRITYGETGGNSPV |
| | leader and C tail | QEFTVPPDQKTATISGLKPGVDYTITVYAVRLEEAHYNREFPISIN |
| | | YRTEIDKPSQ |
| 87 | ADX_5322_A02 w/ N | GVSDVPRDLEVVAATPTSLLISWSYDGPIDRYYRITYGETGGNSPV |
| | leader and C tail + his | QEFTVPPDQKTATISGLKPGVDYTITVYAVRLEEAHYNREFPISIN |
| | tag | YRTEIDKPSQHHHHHH |
| 88 | ADX_5322_A02 w/ N | GVSDVPRDLEVVAATPTSLLISWSYDGPIDRYYRITYGETGGNSPV |
| | leader and modified C- | QEFTVPPDQKTATISGLKPGVDYTITVYAVRLEEAHYNREFPISIN |
| | terminus including PC | YRTPC |
| 89 | ADX_5322_A02 w/ N | GVSDVPRDLEVVAATPTSLLISWSYDGPIDRYYRITYGETGGNSPV |
| | leader and modified C- | QEFTVPPDQKTATISGLKPGVDYTITVYAVRLEEAHYNREFPISIN |
| | terminus including PC + | YRTPCHHHHHH |
| | his tag | |
| 90 | ADX_5322_A02-Mal- | GVSDVPRDLEVVAATPTSLLISWSYDGPIDRYYRITYGETGGNSPV |
| | DBCO-FFPF18 | QEFTVPPDQKTATISGLKPGVDYTITVYAVRLEEAHYNREFPISIN |
| | | YRTPC-[Maleamide-DBCO-FFP(18F)] |
| 91 | ADX_5322_A02 full | MGVSDVPRDLEVVAATPTSLLISWSYDGPIDRYYRITYGETGGNSP |
| | length | VQEFTVPPDQKTATISGLKPGVDYTITVYAVRLEEAHYNREFPISI |
| | | NYRTPCHHHHHH |
| 92 | ADX_5322_A02-core | GAAGTGGTTGCTGCCACCCCCACCAGCCTGCTGATCAGCTGGTCTT |
| | (nucleotide sequence) | ACGATGGCCCAATTGACCGGTATTACCGCATCACTTACGGCGAAAC |
| | | AGGAGGCAATAGCCCTGTCCAGGAGTTCACTGTGCCTCCGGATCAG |
| | | AAGACAGCTACCATCAGCGGCCTTAAACCTGGCGTTGATTATACCA |
| | | TCACTGTGTATGCTGTCCGGCTGGAAGAAGCTCATTACAATCGAGA |
| | | GTTTCCAATTTCCATTAATTACCGCACA |
| 93 | ADX_5322_A02 w/ N | ATGGGAGTTTCTGATGTGCCGCGCGACCTGGAAGTGGTTGCTGCCA |
| | leader (nucleotide | CCCCCACCAGCCTGCTGATCAGCTGGTCTTACGATGGCCCAATTGA |
| | sequence with N-terminal | CCGGTATTACCGCATCACTTACGGCGAAACAGGAGGCAATAGCCCT |
| | methionine) | GTCCAGGAGTTCACTGTGCCTCCGGATCAGAAGACAGCTACCATCA |
| | | GCGGCCTTAAACCTGGCGTTGATTATACCATCACTGTGTATGCTGT |
| | | CCGGCTGGAAGAAGCTCATTACAATCGAGAGTTTCCAATTTCCATT |
| | | AATTACCGCACA |
| 94 | ADX_5322_A02 w/ N | ATGGGAGTTTCTGATGTGCCGCGCGACCTGGAAGTGGTTGCTGCCA |
| | leader and modified C- | CCCCCACCAGCCTGCTGATCAGCTGGTCTTACGATGGCCCAATTGA |
| | terminus including PC | CCGGTATTACCGCATCACTTACGGCGAAACAGGAGGCAATAGCCCT |
| | (nucleotide sequence with | GTCCAGGAGTTCACTGTGCCTCCGGATCAGAAGACAGCTACCATCA |
| | N-terminal methionine) | GCGGCCTTAAACCTGGCGTTGATTATACCATCACTGTGTATGCTGT |
| | | CCGGCTGGAAGAAGCTCATTACAATCGAGAGTTTCCAATTTCCATT |
| | | AATTACCGCACACCGTGC |
| 95 | ADX_5322_A02 w/ N | ATGGGAGTTTCTGATGTGCCGCGCGACCTGGAAGTGGTTGCTGCCA |
| | leader and C tail + his tag | CCCCCACCAGCCTGCTGATCAGCTGGTCTTACGATGGCCCAATTGA |
| | (nucleotide sequence with | CCGGTATTACCGCATCACTTACGGCGAAACAGGAGGCAATAGCCCT |
| | N-terminal methionine) | GTCCAGGAGTTCACTGTGCCTCCGGATCAGAAGACAGCTACCATCA |
| | | GCGGCCTTAAACCTGGCGTTGATTATACCATCACTGTGTATGCTGT |
| | | CCGGCTGGAAGAAGCTCATTACAATCGAGAGTTTCCAATTTCCATT |
| | | AATTACCGCACACCGTGCCACCATCACCACCACCACTGA |
| 96 | ADX_5417_E01 core | EVVAATPTSLLISWRAQLSPSFYYRITYGETGGNSPVQEFTVPNDV |
| | | MTATISGLKPGVDYTITVYAVTTHGVYFYSPISINYRT |
| 97 | ADX_5417_E01 BC loop | RAQLSPSFY |
| 98 | ADX_5417_E01 DE loop | PNDVMT |
| 99 | ADX_5417_E01 FG loop | VTTHGVYFYSP |
| 100 | ADX_5417_E01 w/ N | GVSDVPRDLEVVAATPTSLLISWRAQLSPSFYYRITYGETGGNSPV |
| | leader | QEFTVPNDVMTATISGLKPGVDYTITVYAVTTHGVYFYSPISINYR |
| | | T |
| 101 | ADX_5417_E01 w/ N | GVSDVPRDLEVVAATPTSLLISWRAQLSPSFYYRITYGETGGNSPV |
| | leader + his tag | QEFTVPNDVMTATISGLKPGVDYTITVYAVTTHGVYFYSPISINYR |
| | | THHHHHH |
| 102 | ADX_5417_E01 w/ N | GVSDVPRDLEVVAATPTSLLISWRAQLSPSFYYRITYGETGGNSPV |
| | leader and C tail | QEFTVPNDVMTATISGLKPGVDYTITVYAVTTHGVYFYSPISINYR |
| | | TEIDKPSQ |
| 103 | ADX_5417_E01 w/ N | GVSDVPRDLEVVAATPTSLLISWRAQLSPSFYYRITYGETGGNSPV |
| | leader and C tail + his | QEFTVPNDVMTATISGLKPGVDYTITVYAVTTHGVYFYSPISINYR |
| | tag | TEIDKPSQHHHHHH |
| 104 | ADX_5417_E01 w/ N | GVSDVPRDLEVVAATPTSLLISWRAQLSPSFYYRITYGETGGNSPV |
| | leader and modified C- | QEFTVPNDVMTATISGLKPGVDYTITVYAVTTHGVYFYSPISINYR |
| | terminus including PC | TPC |
| 105 | ADX_5417_E01 w/ N | GVSDVPRDLEVVAATPTSLLISWRAQLSPSFYYRITYGETGGNSPV |
| | leader and modified C- | QEFTVPNDVMTATISGLKPGVDYTITVYAVTTHGVYFYSPISINYR |
| | terminus including PC + | TPCHHHHHH |
| | his tag | |
| 106 | ADX-5417_E01-Mal- | GVSDVPRDLEVVAATPTSLLISWSYDGPIDRYYRITYGETGGNSPV |
| | DBCO-FFPF18 | QEFTVPPDQKTATISGLKPGVDYTITVYAVRLEEAHYNREFPISIN |
| | | YRTPC-[Maleamide-DBCO-FFP(18F)] |
| 107 | ADX-5417_E01 full length | MGVSDVPRDLEVVAATPTSLLISWRAQLSPSFYYRITYGETGGNSP |
| | | VQEFTVPNDVMTATISGLKPGVDYTITVYAVTTHGVYFYSPISINY |
| | | RTPCHHHHHH |
| 108 | ADX-5417_E01-core | GAAGTGGTTGCTGCCACCCCCACCAGCCTGCTGATCAGCTGGAGGG |
| | (nucleotide sequence) | CTCAGCTGTCTCCGTCTTTCTATTACCGCATCACTTACGGCGAAAC |
| | | AGGAGGCAATAGCCCTGTCCAGGAGTTCACTGTGCCTAATGATGTA |
| | | ATGACAGCTACCATCAGCGGCCTTAAACCTGGCGTTGATTATACCA |
| | | TCACTGTGTATGCTGTCACTACTCATGGTGTTTATTTCTACTCACC |
| | | AATTTCCATTAATTACCGCACA |
| 109 | ADX_5417_E01 w/ N | ATGGGAGTTTCTGATGTGCCGCGCGACCTGGAAGTGGTTGCTGCCA |
| | leader (nucleotide | CCCCCACCAGCCTGCTGATCAGCTGGAGGGCTCAGCTGTCTCCGTC |
| | sequence with N-terminal | TTTCTATTACCGCATCACTTACGGCGAAACAGGAGGCAATAGCCCT |
| | methionine) | GTCCAGGAGTTCACTGTGCCTAATGATGTAATGACAGCTACCATCA |
| | | GCGGCCTTAAACCTGGCGTTGATTATACCATCACTGTGTATGCTGT |
| | | CACTACTCATGGTGTTTATTTCTACTCACCAATTTCCATTAATTAC |
| | | CGCACA |
| 110 | ADX_5417_E01 w/ N | ATGGGAGTTTCTGATGTGCCGCGCGACCTGGAAGTGGTTGCTGCCA |
| | leader and modified C- | CCCCCACCAGCCTGCTGATCAGCTGGAGGGCTCAGCTGTCTCCGTC |
| | terminus including PC | TTTCTATTACCGCATCACTTACGGCGAAACAGGAGGCAATAGCCCT |
| | (nucleotide sequence with | GTCCAGGAGTTCACTGTGCCTAATGATGTAATGACAGCTACCATCA |
| | N-terminal methionine) | GCGGCCTTAAACCTGGCGTTGATTATACCATCACTGTGTATGCTGT |
| | | CACTACTCATGGTGTTTATTTCTACTCACCAATTTCCATTAATTAC |
| | | CGCACACCGTGC |
| 111 | ADX_5417_E01 w/ N | ATGGGAGTTTCTGATGTGCCGCGCGACCTGGAAGTGGTTGCTGCCA |
| | leader and C tail + his tag | CCCCCACCAGCCTGCTGATCAGCTGGAGGGCTCAGCTGTCTCCGTC |
| | (nucleotide sequence with | TTTCTATTACCGCATCACTTACGGCGAAACAGGAGGCAATAGCCCT |
| | N-terminal methionine) | GTCCAGGAGTTCACTGTGCCTAATGATGTAATGACAGCTACCATCA |
| | | GCGGCCTTAAACCTGGCGTTGATTATACCATCACTGTGTATGCTGT |
| | | CACTACTCATGGTGTTTATTTCTACTCACCAATTTCCATTAATTAC |
| | | CGCACACCGTGCCACCATCACCACCACCACTGA |
| 112 | ATI_1420_A10 core | EVVAATPTSLLISWPYPSYYIEYRITYGETGGNSPVQEFTVQSMKA |
| | | TISGLKPGVDYTITVYAIRHPGMLEFGISINYRT |
| 113 | ATI_1420_A10 BC loop | PYPSYYIE |
| 114 | ATI_1420_A10 DE loop | IRHPGMLEFG |
| 115 | ATI_1420_A10 FG loop | VTDGASIASYAFP |
| 116 | ATI_1420_A10 w/ N | GVSDVPRDLEVVAATPTSLLISWPYPSYYIEYRITYGETGGNSPVQ |
| | leader | EFTVQSMKATISGLKPGVDYTITVYAIRHPGMLEFGISINYRT |
| 117 | ATI_1420_A10 w/ N | GVSDVPRDLEVVAATPTSLLISWPYPSYYIEYRITYGETGGNSPVQ |
| | leader + his tag | EFTVQSMKATISGLKPGVDYTITVYAIRHPGMLEFGISINYRTHHH |
| | | HHH |
| 118 | ATI_1420_A10 w/ N | GVSDVPRDLEVVAATPTSLLISWPYPSYYIEYRITYGETGGNSPVQ |
| | leader and C tail | EFTVQSMKATISGLKPGVDYTITVYAIRHPGMLEFGISINYRTEID |
| | | KPSQ |
| 119 | ATI_1420_A10 w/ N leader | GVSDVPRDLEVVAATPTSLLISWPYPSYYIEYRITYGETGGNSPVQ |
| | and C tail + his tag | EFTVQSMKATISGLKPGVDYTITVYAIRHPGMLEFGISINYRTEID |
| | | KPSQHHHHHH |
| 120 | ATI_1420_A10 w/ N leader | GVSDVPRDLEVVAATPTSLLISWPYPSYYIEYRITYGETGGNSPVQ |
| | and modified C-terminus | EFTVQSMKATISGLKPGVDYTITVYAIRHPGMLEFGISINYRTPC |
| | including PC | |
| 121 | ATI_1420_A10 w/ N | GVSDVPRDLEVVAATPTSLLISWPYPSYYIEYRITYGETGGNSPVQ |
| | leader and modified C- | EFTVQSMKATISGLKPGVDYTITVYAIRHPGMLEFGISINYRTPCH |
| | terminus including PC + | HHHHH |
| | his tag | |
| 122 | ATI_1420_A10-full | MGVSDVPRDLEVVAATPTSLLISWPYPSYYIEYRITYGETGGNSPV |
| | length | QEFTVQSMKATISGLKPGVDYTITVYAIRHPGMLEFGISINYRTEI |
| | | DKPSQHHHHHH |
| 123 | ATI_1420_B09 core | EVVAATPTSLLISWHKFSSLMSYRITYGETGGNSPVQEFTVGSV |
| | | NATISGLKPGVDYTITVYAIHNVGFISINYRT |
| 124 | ATI_1420_B09 BC loop | HKFSSLMS |
| 125 | ATI_1420_B09 DE loop | GSVN |
| 126 | ATI_1420_B09 FG loop | IHNVGF |
| 127 | ATI_1420_B09 w/ N | GVSDVPRDLEVVAATPTSLLISWHKFSSLMSYRITYGETGGNSP |
| | leader | VQEFTVGSVNATISGLKPGVDYTITVYAIHNVGFISINYRT |
| 128 | ATI_1420_B09 w/ N | GVSDVPRDLEVVAATPTSLLISWHKFSSLMSYRITYGETGGNSP |
| | leader + his tag | VQEFTVGSVNATISGLKPGVDYTITVYAIHNVGFISINYRTHHH |
| | | HHH |
| 129 | AATI_1420_B09 w/ N | GVSDVPRDLEVVAATPTSLLISWHKFSSLMSYRITYGETGGNSP |
| | leader and C tail | VQEFTVGSVNATISGLKPGVDYTITVYAIHNVGFISINYRTEID |
| | | KPSQ |
| 130 | ATI_1420_B09 w/ N | GVSDVPRDLEVVAATPTSLLISWHKFSSLMSYRITYGETGGNSP |
| | leader and C tail + his | VQEFTVGSVNATISGLKPGVDYTITVYAIHNVGFISINYRTEID |
| | tag | KPSQHHHHHH |
| 131 | ATI_1420_B09 w/ N | GVSDVPRDLEVVAATPTSLLISWHKFSSLMSYRITYGETGGNSP |
| | leader and modified C- | VQEFTVGSVNATISGLKPGVDYTITVYAIHNVGFISINYRTPC |
| | terminus including PC | |
| 132 | ATI_1420_B09 w/ N | GVSDVPRDLEVVAATPTSLLISWHKFSSLMSYRITYGETGGNSP |
| | leader and modified C- | VQEFTVGSVNATISGLKPGVDYTITVYAIHNVGFISINYRTPCH |
| | terminus including PC + | HHHHH |
| | his tag | |
| 133 | ATI_1420_B09-full | MGVSDVPRDLEVVAATPTSLLISWHKFSSLMSYRITYGETGGNS |
| | length | PVQEFTVGSVNATISGLKPGVDYTITVYAIHNVGFISINYRTEI |
| | | DKPSQHHHHHH |
| 134 | ATI_1420_C02 core | EVVAATPTSLLISWRIKSYYAYRITYGETGGNSPVQEFTVRQHV |
| | | ATISGLKPGVDYTITVYARLGDVELVYEISINYRT |
| 135 | ATI_1420_C02 BC loop | RIKSYYA |
| 136 | ATI_1420_C02 DE loop | RQHV |
| 137 | ATI_1420_C02 FG loop | RLGDVELVYE |
| 138 | ATI_1420_C02 w/ N | GVSDVPRDLEVVAATPTSLLISWRIKSYYAYRITYGETGGNSPV |
| | leader | QEFTVRQHVATISGLKPGVDYTITVYARLGDVELVYEISINYRT |
| 139 | ATI_1420_C02 w/ N | GVSDVPRDLEVVAATPTSLLISWHKFSSLMSYRITYGETGGNSP |
| | leader + his tag | VQEFTVGSVNATISGLKPGVDYTITVYAIHNVGFISINYRTHHH |
| | | HHH |
| 140 | ATI_1420_C02 w/ N | GVSDVPRDLEVVAATPTSLLISWRIKSYYAYRITYGETGGNSPV |
| | leader and C tail | QEFTVRQHVATISGLKPGVDYTITVYARLGDVELVYEISINYRT |
| | | EIDKPSQ |
| 141 | ATI_1420_C02 w/ N | GVSDVPRDLEVVAATPTSLLISWRIKSYYAYRITYGETGGNSPV |
| | leader and C tail + his | QEFTVRQHVATISGLKPGVDYTITVYARLGDVELVYEISINYRT |
| | tag | EIDKPSQHHHHHH |
| 142 | ATI_1420_C02 w/ N | GVSDVPRDLEVVAATPTSLLISWRIKSYYAYRITYGETGGNSPV |
| | leader and modified C- | QEFTVRQHVATISGLKPGVDYTITVYARLGDVELVYEISINYRT |
| | terminus including PC | PC |
| 143 | ATI_1420_C02 w/ N | GVSDVPRDLEVVAATPTSLLISWHKFSSLMSYRITYGETGGNSP |
| | leader and modified C- | VQEFTVGSVNATISGLKPGVDYTITVYAIHNVGFISINYRTPCH |
| | terminus including PC + | HHHHH |
| | his tag | |
| 144 | ATI_1420_C02-full | MGVSDVPRDLEVVAATPTSLLISWRIKSYYAYRITYGETGGNSP |
| | length | VQEFTVRQHVATISGLKPGVDYTITVYARLGDVELVYEISINYR |
| | | TEIDKPSQHHHHHH |
| 145 | ATI_1420_C11 core | EVVAATPTSLLISWMYPLKSVPYRITYGETGGNSPVQEFTVYSS |
| | | GATISGLKPGVDYTITVYAMSYSTYHAFMISINYRT |
| 146 | ATI_1420_C11 BC loop | MYPLKSVP |
| 147 | ATI_1420_C11 DE loop | YSSG |
| 148 | ATI_1420_C11 FG loop | MSYSTYHAFM |
| 149 | ATI_1420_C11 w/ N leader | GVSDVPRDLEVVAATPTSLLISWMYPLKSVPYRITYGETGGNSP |
| | | VQEFTVYSSGATISGLKPGVDYTITVYAMSYSTYHAFMISINYR |
| | | T |
| 150 | ATI_1420_C11 w/ N leader | GVSDVPRDLEVVAATPTSLLISWMYPLKSVPYRITYGETGGNSP |
| | + his tag | VQEFTVYSSGATISGLKPGVDYTITVYAMSYSTYHAFMISINYR |
| | | THHHHHH |
| 151 | ATI_1420_C11 w/ N leader | GVSDVPRDLEVVAATPTSLLISWMYPLKSVPYRITYGETGGNSP |
| | and C tail | VQEFTVYSSGATISGLKPGVDYTITVYAMSYSTYHAFMISINYR |
| | | TEIDKPSQ |
| 152 | ATI_1420_C11 w/ N leader | GVSDVPRDLEVVAATPTSLLISWMYPLKSVPYRITYGETGGNSP |
| | and C tail + his tag | VQEFTVYSSGATISGLKPGVDYTITVYAMSYSTYHAFMISINYR |
| | | TEIDKPSQHHHHHH |
| 153 | ATI_1420_C11 w/ N leader | GVSDVPRDLEVVAATPTSLLISWMYPLKSVPYRITYGETGGNSP |
| | and modified C-terminus | VQEFTVYSSGATISGLKPGVDYTITVYAMSYSTYHAFMISINYR |
| | including PC | TPC |
| 154 | ATI_1420_C11 w/ N leader | GVSDVPRDLEVVAATPTSLLISWMYPLKSVPYRITYGETGGNSP |
| | and modified C-terminus | VQEFTVYSSGATISGLKPGVDYTITVYAMSYSTYHAFMISINYR |
| | including PC + his tag | TPCHHHHHH |
| 155 | AATI_1420_C11-full | MGVSDVPRDLEVVAATPTSLLISWMYPLKSVPYRITYGETGGNS |
| | length | PVQEFTVYSSGATISGLKPGVDYTITVYAMSYSTYHAFMISINY |
| | | RTEIDKPSQHHHHHH |
| 156 | ATI_1420_D01 core | EVVAATPTSLLISWRTVPETDYRITYGETGGNSPVQEFTVPDNT |
| | | ATISGLKPGVDYTITVYALETAHYNRDYISINYRT |
| 157 | ATI_1420_D01 BC loop | RTVPETD |
| 158 | ATI_1420_D01 DE loop | PDNT |
| 159 | ATI_1420_D01 FG loop | LETAHYNRDY |
| 160 | ATI_1420_D01 w/ N | GVSDVPRDLEVVAATPTSLLISWRTVPETDYRITYGETGGNSPV |
| | leader | QEFTVPDNTATISGLKPGVDYTITVYALETAHYNRDYISINYRT |
| 161 | ATI_1420_D01 w/ N | GVSDVPRDLEVVAATPTSLLISWRTVPETDYRITYGETGGNSPV |
| | leader + his tag | QEFTVPDNTATISGLKPGVDYTITVYALETAHYNRDYISINYRT |
| | | HHHHHH |
| 162 | ATI_1420_D01 w/ N | GVSDVPRDLEVVAATPTSLLISWRTVPETDYRITYGETGGNSPV |
| | leader and C tail | QEFTVPDNTATISGLKPGVDYTITVYALETAHYNRDYISINYRT |
| | | EIDKPSQ |
| 163 | ATI_1420_D01 w/ N | GVSDVPRDLEVVAATPTSLLISWRTVPETDYRITYGETGGNSPV |
| | leader and C tail + his | QEFTVPDNTATISGLKPGVDYTITVYALETAHYNRDYISINYRT |
| | tag | EIDKPSQHHHHHH |
| 164 | ATI_1420_D01 w/ N | GVSDVPRDLEVVAATPTSLLISWRTVPETDYRITYGETGGNSPV |
| | leader and modified C- | QEFTVPDNTATISGLKPGVDYTITVYALETAHYNRDYISINYRT |
| | terminus including PC | PC |
| 165 | ATI_1420_D01 w/ N | GVSDVPRDLEVVAATPTSLLISWRTVPETDYRITYGETGGNSPV |
| | leader and modified C- | QEFTVPDNTATISGLKPGVDYTITVYALETAHYNRDYISINYRT |
| | terminus including PC + | PCHHHHHH |
| | his tag | |
| 166 | ATI_1420_D01-full | MGVSDVPRDLEVVAATPTSLLISWRTVPETDYRITYGETGGNSP |
| | length | VQEFTVPDNTATISGLKPGVDYTITVYALETAHYNRDYISINYR |
| | | TEIDKPSQHHHHHH |
| 167 | ATI_1420_D05 core | EVVAATPTSLLISWTAYYSTIKYRITYGETGGNSPVQEFTVGPK |
| | | HHATISGLKPGVDYTITVYAYNTKPGYHAHQISINYRT |
| 168 | ATI_1420_D05 BC loop | TAYYSTIK |
| 169 | ATI_1420_D05 DE loop | GPKHH |
| 170 | ATI_1420_D05 FG loop | YNTKPGYHAHQ |
| 171 | ATI_1420_D05 w/ N | GVSDVPRDLEVVAATPTSLLISWTAYYSTIKYRITYGETGGNSP |
| | leader | VQEFTVGPKHHATISGLKPGVDYTITVYAYNTKPGYHAHQISIN |
| | | YRT |
| 172 | ATI_1420_D05 w/ N | GVSDVPRDLEVVAATPTSLLISWTAYYSTIKYRITYGETGGNSP |
| | leader + his tag | VQEFTVGPKHHATISGLKPGVDYTITVYAYNTKPGYHAHQISIN |
| | | YRTHHHHHH |
| 173 | ATI_1420_D05 w/ N | GVSDVPRDLEVVAATPTSLLISWTAYYSTIKYRITYGETGGNSP |
| | leader and C tail | VQEFTVGPKHHATISGLKPGVDYTITVYAYNTKPGYHAHQISIN |
| | | YRTEIDKPSQ |
| 174 | ATI_1420_D05 w/ N | GVSDVPRDLEVVAATPTSLLISWTAYYSTIKYRITYGETGGNSP |
| | leader and C tail + his | VQEFTVGPKHHATISGLKPGVDYTITVYAYNTKPGYHAHQISIN |
| | tag | YRTEIDKPSQHHHHHH |
| 175 | ATI_1420_D05 w/ N | GVSDVPRDLEVVAATPTSLLISWTAYYSTIKYRITYGETGGNSP |
| | leader and modified C- | VQEFTVGPKHHATISGLKPGVDYTITVYAYNTKPGYHAHQISIN |
| | terminus including PC | YRTPC |
| 176 | ATI_1420_D05 w/ N | GVSDVPRDLEVVAATPTSLLISWTAYYSTIKYRITYGETGGNSP |
| | leader and modified C- | VQEFTVGPKHHATISGLKPGVDYTITVYAYNTKPGYHAHQISIN |
| | terminus including PC + | YRTPCHHHHHH |
| | his tag | |
| 177 | ATI_1420_D05-full | MGVSDVPRDLEVVAATPTSLLISWTAYYSTIKYRITYGETGGNS |
| | length | PVQEFTVGPKHHATISGLKPGVDYTITVYAYNTKPGYHAHQISI |
| | | NYRTEIDKPSQHHHHHH |
| 178 | ATI_1420_D10 core | EVVAATPTSLLISWRIPSYHIQYRITYGETGGNSPVQEFTVYQK |
| | | YATISGLKPGVDYTITVYAVSPPKQLRFGISINYRT |
| 179 | ATI_1420_D10 BC loop | RIPSYHIQ |
| 180 | ATI_1420_D10 DE loop | YQKY |
| 181 | ATI_1420_D10 FG loop | VSPPKQLRFG |
| 182 | ATI_1420_D10 w/ N | GVSDVPRDLEVVAATPTSLLISWRIPSYHIQYRITYGETGGNSP |
| | leader | VQEFTVYQKYATISGLKPGVDYTITVYAVSPPKOLRFGISINYR |
| | | T |
| 183 | ATI_1420_D10 w/ N | GVSDVPRDLEVVAATPTSLLISWRIPSYHIQYRITYGETGGNSP |
| | leader + his tag | VQEFTVYQKYATISGLKPGVDYTITVYAVSPPKOLREGISINYR |
| | | THHHHHH |
| 184 | ATI_1420_D10 w/ N | GVSDVPRDLEVVAATPTSLLISWRIPSYHIQYRITYGETGGNSP |
| | leader and C tail | VQEFTVYQKYATISGLKPGVDYTITVYAVSPPKOLRFGISINYR |
| | | TEIDKPSQ |
| 185 | ATI_1420_D10 w/ N | GVSDVPRDLEVVAATPTSLLISWRIPSYHIQYRITYGETGGNSP |
| | leader and C tail + his | VQEFTVYQKYATISGLKPGVDYTITVYAVSPPKOLRFGISINYR |
| | tag | TEIDKPSQHHHHHH |
| 186 | ATI_1420_D10 w/ N | GVSDVPRDLEVVAATPTSLLISWRIPSYHIQYRITYGETGGNSP |
| | leader and modified C- | VQEFTVYQKYATISGLKPGVDYTITVYAVSPPKOLRFGISINYR |
| | terminus including PC | TPC |
| 187 | ATI_1420_D10 w/ N | GVSDVPRDLEVVAATPTSLLISWRIPSYHIQYRITYGETGGNSP |
| | leader and modified C- | VQEFTVYQKYATISGLKPGVDYTITVYAVSPPKOLRFGISINYR |
| | terminus including PC + | TPCHHHHHH |
| | his tag | |
| 188 | ATI_1420_D010-full | MGVSDVPRDLEVVAATPTSLLISWRIPSYHIQYRITYGETGGNS |
| | length | PVQEFTVYQKYATISGLKPGVDYTITVYAVSPPKQLRFGISINY |
| | | RTEIDKPSQHHHHHH |
| 189 | ATI_1420_F10_core | EVVAATPTSLLISWPAPPSYVFYRITYGETGGNSPVQEFTVYPY |
| | | MATISGLKPGVDYTITVYAYTSGFSISINYRT |
| 190 | ATI_1420_F10 BC loop | PAPPSYVF |
| 191 | ATI_1420_F10 DE loop | YPYM |
| 192 | ATI_1420_F10 FG loop | YTSGFS |
| 193 | ATI_1420_F10 w/ N leader | GVSDVPRDLEVVAATPTSLLISWPAPPSYVFYRITYGETGGNSP |
| | | VQEFTVYPYMATISGLKPGVDYTITVYAYTSGFSISINYRT |
| 194 | ATI_1420_F10 w/ N | GVSDVPRDLEVVAATPTSLLISWPAPPSYVFYRITYGETGGNSP |
| | leader + his tag | VQEFTVYPYMATISGLKPGVDYTITVYAYTSGFSISINYRTHHH |
| | | HHH |
| 195 | ATI_1420_F10 w/ N leader | GVSDVPRDLEVVAATPTSLLISWPAPPSYVFYRITYGETGGNSP |
| | and C tail | VQEFTVYPYMATISGLKPGVDYTITVYAYTSGFSISINYRTEID |
| | | KPSQ |
| 196 | ATI_1420_F10 w/ N | GVSDVPRDLEVVAATPTSLLISWPAPPSYVFYRITYGETGGNSP |
| | leader and C tail + his | VQEFTVYPYMATISGLKPGVDYTITVYAYTSGFSISINYRTEID |
| | tag | KPSQHHHHHH |
| 197 | ATI_1420_F10 w/ N | GVSDVPRDLEVVAATPTSLLISWPAPPSYVFYRITYGETGGNSP |
| | leader and modified C- | VQEFTVYPYMATISGLKPGVDYTITVYAYTSGFSISINYRTPC |
| | terminus including PC | |
| 198 | ATI_1420_F10 w/ N | GVSDVPRDLEVVAATPTSLLISWPAPPSYVFYRITYGETGGNSP |
| | leader and modified C- | VQEFTVYPYMATISGLKPGVDYTITVYAYTSGFSISINYRTPCH |
| | terminus including PC + | HHHHH |
| | his tag | |
| 199 | ATI_1420_F10-full length | MGVSDVPRDLEVVAATPTSLLISWPAPPSYVFYRITYGETGGNS |
| | | PVQEFTVYPYMATISGLKPGVDYTITVYAYTSGFSISINYRTEI |
| | | DKPSQHHHHHH |
| 200 | ATI_1421_C05_core | EVVAATPTSLLISWYMDHKSKYRITYGETGGNSPVQEFTVPDOR |
| | | ATISGLKPGVDYTITVYALSEAHYLRDKISINYRT |
| 201 | ATI_1421_C05 BC loop | YMDHKSK |
| 202 | ATI_1421_C05 DE loop | PDQR |
| 203 | ATI_1421_C05 FG loop | LSEAHYLRDK |
| 204 | ATI_1421_C05 w/ N | GVSDVPRDLEVVAATPTSLLISWYMDHKSKYRITYGETGGNSPV |
| | leader | QEFTVPDQRATISGLKPGVDYTITVYALSEAHYLRDKISINYRT |
| 205 | ATI_1421_C05 w/ N | GVSDVPRDLEVVAATPTSLLISWYMDHKSKYRITYGETGGNSPV |
| | leader + his tag | QEFTVPDQRATISGLKPGVDYTITVYALSEAHYLRDKISINYRT |
| | | HHHHHH |
| 206 | ATI_1421_C05 w/ N | GVSDVPRDLEVVAATPTSLLISWYMDHKSKYRITYGETGGNSPV |
| | leader and C tail | QEFTVPDQRATISGLKPGVDYTITVYALSEAHYLRDKISINYRT |
| | | EIDKPSQ |
| 207 | ATI_1421_C05 w/ N | GVSDVPRDLEVVAATPTSLLISWYMDHKSKYRITYGETGGNSPV |
| | leader and C tail + his | QEFTVPDQRATISGLKPGVDYTITVYALSEAHYLRDKISINYRT |
| | tag | EIDKPSQHHHHHH |
| 208 | ATI_1421_C05 w/ N | GVSDVPRDLEVVAATPTSLLISWYMDHKSKYRITYGETGGNSPV |
| | leader and modified C- | QEFTVPDQRATISGLKPGVDYTITVYALSEAHYLRDKISINYRT |
| | terminus including PC | PC |
| 209 | ATI_1421_C05 w/ N | GVSDVPRDLEVVAATPTSLLISWYMDHKSKYRITYGETGGNSPV |
| | leader and modified C- | QEFTVPDQRATISGLKPGVDYTITVYALSEAHYLRDKISINYRT |
| | terminus including PC + | PCHHHHHH |
| | his tag | |
| 210 | ATI_1421_C05-full | MGVSDVPRDLEVVAATPTSLLISWYMDHKSKYRITYGETGGNSP |
| | length | VQEFTVPDQRATISGLKPGVDYTITVYALSEAHYLRDKISINYR |
| | | TEIDKPSQHHHHHH |
| 211 | ATI_1421_C06 core | EVVAATPTSLLISWENLASYQYRITYGETGGNSPVQEFTVPDQA |
| | | ATISGLKPGVDYTITVYALQTAHYYRQHISINYRT |
| 212 | ATI_1421_C06 BC loop | ENLASYQ |
| 213 | ATI_1421_C06 DE loop | PDQA |
| 214 | ATI_1421_C06 FG loop | LQTAHYYRQH |
| 215 | ATI_1421_C06 w/ N | GVSDVPRDLEVVAATPTSLLISWENLASYQYRITYGETGGNSPV |
| | leader | QEFTVPDQAATISGLKPGVDYTITVYALQTAHYYRQHISINYRT |
| 216 | ATI_1421_C06 w/ N | GVSDVPRDLEVVAATPTSLLISWENLASYQYRITYGETGGNSPV |
| | leader + his tag | QEFTVPDQAATISGLKPGVDYTITVYALQTAHYYRQHISINYRT |
| | | HHHHHH |
| 217 | ATI_1421_C06 w/ N leader | GVSDVPRDLEVVAATPTSLLISWENLASYQYRITYGETGGNSPV |
| | and C tail | QEFTVPDQAATISGLKPGVDYTITVYALQTAHYYRQHISINYRT |
| | | EIDKPSQ |
| 218 | ATI_1421_C06 w/ N | GVSDVPRDLEVVAATPTSLLISWENLASYQYRITYGETGGNSPV |
| | leader and C tail + his | QEFTVPDQAATISGLKPGVDYTITVYALQTAHYYRQHISINYRT |
| | tag | EIDKPSQHHHHHH |
| 219 | ATI_1421_C06 w/ N | GVSDVPRDLEVVAATPTSLLISWENLASYQYRITYGETGGNSPV |
| | leader and modified C- | QEFTVPDQAATISGLKPGVDYTITVYALQTAHYYRQHISINYRT |
| | terminus including PC | PC |
| 220 | ATI_1421_C06 w/ N | GVSDVPRDLEVVAATPTSLLISWENLASYQYRITYGETGGNSPV |
| | leader and modified C- | QEFTVPDQAATISGLKPGVDYTITVYALQTAHYYRQHISINYRT |
| | terminus including PC + | PCHHHHHH |
| | his tag | |
| 221 | ATI_1421_C06-full | MGVSDVPRDLEVVAATPTSLLISWENLASYQYRITYGETGGNSP |
| | length | VQEFTVPDQAATISGLKPGVDYTITVYALQTAHYYRQHISINYR |
| | | TEIDKPSQHHHHHH |
| 222 | ATI 1421_D05 core | EVVAATPTSLLISWYYVQYNDYRITYGETGGNSPVQEFTVPDOS |
| | | ATISGLKPGVDYTITVYALEKAHYYRQNISINYRT |
| 223 | ATI 1421_D05 BC loop | YYVQYND |
| 224 | ATI 1421_D05 DE loop | PDQS |
| 225 | ATI 1421_D05 FG loop | LEKAHYYRON |
| 226 | ATI 1421_D05 w/ N leader | GVSDVPRDLEVVAATPTSLLISWYYVQYNDYRITYGETGGNSPV |
| | | QEFTVPDQSATISGLKPGVDYTITVYALEKAHYYRQNISINYRT |
| 227 | ATI 1421_D05 w/ N leader | GVSDVPRDLEVVAATPTSLLISWYYVQYNDYRITYGETGGNSPV |
| | + his tag | QEFTVPDQSATISGLKPGVDYTITVYALEKAHYYRQNISINYRT |
| | | HHHHHH |
| 228 | ATI 1421_D05 w/ N leader | GVSDVPRDLEVVAATPTSLLISWYYVQYNDYRITYGETGGNSPV |
| | and C tail | QEFTVPDQSATISGLKPGVDYTITVYALEKAHYYRQNISINYRT |
| | | EIDKPSQ |
| 229 | ATI 1421_D05 w/ N leader | GVSDVPRDLEVVAATPTSLLISWYYVQYNDYRITYGETGGNSPV |
| | and C tail + his tag | QEFTVPDQSATISGLKPGVDYTITVYALEKAHYYRQNISINYRT |
| | | EIDKPSQHHHHHH |
| 230 | ATI 1421_D05 w/ N leader | GVSDVPRDLEVVAATPTSLLISWYYVQYNDYRITYGETGGNSPV |
| | and modified C-terminus | QEFTVPDQSATISGLKPGVDYTITVYALEKAHYYRQNISINYRT |
| | including PC | PC |
| 231 | ATI 1421_D05 w/ N leader | GVSDVPRDLEVVAATPTSLLISWYYVQYNDYRITYGETGGNSPV |
| | and modified C-terminus | QEFTVPDQSATISGLKPGVDYTITVYALEKAHYYRQNISINYRT |
| | including PC + his tag | PCHHHHHH |
| 232 | ATI 1421_D05-full | MGVSDVPRDLEVVAATPTSLLISWYYVQYNDYRITYGETGGNSP |
| | length | VQEFTVPDQSATISGLKPGVDYTITVYALEKAHYYRONISINYR |
| | | TEIDKPSQHHHHHH |
| 233 | ATI_1421_D06 core | EVVAATPTSLLISWGHNYDDEYRITYGETGGNSPVQEFTVPDQY |
| | | ATISGLKPGVDYTITVYALAEAHVRKNHISINYRT |
| 234 | ATI_1421_D06 BC loop | GHNYDDE |
| 235 | ATI_1421_D06 DE loop | PDQY |
| 236 | ATI_1421_D06 FG loop | LAEAHVRKNH |
| 237 | ATI_1421_D06 w/ N | GVSDVPRDLEVVAATPTSLLISWGHNYDDEYRITYGETGGNSPV |
| | leader | QEFTVPDQYATISGLKPGVDYTITVYALAEAHVRKNHISINYRT |
| 238 | ATI_1421_D06 w/ N | GVSDVPRDLEVVAATPTSLLISWGHNYDDEYRITYGETGGNSPV |
| | leader + his tag | QEFTVPDQYATISGLKPGVDYTITVYALAEAHVRKNHISINYRT |
| | | HHHHHH |
| 239 | ATI_1421_D06 w/ N | GVSDVPRDLEVVAATPTSLLISWGHNYDDEYRITYGETGGNSPV |
| | leader and C tail | QEFTVPDQYATISGLKPGVDYTITVYALAEAHVRKNHISINYRT |
| | | EIDKPSQ |
| 240 | ATI_1421_D06 w/ N | GVSDVPRDLEVVAATPTSLLISWGHNYDDEYRITYGETGGNSPV |
| | leader and C tail + his | QEFTVPDQYATISGLKPGVDYTITVYALAEAHVRKNHISINYRT |
| | tag | EIDKPSQHHHHHH |
| 241 | ATI_1421_D06 w/ N | GVSDVPRDLEVVAATPTSLLISWGHNYDDEYRITYGETGGNSPV |
| | leader and modified C- | QEFTVPDQYATISGLKPGVDYTITVYALAEAHVRKNHISINYRT |
| | terminus including PC | PC |
| 242 | ATI_1421_D06 w/ N | GVSDVPRDLEVVAATPTSLLISWGHNYDDEYRITYGETGGNSPV |
| | leader and modified C- | QEFTVPDQYATISGLKPGVDYTITVYALAEAHVRKNHISINYRT |
| | terminus including PC + | PCHHHHHH |
| | his tag | |
| 243 | ATI_1421_D06 - full | MGVSDVPRDLEVVAATPTSLLISWGHNYDDEYRITYGETGGNSP |
| | length | VQEFTVPDQYATISGLKPGVDYTITVYALAEAHVRKNHISINYR |
| | | TEIDKPSQHHHHHH |
| 244 | ATI_1421_E03 core | EVVAATPTSLLISWVYHYDAQYRITYGETGGNSPVQEFTVPDQK |
| | | ATISGLKPGVDYTITVYALSEAHHKRDSISINYRT |
| 245 | ATI_1421_E03 BC loop | VYHYDAQ |
| 246 | ATI_1421_E03 DE loop | PDQK |
| 247 | ATI_1421_E03 FG loop | LSEAHHKRDS |
| 248 | ATI_1421_E03 w/ N leader | GVSDVPRDLEVVAATPTSLLISWVYHYDAQYRITYGETGGNSPV |
| | | QEFTVPDQKATISGLKPGVDYTITVYALSEAHHKRDSISINYRT |
| | | HHHHHH |
| 249 | ATI_1421_E03 w/ N leader | GVSDVPRDLEVVAATPTSLLISWVYHYDAQYRITYGETGGNSPV |
| | + his tag | QEFTVPDQKATISGLKPGVDYTITVYALSEAHHKRDSISINYRT |
| | | EIDKPSQ |
| 250 | ATI_1421_E03 w/ N leader | GVSDVPRDLEVVAATPTSLLISWVYHYDAQYRITYGETGGNSPV |
| | and C tail | QEFTVPDQKATISGLKPGVDYTITVYALSEAHHKRDSISINYRT |
| | | EIDKPSQHHHHHH |
| 251 | ATI_1421_E03 w/ N leader | GVSDVPRDLEVVAATPTSLLISWVYHYDAQYRITYGETGGNSPV |
| | and C tail + his tag | QEFTVPDQKATISGLKPGVDYTITVYALSEAHHKRDSISINYRT |
| | | PC |
| 252 | ATI_1421_E03 w/ N leader | GVSDVPRDLEVVAATPTSLLISWVYHYDAQYRITYGETGGNSPV |
| | and modified C-terminus | QEFTVPDQKATISGLKPGVDYTITVYALSEAHHKRDSISINYRT |
| | including PC | PCHHHHHH |
| 253 | ATI_1421_E03 w/ N leader | GVSDVPRDLEVVAATPTSLLISWVYHYDAQYRITYGETGGNSPV |
| | and modified C-terminus | QEFTVPDQKATISGLKPGVDYTITVYALSEAHHKRDSISINYRT |
| | including PC + his tag | |
| 254 | ATI_1421_E03-full | MGVSDVPRDLEVVAATPTSLLISWVYHYDAQYRITYGETGGNSP |
| | length | VQEFTVPDQKATISGLKPGVDYTITVYALSEAHHKRDSISINYR |
| | | TEIDKPSQHHHHHH |
| 255 | ATI_1421_E04 core | EVVAATPTSLLISWSYNGPIEYRITYGETGGNSPVQEFTVPDQQ |
| | | ATISGLKPGVDYTITVYALEEAHYSROSISINYRT |
| 256 | ATI_1421_E04 BC loop | SYNGPIE |
| 257 | ATI_1421_E04 DE loop | PDQQ |
| 258 | ATI_1421_E04 FG loop | LEEAHYSRQS |
| 259 | ATI_1421_E04 w/ N leader | GVSDVPRDLEVVAATPTSLLISWSYNGPIEYRITYGETGGNSPV |
| | | QEFTVPDQQATISGLKPGVDYTITVYALEEAHYSROSISINYRT |
| 260 | ATI_1421_E04 w/ N leader | GVSDVPRDLEVVAATPTSLLISWSYNGPIEYRITYGETGGNSPV |
| | + his tag | QEFTVPDQQATISGLKPGVDYTITVYALEEAHYSRQSISINYRT |
| | | HHHHHH |
| 261 | ATI_1421_E04 w/ N | GVSDVPRDLEVVAATPTSLLISWSYNGPIEYRITYGETGGNSPV |
| | leader and C tail | QEFTVPDQQATISGLKPGVDYTITVYALEEAHYSRQSISINYRT |
| | | EIDKPSQ |
| 262 | ATI_1421_E04 w/ N leader | GVSDVPRDLEVVAATPTSLLISWSYNGPIEYRITYGETGGNSPV |
| | and C tail + his tag | QEFTVPDQQATISGLKPGVDYTITVYALEEAHYSRQSISINYRT |
| | | EIDKPSQHHHHHH |
| 263 | ATI_1421_E04 w/ N leader | GVSDVPRDLEVVAATPTSLLISWSYNGPIEYRITYGETGGNSPV |
| | and modified C-terminus | QEFTVPDQQATISGLKPGVDYTITVYALEEAHYSROSISINYRT |
| | including PC | PC |
| 264 | ATI_1421_E04 w/ N leader | GVSDVPRDLEVVAATPTSLLISWSYNGPIEYRITYGETGGNSPV |
| | and modified C-terminus | QEFTVPDQQATISGLKPGVDYTITVYALEEAHYSRQSISINYRT |
| | including PC + his tag | PCHHHHHH |
| 265 | ATI_1421_E04-full length | MGVSDVPRDLEVVAATPTSLLISWSYNGPIEYRITYGETGGNSP |
| | | VQEFTVPDQQATISGLKPGVDYTITVYALEEAHYSRQSISINYR |
| | | TEIDKPSQHHHHHH |
| 266 | ATI_1421_F03 core | EVVAATPTSLLISWISVQTYDYRITYGETGGNSPVQEFTVPDQS |
| | | ATISGLKPGVDYTITVYALEKAHYYRQNISINYRT |
| 267 | ATI_1421_F03 BC loop | ISVQTYD |
| 268 | ATI_1421_F03 DE loop | PDQS |
| 269 | ATI_1421_F03 FG loop | LEKAHYYRON |
| 270 | ATI_1421_F03 w/ N leader | GVSDVPRDLEVVAATPTSLLISWISVQTYDYRITYGETGGNSPV |
| | | QEFTVPDQSATISGLKPGVDYTITVYALEKAHYYRQNISINYRT |
| 271 | ATI_1421_F03 w/ N leader | GVSDVPRDLEVVAATPTSLLISWISVQTYDYRITYGETGGNSPV |
| | + his tag | QEFTVPDQSATISGLKPGVDYTITVYALEKAHYYRQNISINYRT |
| | | HHHHHH |
| 272 | ATI_1421_F03 w/ N | GVSDVPRDLEVVAATPTSLLISWISVQTYDYRITYGETGGNSPV |
| | leader and C tail | QEFTVPDQSATISGLKPGVDYTITVYALEKAHYYRQNISINYRT |
| | | EIDKPSQ |
| 273 | ATI_1421_F03 w/ N leader | GVSDVPRDLEVVAATPTSLLISWISVQTYDYRITYGETGGNSPV |
| | and C tail + his tag | QEFTVPDQSATISGLKPGVDYTITVYALEKAHYYRQNISINYRT |
| | | EIDKPSQHHHHHH |
| 274 | ATI_1421_F03 w/ N leader | GVSDVPRDLEVVAATPTSLLISWISVQTYDYRITYGETGGNSPV |
| | and modified C-terminus | QEFTVPDQSATISGLKPGVDYTITVYALEKAHYYRQNISINYRT |
| | including PC | PC |
| 275 | ATI_1421_F03 w/ N leader | GVSDVPRDLEVVAATPTSLLISWISVQTYDYRITYGETGGNSPV |
| | and modified C-terminus | QEFTVPDQSATISGLKPGVDYTITVYALEKAHYYRQNISINYRT |
| | including PC + his tag | PCHHHHHH |
| 276 | ATI_1421_F03-full length | MGVSDVPRDLEVVAATPTSLLISWISVQTYDYRITYGETGGNSP |
| | | VQEFTVPDQSATISGLKPGVDYTITVYALEKAHYYRONISINYR |
| | | TEIDKPSQHHHHHH |
| 277 | ATI_1421_F05 core | EVVAATPTSLLISWLARHDARYRITYGETGGNSPVQEFTVPDRM |
| | | ATISGLKPGVDYTITVYALEQAHYYRLYISINYRT |
| 278 | ATI_1421_F05 BC loop | LARHDAR |
| 279 | ATI_1421_F05 DE loop | PDRM |
| 280 | ATI_1421_F05 FG loop | LEQAHYYRLY |
| 281 | ATI_1421_F05 w/ N leader | GVSDVPRDLEVVAATPTSLLISWLARHDARYRITYGETGGNSPV |
| | | QEFTVPDRMATISGLKPGVDYTITVYALEQAHYYRLYISINYRT |
| 282 | ATI_1421_F05 w/ N leader | GVSDVPRDLEVVAATPTSLLISWLARHDARYRITYGETGGNSPV |
| | + his tag | QEFTVPDRMATISGLKPGVDYTITVYALEQAHYYRLYISINYRT |
| | | HHHHHH |
| 283 | ATI_1421_F05 w/ N leader | GVSDVPRDLEVVAATPTSLLISWLARHDARYRITYGETGGNSPV |
| | and C tail | QEFTVPDRMATISGLKPGVDYTITVYALEQAHYYRLYISINYRT |
| | | EIDKPSQ |
| 284 | ATI_1421_F05 w/ N leader | GVSDVPRDLEVVAATPTSLLISWLARHDARYRITYGETGGNSPV |
| | and C tail + his tag | QEFTVPDRMATISGLKPGVDYTITVYALEQAHYYRLYISINYRT |
| | | EIDKPSQHHHHHH |
| 285 | ATI_1421_F05 w/ N leader | GVSDVPRDLEVVAATPTSLLISWLARHDARYRITYGETGGNSPV |
| | and modified C-terminus | QEFTVPDRMATISGLKPGVDYTITVYALEQAHYYRLYISINYRT |
| | including PC | PC |
| 286 | ATI_1421_F05 w/ N leader | GVSDVPRDLEVVAATPTSLLISWLARHDARYRITYGETGGNSPV |
| | and modified C-terminus | QEFTVPDRMATISGLKPGVDYTITVYALEQAHYYRLYISINYRT |
| | including PC + his tag | PCHHHHHH |
| 287 | ATI_1421_F05-full length | MGVSDVPRDLEVVAATPTSLLISWLARHDARYRITYGETGGNSP |
| | | VQEFTVPDRMATISGLKPGVDYTITVYALEQAHYYRLYISINYR |
| | | TEIDKPSQHHHHHH |
| 288 | ATI_1421_G07 core | EVVAATPTSLLISWHSPTSGITYRITYGETGGNSPVQEFTVPYD |
| | | PSATISGLKPGVDYTITVYAPYGSQYYPGYHISINYRT |
| 289 | ATI_1421_G07 BC loop | HSPTSGIT |
| 290 | ATI_1421_G07 DE loop | PYDPS |
| 291 | ATI_1421_G07 FG loop | PYGSQYYPGYH |
| 292 | ATI_1421_G07 w/ N | GVSDVPRDLEVVAATPTSLLISWHSPTSGITYRITYGETGGNSP |
| | leader | VQEFTVPYDPSATISGLKPGVDYTITVYAPYGSQYYPGYHISIN |
| | | YRT |
| 293 | ATI_1421_G07 w/ N | GVSDVPRDLEVVAATPTSLLISWHSPTSGITYRITYGETGGNSP |
| | leader + his tag | VQEFTVPYDPSATISGLKPGVDYTITVYAPYGSQYYPGYHISIN |
| | | YRTHHHHHH |
| 294 | ATI_1421_G07 w/ N | GVSDVPRDLEVVAATPTSLLISWHSPTSGITYRITYGETGGNSP |
| | leader and C tail | VQEFTVPYDPSATISGLKPGVDYTITVYAPYGSQYYPGYHISIN |
| | | YRTEIDKPSQ |
| 295 | ATI_1421_G07 w/ N | GVSDVPRDLEVVAATPTSLLISWHSPTSGITYRITYGETGGNSP |
| | leader and C tail + his | VQEFTVPYDPSATISGLKPGVDYTITVYAPYGSQYYPGYHISIN |
| | tag | YRTEIDKPSQHHHHHH |
| 296 | ATI_1421_G07 w/ N | GVSDVPRDLEVVAATPTSLLISWHSPTSGITYRITYGETGGNSP |
| | leader and modified C- | VQEFTVPYDPSATISGLKPGVDYTITVYAPYGSQYYPGYHISIN |
| | terminus including PC | YRTPC |
| 297 | ATI_1421_G07 w/ N | GVSDVPRDLEVVAATPTSLLISWHSPTSGITYRITYGETGGNSP |
| | leader and modified C- | VQEFTVPYDPSATISGLKPGVDYTITVYAPYGSQYYPGYHISIN |
| | terminus including PC + | YRTPCHHHHHH |
| | his tag | |
| 298 | ATI_1421_G07-full | MGVSDVPRDLEVVAATPTSLLISWHSPTSGITYRITYGETGGNS |
| | length | PVQEFTVPYDPSATISGLKPGVDYTITVYAPYGSQYYPGYHISI |
| | | NYRTEIDKPSQHHHHHH |
| 299 | ATI_1421_H03 core | EVVAATPTSLLISWVYHYDAQYRITYGETGGNSPVQEFTVPDSS |
| | | ATISGLKPGVDYTITVYALEQAHIDRTTISINYRT |
| 300 | ATI_1421_H03 BC loop | VYHYDAQ |
| 301 | ATI_1421_H03 DE loop | PDSS |
| 302 | ATI_1421_H03 FG loop | LEQAHIDRTT |
| 303 | ATI_1421_H03 w/ N | GVSDVPRDLEVVAATPTSLLISWVYHYDAQYRITYGETGGNSPV |
| | leader | QEFTVPDSSATISGLKPGVDYTITVYALEQAHIDRTTISINYRT |
| 304 | ATI_1421_H03 w/ N | GVSDVPRDLEVVAATPTSLLISWVYHYDAQYRITYGETGGNSPV |
| | leader + his tag | QEFTVPDSSATISGLKPGVDYTITVYALEQAHIDRTTISINYRT |
| | | HHHHHH |
| 305 | ATI_1421_H03 w/ N | GVSDVPRDLEVVAATPTSLLISWVYHYDAQYRITYGETGGNSPV |
| | leader and C tail | QEFTVPDSSATISGLKPGVDYTITVYALEQAHIDRTTISINYRT |
| | | EIDKPSQ |
| 306 | ATI_1421_H03 w/ N | GVSDVPRDLEVVAATPTSLLISWVYHYDAQYRITYGETGGNSPV |
| | leader and C tail + his | QEFTVPDSSATISGLKPGVDYTITVYALEQAHIDRTTISINYRT |
| | tag | EIDKPSQHHHHHH |
| 307 | ATI_1421_H03 w/ N | GVSDVPRDLEVVAATPTSLLISWVYHYDAQYRITYGETGGNSPV |
| | leader and modified C- | QEFTVPDSSATISGLKPGVDYTITVYALEQAHIDRTTISINYRT |
| | terminus including PC | PC |
| 308 | ATI_1421_H03 w/ N | GVSDVPRDLEVVAATPTSLLISWVYHYDAQYRITYGETGGNSPV |
| | leader and modified C- | QEFTVPDSSATISGLKPGVDYTITVYALEQAHIDRTTISINYRT |
| | terminus including PC + | PCHHHHHH |
| | his tag | |
| 309 | ATI_1421_H03-full | MGVSDVPRDLEVVAATPTSLLISWVYHYDAQYRITYGETGGNSP |
| | length | VQEFTVPDSSATISGLKPGVDYTITVYALEQAHIDRTTISINYR |
| | | TEIDKPSQHHHHHH |
| 310 | ATI_1421_H05 core | EVVAATPTSLLISWTSVLLKDYRITYGETGGNSPVQEFTVPDQH |
| | | ATISGLKPGVDYTITVYALQNAHHERLYISINYRT |
| 311 | ATI_1421_H05 BC loop | TSVLLKD |
| 312 | ATI_1421_H05 DE loop | PDQH |
| 313 | ATI_1421_H05 FG loop | LQNAHHERLY |
| 314 | ATI_1421_H05 w/ N | GVSDVPRDLEVVAATPTSLLISWTSVLLKDYRITYGETGGNSPV |
| | leader | QEFTVPDQHATISGLKPGVDYTITVYALQNAHHERLYISINYRT |
| 315 | ATI_1421_H05 w/ N | GVSDVPRDLEVVAATPTSLLISWTSVLLKDYRITYGETGGNSPV |
| | leader + his tag | QEFTVPDQHATISGLKPGVDYTITVYALQNAHHERLYISINYRT |
| | | HHHHHH |
| 316 | ATI_1421_H05 w/ N | GVSDVPRDLEVVAATPTSLLISWTSVLLKDYRITYGETGGNSPV |
| | leader and C tail | QEFTVPDQHATISGLKPGVDYTITVYALQNAHHERLYISINYRT |
| | | EIDKPSQ |
| 317 | ATI_1421_H05 w/ N | GVSDVPRDLEVVAATPTSLLISWTSVLLKDYRITYGETGGNSPV |
| | leader and C tail + his | QEFTVPDQHATISGLKPGVDYTITVYALQNAHHERLYISINYRT |
| | tag | EIDKPSQHHHHHH |
| 318 | ATI_1421_H05 w/ N | GVSDVPRDLEVVAATPTSLLISWTSVLLKDYRITYGETGGNSPV |
| | leader and modified C- | QEFTVPDQHATISGLKPGVDYTITVYALQNAHHERLYISINYRT |
| | terminus including PC | PC |
| 319 | ATI_1421_H05 w/ N | GVSDVPRDLEVVAATPTSLLISWTSVLLKDYRITYGETGGNSPV |
| | leader and modified C- | QEFTVPDQHATISGLKPGVDYTITVYALQNAHHERLYISINYRT |
| | terminus including PC + | PCHHHHHH |
| | his tag | |
| 320 | ATI_1421_H05-full | MGVSDVPRDLEVVAATPTSLLISWTSVLLKDYRITYGETGGNSP |
| | length | VQEFTVPDQHATISGLKPGVDYTITVYALQNAHHERLYISINYR |
| | | TEIDKPSQHHHHHH |
| 321 | ATI_1422_E06 core | EVVAATPTSLLISWLPSYYITYRITYGETGGNSPVQEFTVSKDL |
| | | ATISGLKPGVDYTITVYAFNGSSYYTFGISINYRT |
| 322 | ATI_1422_E06 BC loop | LPSYYIT |
| 323 | ATI_1422_E06 DE loop | SKDL |
| 324 | ATI_1422_E06 FG loop | FNGSSYYTFG |
| 325 | ATI_1422_E06 w/ N leader | GVSDVPRDLEVVAATPTSLLISWLPSYYITYRITYGETGGNSPV |
| | | QEFTVSKDLATISGLKPGVDYTITVYAFNGSSYYTFGISINYRT |
| 326 | ATI_1422_E06 w/ N leader | GVSDVPRDLEVVAATPTSLLISWLPSYYITYRITYGETGGNSPV |
| | + his tag | QEFTVSKDLATISGLKPGVDYTITVYAFNGSSYYTFGISINYRT |
| | | EIDKPSQHHHHHH |
| 327 | ATI_1422_E06 w/ N leader | GVSDVPRDLEVVAATPTSLLISWLPSYYITYRITYGETGGNSPV |
| | and C tail | QEFTVSKDLATISGLKPGVDYTITVYAFNGSSYYTFGISINYRT |
| | | EIDKPSQHHHHHH |
| 328 | ATI_1422_E06 w/ N leader | GVSDVPRDLEVVAATPTSLLISWLPSYYITYRITYGETGGNSPV |
| | and C tail + his tag | QEFTVSKDLATISGLKPGVDYTITVYAFNGSSYYTFGISINYRT |
| | | PC |
| 329 | ATI_1422_E06 w/ N leader | GVSDVPRDLEVVAATPTSLLISWLPSYYITYRITYGETGGNSPV |
| | and modified C-terminus | QEFTVSKDLATISGLKPGVDYTITVYAFNGSSYYTFGISINYRT |
| | including PC | PCHHHHHH |
| 330 | ATI_1422_E06 w/ N leader | GVSDVPRDLEVVAATPTSLLISWLPSYYITYRITYGETGGNSPV |
| | and modified C-terminus | QEFTVSKDLATISGLKPGVDYTITVYAFNGSSYYTFGISINYRT |
| | including PC + his tag | EIDKPSQHHHHHH |
| 331 | ATI_1422_E06-full length | MGVSDVPRDLEVVAATPTSLLISWLPSYYITYRITYGETGGNSP |
| | | VQEFTVSKDLATISGLKPGVDYTITVYAFNGSSYYTFGISINYR |
| | | T |
| 332 | ATI_1422_F04 core | EVVAATPTSLLISWSIPSYFISYRITYGETGGNSPVQEFTVYKN |
| | | YATISGLKPGVDYTITVYASEGIMFYNISINYRT |
| 333 | ATI_1422_F04 BC loop | SIPSYFIS |
| 334 | ATI_1422_F04 DE loop | YKNY |
| 335 | ATI_1422_F04 FG loop | SEGIMFYN |
| 336 | ATI_1422_F04 w/ N leader | GVSDVPRDLEVVAATPTSLLISWSIPSYFISYRITYGETGGNSP |
| | | VQEFTVYKNYATISGLKPGVDYTITVYASEGIMFYNISINYRT |
| 337 | ATI_1422_F04 w/ N leader | GVSDVPRDLEVVAATPTSLLISWSIPSYFISYRITYGETGGNSP |
| | + his tag | VQEFTVYKNYATISGLKPGVDYTITVYASEGIMFYNISINYRTH |
| | | HHHHH |
| 338 | ATI_1422_F04 w/ N | GVSDVPRDLEVVAATPTSLLISWSIPSYFISYRITYGETGGNSP |
| | leader and C tail | VQEFTVYKNYATISGLKPGVDYTITVYASEGIMFYNISINYRTE |
| | | IDKPSQ |
| 339 | ATI_1422_F04 w/ N leader | GVSDVPRDLEVVAATPTSLLISWSIPSYFISYRITYGETGGNSP |
| | and C tail + his tag | VQEFTVYKNYATISGLKPGVDYTITVYASEGIMFYNISINYRTE |
| | | IDKPSQHHHHHH |
| 340 | ATI_1422_F04 w/ N leader | GVSDVPRDLEVVAATPTSLLISWSIPSYFISYRITYGETGGNSP |
| | and modified C-terminus | VQEFTVYKNYATISGLKPGVDYTITVYASEGIMFYNISINYRTP |
| | including PC | C |
| 341 | ATI_1422_F04 w/ N leader | GVSDVPRDLEVVAATPTSLLISWSIPSYFISYRITYGETGGNSP |
| | and modified C-terminus | VQEFTVYKNYATISGLKPGVDYTITVYASEGIMFYNISINYRTP |
| | including PC + his tag | CHHHHHH |
| 342 | ATI_1422_F04-full length | MGVSDVPRDLEVVAATPTSLLISWSIPSYFISYRITYGETGGNS |
| | | PVQEFTVYKNYATISGLKPGVDYTITVYASEGIMFYNISINYRT |
| | | EIDKPSQHHHHHH |
| 343 | ATI_1422_F05 core | EVVAATPTSLLISWPYPRGPYVFYRITYGETGGNSPVQEFTVYP |
| | | GQATISGLKPGVDYTITVYAYTSGYVISINYRT |
| 344 | ATI_1422_F05 BC loop | PYPRGPYVF |
| 345 | ATI_1422_F05 DE loop | YPGQ |
| 346 | ATI_1422_F05 FG loop | YTSGYV |
| 347 | ATI_1422_F05 w/ N leader | GVSDVPRDLEVVAATPTSLLISWPYPRGPYVFYRITYGETGGNS |
| | | PVQEFTVYPGQATISGLKPGVDYTITVYAYTSGYVISINYRT |
| 348 | ATI_1422_F05 w/ N leader | GVSDVPRDLEVVAATPTSLLISWPYPRGPYVFYRITYGETGGNS |
| | + his tag | PVQEFTVYPGQATISGLKPGVDYTITVYAYTSGYVISINYRTEI |
| | | DKPSQHHHHHH |
| 349 | ATI_1422_F05 w/ N | GVSDVPRDLEVVAATPTSLLISWPYPRGPYVFYRITYGETGGNS |
| | leader and C tail | PVQEFTVYPGQATISGLKPGVDYTITVYAYTSGYVISINYRTEI |
| | | DKPSQ |
| 350 | ATI_1422_F05 w/ N leader | GVSDVPRDLEVVAATPTSLLISWPYPRGPYVFYRITYGETGGNS |
| | and C tail + his tag | PVQEFTVYPGQATISGLKPGVDYTITVYAYTSGYVISINYRTEI |
| | | DKPSQHHHHHH |
| 351 | ATI_1422_F05 w/ N leader | GVSDVPRDLEVVAATPTSLLISWPYPRGPYVFYRITYGETGGNS |
| | and modified C-terminus | PVQEFTVYPGQATISGLKPGVDYTITVYAYTSGYVISINYRTPC |
| | including PC | |
| 352 | ATI_1422_F05 w/ N leader | GVSDVPRDLEVVAATPTSLLISWPYPRGPYVFYRITYGETGGNS |
| | and modified C-terminus | PVQEFTVYPGQATISGLKPGVDYTITVYAYTSGYVISINYRTPC |
| | including PC + his tag | HHHHHH |
| 353 | ATI_1422_F05- | MGVSDVPRDLEVVAATPTSLLISWPYPRGPYVFYRITYGETGGN |
| | full length | SPVQEFTVYPGQATISGLKPGVDYTITVYAYTSGYVISINYRTE |
| | | IDKPSQHHHHHH |
| 354 | ATI_1422_H04 core | EVVAATPTSLLISWYLPSYYVQYRITYGETGGNSPVQEFTVKSY |
| | | NATISGLKPGVDYTITVYARMGVYYLSYSISINYRT |
| 355 | ATI_1422_H04 BC loop | YLPSYYVQ |
| 356 | ATI_1422_H04 DE loop | KSYN |
| 357 | ATI_1422_H04 FG loop | RMGVYYLSYS |
| 358 | ATI_1422_H04 w/ N | GVSDVPRDLEVVAATPTSLLISWYLPSYYVQYRITYGETGGNSP |
| | leader | VQEFTVKSYNATISGLKPGVDYTITVYARMGVYYLSYSISINYR |
| | | T |
| 359 | ATI_1422_H04 w/ N | GVSDVPRDLEVVAATPTSLLISWYLPSYYVQYRITYGETGGNSP |
| | leader + his tag | VQEFTVKSYNATISGLKPGVDYTITVYARMGVYYLSYSISINYR |
| | | THHHHHH |
| 360 | ATI_1422_H04 w/ N | GVSDVPRDLEVVAATPTSLLISWYLPSYYVQYRITYGETGGNSP |
| | leader and C tail | VQEFTVKSYNATISGLKPGVDYTITVYARMGVYYLSYSISINYR |
| | | TEIDKPSQ |
| 361 | ATI_1422_H04 w/ N | GVSDVPRDLEVVAATPTSLLISWYLPSYYVQYRITYGETGGNSP |
| | leader and C tail + his | VQEFTVKSYNATISGLKPGVDYTITVYARMGVYYLSYSISINYR |
| | tag | TEIDKPSQHHHHHH |
| 362 | ATI_1422_H04 w/ N | GVSDVPRDLEVVAATPTSLLISWYLPSYYVQYRITYGETGGNSP |
| | leader and modified C- | VQEFTVKSYNATISGLKPGVDYTITVYARMGVYYLSYSISINYR |
| | terminus including PC | TPC |
| 363 | ATI_1422_H04 w/ N | GVSDVPRDLEVVAATPTSLLISWYLPSYYVQYRITYGETGGNSP |
| | leader and modified C- | VQEFTVKSYNATISGLKPGVDYTITVYARMGVYYLSYSISINYR |
| | terminus including PC + | TPCHHHHHH |
| | his tag | |
| 364 | ATI_1422_H04-full | MGVSDVPRDLEVVAATPTSLLISWYLPSYYVQYRITYGETGGNS |
| | length | PVQEFTVKSYNATISGLKPGVDYTITVYARMGVYYLSYSISINY |
| | | RTEIDKPSQHHHHHH |
| 365 | ATI_1422_H05 core | EVVAATPTSLLISWQGQLSPSFYRITYGETGGNSPVQEFTVVAG |
| | | MATISGLKPGVDYTITVYATSDVYFYSISINYRT |
| 366 | ATI_1422_H05 BC loop | QGQLSPSF |
| 367 | ATI_1422_H05 DE loop | VAGM |
| 368 | ATI_1422_H05 FG loop | TSDVYFYS |
| 369 | ATI_1422_H05 w/ N | GVSDVPRDLEVVAATPTSLLISWQGQLSPSFYRITYGETGGNSP |
| | leader | VQEFTVVAGMATISGLKPGVDYTITVYATSDVYFYSISINYRT |
| 370 | ATI_1422_H05 w/ N | GVSDVPRDLEVVAATPTSLLISWQGQLSPSFYRITYGETGGNSP |
| | leader + his tag | VQEFTVVAGMATISGLKPGVDYTITVYATSDVYFYSISINYRTH |
| | | HHHHH |
| 371 | ATI_1422_H05 w/ N | GVSDVPRDLEVVAATPTSLLISWQGQLSPSFYRITYGETGGNSP |
| | leader and C tail | VQEFTVVAGMATISGLKPGVDYTITVYATSDVYFYSISINYRTE |
| | | IDKPSQ |
| 372 | ATI_1422_H05 w/ N | GVSDVPRDLEVVAATPTSLLISWQGQLSPSFYRITYGETGGNSP |
| | leader and C tail + his | VQEFTVVAGMATISGLKPGVDYTITVYATSDVYFYSISINYRTE |
| | tag | IDKPSQHHHHHH |
| 373 | ATI_1422_H05 w/ N | GVSDVPRDLEVVAATPTSLLISWQGQLSPSFYRITYGETGGNSP |
| | leader and modified C- | VQEFTVVAGMATISGLKPGVDYTITVYATSDVYFYSISINYRTP |
| | terminus including PC | C |
| 374 | ATI_1422_H05 w/ N | GVSDVPRDLEVVAATPTSLLISWQGQLSPSFYRITYGETGGNSP |
| | leader and modified C- | VQEFTVVAGMATISGLKPGVDYTITVYATSDVYFYSISINYRTP |
| | terminus including PC + | CHHHHHH |
| | his tag | |
| 375 | -full length | MGVSDVPRDLEVVAATPTSLLISWQGQLSPSFYRITYGETGGNS |
| | | PVQEFTVVAGMATISGLKPGVDYTITVYATSDVYFYSISINYRT |
| | | EIDKPSQHHHHHH |
| 376 | ATI_1422_G05 core | EVVAATPTSLLISWIAPYYSVIYRITYGETGGNSPVQEFTVTGS |
| | | GYATISGLKPGVDYTITVYATYCASVASYAFISINYRT |
| 377 | ATI_1422_G05 BC loop | IAPYYSVI |
| 378 | ATI_1422_G05 DE loop | TGSGY |
| 379 | ATI_1422_G05 FG loop | TYCASVASYAF |
| 380 | ATI_1422_G05 w/ N | GVSDVPRDLEVVAATPTSLLISWIAPYYSVIYRITYGETGGNSP |
| | leader | VQEFTVTGSGYATISGLKPGVDYTITVYATYCASVASYAFISIN |
| | | YRT |
| 381 | ATI_1422_G05 w/ N | GVSDVPRDLEVVAATPTSLLISWIAPYYSVIYRITYGETGGNSP |
| | leader + his tag | VQEFTVTGSGYATISGLKPGVDYTITVYATYCASVASYAFISIN |
| | | YRTHHHHHH |
| 382 | ATI_1422_G05 w/ N | GVSDVPRDLEVVAATPTSLLISWIAPYYSVIYRITYGETGGNSP |
| | leader and C tail | VQEFTVTGSGYATISGLKPGVDYTITVYATYCASVASYAFISIN |
| | | YRTEIDKPSQ |
| 383 | ATI_1422_G05 w/ N | GVSDVPRDLEVVAATPTSLLISWIAPYYSVIYRITYGETGGNSP |
| | leader and C tail + his | VQEFTVTGSGYATISGLKPGVDYTITVYATYCASVASYAFISIN |
| | tag | YRTEIDKPSQHHHHHH |
| 384 | ATI_1422_G05 w/ N | GVSDVPRDLEVVAATPTSLLISWIAPYYSVIYRITYGETGGNSP |
| | leader and modified C- | VQEFTVIGSGYATISGLKPGVDYTITVYATYCASVASYAFISIN |
| | terminus including PC | YRTPC |
| 385 | ATI_1422_G05 w/ N | GVSDVPRDLEVVAATPTSLLISWIAPYYSVIYRITYGETGGNSP |
| | leader and modified C- | VQEFTVTGSGYATISGLKPGVDYTITVYATYCASVASYAFISIN |
| | terminus including PC + | YRTPCHHHHHH |
| | his tag | |
| 386 | ATI_1422_G05-full | MGVSDVPRDLEVVAATPTSLLISWIAPYYSVIYRITYGETGGNS |
| | length | PVQEFTVTGSGYATISGLKPGVDYTITVYATYCASVASYAFISI |
| | | NYRTEIDKPSQHHHHHH |
| 387 | ATI_1760_C02 core | EVVAATPTSLLISWIAPYYSVIYRITYGETGGNSPVQEFTVPGS |
| | | AYATISGLKPGVDYTITVYASSGASIAAYAFISINYRT |
| 388 | ATI_1760_C02 BC loop | IAPYYSVI |
| 389 | ATI_1760_C02 DE loop | PGSAY |
| 390 | ATI_1760_C02 FG loop | SSGASIAAYAF |
| 391 | ATI_1760_C02 w/ N | GVSDVPRDLEVVAATPTSLLISWIAPYYSVIYRITYGETGGNSP |
| | leader | VQEFTVPGSAYATISGLKPGVDYTITVYASSGASIAAYAFISIN |
| | | YRT |
| 392 | ATI_1760_C02 w/ N | GVSDVPRDLEVVAATPTSLLISWIAPYYSVIYRITYGETGGNSP |
| | leader + his tag | VQEFTVPGSAYATISGLKPGVDYTITVYASSGASIAAYAFISIN |
| | | YRTHHHHHH |
| 393 | ATI_1760_C02 w/ N | GVSDVPRDLEVVAATPTSLLISWIAPYYSVIYRITYGETGGNSP |
| | leader and C tail | VQEFTVPGSAYATISGLKPGVDYTITVYASSGASIAAYAFISIN |
| | | YRTEIDKPSQ |
| 394 | ATI_1760_C02 w/ N | GVSDVPRDLEVVAATPTSLLISWIAPYYSVIYRITYGETGGNSP |
| | leader and C tail + his | VQEFTVPGSAYATISGLKPGVDYTITVYASSGASIAAYAFISIN |
| | tag | YRTEIDKPSQHHHHHH |
| 395 | ATI_1760_C02 w/ N | GVSDVPRDLEVVAATPTSLLISWIAPYYSVIYRITYGETGGNSP |
| | leader and modified C- | VQEFTVPGSAYATISGLKPGVDYTITVYASSGASIAAYAFISIN |
| | terminus including PC | YRTPC |
| 396 | ATI_1760_C02 w/ N | GVSDVPRDLEVVAATPTSLLISWIAPYYSVIYRITYGETGGNSP |
| | leader and modified C- | VQEFTVPGSAYATISGLKPGVDYTITVYASSGASIAAYAFISIN |
| | terminus including PC + | YRTPCHHHHHH |
| | his tag | |
| 397 | ATI_1760_C02-full | MGVSDVPRDLEVVAATPTSLLISWIAPYYSVIYRITYGETGGNS |
| | length | PVQEFTVPGSAYATISGLKPGVDYTITVYASSGASIAAYAFISI |
| | | NYRTEIDKPSQHHHHHH |
| 398 | ATI_1760_E01 core | EVVAATPTSLLISWIAPYYSVKYRITYGETGGNSPVQEFTVAGA |
| | | DYATISGLKPGVDYTITVYATYGASIASYAFISINYRT |
| 399 | ATI_1760_E01 BC loop | IAPYYSVK |
| 400 | ATI_1760_E01 DE loop | AGADY |
| 401 | ATI_1760_E01 FG loop | TYGASIASYAF |
| 402 | ATI_1760_E01 w/ N leader | GVSDVPRDLEVVAATPTSLLISWIAPYYSVKYRITYGETGGNSP |
| | | VQEFTVAGADYATISGLKPGVDYTITVYATYGASIASYAFISIN |
| | | YRT |
| 403 | ATI_1760_E01 w/ N leader | GVSDVPRDLEVVAATPTSLLISWIAPYYSVKYRITYGETGGNSP |
| | + his tag | VQEFTVAGADYATISGLKPGVDYTITVYATYGASIASYAFISIN |
| | | YRTHHHHHH |
| 404 | ATI_1760_E01 w/ N | GVSDVPRDLEVVAATPTSLLISWIAPYYSVKYRITYGETGGNSP |
| | leader and C tail | VQEFTVAGADYATISGLKPGVDYTITVYATYGASIASYAFISIN |
| | | YRTEIDKPSQ |
| 405 | ATI_1760_E01 w/ N leader | GVSDVPRDLEVVAATPTSLLISWIAPYYSVKYRITYGETGGNSP |
| | and C tail + his tag | VQEFTVAGADYATISGLKPGVDYTITVYATYGASIASYAFISIN |
| | | YRTEIDKPSQHHHHHH |
| 406 | ATI_1760_E01 w/ N leader | GVSDVPRDLEVVAATPTSLLISWIAPYYSVKYRITYGETGGNSP |
| | and modified C-terminus | VQEFTVAGADYATISGLKPGVDYTITVYATYGASIASYAFISIN |
| | including PC | YRTPC |
| 407 | ATI_1760_E01 w/ N leader | GVSDVPRDLEVVAATPTSLLISWIAPYYSVKYRITYGETGGNSP |
| | and modified C-terminus | VQEFTVAGADYATISGLKPGVDYTITVYATYGASIASYAFISIN |
| | including PC + his tag | YRTPCHHHHHH |
| 408 | ATI_1760_E01-full length | MGVSDVPRDLEVVAATPTSLLISWIAPYYSVKYRITYGETGGNS |
| | | PVQEFTVAGADYATISGLKPGVDYTITVYATYGASIASYAFISI |
| | | NYRTEIDKPSQHHHHHH |
| 409 | ATI_1760_F01 core | EVVAATPTSLLISWIAPYYAVMYRITYGETGGNSPVQEFTVPGG |
| | | GYATISGLKPGVDYTITVYATGGASIAAYAFISINYRT |
| 410 | ATI_1760_F01 BC loop | IAPYYAVM |
| 411 | ATI_1760_F01 DE loop | PGGGY |
| 412 | ATI_1760_F01 FG loop | TGGASIAAYAF |
| 413 | ATI_1760_F01 w/ N leader | GVSDVPRDLEVVAATPTSLLISWIAPYYAVMYRITYGETGGNSP |
| | | VQEFTVPGGGYATISGLKPGVDYTITVYATGGASIAAYAFISIN |
| | | YRT |
| 414 | ATI_1760_F01 w/ N leader | GVSDVPRDLEVVAATPTSLLISWIAPYYAVMYRITYGETGGNSP |
| | + his tag | VQEFTVPGGGYATISGLKPGVDYTITVYATGGASIAAYAFISIN |
| | | YRTHHHHHH |
| 415 | ATI_1760_F01 w/ N | GVSDVPRDLEVVAATPTSLLISWIAPYYAVMYRITYGETGGNSP |
| | leader and C tail | VQEFTVPGGGYATISGLKPGVDYTITVYATGGASIAAYAFISIN |
| | | YRTEIDKPSQ |
| 416 | ATI_1760_F01 w/ N leader | GVSDVPRDLEVVAATPTSLLISWIAPYYAVMYRITYGETGGNSP |
| | and C tail + his tag | VQEFTVPGGGYATISGLKPGVDYTITVYATGGASIAAYAFISIN |
| | | YRTEIDKPSQHHHHHH |
| 417 | ATI_1760_F01 w/ N leader | GVSDVPRDLEVVAATPTSLLISWIAPYYAVMYRITYGETGGNSP |
| | and modified C-terminus | VQEFTVPGGGYATISGLKPGVDYTITVYATGGASIAAYAFISIN |
| | including PC | YRTPC |
| 418 | ATI_1760_F01 w/ N leader | GVSDVPRDLEVVAATPTSLLISWIAPYYAVMYRITYGETGGNSP |
| | and modified C-terminus | VQEFTVPGGGYATISGLKPGVDYTITVYATGGASIAAYAFISIN |
| | including PC + his tag | YRTPCHHHHHH |
| 419 | ATI_1760_F01-full length | MGVSDVPRDLEVVAATPTSLLISWIAPYYAVMYRITYGETGGNS |
| | | PVQEFTVPGGGYATISGLKPGVDYTITVYATGGASIAAYAFISI |
| | | NYRTEIDKPSQHHHHHH |
| 420 | ATI_1494_D03 core | EVVAATPTSLLISWSYPSYHLYRITYGETGGNSPVQEFTVHIDY |
| | | ATISGLKPGVDYTITVYAQSPPYDIYYEISINYRT |
| 421 | ATI_1494_D03 BC loop | SYPSYHL |
| 422 | ATI_1494_D03 DE loop | HIDY |
| 423 | ATI_1494_D03 FG loop | QSPPYDIYYE |
| 424 | ATI_1494_D03 w/ N | GVSDVPRDLEVVAATPTSLLISWSYPSYHLYRITYGETGGNSPV |
| | leader | QEFTVHIDYATISGLKPGVDYTITVYAQSPPYDIYYEISINYRT |
| 425 | ATI_1494_D03 w/ N | GVSDVPRDLEVVAATPTSLLISWSYPSYHLYRITYGETGGNSPV |
| | leader + his tag | QEFTVHIDYATISGLKPGVDYTITVYAQSPPYDIYYEISINYRT |
| | | HHHHHH |
| 426 | ATI_1494_D03 w/ N | GVSDVPRDLEVVAATPTSLLISWSYPSYHLYRITYGETGGNSPV |
| | leader and C tail | QEFTVHIDYATISGLKPGVDYTITVYAQSPPYDIYYEISINYRT |
| | | EIDKPSQ |
| 427 | ATI_1494_D03 w/ N | GVSDVPRDLEVVAATPTSLLISWSYPSYHLYRITYGETGGNSPV |
| | leader and C tail + his | QEFTVHIDYATISGLKPGVDYTITVYAQSPPYDIYYEISINYRT |
| | tag | EIDKPSQHHHHHH |
| 428 | ATI_1494_D03 w/ N | GVSDVPRDLEVVAATPTSLLISWSYPSYHLYRITYGETGGNSPV |
| | leader and modified C- | QEFTVHIDYATISGLKPGVDYTITVYAQSPPYDIYYEISINYRT |
| | terminus including PC | PC |
| 429 | ATI_1494_D03 w/ N | GVSDVPRDLEVVAATPTSLLISWSYPSYHLYRITYGETGGNSPV |
| | leader and modified C- | QEFTVHIDYATISGLKPGVDYTITVYAQSPPYDIYYEISINYRT |
| | terminus including PC + | PCHHHHHH |
| | his tag | |
| 430 | ATI_1494_D03-full | MGVSDVPRDLEVVAATPTSLLISWSYPSYHLYRITYGETGGNSP |
| | length | VQEFTVHIDYATISGLKPGVDYTITVYAQSPPYDIYYEISINYR |
| | | TEIDKPSQHHHHHH |
| 431 | ATI_1494_D04 core | EVVAATPTSLLISWMESSSNSYRITYGETGGNSPVQEFTVPDQL |
| | | ATISGLKPGVDYTITVYALANAHYMRVGISINYRT |
| 432 | ATI_1494_D04 BC loop | MESSSNS |
| 433 | ATI_1494_D04 DE loop | PDQL |
| 434 | ATI_1494_D04 FG loop | LANAHYMRVG |
| 435 | ATI_1494_D04 w/ N | GVSDVPRDLEVVAATPTSLLISWMESSSNSYRITYGETGGNSPV |
| | leader | QEFTVPDQLATISGLKPGVDYTITVYALANAHYMRVGISINYRT |
| 436 | ATI_1494_D04 w/ N | GVSDVPRDLEVVAATPTSLLISWMESSSNSYRITYGETGGNSPV |
| | leader + his tag | QEFTVPDQLATISGLKPGVDYTITVYALANAHYMRVGISINYRT |
| | | HHHHHH |
| 437 | ATI_1494_D04 w/ N | GVSDVPRDLEVVAATPTSLLISWMESSSNSYRITYGETGGNSPV |
| | leader and C tail | QEFTVPDQLATISGLKPGVDYTITVYALANAHYMRVGISINYRT |
| | | EIDKPSQ |
| 438 | ATI_1494_D04 w/ N | GVSDVPRDLEVVAATPTSLLISWMESSSNSYRITYGETGGNSPV |
| | leader and C tail + his | QEFTVPDQLATISGLKPGVDYTITVYALANAHYMRVGISINYRT |
| | tag | EIDKPSQHHHHHH |
| 439 | ATI_1494_D04 w/ N | GVSDVPRDLEVVAATPTSLLISWMESSSNSYRITYGETGGNSPV |
| | leader and modified C- | QEFTVPDQLATISGLKPGVDYTITVYALANAHYMRVGISINYRT |
| | terminus including PC | PC |
| 440 | ATI_1494_D04 w/ N | GVSDVPRDLEVVAATPTSLLISWMESSSNSYRITYGETGGNSPV |
| | leader and modified C- | QEFTVPDQLATISGLKPGVDYTITVYALANAHYMRVGISINYRT |
| | terminus including PC + | PCHHHHHH |
| | his tag | |
| 441 | ATI_1494_D04-full | MGVSDVPRDLEVVAATPTSLLISWMESSSNSYRITYGETGGNSP |
| | length | VQEFTVPDQLATISGLKPGVDYTITVYALANAHYMRVGISINYR |
| | | TEIDKPSQHHHHHH |
| 442 | ATI_1523_A08 core | EVVAATPTSLLISWISVQTYXYRITYGETGGNSPVQEFTVPDQS |
| | | ATISGLKPGVDYTITVYALEKAHYYRQNISINYRT |
| 443 | ATI_1523_A08 BC loop | ISVQTYX |
| 444 | ATI_1523_A08 DE loop | PDQS |
| 445 | ATI_1523_A08 FG loop | LEKAHYYRON |
| 446 | ATI_1523_A08 w/ N | GVSDVPRDLEVVAATPTSLLISWISVQTYXYRITYGETGGNSPV |
| | leader | QEFTVPDQSATISGLKPGVDYTITVYALEKAHYYRQNISINYRT |
| 447 | ATI_1523_A08 w/ N | GVSDVPRDLEVVAATPTSLLISWISVQTYXYRITYGETGGNSPV |
| | leader + his tag | QEFTVPDQSATISGLKPGVDYTITVYALEKAHYYRQNISINYRT |
| | | HHHHHH |
| 448 | ATI_1523_A08 w/ N | GVSDVPRDLEVVAATPTSLLISWISVQTYXYRITYGETGGNSPV |
| | leader and C tail | QEFTVPDQSATISGLKPGVDYTITVYALEKAHYYRQNISINYRT |
| | | EIDKPSQ |
| 449 | ATI_1523_A08 w/ N | GVSDVPRDLEVVAATPTSLLISWISVQTYXYRITYGETGGNSPV |
| | leader and C tail + his | QEFTVPDQSATISGLKPGVDYTITVYALEKAHYYRQNISINYRT |
| | tag | EIDKPSQHHHHHH |
| 450 | ATI_1523_A08 w/ N | GVSDVPRDLEVVAATPTSLLISWISVQTYXYRITYGETGGNSPV |
| | leader and modified C- | QEFTVPDQSATISGLKPGVDYTITVYALEKAHYYRQNISINYRT |
| | terminus including PC | PC |
| 451 | ATI_1523_A08 w/ N | GVSDVPRDLEVVAATPTSLLISWISVQTYXYRITYGETGGNSPV |
| | leader and modified C- | QEFTVPDQSATISGLKPGVDYTITVYALEKAHYYRQNISINYRT |
| | terminus including PC + | PCHHHHHH |
| | his tag | |
| 452 | ATI_1523_A08-full | MGVSDVPRDLEVVAATPTSLLISWISVQTYXYRITYGETGGNSP |
| | length | VQEFTVPDQSATISGLKPGVDYTITVYALEKAHYYRONISINYR |
| | | TEIDKPSQHHHHHH |
| 453 | ATI_1523_B10 core | EVVAATPTSLLISWVYHYDXQYRITYGETGGNSPVQEFTVPDQK |
| | | ATISGLKPGVDYTITVYALSEAHHKRDSISINYRT |
| 454 | ATI_1523_B10 BC loop | VYHYDXQ |
| 455 | ATI_1523_B10 DE loop | PDQK |
| 456 | ATI_1523_B10 FG loop | LSEAHHKRDS |
| 457 | ATI_1523_B10 w/ N | GVSDVPRDLEVVAATPTSLLISWVYHYDXQYRITYGETGGNSPV |
| | leader | QEFTVPDQKATISGLKPGVDYTITVYALSEAHHKRDSISINYRT |
| 458 | ATI_1523_B10 w/ N | GVSDVPRDLEVVAATPTSLLISWVYHYDXQYRITYGETGGNSPV |
| | leader + his tag | QEFTVPDQKATISGLKPGVDYTITVYALSEAHHKRDSISINYRT |
| | | HHHHHH |
| 459 | ATI_1523_B10 w/ N | GVSDVPRDLEVVAATPTSLLISWVYHYDXQYRITYGETGGNSPV |
| | leader and C tail | QEFTVPDQKATISGLKPGVDYTITVYALSEAHHKRDSISINYRT |
| | | EIDKPSQ |
| 460 | ATI_1523_B10 w/ N | GVSDVPRDLEVVAATPTSLLISWVYHYDXQYRITYGETGGNSPV |
| | leader and C tail + his | QEFTVPDQKATISGLKPGVDYTITVYALSEAHHKRDSISINYRT |
| | tag | EIDKPSQHHHHHH |
| 461 | ATI_1523_B10 w/ N | GVSDVPRDLEVVAATPTSLLISWVYHYDXQYRITYGETGGNSPV |
| | leader and modified C- | QEFTVPDQKATISGLKPGVDYTITVYALSEAHHKRDSISINYRT |
| | terminus including PC | PC |
| 462 | ATI_1523_B10 w/ N | GVSDVPRDLEVVAATPTSLLISWVYHYDXQYRITYGETGGNSPV |
| | leader and modified C- | QEFTVPDQKATISGLKPGVDYTITVYALSEAHHKRDSISINYRT |
| | terminus including PC + | PCHHHHHH |
| | his tag | |
| 463 | ATI_1523_B10-full | MGVSDVPRDLEVVAATPTSLLISWVYHYDXQYRITYGETGGNSP |
| | length | VQEFTVPDQKATISGLKPGVDYTITVYALSEAHHKRDSISINYR |
| | | TEIDKPSQHHHHHH |
| 464 | ATI_1523_C07 core | EVVAATPTSLLISWRMHTDPDYRITYGETGGNSPVQEFTVPDQE |
| | | ATISGLKPGVDYTITVYAIQTAHYYRINISINYRT |
| 465 | ATI_1523_C07 BC loop | RMHTDPD |
| 466 | ATI_1523_C07 DE loop | PDQE |
| 467 | ATI_1523_C07 FG loop | IQTAHYYRIN |
| 468 | ATI_1523_C07 w/ N | GVSDVPRDLEVVAATPTSLLISWRMHTDPDYRITYGETGGNSPV |
| | leader | QEFTVPDQEATISGLKPGVDYTITVYAIQTAHYYRINISINYRT |
| 469 | ATI_1523_C07 w/ N | GVSDVPRDLEVVAATPTSLLISWRMHTDPDYRITYGETGGNSPV |
| | leader + his tag | QEFTVPDQEATISGLKPGVDYTITVYAIQTAHYYRINISINYRT |
| | | HHHHHH |
| 470 | ATI_1523_C07 w/ N | GVSDVPRDLEVVAATPTSLLISWRMHTDPDYRITYGETGGNSPV |
| | leader and C tail | QEFTVPDQEATISGLKPGVDYTITVYAIQTAHYYRINISINYRT |
| | | EIDKPSQ |
| 471 | ATI_1523_C07 w/ N | GVSDVPRDLEVVAATPTSLLISWRMHTDPDYRITYGETGGNSPV |
| | leader and C tail + his | QEFTVPDQEATISGLKPGVDYTITVYAIQTAHYYRINISINYRT |
| | tag | EIDKPSQHHHHHH |
| 472 | ATI_1523_C07 w/ N | GVSDVPRDLEVVAATPTSLLISWRMHTDPDYRITYGETGGNSPV |
| | leader and modified C- | QEFTVPDQEATISGLKPGVDYTITVYAIQTAHYYRINISINYRT |
| | terminus including PC | PC |
| 473 | ATI_1523_C07 w/ N | GVSDVPRDLEVVAATPTSLLISWRMHTDPDYRITYGETGGNSPV |
| | leader and modified C- | QEFTVPDQEATISGLKPGVDYTITVYAIQTAHYYRINISINYRT |
| | terminus including PC + | PCHHHHHH |
| | his tag | |
| 474 | ATI_1523_C07-full | MGVSDVPRDLEVVAATPTSLLISWRMHTDPDYRITYGETGGNSP |
| | length | VQEFTVPDQEATISGLKPGVDYTITVYAIQTAHYYRINISINYR |
| | | TEIDKPSQHHHHHH |
| 475 | ATI_1523_D07 core | EVVAATPTSLLISWENLASYQYRITYGETGGNSPVQEFTVPDVQ |
| | | ATISGLKPGVDYTITVYALPYIHMKQRVISINYRT |
| 476 | ATI_1523_D07 BC loop | ENLASYQ |
| 477 | ATI_1523_D07 DE loop | PDVQ |
| 478 | ATI_1523_D07 FG loop | LPYIHMKQRV |
| 479 | ATI_1523_D07 w/ N | GVSDVPRDLEVVAATPTSLLISWENLASYQYRITYGETGGNSPV |
| | leader | QEFTVPDVQATISGLKPGVDYTITVYALPYIHMKORVISINYRT |
| 480 | ATI_1523_D07 w/ N | GVSDVPRDLEVVAATPTSLLISWENLASYQYRITYGETGGNSPV |
| | leader + his tag | QEFTVPDVQATISGLKPGVDYTITVYALPYIHMKORVISINYRT |
| | | HHHHHH |
| 481 | ATI_1523_D07 w/ N | GVSDVPRDLEVVAATPTSLLISWENLASYQYRITYGETGGNSPV |
| | leader and C tail | QEFTVPDVQATISGLKPGVDYTITVYALPYIHMKORVISINYRT |
| | | EIDKPSQ |
| 482 | ATI_1523_D07 w/ N | GVSDVPRDLEVVAATPTSLLISWENLASYQYRITYGETGGNSPV |
| | leader and C tail + his | QEFTVPDVQATISGLKPGVDYTITVYALPYIHMKORVISINYRT |
| | tag | EIDKPSQHHHHHH |
| 483 | ATI_1523_D07 w/ N | GVSDVPRDLEVVAATPTSLLISWENLASYQYRITYGETGGNSPV |
| | leader and modified C- | QEFTVPDVQATISGLKPGVDYTITVYALPYIHMKORVISINYRT |
| | terminus including PC | PC |
| 484 | ATI_1523_D07 w/ N | GVSDVPRDLEVVAATPTSLLISWENLASYQYRITYGETGGNSPV |
| | leader and modified C- | QEFTVPDVQATISGLKPGVDYTITVYALPYIHMKQRVISINYRT |
| | terminus including PC + | PCHHHHHH |
| | his tag | |
| 485 | ATI_1523_D07-full | MGVSDVPRDLEVVAATPTSLLISWENLASYQYRITYGETGGNSP |
| | length | VQEFTVPDVQATISGLKPGVDYTITVYALPYIHMKORVISINYR |
| | | TEIDKPSQHHHHHH |
| 486 | ATI_1523_D08 core | EVVAATPTSLLISWMRYYDAYYRITYGETGGNSPVQEFTVPDQS |
| | | ATISGLKPGVDYTITVYALEKAHYYRQNISINYRT |
| 487 | ATI_1523_D08 BC loop | MRYYDAY |
| 488 | ATI_1523_D08 DE loop | PDQS |
| 489 | ATI_1523_D08 FG loop | LEKAHYYRON |
| 490 | ATI_1523_D08 w/ N | GVSDVPRDLEVVAATPTSLLISWMRYYDAYYRITYGETGGNSPV |
| | leader | QEFTVPDQSATISGLKPGVDYTITVYALEKAHYYRQNISINYRT |
| 491 | ATI_1523_D08 w/ N | GVSDVPRDLEVVAATPTSLLISWMRYYDAYYRITYGETGGNSPV |
| | leader + his tag | QEFTVPDQSATISGLKPGVDYTITVYALEKAHYYRQNISINYRT |
| | | HHHHHH |
| 492 | ATI_1523_D08 w/ N | GVSDVPRDLEVVAATPTSLLISWMRYYDAYYRITYGETGGNSPV |
| | leader and C tail | QEFTVPDQSATISGLKPGVDYTITVYALEKAHYYRQNISINYRT |
| | | EIDKPSQ |
| 493 | ATI_1523_D08 w/ N | GVSDVPRDLEVVAATPTSLLISWMRYYDAYYRITYGETGGNSPV |
| | leader and C tail + his | QEFTVPDQSATISGLKPGVDYTITVYALEKAHYYRQNISINYRT |
| | tag | EIDKPSQHHHHHH |
| 494 | ATI_1523_D08 w/ N | GVSDVPRDLEVVAATPTSLLISWMRYYDAYYRITYGETGGNSPV |
| | leader and modified C- | QEFTVPDQSATISGLKPGVDYTITVYALEKAHYYRONISINYRT |
| | terminus including PC | PC |
| 495 | ATI_1523_D08 w/ N | GVSDVPRDLEVVAATPTSLLISWMRYYDAYYRITYGETGGNSPV |
| | leader and modified C- | QEFTVPDQSATISGLKPGVDYTITVYALEKAHYYRQNISINYRT |
| | terminus including PC + | PCHHHHHH |
| | his tag | |
| 496 | ATI_1523_D08-full | MGVSDVPRDLEVVAATPTSLLISWMRYYDAYYRITYGETGGNSP |
| | length | VQEFTVPDQSATISGLKPGVDYTITVYALEKAHYYRQNISINYR |
| | | TEIDKPSQHHHHHH |
| 497 | ATI_1523_E08 core | EVVAATPTSLLISWHHYQHYEYRITYGETGGNSPVQEFTVPDMG |
| | | ATISGLKPGVDYTITVYALEEAHSDRSSISINYRT |
| 498 | ATI_1523_E08 BC loop | HHYQHYE |
| 499 | ATI_1523_E08 DE loop | PDMG |
| 500 | ATI_1523_E08 FG loop | LEEAHSDRSS |
| 501 | ATI_1523_E08 w/ N leader | GVSDVPRDLEVVAATPTSLLISWHHYQHYEYRITYGETGGNSPV |
| | | QEFTVPDMGATISGLKPGVDYTITVYALEEAHSDRSSISINYRT |
| 502 | ATI_1523_E08 w/ N leader | GVSDVPRDLEVVAATPTSLLISWHHYQHYEYRITYGETGGNSPV |
| | + his tag | QEFTVPDMGATISGLKPGVDYTITVYALEEAHSDRSSISINYRT |
| | | HHHHHH |
| 503 | ATI_1523_E08 w/ N leader | GVSDVPRDLEVVAATPTSLLISWHHYQHYEYRITYGETGGNSPV |
| | and C tail | QEFTVPDMGATISGLKPGVDYTITVYALEEAHSDRSSISINYRT |
| | | EIDKPSQ |
| 504 | ATI_1523_E08 w/ N leader | GVSDVPRDLEVVAATPTSLLISWHHYQHYEYRITYGETGGNSPV |
| | and C tail + his tag | QEFTVPDMGATISGLKPGVDYTITVYALEEAHSDRSSISINYRT |
| | | EIDKPSQHHHHHH |
| 505 | ATI_1523_E08 w/ N leader | GVSDVPRDLEVVAATPTSLLISWHHYQHYEYRITYGETGGNSPV |
| | and modified C-terminus | QEFTVPDMGATISGLKPGVDYTITVYALEEAHSDRSSISINYRT |
| | including PC | PC |
| 506 | ATI_1523_E08 w/ N leader | GVSDVPRDLEVVAATPTSLLISWHHYQHYEYRITYGETGGNSPV |
| | and modified C-terminus | QEFTVPDMGATISGLKPGVDYTITVYALEEAHSDRSSISINYRT |
| | including PC + his tag | PCHHHHHH |
| 507 | ATI_1523_E08-full length | MGVSDVPRDLEVVAATPTSLLISWHHYQHYEYRITYGETGGNSP |
| | | VQEFTVPDMGATISGLKPGVDYTITVYALEEAHSDRSSISINYR |
| | | TEIDKPSQHHHHHH |
| 508 | ATI_1523_F01 core | EVVAATPTSLLISWYKPSTIVTYRITYGETGGNSPVQEFTVYGY |
| | | NATISGLKPGVDYTITVYAVHGVRFISINYRT |
| 509 | ATI_1523_F01 BC loop | YKPSTIVT |
| 510 | ATI_1523_F01 DE loop | YGYN |
| 511 | ATI_1523_F01 FG loop | VHGVRF |
| 512 | ATI_1523_F01 w/ N leader | GVSDVPRDLEVVAATPTSLLISWYKPSTIVTYRITYGETGGNSP |
| | | VQEFTVYGYNATISGLKPGVDYTITVYAVHGVRFISINYRT |
| 513 | ATI_1523_F01 w/ N leader | GVSDVPRDLEVVAATPTSLLISWYKPSTIVTYRITYGETGGNSP |
| | + his tag | VQEFTVYGYNATISGLKPGVDYTITVYAVHGVRFISINYRTHHH |
| | | HHH |
| 514 | ATI_1523_F01 w/ N leader | GVSDVPRDLEVVAATPTSLLISWYKPSTIVTYRITYGETGGNSP |
| | and C tail | VQEFTVYGYNATISGLKPGVDYTITVYAVHGVRFISINYRTEID |
| | | KPSQ |
| 515 | ATI_1523_F01 w/ N leader | GVSDVPRDLEVVAATPTSLLISWYKPSTIVTYRITYGETGGNSP |
| | and C tail + his tag | VQEFTVYGYNATISGLKPGVDYTITVYAVHGVRFISINYRTEID |
| | | KPSQHHHHHH |
| 516 | ATI_1523_F01 w/ N leader | GVSDVPRDLEVVAATPTSLLISWYKPSTIVTYRITYGETGGNSP |
| | and modified C-terminus | VQEFTVYGYNATISGLKPGVDYTITVYAVHGVRFISINYRTPC |
| | including PC | |
| 517 | ATI_1523_F01 w/ N leader | GVSDVPRDLEVVAATPTSLLISWYKPSTIVTYRITYGETGGNSP |
| | and modified C-terminus | VQEFTVYGYNATISGLKPGVDYTITVYAVHGVRFISINYRTPCH |
| | including PC + his tag | HHHHH |
| 518 | ATI_1523_F01-full length | MGVSDVPRDLEVVAATPTSLLISWYKPSTIVTYRITYGETGGNS |
| | | PVQEFTVYGYNATISGLKPGVDYTITVYAVHGVRFISINYRTEI |
| | | DKPSQHHHHHH |
| 519 | ATI_1523_F04 core | EVVAATPTSLLISWGGSLSPTFYRITYGETGGNSPVQEFTVTYQ |
| | | GATISGLKPGVDYTITVYATEGIVYYQISINYRT |
| 520 | ATI_1523_F04 BC loop | GGSLSPTF |
| 521 | ATI_1523_F04 DE loop | TYQG |
| 522 | ATI_1523_F04 FG loop | TEGIVYYQ |
| 523 | ATI_1523_F04 w/ N leader | GVSDVPRDLEVVAATPTSLLISWGGSLSPTFYRITYGETGGNSP |
| | | VQEFTVTYQGATISGLKPGVDYTITVYATEGIVYYQISINYRT |
| 524 | ATI_1523_F04 w/ N leader | GVSDVPRDLEVVAATPTSLLISWGGSLSPTFYRITYGETGGNSP |
| | + his tag | VQEFTVTYQGATISGLKPGVDYTITVYATEGIVYYQISINYRTH |
| | | HHHHH |
| 525 | ATI_1523_F04 w/ N leader | GVSDVPRDLEVVAATPTSLLISWGGSLSPTFYRITYGETGGNSP |
| | and C tail | VQEFTVTYQGATISGLKPGVDYTITVYATEGIVYYQISINYRTE |
| | | IDKPSQ |
| 526 | ATI_1523_F04 w/ N leader | GVSDVPRDLEVVAATPTSLLISWGGSLSPTFYRITYGETGGNSP |
| | and C tail + his tag | VQEFTVTYQGATISGLKPGVDYTITVYATEGIVYYQISINYRTE |
| | | IDKPSQHHHHHH |
| 527 | ATI_1523_F04 w/ N leader | GVSDVPRDLEVVAATPTSLLISWGGSLSPTFYRITYGETGGNSP |
| | and modified C-terminus | VQEFTVTYQGATISGLKPGVDYTITVYATEGIVYYQISINYRTP |
| | including PC | C |
| 528 | ATI_1523_F04 w/ N leader | GVSDVPRDLEVVAATPTSLLISWGGSLSPTFYRITYGETGGNSP |
| | and modified C-terminus | VQEFTVTYQGATISGLKPGVDYTITVYATEGIVYYQISINYRTP |
| | including PC + his tag | CHHHHHH |
| 529 | ATI_1523_F04-full length | MGVSDVPRDLEVVAATPTSLLISWGGSLSPTFYRITYGETGGNS |
| | | PVQEFTVTYQGATISGLKPGVDYTITVYATEGIVYYQISINYRT |
| | | EIDKPSQHHHHHH |
| 530 | ATI_1523_F08 core | EVVAATPTSLLISWVYHYDAQYRITYGETGGNSPVQEFTVPDQK |
| | | ATISGLKPGVDYTITVYALPRAHMDRSHISINYRT |
| 531 | ATI_1523_F08 BC loop | VYHYDAQ |
| 532 | ATI_1523_F08 DE loop | PDQK |
| 533 | ATI_1523_F08 FG loop | LPRAHMDRSH |
| 534 | ATI_1523_F08 w/ N leader | GVSDVPRDLEVVAATPTSLLISWVYHYDAQYRITYGETGGNSPV |
| | | QEFTVPDQKATISGLKPGVDYTITVYALPRAHMDRSHISINYRT |
| 535 | ATI_1523_F08 w/ N leader | GVSDVPRDLEVVAATPTSLLISWVYHYDAQYRITYGETGGNSPV |
| | + his tag | QEFTVPDQKATISGLKPGVDYTITVYALPRAHMDRSHISINYRT |
| | | HHHHHH |
| 536 | ATI_1523_F08 w/ N leader | GVSDVPRDLEVVAATPTSLLISWVYHYDAQYRITYGETGGNSPV |
| | and C tail | QEFTVPDQKATISGLKPGVDYTITVYALPRAHMDRSHISINYRT |
| | | EIDKPSQ |
| 537 | ATI_1523_F08 w/ N leader | GVSDVPRDLEVVAATPTSLLISWVYHYDAQYRITYGETGGNSPV |
| | and C tail + his tag | QEFTVPDQKATISGLKPGVDYTITVYALPRAHMDRSHISINYRT |
| | | EIDKPSQHHHHHH |
| 538 | ATI_1523_F08 w/ N leader | GVSDVPRDLEVVAATPTSLLISWVYHYDAQYRITYGETGGNSPV |
| | and modified C-terminus | QEFTVPDQKATISGLKPGVDYTITVYALPRAHMDRSHISINYRT |
| | including PC | PC |
| 539 | ATI_1523_F08 w/ N leader | GVSDVPRDLEVVAATPTSLLISWVYHYDAQYRITYGETGGNSPV |
| | and modified C-terminus | QEFTVPDQKATISGLKPGVDYTITVYALPRAHMDRSHISINYRT |
| | including PC + his tag | PCHHHHHH |
| 540 | ATI_1523_F08-full length | MGVSDVPRDLEVVAATPTSLLISWVYHYDAQYRITYGETGGNSP |
| | | VQEFTVPDQKATISGLKPGVDYTITVYALPRAHMDRSHISINYR |
| | | TEIDKPSQHHHHHH |
| 541 | ATI_1523_G06 core | EVVAATPTSLLISWRIKSYHKYRITYGETGGNSPVQEFTVRSYA |
| | | ATISGLKPGVDYTITVYAIMEETHLAYAISINYRT |
| 542 | ATI_1523_G06 BC loop | RIKSYHK |
| 543 | ATI_1523_G06 DE loop | RSYA |
| 544 | ATI_1523_G06 FG loop | IMEETHLAYA |
| 545 | ATI_1523_G06 w/ N | GVSDVPRDLEVVAATPTSLLISWRIKSYHKYRITYGETGGNSPV |
| | leader | QEFTVRSYAATISGLKPGVDYTITVYAIMEETHLAYAISINYRT |
| 546 | ATI_1523_G06 w/ N | GVSDVPRDLEVVAATPTSLLISWRIKSYHKYRITYGETGGNSPV |
| | leader + his tag | QEFTVRSYAATISGLKPGVDYTITVYAIMEETHLAYAISINYRT |
| | | HHHHHH |
| 547 | ATI_1523_G06 w/ N | GVSDVPRDLEVVAATPTSLLISWRIKSYHKYRITYGETGGNSPV |
| | leader and C tail | QEFTVRSYAATISGLKPGVDYTITVYAIMEETHLAYAISINYRT |
| | | EIDKPSQ |
| 548 | ATI_1523_G06 w/ N | GVSDVPRDLEVVAATPTSLLISWRIKSYHKYRITYGETGGNSPV |
| | leader and C tail + his | QEFTVRSYAATISGLKPGVDYTITVYAIMEETHLAYAISINYRT |
| | tag | EIDKPSQHHHHHH |
| 549 | ATI_1523_G06 w/ N | GVSDVPRDLEVVAATPTSLLISWRIKSYHKYRITYGETGGNSPV |
| | leader and modified C- | QEFTVRSYAATISGLKPGVDYTITVYAIMEETHLAYAISINYRT |
| | terminus including PC | PC |
| 550 | ATI_1523_G06 w/ N | GVSDVPRDLEVVAATPTSLLISWRIKSYHKYRITYGETGGNSPV |
| | leader and modified C- | QEFTVRSYAATISGLKPGVDYTITVYAIMEETHLAYAISINYRT |
| | terminus including PC + | PCHHHHHH |
| | his tag | |
| 551 | ATI_1523_G06-full | MGVSDVPRDLEVVAATPTSLLISWRIKSYHKYRITYGETGGNSP |
| | length | VQEFTVRSYAATISGLKPGVDYTITVYAIMEETHLAYAISINYR |
| | | TEIDKPSQHHHHHH |
| 552 | ATI_1523_G07 core | EVVAATPTSLLISWVYPQADDYRITYGETGGNSPVQEFTVPDON |
| | | ATISGLKPGVDYTITVYALAEAHLVRIYISINYRT |
| 553 | ATI_1523_G07 BC loop | VYPQADD |
| 554 | ATI_1523_G07 DE loop | PDQN |
| 555 | ATI_1523_G07 FG loop | LAEAHLVRIY |
| 556 | ATI_1523_G07 w/ N | GVSDVPRDLEVVAATPTSLLISWVYPQADDYRITYGETGGNSPV |
| | leader | QEFTVPDQNATISGLKPGVDYTITVYALAEAHLVRIYISINYRT |
| 557 | ATI_1523_G07 w/ N | GVSDVPRDLEVVAATPTSLLISWVYPQADDYRITYGETGGNSPV |
| | leader + his tag | QEFTVPDQNATISGLKPGVDYTITVYALAEAHLVRIYISINYRT |
| | | HHHHHH |
| 558 | ATI_1523_G07 w/ N | GVSDVPRDLEVVAATPTSLLISWVYPQADDYRITYGETGGNSPV |
| | leader and C tail | QEFTVPDQNATISGLKPGVDYTITVYALAEAHLVRIYISINYRT |
| | | EIDKPSQ |
| 559 | ATI_1523_G07 w/ N | GVSDVPRDLEVVAATPTSLLISWVYPQADDYRITYGETGGNSPV |
| | leader and C tail + his | QEFTVPDQNATISGLKPGVDYTITVYALAEAHLVRIYISINYRT |
| | tag | EIDKPSQHHHHHH |
| 560 | ATI_1523_G07 w/ N | GVSDVPRDLEVVAATPTSLLISWVYPQADDYRITYGETGGNSPV |
| | leader and modified C- | QEFTVPDQNATISGLKPGVDYTITVYALAEAHLVRIYISINYRT |
| | terminus including PC | PC |
| 561 | ATI_1523_G07 w/ N | GVSDVPRDLEVVAATPTSLLISWVYPQADDYRITYGETGGNSPV |
| | leader and modified C- | QEFTVPDQNATISGLKPGVDYTITVYALAEAHLVRIYISINYRT |
| | terminus including PC + | PCHHHHHH |
| | his tag | |
| 562 | ATI_1523_G07-full | MGVSDVPRDLEVVAATPTSLLISWVYPQADDYRITYGETGGNSP |
| | length | VQEFTVPDQNATISGLKPGVDYTITVYALAEAHLVRIYISINYR |
| | | TEIDKPSQHHHHHH |
| 563 | ATI_1523_H07 core | EVVAATPTSLLISWVYHYDAXYRITYGETGGNSPVQEFTVPDQK |
| | | ATISGLKPGVDYTITVYALSEAHHKRDSISINYRT |
| 564 | ATI_1523_H07 BC loop | VYHYDAX |
| 565 | ATI_1523_H07 DE loop | PDQK |
| 566 | ATI_1523_H07 FG loop | LSEAHHKRDS |
| 567 | ATI_1523_H07 w/ N | GVSDVPRDLEVVAATPTSLLISWVYHYDAXYRITYGETGGNSPV |
| | leader | QEFTVPDQKATISGLKPGVDYTITVYALSEAHHKRDSISINYRT |
| 568 | ATI_1523_H07 w/ N | GVSDVPRDLEVVAATPTSLLISWVYHYDAXYRITYGETGGNSPV |
| | leader + his tag | QEFTVPDQKATISGLKPGVDYTITVYALSEAHHKRDSISINYRT |
| | | HHHHHH |
| 569 | ATI_1523_H07 w/ N | GVSDVPRDLEVVAATPTSLLISWVYHYDAXYRITYGETGGNSPV |
| | leader and C tail | QEFTVPDQKATISGLKPGVDYTITVYALSEAHHKRDSISINYRT |
| | | EIDKPSQ |
| 570 | ATI_1523_H07 w/ N | GVSDVPRDLEVVAATPTSLLISWVYHYDAXYRITYGETGGNSPV |
| | leader and C tail + his | QEFTVPDQKATISGLKPGVDYTITVYALSEAHHKRDSISINYRT |
| | tag | EIDKPSQHHHHHH |
| 571 | ATI_1523_H07 w/ N | GVSDVPRDLEVVAATPTSLLISWVYHYDAXYRITYGETGGNSPV |
| | leader and modified C- | QEFTVPDQKATISGLKPGVDYTITVYALSEAHHKRDSISINYRT |
| | terminus including PC | PC |
| 572 | ATI_1523_H07 w/ N | GVSDVPRDLEVVAATPTSLLISWVYHYDAXYRITYGETGGNSPV |
| | leader and modified C- | QEFTVPDQKATISGLKPGVDYTITVYALSEAHHKRDSISINYRT |
| | terminus including PC + | PCHHHHHH |
| | his tag | |
| 573 | ATI_1523_H07-full | GVSDVPRDLEVVAATPTSLLISWVYHYDAXYRITYGETGGNSPV |
| | length | QEFTVPDQKATISGLKPGVDYTITVYALSEAHHKRDSISINYRT |
| | | EIDKPSQHHHHHH |
| 574 | N-terminal leader | MGVSDVPRDL |
| 575 | N-terminal leader | GVSDVPRDL |
| 576 | N-terminal leader | XₙSDVPRDL |
| 577 | N-terminal leader | XₙDVPRDL |
| 578 | N-terminal leader | XₙVPRDL |
| 579 | N-terminal leader | XₙPRDL |
| 580 | N-terminal leader | XₙRDL |
| 581 | N-terminal leader | XₙDL |
| 582 | N-terminal leader | MASTSG |
| 583 | N-terminal leader | METDTLLLWVLLLWVPGSTG |
| 584 | C-terminal tail | EIEK |
| 585 | C-terminal tail | EGSGC |
| 586 | C-terminal tail | EIEKPCQ |
| 587 | C-terminal tail | EIEKPSQ |
| 588 | C-terminal tail | EIEKP |
| 589 | C-terminal tail | EIEKPS |
| 590 | C-terminal tail | EIEKPC |
| 591 | C-terminal tail | EIDK |
| 592 | C-terminal tail | EIDKPCQ |
| 593 | C-terminal tail | EIDKPSQ |
| 594 | C-terminal tail | EIEPKSS |
| 595 | C-terminal tail | EIDKPC |
| 596 | C-terminal tail | EIDKP |
| 597 | C-terminal tail | EIDKPS |
| 598 | C-terminal tail | EIDKPSQLE |
| 599 | C-terminal tail | EIEDEDEDEDED |
| 600 | C-terminal tail | EGSGS |
| 601 | C-terminal tail | EIDKPCQLE |
| 602 | C-terminal tail | EIDKPSQHHHHHH |
| 603 | C-terminal tail | GSGCHHHHHH |
| 604 | C-terminal tail | EGSGCHHHHHH |
| 605 | C-terminal tail | PIDK |
| 606 | C-terminal tail | PIEK |
| 607 | C-terminal tail | PIDKP |
| 608 | C-terminal tail | PIEKP |
| 609 | C-terminal tail | PIDKPS |
| 610 | C-terminal tail | PIEKPS |
| 611 | C-terminal tail | PIDKPC |
| 612 | C-terminal tail | PIEKPC |
| 613 | C-terminal tail | PIDKPSQ |
| 614 | C-terminal tail | PIEKPSQ |
| 615 | C-terminal tail | PIDKPCQ |
| 616 | C-terminal tail | PIEKPCQ |
| 617 | C-terminal tail | PHHHHHH |
| 618 | C-terminal tail | PCHHHHHH |
| 619 | 6X-His tag | HHHHHH |
| 620 | Human IgG1 Fc domain | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 621 | Core hinge region of Fc | DKTHTCPPCPAPELLG |
| 622 | Exemplary hinge sequence | EPKSSDKTHTCPPCPAPELLGGPS |
| 623 | Exemplary hinge sequence | EPKSSDKTHTCPPCPAPELLGGSS |
| 624 | Exemplary hinge sequence | EPKSSGSTHTCPPCPAPELLGGSS |
| 625 | Exemplary hinge sequence | DKTHTCPPCPAPELLGGPS |
| 626 | Exemplary hinge sequence | DKTHTCPPCPAPELLGGSS |
| 627 | Fc with CH2 and CH3 | VFLFPPKPKDTLMISRTPEVTCVVVDVSHEDPEVKENWYVDGVEVH |
| | regions of IgG1 for | NAKTKPREEQYNSTYRVVSVLTVLHQDWLNGKEYKCKVSNKALPAP |
| | Adnectin-hinge-Fc | IEKTISKAKGQPREPQVYTLPPSRDELTKNQVSLTCLVKGFYPSDI |
| | construct | AVEWESNGQPENNYKTTPPVLDSDGSFFLYSKLTVDKSRWQQGNVE |
| | | SCSVMHEALHNHYTQKSLSLSPGK |
| 628 | Fc with CH2 and CH3 | VFLFPPKPKDTLMISRTPEVTCVVVDVSHEDPEVKFNWYVDGVEVH |
| | regions of IgG1 for Fc- | NAKTKPREEQYNSTYRVVSVLTVLHQDWLNGKEYKCKVSNKALPAP |
| | hinge-Adnectin construct | IEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVE |
| | | SCSVMHEALHNHYTQKSLSLSP |
| 629 | Linker 1 | GAGGGGSG |
| 630 | Linker 2 | EPKSSD |
| 631 | Linker 3 | PVGVV |
| 632 | Linker 4 | ESPKAQASSVPTAQPQAEGLA |
| 633 | Linker 5 | ELQLEESAAEAQDGELD |
| 634 | Linker 6 | GQPDEPGGS |
| 635 | Linker 7 | GGSGSGSGSGSGS |
| 636 | Linker 8 | ELQLEESAAEAQEGELE |
| 637 | Linker 9 | GSGSG |
| 638 | Linker 10 | GSGC |
| 639 | Linker 11 | AGGGGSG |
| 640 | Linker 12 | GSGS |
| 641 | Linker 13 | QPDEPGGS |
| 642 | Linker 14 | GSGSGS |
| 643 | Linker 15 | TVAAPS |
| 644 | Linker 16 | KAGGGGSG |
| 645 | Linker 17 | KGSGSGSGSGSGS |
| 646 | Linker 18 | KOPDEPGGS |
| 647 | Linker 19 | KELQLEESAAEAQDGELD |
| 648 | Linker 20 | KTVAAPS |
| 649 | Linker 21 | KAGGGGSGG |
| 650 | Linker 22 | KGSGSGSGSGSGSG |
| 651 | Linker 23 | KOPDEPGGSG |
| 652 | Linker 24 | KELQLEESAAEAQDGELDG |
| 653 | Linker 25 | KTVAAPSG |
| 654 | Linker 26 | AGGGGSGG |
| 655 | Linker 27 | AGGGGSG |
| 656 | Linker 28 | GSGSGSGSGSGSG |
| 657 | Linker 29 | QPDEPGGSG |
| 658 | Linker 30 | TVAAPSG |
| 659 | Linker 31 | PSTSTST |
| 660 | Linker 32 | EIDKPSQ |
| 661 | Linker 33 | GSGSGSGS |
| 662 | Linker 34 | GSGSGSGSGS |
| 663 | Linker 35 | GSGSGSGSGSGS |
| 664 | Linker 36 | GSGSGSGSGSGSGS |
| 665 | Linker 37 | GGSGSGSGSGSGS |
| 666 | Linker 38 | GGSGSGSGSGSGSGSG |
| 667 | Linker 39 | GSEGSEGSEGSEGSE |
| 668 | Linker 40 | GGSEGGSE |
| 669 | Linker 41 | GGGGSGGGGSGGGGSGGGGSGGGGSGGGGSGGGGS |
| 670 | Linker 42 | GGGGSGGGGSGGGGSGGGGSGGGGS |
| 671 | Linker 43 | GGGGSGGGGSGGGGSG |
| 672 | Linker 44 | GPGPGPG |
| 673 | Linker 45 | GPGPGPGPGPG |
| 674 | Linker 46 | PAPAPA |
| 675 | Linker 47 | PAPAPAPAPAPA |
| 676 | Linker 48 | PAPAPAPAPAPAPAPAPA |
| 677 | Linker 49 | GSGSGSGSGSGSGSGSGSGS |
| 678 | Linker 50 | GGGGSGGGGSGGGGSGGGGS |
| 679 | 12A4 VH | QVQLVQSGAEVKKPGSSVKVSCKTSGDTFSTYAISWVRQAPGOGLE |
| | | WMGGIIPIFGKAHYAQKFQGRVTITADESTSTAYMELSSLRSEDTA |
| | | VYFCARKFHFVSGSPFGMDVWGQGTTVT |
| | | VSS |
| 680 | 12A4 VL | EIVLTQSPATLSLSPGERATLSCRASQSVSSYLAWYQQKPGQAPRL |
| | | LIYDASNRATGIPA |
| | | RFSGSGSGTDFTLTISSLEPEDFAVYYCQQRSNWPTFGQGTKVEIK |
| 681 | 12A4 VH CDR1 | TYAIS |
| 682 | 12A4 VH CDR2 | GIIPIFGKAHYAQKFQ |
| 683 | 12A4 VH CDR3 | KFHFVSGSPFGMDV |
| 684 | 12A4 VL CDR1 | RASQSVSSYLA |
| 685 | 12A4 VL CDR2 | DASNRAT |
| 686 | 12A4 VL CDR3 | QQRSNWPT |

### EQUIVALENTS

Those skilled in the art will recognize or be able to ascertain using no more than routine experimentation, many equivalents of the specific embodiments described herein. Such equivalents are intended to be encompassed by the following claims.

1. A combination for use in detecting and treating cancer in a subject, comprising
(a) a radioimaging agent comprising a fibronectin based scaffold (FBS) polypeptide comprising a ¹⁰Fn3 domain which binds to PD-L1 and a radionuclide; and
(b) a radiotherapeutic agent comprising a PD-L1 antibody or antigen binding fragment thereof and a radionuclide.

2-58. (canceled)

59. An article of manufacture comprising the combination of claim 1.

60. A method of detecting and treating a subject having a PD-L1 expressing cancer, comprising
a. determining the presence of PD-L1 in a subject having cancer, comprising administering to radioimaging agent of claim 1, and if PD-L1 is found to be present in one or more tumors of the subject,
b. administering to the subject the radiotherapeutic agent of claim 1.

61-68. (canceled)

69. The method of claim 60, wherein the radioimaging agent is also administered after the radiotherapeutic agent, e.g., to monitor PD-L1 levels in the subject, and further administration of the radiotherapeutic agent is determined based on the PD-L1 levels identified with the radioimaging agent.

70. A radiotherapeutic agent comprising an anti-PD-L1 antibody or antigen binding fragment thereof and a radionuclide.

71. The radiotherapeutic agent of claim of claim 70, wherein the anti-PD-L1 antibody or antigen binding fragment thereof comprises the amino acid sequences of VH CDR1, CDR2 and CDR3 set forth in SEQ ID Nos: 681, 682 and 683, respectively.

72-91. (canceled)

92. A pharmaceutical composition comprising the radiotherapeutic agent of claim 70.

93. A method of treating cancer comprising administering to a subject in need thereof the radiolabeled anti-PD-L1 antibody or antigen binding fragment thereof of claim 70.

94. (canceled)

95. An article of manufacture comprising the radiotherapeutic agent of claim 70 and one or more containers.

96. A combination for use in detecting and treating cancer in a subject, comprising
(a) a radioimaging agent comprising a PD-L1 antibody or antigen binding fragment thereof and a radionuclide; and
(b) a radiotherapeutic agent comprising a PD-L1 antibody or antigen binding fragment thereof and a radionuclide,
wherein the PD-L1 antibody of the radioimaging agent and the radiotherapeutic agent have the same antigen binding specificity.

97-139. (canceled)

140. A kit comprising the combination of claim 1, and instructions for use.

141. A method for treating cancer in a subject, comprising administering to the subject
(a) at a first time, a radioimaging agent comprising a PD-L1 antibody or antigen binding fragment a radionuclide; and
(b) at a second time, a radiotherapeutic agent comprising a PD-L1 antibody or antigen binding fragment and a radionuclide,
wherein the PD-L1 antibody of the radioimaging agent and the radiotherapeutic agent have the same antigen binding specificity.

142-147. (canceled)

148. The method of claim 141, wherein the radioimaging agent is also administered after the radiotherapeutic agent, e.g., to monitor PD-L1 levels in the subject, and further administration of the radiotherapeutic agent is determined based on the PD-L1 levels identified with the radioimaging agent.

149. A combination for use in diagnosis, monitoring and treatment of cancer in a subject, the combination comprising
(a) a radioimaging agent comprising a fibronectin based scaffold (FBS) polypeptide which binds to a target expressed by the cancer and a radionuclide; and
(b) a radiotherapeutic agent comprising the FBS polypeptide and a radionuclide,
wherein the FBS polypeptide of the radioimaging agent and the radiotherapeutic agent bind to the target.

150-187. (canceled)

188. A combination for diagnosis, monitoring and treating a PD-L1 expressing cancer, wherein the combination comprises
(a) a radioimaging agent comprising an FBS polypeptide comprising a human ¹⁰Fn3 domain which binds to human PD-L1; and
(b) a radiotherapeutic agent comprising an FBS polypeptide comprising a human ¹⁰Fn3 domain which binds to human PD-L1.

189-193. (canceled)

194. A pharmaceutical composition comprising the radioimaging agent of claim 149.

195. A pharmaceutical composition comprising the radiotherapeutic agent of claim 149.

196. A kit for use in radioimaging and radiotherapy, comprising
(a) a fibronectin based scaffold (FBS) polypeptide which binds to a target and a chelating agent for a radionuclide suitable for radioimaging; and
(b) an FBS polypeptide which binds to a target and a chelating agent for a radionuclide suitable for radiotherapy,
wherein the FBS polypeptide is the same in (a) and (b), and wherein the kit contains instructions for chelation of the FBS polypeptide to the radionuclides.

197-215. (canceled)

216. A method of diagnosing and treating cancer in a subject comprising:
(a) administering to the subject an radioimaging agent comprising a fibronectin based scaffold (FBS) polypeptide which binds to a target expressed by cancer cells and a radionuclide suitable for radioimaging;
(b) obtaining a radioimage of all or a portion of the subject to determine the presence of the target in the subject;
(c) administering a radiotherapeutic agent comprising an FBS polypeptide and a radionuclide suitable for radiotherapy
wherein the radioimaging agent and radiotherapeutic agent bind to the same target.

217-218. (canceled)

219. A method of treating a subject having a PD-L1 expressing cancer, comprising
a. determining the presence of PD-L1 in a subject having cancer, comprising administering to the subject a radioimaging agent comprising an FBS polypeptide comprising a human ¹⁰Fn3 domain which binds to human PD-L1 linked to radionuclide, and if PD-L1 is found to be present in one or more tumors of the subject,
b. administering to the subject an FBS polypeptide comprising a human ¹⁰Fn3 domain which binds to human PD-L1 linked to a radionuclide.

220. (canceled)


SEQUENCE LISTING (686 sequences)

SEQ ID NO: 1 - protein, 94 aa, Homo sapiens
  misc_feature: Full length wild-type human 10Fn3 domain
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 86 aa, Homo sapiens
  misc_feature: Core wild-type human 10Fn3 domain
EVVAATPTSLLISWDAPAVTVRYYRITYGETGGNSPVQEFTVPGSKSTATISGLKPGVDYTITVYAVTGRGDSPASSKPISINYRT

SEQ ID NO: 3 - protein, 153 aa, Artificial Sequence
  Synthetic Core 10Fn3-based scaffold with variable AB, BC, CD, DE, EF, and FG loops
  misc_feature: see specification as filed for detailed description of substitutions and preferred embodiments
  misc_feature (6)..(20): Xaa can be any naturally occurring amino acid; at least one or two Xaa are present and the rest may be present or absent
  misc_feature (26)..(45): Xaa can be any naturally occurring amino acid; at least two, five or six Xaa are present and the rest may be present or absent
  misc_feature (51)..(70): Xaa can be any naturally occurring amino acid; at least two, five or six Xaa are present and the rest may be present or absent
  misc_feature (74)..(93): Xaa can be any naturally occurring amino acid; at least two, five or six Xaa are present and the rest may be present or absent
  misc_feature (100)..(119): Xaa can be any naturally occurring amino acid; at least two, five or six Xaa are present and the rest may be present or absent
  misc_feature (127)..(146): Xaa can be any naturally occurring amino acid; at least two, five or six Xaa are present and the rest may be present or absent
EVVAAXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 4 - protein, 118 aa, Artificial Sequence
  Synthetic Core 10Fn3-based scaffold with variable BC, DE, and FG loops
  misc_feature: see specification as filed for detailed description of substitutions and preferred embodiments
  misc_feature (15)..(34): Xaa can be any naturally occurring amino acid; at least two, five or six Xaa are present and the rest may be present or absent
  misc_feature (54)..(73): Xaa can be any naturally occurring amino acid; at least two, five or six Xaa are present and the rest may be present or absent
  misc_feature (92)..(111): Xaa can be any naturally occurring amino acid; at least two, five or six Xaa are present and the rest may be present or absent
EVVAATPTSLLISWXXXXXXXXXXXXXXXXXXXXYRITYGETGGNSPVQEFTVXXXXXXXXXXXXXXXXXXXXATISGLKPGVDYTITVYAXXXXXXXXXXXXXXXXXXXXISINYRT

SEQ ID NO: 5 - protein, 86 aa, Artificial Sequence
  Synthetic ATI-968 core (aka ADX_1760_C01)
EVVAATPTSLLISWIAPFYNVIYYRITYGETGGNSPVQEFTVPGTGYTATISGLKPGVDYTITVYAVTDGASIASYAFPISINYRT

SEQ ID NO: 6 - protein, 9 aa, Artificial Sequence
  Synthetic ATI-968 BC loop
IAPFYNVIY

SEQ ID NO: 7 - protein, 6 aa, Artificial Sequence
  Synthetic ATI-968 DE loop
PGTGYT

SEQ ID NO: 8 - protein, 13 aa, Artificial Sequence
  Synthetic ATI-968 FG loop
VTDGASIASYAFP

SEQ ID NO: 9 - protein, 95 aa, Artificial Sequence
  Synthetic ATI-968 w/ N leader
GVSDVPRDLEVVAATPTSLLISWIAPFYNVIYYRITYGETGGNSPVQEFTVPGTGYTATISGLKPGVDYTITVYAVTDGASIASYAFPISINYRT

SEQ ID NO: 10 - protein, 101 aa, Artificial Sequence
  Synthetic ATI-968 w/ N leader + his tag
GVSDVPRDLEVVAATPTSLLISWIAPFYNVIYYRITYGETGGNSPVQEFTVPGTGYTATISGLKPGVDYTITVYAVTDGASIASYAFPISINYRTHHHHHH

SEQ ID NO: 11 - protein, 102 aa, Artificial Sequence
  Synthetic ATI-968 w/ N leader and C tail
GVSDVPRDLEVVAATPTSLLISWIAPFYNVIYYRITYGETGGNSPVQEFTVPGTGYTATISGLKPGVDYTITVYAVTDGASIASYAFPISINYRTEIDKPSQ

SEQ ID NO: 12 - protein, 108 aa, Artificial Sequence
  Synthetic ATI-968 w/ N leader and C tail + his tag
GVSDVPRDLEVVAATPTSLLISWIAPFYNVIYYRITYGETGGNSPVQEFTVPGTGYTATISGLKPGVDYTITVYAVTDGASIASYAFPISINYRTEIDKPSQHHHHHH

SEQ ID NO: 13 - protein, 97 aa, Artificial Sequence
  Synthetic ATI-968 w/ N leader and modified C-terminus including PC
GVSDVPRDLEVVAATPTSLLISWIAPFYNVIYYRITYGETGGNSPVQEFTVPGTGYTATISGLKPGVDYTITVYAVTDGASIASYAFPISINYRTPC

SEQ ID NO: 14 - protein, 103 aa, Artificial Sequence
  Synthetic ATI-968 w/ N leader and modified C-terminus including PC + his tag
GVSDVPRDLEVVAATPTSLLISWIAPFYNVIYYRITYGETGGNSPVQEFTVPGTGYTATISGLKPGVDYTITVYAVTDGASIASYAFPISINYRTPCHHHHHH

SEQ ID NO: 15 - protein, 109 aa, Artificial Sequence
  Synthetic ATI-968  full length
MGVSDVPRDLEVVAATPTSLLISWIAPFYNVIYYRITYGETGGNSPVQEFTVPGTGYTATISGLKPGVDYTITVYAVTDGASIASYAFPISINYRTEIDKPSQHHHHHH

SEQ ID NO: 16 - DNA, 258 bp, Artificial Sequence
  Synthetic ATI-968  core (nucleotide sequence)
gaagtggttgctgccacccccaccagcctgctgatcagctggatcgctccgttctacaatgtcatctattaccgcatcacttacggcgaaacaggaggcaatagccctgtccaggagttcactgtgcctggtactggttatacagctacaatcagcggccttaaacctggcgttgattataccatcactgtgtatgctgtcactgatggagcatccattgcttcatacgcgtttccaatttccattaattaccgcaca

SEQ ID NO: 17 - DNA, 288 bp, Artificial Sequence
  Synthetic ATI-968 w/ N leader (nucleotide sequence with N-terminal methionine)
atgggagtttctgatgtgccgcgcgacctggaagtggttgctgccacccccaccagcctgctgatcagctggatcgctccgttctacaatgtcatctattaccgcatcacttacggcgaaacaggaggcaatagccctgtccaggagttcactgtgcctggtactggttatacagctacaatcagcggccttaaacctggcgttgattataccatcactgtgtatgctgtcactgatggagcatccattgcttcatacgcgtttccaatttccattaattaccgcaca

SEQ ID NO: 18 - DNA, 294 bp, Artificial Sequence
  Synthetic ATI-968 w/ N leader and modified C-terminus including PC (nucleotide sequence with N-terminal methionine)
atgggagtttctgatgtgccgcgcgacctggaagtggttgctgccacccccaccagcctgctgatcagctggatcgctccgttctacaatgtcatctattaccgcatcacttacggcgaaacaggaggcaatagccctgtccaggagttcactgtgcctggtactggttatacagctacaatcagcggccttaaacctggcgttgattataccatcactgtgtatgctgtcactgatggagcatccattgcttcatacgcgtttccaatttccattaattaccgcacaccgtgc

SEQ ID NO: 19 - DNA, 330 bp, Artificial Sequence
  Synthetic ATI-968 w/ N leader and C tail + his tag (nucleotide sequence with N-terminal methionine)
atgggagtttctgatgtgccgcgcgacctggaagtggttgctgccacccccaccagcctgctgatcagctggatcgctccgttctacaatgtcatctattaccgcatcacttacggcgaaacaggaggcaatagccctgtccaggagttcactgtgcctggtactggttatacagctacaatcagcggccttaaacctggcgttgattataccatcactgtgtatgctgtcactgatggagcatccattgcttcatacgcgtttccaatttccattaattaccgcacagaaattgacaaaccatcccagcaccatcaccaccaccactga

SEQ ID NO: 20 - protein, 86 aa, Artificial Sequence
  Synthetic ATI-964 core (parent of ADX_5322_A02)
EVVAATPTSLLISWSYDGSIERYYRITYGETGGNSPVQEFTVPPDQKTATISGLKPGVDYTITVYAVRLEEAHYYRESPISINYRT

SEQ ID NO: 21 - protein, 9 aa, Artificial Sequence
  Synthetic ATI-964 BC loop
SYDGSIERY

SEQ ID NO: 22 - protein, 6 aa, Artificial Sequence
  Synthetic ATI-964 DE loop
PPDQKT

SEQ ID NO: 23 - protein, 13 aa, Artificial Sequence
  Synthetic ATI-964 FG loop
VRLEEAHYYRESP

SEQ ID NO: 24 - protein, 95 aa, Artificial Sequence
  Synthetic ATI-964 w/ N leader
GVSDVPRDLEVVAATPTSLLISWSYDGSIERYYRITYGETGGNSPVQEFTVPPDQKTATISGLKPGVDYTITVYAVRLEEAHYYRESPISINYRT

SEQ ID NO: 25 - protein, 101 aa, Artificial Sequence
  Synthetic ATI-964 w/ N leader + his tag
GVSDVPRDLEVVAATPTSLLISWSYDGSIERYYRITYGETGGNSPVQEFTVPPDQKTATISGLKPGVDYTITVYAVRLEEAHYYRESPISINYRTHHHHHH

SEQ ID NO: 26 - protein, 102 aa, Artificial Sequence
  Synthetic ATI-964 w/ N leader and C tail
GVSDVPRDLEVVAATPTSLLISWSYDGSIERYYRITYGETGGNSPVQEFTVPPDQKTATISGLKPGVDYTITVYAVRLEEAHYYRESPISINYRTEIDKPSQ

SEQ ID NO: 27 - protein, 108 aa, Artificial Sequence
  Synthetic ATI-964 w/ N leader and C tail + his tag
GVSDVPRDLEVVAATPTSLLISWSYDGSIERYYRITYGETGGNSPVQEFTVPPDQKTATISGLKPGVDYTITVYAVRLEEAHYYRESPISINYRTEIDKPSQHHHHHH

SEQ ID NO: 28 - protein, 97 aa, Artificial Sequence
  Synthetic ATI-964 w/ N leader and modified C-terminus including PC
GVSDVPRDLEVVAATPTSLLISWSYDGSIERYYRITYGETGGNSPVQEFTVPPDQKTATISGLKPGVDYTITVYAVRLEEAHYYRESPISINYRTPC

SEQ ID NO: 29 - protein, 103 aa, Artificial Sequence
  Synthetic ATI-964 w/ N leader and modified C-terminus including PC + his tag
GVSDVPRDLEVVAATPTSLLISWSYDGSIERYYRITYGETGGNSPVQEFTVPPDQKTATISGLKPGVDYTITVYAVRLEEAHYYRESPISINYRTPCHHHHHH

SEQ ID NO: 30 - protein, 109 aa, Artificial Sequence
  Synthetic ATI-964  full length
MGVSDVPRDLEVVAATPTSLLISWSYDGSIERYYRITYGETGGNSPVQEFTVPPDQKTATISGLKPGVDYTITVYAVRLEEAHYYRESPISINYRTEIDKPSQHHHHHH

SEQ ID NO: 31 - DNA, 258 bp, Artificial Sequence
  Synthetic ATI-964  core (nucleotide sequence)
gaagtggttgctgccacccccaccagcctgctgatcagctggtcttacgacggttcgattgaacgttattaccgcatcacttacggcgaaacaggaggcaatagccctgtccaggagttcactgtgcctccggatcagaagacagctaccatcagcggccttaaacctggcgttgattataccatcactgtgtatgctgtcaggctggaagaagctcattactatcgagagtctccaatttccattaattaccgcaca

SEQ ID NO: 32 - DNA, 288 bp, Artificial Sequence
  Synthetic ATI-964 w/ N leader (nucleotide sequence with N-terminal methionine)
atgggagtttctgatgtgccgcgcgacctggaagtggttgctgccacccccaccagcctgctgatcagctggtcttacgacggttcgattgaacgttattaccgcatcacttacggcgaaacaggaggcaatagccctgtccaggagttcactgtgcctccggatcagaagacagctaccatcagcggccttaaacctggcgttgattataccatcactgtgtatgctgtcaggctggaagaagctcattactatcgagagtctccaatttccattaattaccgcaca

SEQ ID NO: 33 - DNA, 294 bp, Artificial Sequence
  Synthetic ATI-964 w/ N leader and modified C-terminus including PC (nucleotide sequence with N-terminal methionine)
atgggagtttctgatgtgccgcgcgacctggaagtggttgctgccacccccaccagcctgctgatcagctggtcttacgacggttcgattgaacgttattaccgcatcacttacggcgaaacaggaggcaatagccctgtccaggagttcactgtgcctccggatcagaagacagctaccatcagcggccttaaacctggcgttgattataccatcactgtgtatgctgtcaggctggaagaagctcattactatcgagagtctccaatttccattaattaccgcacaccgtgc

SEQ ID NO: 34 - DNA, 330 bp, Artificial Sequence
  Synthetic ATI-964 w/ N leader and C tail + his tag (nucleotide sequence with N-terminal methionine)
atgggagtttctgatgtgccgcgcgacctggaagtggttgctgccacccccaccagcctgctgatcagctggtcttacgacggttcgattgaacgttattaccgcatcacttacggcgaaacaggaggcaatagccctgtccaggagttcactgtgcctccggatcagaagacagctaccatcagcggccttaaacctggcgttgattataccatcactgtgtatgctgtcaggctggaagaagctcattactatcgagagtctccaatttccattaattaccgcacagaaattgacaaaccatcccagcaccatcaccaccaccactga

SEQ ID NO: 35 - protein, 86 aa, Artificial Sequence
  Synthetic ATI-965 core
EVVAATPTSLLISWTAYDSVDKYYRITYGETGGNSPVQEFTVGPRHHTATISGLKPGVDYTITVYAVYHTEPGYHAHMPISINYRT

SEQ ID NO: 36 - protein, 9 aa, Artificial Sequence
  Synthetic ATI-965 BC loop
TAYDSVDKY

SEQ ID NO: 37 - protein, 6 aa, Artificial Sequence
  Synthetic ATI-965 DE loop
GPRHHT

SEQ ID NO: 38 - protein, 13 aa, Artificial Sequence
  Synthetic ATI-965 FG loop
VYHTEPGYHAHMP

SEQ ID NO: 39 - protein, 95 aa, Artificial Sequence
  Synthetic ATI-965 w/ N leader
GVSDVPRDLEVVAATPTSLLISWTAYDSVDKYYRITYGETGGNSPVQEFTVGPRHHTATISGLKPGVDYTITVYAVYHTEPGYHAHMPISINYRT

SEQ ID NO: 40 - protein, 101 aa, Artificial Sequence
  Synthetic ATI-965 w/ N leader + his tag
GVSDVPRDLEVVAATPTSLLISWTAYDSVDKYYRITYGETGGNSPVQEFTVGPRHHTATISGLKPGVDYTITVYAVYHTEPGYHAHMPISINYRTHHHHHH

SEQ ID NO: 41 - protein, 102 aa, Artificial Sequence
  Synthetic ATI-965 w/ N leader and C tail
GVSDVPRDLEVVAATPTSLLISWTAYDSVDKYYRITYGETGGNSPVQEFTVGPRHHTATISGLKPGVDYTITVYAVYHTEPGYHAHMPISINYRTEIDKPSQ

SEQ ID NO: 42 - protein, 108 aa, Artificial Sequence
  Synthetic ATI-965 w/ N leader and C tail + his tag
GVSDVPRDLEVVAATPTSLLISWTAYDSVDKYYRITYGETGGNSPVQEFTVGPRHHTATISGLKPGVDYTITVYAVYHTEPGYHAHMPISINYRTEIDKPSQHHHHHH

SEQ ID NO: 43 - protein, 97 aa, Artificial Sequence
  Synthetic ATI-965 w/ N leader and modified C-terminus including PC
GVSDVPRDLEVVAATPTSLLISWTAYDSVDKYYRITYGETGGNSPVQEFTVGPRHHTATISGLKPGVDYTITVYAVYHTEPGYHAHMPISINYRTPC

SEQ ID NO: 44 - protein, 103 aa, Artificial Sequence
  Synthetic ATI-965 w/ N leader and modified C-terminus including PC + his tag
GVSDVPRDLEVVAATPTSLLISWTAYDSVDKYYRITYGETGGNSPVQEFTVGPRHHTATISGLKPGVDYTITVYAVYHTEPGYHAHMPISINYRTPCHHHHHH

SEQ ID NO: 45 - protein, 109 aa, Artificial Sequence
  Synthetic ATI-965  full length
MGVSDVPRDLEVVAATPTSLLISWTAYDSVDKYYRITYGETGGNSPVQEFTVGPRHHTATISGLKPGVDYTITVYAVYHTEPGYHAHMPISINYRTEIDKPSQHHHHHH

SEQ ID NO: 46 - DNA, 258 bp, Artificial Sequence
  Synthetic ATI-965  core (nucleotide sequence)
gaagtggttgctgccacccccaccagcctgctgatcagctggactgcatacgactctgttgacaaatattaccgcatcacttacggcgaaacaggaggcaatagccctgtccaggagttcactgtgggccctagacatcacacagctaccatcagcggccttaaacctggcgttgattataccatcactgtgtatgctgtctatcacactgaaccgggctatcatgctcatatgccaatttccattaattaccgcaca

SEQ ID NO: 47 - DNA, 288 bp, Artificial Sequence
  Synthetic ATI-965 w/ N leader (nucleotide sequence with N-terminal methionine)
atgggagtttctgatgtgccgcgcgacctggaagtggttgctgccacccccaccagcctgctgatcagctggactgcatacgactctgttgacaaatattaccgcatcacttacggcgaaacaggaggcaatagccctgtccaggagttcactgtgggccctagacatcacacagctaccatcagcggccttaaacctggcgttgattataccatcactgtgtatgctgtctatcacactgaaccgggctatcatgctcatatgccaatttccattaattaccgcaca

SEQ ID NO: 48 - DNA, 294 bp, Artificial Sequence
  Synthetic ATI-965 w/ N leader and modified C-terminus including PC (nucleotide sequence with N-terminal methionine)
atgggagtttctgatgtgccgcgcgacctggaagtggttgctgccacccccaccagcctgctgatcagctggactgcatacgactctgttgacaaatattaccgcatcacttacggcgaaacaggaggcaatagccctgtccaggagttcactgtgggccctagacatcacacagctaccatcagcggccttaaacctggcgttgattataccatcactgtgtatgctgtctatcacactgaaccgggctatcatgctcatatgccaatttccattaattaccgcacaccgtgc

SEQ ID NO: 49 - DNA, 330 bp, Artificial Sequence
  Synthetic ATI-965 w/ N leader and C tail + his tag (nucleotide sequence with N-terminal methionine)
atgggagtttctgatgtgccgcgcgacctggaagtggttgctgccacccccaccagcctgctgatcagctggactgcatacgactctgttgacaaatattaccgcatcacttacggcgaaacaggaggcaatagccctgtccaggagttcactgtgggccctagacatcacacagctaccatcagcggccttaaacctggcgttgattataccatcactgtgtatgctgtctatcacactgaaccgggctatcatgctcatatgccaatttccattaattaccgcacagaaattgacaaaccatcccagcaccatcaccaccaccactga

SEQ ID NO: 50 - protein, 82 aa, Artificial Sequence
  Synthetic ATI-966 core
EVVAATPTSLLISWHRFSSIMAYYRITYGETGGNSPVQEFTVAGSVNTATISGLKPGVDYTITVYAVTIHNVSFPISINYRT

SEQ ID NO: 51 - protein, 9 aa, Artificial Sequence
  Synthetic ATI-966 BC loop
HRFSSIMAY

SEQ ID NO: 52 - protein, 6 aa, Artificial Sequence
  Synthetic ATI-966 DE loop
AGSVNT

SEQ ID NO: 53 - protein, 9 aa, Artificial Sequence
  Synthetic ATI-966 FG loop
VTIHNVSFP

SEQ ID NO: 54 - protein, 91 aa, Artificial Sequence
  Synthetic ATI-966 w/ N leader
GVSDVPRDLEVVAATPTSLLISWHRFSSIMAYYRITYGETGGNSPVQEFTVAGSVNTATISGLKPGVDYTITVYAVTIHNVSFPISINYRT

SEQ ID NO: 55 - protein, 97 aa, Artificial Sequence
  Synthetic ATI-966 w/ N leader + his tag
GVSDVPRDLEVVAATPTSLLISWHRFSSIMAYYRITYGETGGNSPVQEFTVAGSVNTATISGLKPGVDYTITVYAVTIHNVSFPISINYRTHHHHHH

SEQ ID NO: 56 - protein, 98 aa, Artificial Sequence
  Synthetic ATI-966 w/ N leader and C tail
GVSDVPRDLEVVAATPTSLLISWHRFSSIMAYYRITYGETGGNSPVQEFTVAGSVNTATISGLKPGVDYTITVYAVTIHNVSFPISINYRTEIDKPSQ

SEQ ID NO: 57 - protein, 104 aa, Artificial Sequence
  Synthetic ATI-966 w/ N leader and C tail + his tag
GVSDVPRDLEVVAATPTSLLISWHRFSSIMAYYRITYGETGGNSPVQEFTVAGSVNTATISGLKPGVDYTITVYAVTIHNVSFPISINYRTEIDKPSQHHHHHH

SEQ ID NO: 58 - protein, 93 aa, Artificial Sequence
  Synthetic ATI-966 w/ N leader and modified C-terminus including PC
GVSDVPRDLEVVAATPTSLLISWHRFSSIMAYYRITYGETGGNSPVQEFTVAGSVNTATISGLKPGVDYTITVYAVTIHNVSFPISINYRTPC

SEQ ID NO: 59 - protein, 99 aa, Artificial Sequence
  Synthetic ATI-966 w/ N leader and modified C-terminus including PC + his tag
GVSDVPRDLEVVAATPTSLLISWHRFSSIMAYYRITYGETGGNSPVQEFTVAGSVNTATISGLKPGVDYTITVYAVTIHNVSFPISINYRTPCHHHHHH

SEQ ID NO: 60 - protein, 105 aa, Artificial Sequence
  Synthetic ATI-966  full length
MGVSDVPRDLEVVAATPTSLLISWHRFSSIMAYYRITYGETGGNSPVQEFTVAGSVNTATISGLKPGVDYTITVYAVTIHNVSFPISINYRTEIDKPSQHHHHHH

SEQ ID NO: 61 - DNA, 246 bp, Artificial Sequence
  Synthetic ATI-966  core (nucleotide sequence)
gaagtggttgctgccacccccaccagcctgctgatcagctggcataggttctcttctatcatggcgtattaccgcatcacttacggcgaaacaggaggcaatagccctgtccaggagttcactgtggctggctctgttaacacagctaccatcagcggccttaaacctggcgttgattataccatcactgtgtatgctgtcacgatccataacgtttctttcccaatttccattaattaccgcaca

SEQ ID NO: 62 - DNA, 276 bp, Artificial Sequence
  Synthetic ATI-966w/ N leader (nucleotide sequence with N-terminal methionine)
atgggagtttctgatgtgccgcgcgacctggaagtggttgctgccacccccaccagcctgctgatcagctggcataggttctcttctatcatggcgtattaccgcatcacttacggcgaaacaggaggcaatagccctgtccaggagttcactgtggctggctctgttaacacagctaccatcagcggccttaaacctggcgttgattataccatcactgtgtatgctgtcacgatccataacgtttctttcccaatttccattaattaccgcaca

SEQ ID NO: 63 - DNA, 282 bp, Artificial Sequence
  Synthetic ATI-966 w/ N leader and modified C-terminus including PC (nucleotide sequence with N-terminal methionine)
atgggagtttctgatgtgccgcgcgacctggaagtggttgctgccacccccaccagcctgctgatcagctggcataggttctcttctatcatggcgtattaccgcatcacttacggcgaaacaggaggcaatagccctgtccaggagttcactgtggctggctctgttaacacagctaccatcagcggccttaaacctggcgttgattataccatcactgtgtatgctgtcacgatccataacgtttctttcccaatttccattaattaccgcacaccgtgc

SEQ ID NO: 64 - DNA, 318 bp, Artificial Sequence
  Synthetic ATI-966 w/ N leader and C tail + his tag (nucleotide sequence with N-terminal methionine)
atgggagtttctgatgtgccgcgcgacctggaagtggttgctgccacccccaccagcctgctgatcagctggcataggttctcttctatcatggcgtattaccgcatcacttacggcgaaacaggaggcaatagccctgtccaggagttcactgtggctggctctgttaacacagctaccatcagcggccttaaacctggcgttgattataccatcactgtgtatgctgtcacgatccataacgtttctttcccaatttccattaattaccgcacagaaattgacaaaccatcccagcaccatcaccaccaccactga

SEQ ID NO: 65 - protein, 84 aa, Artificial Sequence
  Synthetic ATI-967 core (parent of ADX_5417_E01)
EVVAATPTSLLISWQGQLSPSFYYRITYGETGGNSPVQEFTVPVASGTATISGLKPGVDYTITVYAVTSHGIYFYAPISINYRT

SEQ ID NO: 66 - protein, 9 aa, Artificial Sequence
  Synthetic ATI-967 BC loop
QGQLSPSFY

SEQ ID NO: 67 - protein, 6 aa, Artificial Sequence
  Synthetic ATI-967 DE loop
PVASGT

SEQ ID NO: 68 - protein, 11 aa, Artificial Sequence
  Synthetic ATI-967 FG loop
VTSHGIYFYAP

SEQ ID NO: 69 - protein, 93 aa, Artificial Sequence
  Synthetic ATI-967 w/ N leader
GVSDVPRDLEVVAATPTSLLISWQGQLSPSFYYRITYGETGGNSPVQEFTVPVASGTATISGLKPGVDYTITVYAVTSHGIYFYAPISINYRT

SEQ ID NO: 70 - protein, 99 aa, Artificial Sequence
  Synthetic ATI-967 w/ N leader + his tag
GVSDVPRDLEVVAATPTSLLISWQGQLSPSFYYRITYGETGGNSPVQEFTVPVASGTATISGLKPGVDYTITVYAVTSHGIYFYAPISINYRTHHHHHH

SEQ ID NO: 71 - protein, 100 aa, Artificial Sequence
  Synthetic ATI-967 w/ N leader and C tail
GVSDVPRDLEVVAATPTSLLISWQGQLSPSFYYRITYGETGGNSPVQEFTVPVASGTATISGLKPGVDYTITVYAVTSHGIYFYAPISINYRTEIDKPSQ

SEQ ID NO: 72 - protein, 106 aa, Artificial Sequence
  Synthetic ATI-967 w/ N leader and C tail + his tag
GVSDVPRDLEVVAATPTSLLISWQGQLSPSFYYRITYGETGGNSPVQEFTVPVASGTATISGLKPGVDYTITVYAVTSHGIYFYAPISINYRTEIDKPSQHHHHHH

SEQ ID NO: 73 - protein, 95 aa, Artificial Sequence
  Synthetic ATI-967 w/ N leader and modified C-terminus including PC
GVSDVPRDLEVVAATPTSLLISWQGQLSPSFYYRITYGETGGNSPVQEFTVPVASGTATISGLKPGVDYTITVYAVTSHGIYFYAPISINYRTPC

SEQ ID NO: 74 - protein, 101 aa, Artificial Sequence
  Synthetic ATI-967 w/ N leader and modified C-terminus including PC + his tag
GVSDVPRDLEVVAATPTSLLISWQGQLSPSFYYRITYGETGGNSPVQEFTVPVASGTATISGLKPGVDYTITVYAVTSHGIYFYAPISINYRTPCHHHHHH

SEQ ID NO: 75 - protein, 107 aa, Artificial Sequence
  Synthetic ATI-967  full length
MGVSDVPRDLEVVAATPTSLLISWQGQLSPSFYYRITYGETGGNSPVQEFTVPVASGTATISGLKPGVDYTITVYAVTSHGIYFYAPISINYRTEIDKPSQHHHHHH

SEQ ID NO: 76 - DNA, 252 bp, Artificial Sequence
  Synthetic ATI-967  core (nucleotide sequence)
gaagtggttgctgccacccccaccagcctgctgatcagctggcagggacagctgtctccgtctttctattaccgaatcacttacggcgaaacaggaggcaatagccctgtccaggagttcactgtgcctgttgctagtgggacagctaccatcagcggccttaaacctggcgttgattataccatcactgtgtatgctgtcacttctcatggcatatacttctacgctccaatttccattaattaccgcaca

SEQ ID NO: 77 - DNA, 282 bp, Artificial Sequence
  Synthetic ATI-967 w/ N leader (nucleotide sequence with N-terminal methionine)
atgggagtttctgatgtgccgcgcgacctggaagtggttgctgccacccccaccagcctgctgatcagctggcagggacagctgtctccgtctttctattaccgaatcacttacggcgaaacaggaggcaatagccctgtccaggagttcactgtgcctgttgctagtgggacagctaccatcagcggccttaaacctggcgttgattataccatcactgtgtatgctgtcacttctcatggcatatacttctacgctccaatttccattaattaccgcaca

SEQ ID NO: 78 - DNA, 288 bp, Artificial Sequence
  Synthetic ATI-967 w/ N leader and modified C-terminus including PC (nucleotide sequence with N-terminal methionine)
atgggagtttctgatgtgccgcgcgacctggaagtggttgctgccacccccaccagcctgctgatcagctggcagggacagctgtctccgtctttctattaccgaatcacttacggcgaaacaggaggcaatagccctgtccaggagttcactgtgcctgttgctagtgggacagctaccatcagcggccttaaacctggcgttgattataccatcactgtgtatgctgtcacttctcatggcatatacttctacgctccaatttccattaattaccgcacaccgtgc

SEQ ID NO: 79 - DNA, 325 bp, Artificial Sequence
  Synthetic ATI-967 w/ N leader and C tail + his tag (nucleotide sequence with N-terminal methionine)
atgggagtttctgatgtgccgcgcgacctggaagtggttgctgccacccccaccagcctgctgatcagctggcagggacagctgtctccgtctttctattaccgaatcacttacggcgaaacaggaggcaatagccctgtccaggagttcactgtgcctgttgctagtgggacagctaccatcagcggccttaaacctggcgttgattataccatcactgtgtatgctgtcacttctcatggcatatacttctacgctccaatttccattaattaccgcacagaaattgacaaaccatcccagcaccatcaccaccaccactgat

SEQ ID NO: 80 - protein, 86 aa, Artificial Sequence
  Synthetic ADX_5322_A02 core
EVVAATPTSLLISWSYDGPIDRYYRITYGETGGNSPVQEFTVPPDQKTATISGLKPGVDYTITVYAVRLEEAHYNREFPISINYRT

SEQ ID NO: 81 - protein, 9 aa, Artificial Sequence
  Synthetic ADX_5322_A02  BC loop
SYDGPIDRY

SEQ ID NO: 82 - protein, 6 aa, Artificial Sequence
  Synthetic ADX_5322_A02  DE loop
PPDQKT

SEQ ID NO: 83 - protein, 13 aa, Artificial Sequence
  Synthetic ADX_5322_A02  FG loop
VRLEEAHYNREFP

SEQ ID NO: 84 - protein, 95 aa, Artificial Sequence
  Synthetic ADX_5322_A02  w/ N leader
GVSDVPRDLEVVAATPTSLLISWSYDGPIDRYYRITYGETGGNSPVQEFTVPPDQKTATISGLKPGVDYTITVYAVRLEEAHYNREFPISINYRT

SEQ ID NO: 85 - protein, 101 aa, Artificial Sequence
  Synthetic ADX_5322_A02  w/ N leader + his tag
GVSDVPRDLEVVAATPTSLLISWSYDGPIDRYYRITYGETGGNSPVQEFTVPPDQKTATISGLKPGVDYTITVYAVRLEEAHYNREFPISINYRTHHHHHH

SEQ ID NO: 86 - protein, 102 aa, Artificial Sequence
  Synthetic ADX_5322_A02  w/ N leader and C tail
GVSDVPRDLEVVAATPTSLLISWSYDGPIDRYYRITYGETGGNSPVQEFTVPPDQKTATISGLKPGVDYTITVYAVRLEEAHYNREFPISINYRTEIDKPSQ

SEQ ID NO: 87 - protein, 108 aa, Artificial Sequence
  Synthetic ADX_5322_A02  w/ N leader and C tail + his tag
GVSDVPRDLEVVAATPTSLLISWSYDGPIDRYYRITYGETGGNSPVQEFTVPPDQKTATISGLKPGVDYTITVYAVRLEEAHYNREFPISINYRTEIDKPSQHHHHHH

SEQ ID NO: 88 - protein, 97 aa, Artificial Sequence
  Synthetic ADX_5322_A02  w/ N leader and modified C-terminus including PC
GVSDVPRDLEVVAATPTSLLISWSYDGPIDRYYRITYGETGGNSPVQEFTVPPDQKTATISGLKPGVDYTITVYAVRLEEAHYNREFPISINYRTPC

SEQ ID NO: 89 - protein, 103 aa, Artificial Sequence
  Synthetic ADX_5322_A02  w/ N leader and modified C-terminus including PC + his tag
GVSDVPRDLEVVAATPTSLLISWSYDGPIDRYYRITYGETGGNSPVQEFTVPPDQKTATISGLKPGVDYTITVYAVRLEEAHYNREFPISINYRTPCHHHHHH

SEQ ID NO: 90 - protein, 97 aa, Artificial Sequence; identical to SEQ ID NO: 88
  Synthetic ADX_5322_A02-Mal-DBCO-FFPF18
  misc_feature: 3′ -[Maleamide-DBCO-FFP(18F)]

SEQ ID NO: 91 - protein, 104 aa, Artificial Sequence
  Synthetic ADX_5322_A02 full length
MGVSDVPRDLEVVAATPTSLLISWSYDGPIDRYYRITYGETGGNSPVQEFTVPPDQKTATISGLKPGVDYTITVYAVRLEEAHYNREFPISINYRTPCHHHHHH

SEQ ID NO: 92 - DNA, 258 bp, Artificial Sequence
  Synthetic ADX_5322_A02  core (nucleotide sequence)
gaagtggttgctgccacccccaccagcctgctgatcagctggtcttacgatggcccaattgaccggtattaccgcatcacttacggcgaaacaggaggcaatagccctgtccaggagttcactgtgcctccggatcagaagacagctaccatcagcggccttaaacctggcgttgattataccatcactgtgtatgctgtccggctggaagaagctcattacaatcgagagtttccaatttccattaattaccgcaca

SEQ ID NO: 93 - DNA, 288 bp, Artificial Sequence
  Synthetic ADX_5322_A02 w/ N leader (nucleotide sequence with N-terminal methionine)
atgggagtttctgatgtgccgcgcgacctggaagtggttgctgccacccccaccagcctgctgatcagctggtcttacgatggcccaattgaccggtattaccgcatcacttacggcgaaacaggaggcaatagccctgtccaggagttcactgtgcctccggatcagaagacagctaccatcagcggccttaaacctggcgttgattataccatcactgtgtatgctgtccggctggaagaagctcattacaatcgagagtttccaatttccattaattaccgcaca

SEQ ID NO: 94 - DNA, 294 bp, Artificial Sequence
  Synthetic ADX_5322_A02 w/ N leader and modified C-terminus including PC (nucleotide sequence with N-terminal methionine)
atgggagtttctgatgtgccgcgcgacctggaagtggttgctgccacccccaccagcctgctgatcagctggtcttacgatggcccaattgaccggtattaccgcatcacttacggcgaaacaggaggcaatagccctgtccaggagttcactgtgcctccggatcagaagacagctaccatcagcggccttaaacctggcgttgattataccatcactgtgtatgctgtccggctggaagaagctcattacaatcgagagtttccaatttccattaattaccgcacaccgtgc

SEQ ID NO: 95 - DNA, 315 bp, Artificial Sequence
  Synthetic ADX_5322_A02 w/ N leader and C tail + his tag (nucleotide sequence with N-terminal methionine)
atgggagtttctgatgtgccgcgcgacctggaagtggttgctgccacccccaccagcctgctgatcagctggtcttacgatggcccaattgaccggtattaccgcatcacttacggcgaaacaggaggcaatagccctgtccaggagttcactgtgcctccggatcagaagacagctaccatcagcggccttaaacctggcgttgattataccatcactgtgtatgctgtccggctggaagaagctcattacaatcgagagtttccaatttccattaattaccgcacaccgtgccaccatcaccaccaccactga

SEQ ID NO: 96 - protein, 84 aa, Artificial Sequence
  Synthetic ADX_5417_E01 core
EVVAATPTSLLISWRAQLSPSFYYRITYGETGGNSPVQEFTVPNDVMTATISGLKPGVDYTITVYAVTTHGVYFYSPISINYRT

SEQ ID NO: 97 - protein, 9 aa, Artificial Sequence
  Synthetic ADX_5417_E01 BC loop
RAQLSPSFY

SEQ ID NO: 98 - protein, 6 aa, Artificial Sequence
  Synthetic ADX_5417_E01 DE loop
PNDVMT

SEQ ID NO: 99 - protein, 11 aa, Artificial Sequence
  Synthetic ADX_5417_E01 FG loop
VTTHGVYFYSP

SEQ ID NO: 100 - protein, 93 aa, Artificial Sequence
  Synthetic ADX_5417_E01 w/ N leader
GVSDVPRDLEVVAATPTSLLISWRAQLSPSFYYRITYGETGGNSPVQEFTVPNDVMTATISGLKPGVDYTITVYAVTTHGVYFYSPISINYRT

SEQ ID NO: 101 - protein, 99 aa, Artificial Sequence
  Synthetic ADX_5417_E01 w/ N leader + his tag
GVSDVPRDLEVVAATPTSLLISWRAQLSPSFYYRITYGETGGNSPVQEFTVPNDVMTATISGLKPGVDYTITVYAVTTHGVYFYSPISINYRTHHHHHH

SEQ ID NO: 102 - protein, 100 aa, Artificial Sequence
  Synthetic ADX_5417_E01 w/ N leader and C tail
GVSDVPRDLEVVAATPTSLLISWRAQLSPSFYYRITYGETGGNSPVQEFTVPNDVMTATISGLKPGVDYTITVYAVTTHGVYFYSPISINYRTEIDKPSQ

SEQ ID NO: 103 - protein, 106 aa, Artificial Sequence
  Synthetic ADX_5417_E01 w/ N leader and C tail + his tag
GVSDVPRDLEVVAATPTSLLISWRAQLSPSFYYRITYGETGGNSPVQEFTVPNDVMTATISGLKPGVDYTITVYAVTTHGVYFYSPISINYRTEIDKPSQHHHHHH

SEQ ID NO: 104 - protein, 95 aa, Artificial Sequence
  Synthetic ADX_5417_E01 w/ N leader and modified C-terminus including PC
GVSDVPRDLEVVAATPTSLLISWRAQLSPSFYYRITYGETGGNSPVQEFTVPNDVMTATISGLKPGVDYTITVYAVTTHGVYFYSPISINYRTPC

SEQ ID NO: 105 - protein, 101 aa, Artificial Sequence
  Synthetic ADX_5417_E01 w/ N leader and modified C-terminus including PC + his tag
GVSDVPRDLEVVAATPTSLLISWRAQLSPSFYYRITYGETGGNSPVQEFTVPNDVMTATISGLKPGVDYTITVYAVTTHGVYFYSPISINYRTPCHHHHHH

SEQ ID NO: 106 - protein, 97 aa, Artificial Sequence; identical to SEQ ID NO: 88
  Synthetic ADX-5417_E01 Mal-DBCO-FFPF18
  misc_feature: 3′ -[Maleamide-DBCO-FFP(18F)]

SEQ ID NO: 107 - protein, 102 aa, Artificial Sequence
  Synthetic ADX-5417_E01 full length
MGVSDVPRDLEVVAATPTSLLISWRAQLSPSFYYRITYGETGGNSPVQEFTVPNDVMTATISGLKPGVDYTITVYAVTTHGVYFYSPISINYRTPCHHHHHH

SEQ ID NO: 108 - DNA, 252 bp, Artificial Sequence
  Synthetic ADX-5417_E01  core (nucleotide sequence)
gaagtggttgctgccacccccaccagcctgctgatcagctggagggctcagctgtctccgtctttctattaccgcatcacttacggcgaaacaggaggcaatagccctgtccaggagttcactgtgcctaatgatgtaatgacagctaccatcagcggccttaaacctggcgttgattataccatcactgtgtatgctgtcactactcatggtgtttatttctactcaccaatttccattaattaccgcaca

SEQ ID NO: 109 - DNA, 282 bp, Artificial Sequence
  Synthetic ADX_5417_E01 w/ N leader (nucleotide sequence with N-terminal methionine)
atgggagtttctgatgtgccgcgcgacctggaagtggttgctgccacccccaccagcctgctgatcagctggagggctcagctgtctccgtctttctattaccgcatcacttacggcgaaacaggaggcaatagccctgtccaggagttcactgtgcctaatgatgtaatgacagctaccatcagcggccttaaacctggcgttgattataccatcactgtgtatgctgtcactactcatggtgtttatttctactcaccaatttccattaattaccgcaca

SEQ ID NO: 110 - DNA, 288 bp, Artificial Sequence
  Synthetic ADX_5417_E01 w/ N leader and modified C-terminus including PC (nucleotide sequence with N-terminal methionine)
atgggagtttctgatgtgccgcgcgacctggaagtggttgctgccacccccaccagcctgctgatcagctggagggctcagctgtctccgtctttctattaccgcatcacttacggcgaaacaggaggcaatagccctgtccaggagttcactgtgcctaatgatgtaatgacagctaccatcagcggccttaaacctggcgttgattataccatcactgtgtatgctgtcactactcatggtgtttatttctactcaccaatttccattaattaccgcacaccgtgc

SEQ ID NO: 111 - DNA, 309 bp, Artificial Sequence
  Synthetic ADX_5417_E01 w/ N leader and C tail + his tag (nucleotide sequence with N-terminal methionine)
atgggagtttctgatgtgccgcgcgacctggaagtggttgctgccacccccaccagcctgctgatcagctggagggctcagctgtctccgtctttctattaccgcatcacttacggcgaaacaggaggcaatagccctgtccaggagttcactgtgcctaatgatgtaatgacagctaccatcagcggccttaaacctggcgttgattataccatcactgtgtatgctgtcactactcatggtgtttatttctactcaccaatttccattaattaccgcacaccgtgccaccatcaccaccaccactga

SEQ ID NO: 112 - protein, 80 aa, Artificial Sequence
  Synthetic ATI_1420_A10 core
EVVAATPTSLLISWPYPSYYIEYRITYGETGGNSPVQEFTVQSMKATISGLKPGVDYTITVYAIRHPGMLEFGISINYRT

SEQ ID NO: 113 - protein, 8 aa, Artificial Sequence
  Synthetic ATI_1420_A10 BC loop
PYPSYYIE

SEQ ID NO: 114 - protein, 10 aa, Artificial Sequence
  Synthetic ATI_1420_A10 DE loop
IRHPGMLEFG

SEQ ID NO: 115 - protein, 13 aa, Artificial Sequence
  Synthetic ATI_1420_A10 FG loop
VTDGASIASYAFP

SEQ ID NO: 116 - protein, 89 aa, Artificial Sequence
  Synthetic ATI_1420_A10 w/ N leader
GVSDVPRDLEVVAATPTSLLISWPYPSYYIEYRITYGETGGNSPVQEFTVQSMKATISGLKPGVDYTITVYAIRHPGMLEFGISINYRT

SEQ ID NO: 117 - protein, 95 aa, Artificial Sequence
  Synthetic ATI_1420_A10 w/ N leader + his tag
GVSDVPRDLEVVAATPTSLLISWPYPSYYIEYRITYGETGGNSPVQEFTVQSMKATISGLKPGVDYTITVYAIRHPGMLEFGISINYRTHHHHHH

SEQ ID NO: 118 - protein, 96 aa, Artificial Sequence
  Synthetic ATI_1420_A10 w/ N leader and C tail
GVSDVPRDLEVVAATPTSLLISWPYPSYYIEYRITYGETGGNSPVQEFTVQSMKATISGLKPGVDYTITVYAIRHPGMLEFGISINYRTEIDKPSQ

SEQ ID NO: 119 - protein, 102 aa, Artificial Sequence
  Synthetic ATI_1420_A10w/ N leader and C tail + his tag
GVSDVPRDLEVVAATPTSLLISWPYPSYYIEYRITYGETGGNSPVQEFTVQSMKATISGLKPGVDYTITVYAIRHPGMLEFGISINYRTEIDKPSQHHHHHH

SEQ ID NO: 120 - protein, 91 aa, Artificial Sequence
  Synthetic ATI_1420_A10w/ N leader and modified C-terminus including PC
GVSDVPRDLEVVAATPTSLLISWPYPSYYIEYRITYGETGGNSPVQEFTVQSMKATISGLKPGVDYTITVYAIRHPGMLEFGISINYRTPC

SEQ ID NO: 121 - protein, 97 aa, Artificial Sequence
  Synthetic ATI_1420_A10 w/ N leader and modified C-terminus including PC + his tag
GVSDVPRDLEVVAATPTSLLISWPYPSYYIEYRITYGETGGNSPVQEFTVQSMKATISGLKPGVDYTITVYAIRHPGMLEFGISINYRTPCHHHHHH

SEQ ID NO: 122 - protein, 103 aa, Artificial Sequence
  Synthetic ATI_1420_A10 full length
MGVSDVPRDLEVVAATPTSLLISWPYPSYYIEYRITYGETGGNSPVQEFTVQSMKATISGLKPGVDYTITVYAIRHPGMLEFGISINYRTEIDKPSQHHHHHH

SEQ ID NO: 123 - protein, 76 aa, Artificial Sequence
  Synthetic ATI_1420_B09 core
EVVAATPTSLLISWHKFSSLMSYRITYGETGGNSPVQEFTVGSVNATISGLKPGVDYTITVYAIHNVGFISINYRT

SEQ ID NO: 124 - protein, 8 aa, Artificial Sequence
  Synthetic ATI_1420_B09 BC loop
HKFSSLMS

SEQ ID NO: 125 - protein, 4 aa, Artificial Sequence
  Synthetic ATI_1420_B09 DE loop
GSVN

SEQ ID NO: 126 - protein, 6 aa, Artificial Sequence
  Synthetic ATI_1420_B09 FG loop
IHNVGF

SEQ ID NO: 127 - protein, 85 aa, Artificial Sequence
  Synthetic ATI_1420_B09 w/ N leader
GVSDVPRDLEVVAATPTSLLISWHKFSSLMSYRITYGETGGNSPVQEFTVGSVNATISGLKPGVDYTITVYAIHNVGFISINYRT

SEQ ID NO: 128 - protein, 91 aa, Artificial Sequence
  Synthetic ATI_1420_B09 w/ N leader + his tag
GVSDVPRDLEVVAATPTSLLISWHKFSSLMSYRITYGETGGNSPVQEFTVGSVNATISGLKPGVDYTITVYAIHNVGFISINYRTHHHHHH

SEQ ID NO: 129 - protein, 92 aa, Artificial Sequence
  Synthetic AATI_1420_B09 w/ N leader and C tail
GVSDVPRDLEVVAATPTSLLISWHKFSSLMSYRITYGETGGNSPVQEFTVGSVNATISGLKPGVDYTITVYAIHNVGFISINYRTEIDKPSQ

SEQ ID NO: 130 - protein, 98 aa, Artificial Sequence
  Synthetic ATI_1420_B09 w/ N leader and C tail + his tag
GVSDVPRDLEVVAATPTSLLISWHKFSSLMSYRITYGETGGNSPVQEFTVGSVNATISGLKPGVDYTITVYAIHNVGFISINYRTEIDKPSQHHHHHH

SEQ ID NO: 131 - protein, 87 aa, Artificial Sequence
  Synthetic ATI_1420_B09 w/ N leader and modified C-terminus including PC
GVSDVPRDLEVVAATPTSLLISWHKFSSLMSYRITYGETGGNSPVQEFTVGSVNATISGLKPGVDYTITVYAIHNVGFISINYRTPC

SEQ ID NO: 132 - protein, 93 aa, Artificial Sequence
  Synthetic ATI_1420_B09 w/ N leader and modified C-terminus including PC + his tag
GVSDVPRDLEVVAATPTSLLISWHKFSSLMSYRITYGETGGNSPVQEFTVGSVNATISGLKPGVDYTITVYAIHNVGFISINYRTPCHHHHHH

SEQ ID NO: 133 - protein, 99 aa, Artificial Sequence
  Synthetic ATI_1420_B09 full length
MGVSDVPRDLEVVAATPTSLLISWHKFSSLMSYRITYGETGGNSPVQEFTVGSVNATISGLKPGVDYTITVYAIHNVGFISINYRTEIDKPSQHHHHHH

SEQ ID NO: 134 - protein, 79 aa, Artificial Sequence
  Synthetic ATI_1420_C02 core
EVVAATPTSLLISWRIKSYYAYRITYGETGGNSPVQEFTVRQHVATISGLKPGVDYTITVYARLGDVELVYEISINYRT

SEQ ID NO: 135 - protein, 7 aa, Artificial Sequence
  Synthetic ATI_1420_C02 BC loop
RIKSYYA

SEQ ID NO: 136 - protein, 4 aa, Artificial Sequence
  Synthetic ATI_1420_C02 DE loop
RQHV

SEQ ID NO: 137 - protein, 10 aa, Artificial Sequence
  Synthetic ATI_1420_C02 FG loop
RLGDVELVYE

SEQ ID NO: 138 - protein, 88 aa, Artificial Sequence
  Synthetic ATI_1420_C02 w/ N leader
GVSDVPRDLEVVAATPTSLLISWRIKSYYAYRITYGETGGNSPVQEFTVRQHVATISGLKPGVDYTITVYARLGDVELVYEISINYRT

SEQ ID NO: 139 - protein, 91 aa, Artificial Sequence; identical to SEQ ID NO: 128
  Synthetic ATI_1420_C02 w/ N leader + his tag

SEQ ID NO: 140 - protein, 95 aa, Artificial Sequence
  Synthetic ATI_1420_C02 w/ N leader and C tail
GVSDVPRDLEVVAATPTSLLISWRIKSYYAYRITYGETGGNSPVQEFTVRQHVATISGLKPGVDYTITVYARLGDVELVYEISINYRTEIDKPSQ

SEQ ID NO: 141 - protein, 101 aa, Artificial Sequence
  Synthetic ATI_1420_C02 w/ N leader and C tail + his tag
GVSDVPRDLEVVAATPTSLLISWRIKSYYAYRITYGETGGNSPVQEFTVRQHVATISGLKPGVDYTITVYARLGDVELVYEISINYRTEIDKPSQHHHHHH

SEQ ID NO: 142 - protein, 90 aa, Artificial Sequence
  Synthetic ATI_1420_C02 w/ N leader and modified C-terminus including PC
GVSDVPRDLEVVAATPTSLLISWRIKSYYAYRITYGETGGNSPVQEFTVRQHVATISGLKPGVDYTITVYARLGDVELVYEISINYRTPC

SEQ ID NO: 143 - protein, 93 aa, Artificial Sequence; identical to SEQ ID NO: 132
  Synthetic ATI_1420_C02 w/ N leader and modified C-terminus including PC + his tag

SEQ ID NO: 144 - protein, 102 aa, Artificial Sequence
  Synthetic ATI_1420_C02 full length
MGVSDVPRDLEVVAATPTSLLISWRIKSYYAYRITYGETGGNSPVQEFTVRQHVATISGLKPGVDYTITVYARLGDVELVYEISINYRTEIDKPSQHHHHHH

SEQ ID NO: 145 - protein, 80 aa, Artificial Sequence
  Synthetic ATI_1420_C11 core
EVVAATPTSLLISWMYPLKSVPYRITYGETGGNSPVQEFTVYSSGATISGLKPGVDYTITVYAMSYSTYHAFMISINYRT

SEQ ID NO: 146 - protein, 8 aa, Artificial Sequence
  Synthetic ATI_1420_C11 BC loop
MYPLKSVP

SEQ ID NO: 147 - protein, 4 aa, Artificial Sequence
  Synthetic ATI_1420_C11 DE loop
YSSG

SEQ ID NO: 148 - protein, 10 aa, Artificial Sequence
  Synthetic ATI_1420_C11 FG loop
MSYSTYHAFM

SEQ ID NO: 149 - protein, 89 aa, Artificial Sequence
  Synthetic ATI_1420_C11 w/ N leader
GVSDVPRDLEVVAATPTSLLISWMYPLKSVPYRITYGETGGNSPVQEFTVYSSGATISGLKPGVDYTITVYAMSYSTYHAFMISINYRT

SEQ ID NO: 150 - protein, 95 aa, Artificial Sequence
  Synthetic ATI_1420_C11 w/ N leader + his tag
GVSDVPRDLEVVAATPTSLLISWMYPLKSVPYRITYGETGGNSPVQEFTVYSSGATISGLKPGVDYTITVYAMSYSTYHAFMISINYRTHHHHHH

SEQ ID NO: 151 - protein, 96 aa, Artificial Sequence
  Synthetic ATI_1420_C11 w/ N leader and C tail
GVSDVPRDLEVVAATPTSLLISWMYPLKSVPYRITYGETGGNSPVQEFTVYSSGATISGLKPGVDYTITVYAMSYSTYHAFMISINYRTEIDKPSQ

SEQ ID NO: 152 - protein, 102 aa, Artificial Sequence
  Synthetic ATI_1420_C11 w/ N leader and C tail + his tag
GVSDVPRDLEVVAATPTSLLISWMYPLKSVPYRITYGETGGNSPVQEFTVYSSGATISGLKPGVDYTITVYAMSYSTYHAFMISINYRTEIDKPSQHHHHHH

SEQ ID NO: 153 - protein, 91 aa, Artificial Sequence
  Synthetic ATI_1420_C11 w/ N leader and modified C-terminus including PC
GVSDVPRDLEVVAATPTSLLISWMYPLKSVPYRITYGETGGNSPVQEFTVYSSGATISGLKPGVDYTITVYAMSYSTYHAFMISINYRTPC

SEQ ID NO: 154 - protein, 97 aa, Artificial Sequence
  Synthetic ATI_1420_C11 w/ N leader and modified C-terminus including PC + his tag
GVSDVPRDLEVVAATPTSLLISWMYPLKSVPYRITYGETGGNSPVQEFTVYSSGATISGLKPGVDYTITVYAMSYSTYHAFMISINYRTPCHHHHHH

SEQ ID NO: 155 - protein, 103 aa, Artificial Sequence
  Synthetic AATI_1420_C11 full length
MGVSDVPRDLEVVAATPTSLLISWMYPLKSVPYRITYGETGGNSPVQEFTVYSSGATISGLKPGVDYTITVYAMSYSTYHAFMISINYRTEIDKPSQHHHHHH

SEQ ID NO: 156 - protein, 79 aa, Artificial Sequence
  Synthetic ATI_1420_D01 core
EVVAATPTSLLISWRTVPETDYRITYGETGGNSPVQEFTVPDNTATISGLKPGVDYTITVYALETAHYNRDYISINYRT

SEQ ID NO: 157 - protein, 7 aa, Artificial Sequence
  Synthetic ATI_1420_D01 BC loop
RTVPETD

SEQ ID NO: 158 - protein, 4 aa, Artificial Sequence
  Synthetic ATI_1420_D01 DE loop
PDNT

SEQ ID NO: 159 - protein, 10 aa, Artificial Sequence
  Synthetic ATI_1420_D01 FG loop
LETAHYNRDY

SEQ ID NO: 160 - protein, 88 aa, Artificial Sequence
  Synthetic ATI_1420_D01 w/ N leader
GVSDVPRDLEVVAATPTSLLISWRTVPETDYRITYGETGGNSPVQEFTVPDNTATISGLKPGVDYTITVYALETAHYNRDYISINYRT

SEQ ID NO: 161 - protein, 94 aa, Artificial Sequence
  Synthetic ATI_1420_D01 w/ N leader + his tag
GVSDVPRDLEVVAATPTSLLISWRTVPETDYRITYGETGGNSPVQEFTVPDNTATISGLKPGVDYTITVYALETAHYNRDYISINYRTHHHHHH

SEQ ID NO: 162 - protein, 95 aa, Artificial Sequence
  Synthetic ATI_1420_D01 w/ N leader and C tail
GVSDVPRDLEVVAATPTSLLISWRTVPETDYRITYGETGGNSPVQEFTVPDNTATISGLKPGVDYTITVYALETAHYNRDYISINYRTEIDKPSQ

SEQ ID NO: 163 - protein, 101 aa, Artificial Sequence
  Synthetic ATI_1420_D01 w/ N leader and C tail + his tag
GVSDVPRDLEVVAATPTSLLISWRTVPETDYRITYGETGGNSPVQEFTVPDNTATISGLKPGVDYTITVYALETAHYNRDYISINYRTEIDKPSQHHHHHH

SEQ ID NO: 164 - protein, 90 aa, Artificial Sequence
  Synthetic ATI_1420_D01 w/ N leader and modified C-terminus including PC
GVSDVPRDLEVVAATPTSLLISWRTVPETDYRITYGETGGNSPVQEFTVPDNTATISGLKPGVDYTITVYALETAHYNRDYISINYRTPC

SEQ ID NO: 165 - protein, 96 aa, Artificial Sequence
  Synthetic ATI_1420_D01 w/ N leader and modified C-terminus including PC + his tag
GVSDVPRDLEVVAATPTSLLISWRTVPETDYRITYGETGGNSPVQEFTVPDNTATISGLKPGVDYTITVYALETAHYNRDYISINYRTPCHHHHHH

SEQ ID NO: 166 - protein, 102 aa, Artificial Sequence
  Synthetic ATI_1420_D01 full length
MGVSDVPRDLEVVAATPTSLLISWRTVPETDYRITYGETGGNSPVQEFTVPDNTATISGLKPGVDYTITVYALETAHYNRDYISINYRTEIDKPSQHHHHHH

SEQ ID NO: 167 - protein, 82 aa, Artificial Sequence
  Synthetic ATI_1420_D05 core
EVVAATPTSLLISWTAYYSTIKYRITYGETGGNSPVQEFTVGPKHHATISGLKPGVDYTITVYAYNTKPGYHAHQISINYRT

SEQ ID NO: 168 - protein, 8 aa, Artificial Sequence
  Synthetic ATI_1420_D05 BC loop
TAYYSTIK

SEQ ID NO: 169 - protein, 5 aa, Artificial Sequence
  Synthetic ATI_1420_D05 DE loop
GPKHH

SEQ ID NO: 170 - protein, 11 aa, Artificial Sequence
  Synthetic ATI_1420_D05 FG loop
YNTKPGYHAHQ

SEQ ID NO: 171 - protein, 91 aa, Artificial Sequence
  Synthetic ATI_1420_D05 w/ N leader
GVSDVPRDLEVVAATPTSLLISWTAYYSTIKYRITYGETGGNSPVQEFTVGPKHHATISGLKPGVDYTITVYAYNTKPGYHAHQISINYRT

SEQ ID NO: 172 - protein, 97 aa, Artificial Sequence
  Synthetic ATI_1420_D05 w/ N leader + his tag
GVSDVPRDLEVVAATPTSLLISWTAYYSTIKYRITYGETGGNSPVQEFTVGPKHHATISGLKPGVDYTITVYAYNTKPGYHAHQISINYRTHHHHHH

SEQ ID NO: 173 - protein, 98 aa, Artificial Sequence
  Synthetic ATI_1420_D05 w/ N leader and C tail
GVSDVPRDLEVVAATPTSLLISWTAYYSTIKYRITYGETGGNSPVQEFTVGPKHHATISGLKPGVDYTITVYAYNTKPGYHAHQISINYRTEIDKPSQ

SEQ ID NO: 174 - protein, 104 aa, Artificial Sequence
  Synthetic ATI_1420_D05 w/ N leader and C tail + his tag
GVSDVPRDLEVVAATPTSLLISWTAYYSTIKYRITYGETGGNSPVQEFTVGPKHHATISGLKPGVDYTITVYAYNTKPGYHAHQISINYRTEIDKPSQHHHHHH

SEQ ID NO: 175 - protein, 93 aa, Artificial Sequence
  Synthetic ATI_1420_D05 w/ N leader and modified C-terminus including PC
GVSDVPRDLEVVAATPTSLLISWTAYYSTIKYRITYGETGGNSPVQEFTVGPKHHATISGLKPGVDYTITVYAYNTKPGYHAHQISINYRTPC

SEQ ID NO: 176 - protein, 99 aa, Artificial Sequence
  Synthetic ATI_1420_D05 w/ N leader and modified C-terminus including PC + his tag
GVSDVPRDLEVVAATPTSLLISWTAYYSTIKYRITYGETGGNSPVQEFTVGPKHHATISGLKPGVDYTITVYAYNTKPGYHAHQISINYRTPCHHHHHH

SEQ ID NO: 177 - protein, 105 aa, Artificial Sequence
  Synthetic ATI_1420_D05 full length
MGVSDVPRDLEVVAATPTSLLISWTAYYSTIKYRITYGETGGNSPVQEFTVGPKHHATISGLKPGVDYTITVYAYNTKPGYHAHQISINYRTEIDKPSQHHHHHH

SEQ ID NO: 178 - protein, 80 aa, Artificial Sequence
  Synthetic ATI_1420_D10  core
EVVAATPTSLLISWRIPSYHIQYRITYGETGGNSPVQEFTVYQKYATISGLKPGVDYTITVYAVSPPKQLRFGISINYRT

SEQ ID NO: 179 - protein, 8 aa, Artificial Sequence
  Synthetic ATI_1420_D10  BC loop
RIPSYHIQ

SEQ ID NO: 180 - protein, 4 aa, Artificial Sequence
  Synthetic ATI_1420_D10  DE loop
YQKY

SEQ ID NO: 181 - protein, 10 aa, Artificial Sequence
  Synthetic ATI_1420_D10  FG loop
VSPPKQLRFG

SEQ ID NO: 182 - protein, 89 aa, Artificial Sequence
  Synthetic ATI_1420_D10  w/ N leader
GVSDVPRDLEVVAATPTSLLISWRIPSYHIQYRITYGETGGNSPVQEFTVYQKYATISGLKPGVDYTITVYAVSPPKQLRFGISINYRT

SEQ ID NO: 183 - protein, 95 aa, Artificial Sequence
  Synthetic ATI_1420_D10  w/ N leader + his tag
GVSDVPRDLEVVAATPTSLLISWRIPSYHIQYRITYGETGGNSPVQEFTVYQKYATISGLKPGVDYTITVYAVSPPKQLRFGISINYRTHHHHHH

SEQ ID NO: 184 - protein, 96 aa, Artificial Sequence
  Synthetic ATI_1420_D10  w/ N leader and C tail
GVSDVPRDLEVVAATPTSLLISWRIPSYHIQYRITYGETGGNSPVQEFTVYQKYATISGLKPGVDYTITVYAVSPPKQLRFGISINYRTEIDKPSQ

SEQ ID NO: 185 - protein, 102 aa, Artificial Sequence
  Synthetic ATI_1420_D10  w/ N leader and C tail + his tag
GVSDVPRDLEVVAATPTSLLISWRIPSYHIQYRITYGETGGNSPVQEFTVYQKYATISGLKPGVDYTITVYAVSPPKQLRFGISINYRTEIDKPSQHHHHHH

SEQ ID NO: 186 - protein, 91 aa, Artificial Sequence
  Synthetic ATI_1420_D10  w/ N leader and modified C-terminus including PC
GVSDVPRDLEVVAATPTSLLISWRIPSYHIQYRITYGETGGNSPVQEFTVYQKYATISGLKPGVDYTITVYAVSPPKQLRFGISINYRTPC

SEQ ID NO: 187 - protein, 97 aa, Artificial Sequence
  Synthetic ATI_1420_D10  w/ N leader and modified C-terminus including PC + his tag
GVSDVPRDLEVVAATPTSLLISWRIPSYHIQYRITYGETGGNSPVQEFTVYQKYATISGLKPGVDYTITVYAVSPPKQLRFGISINYRTPCHHHHHH

SEQ ID NO: 188 - protein, 103 aa, Artificial Sequence
  Synthetic ATI_1420_D010 full length
MGVSDVPRDLEVVAATPTSLLISWRIPSYHIQYRITYGETGGNSPVQEFTVYQKYATISGLKPGVDYTITVYAVSPPKQLRFGISINYRTEIDKPSQHHHHHH

SEQ ID NO: 189 - protein, 76 aa, Artificial Sequence
  Synthetic ATI_1420_F10_core
EVVAATPTSLLISWPAPPSYVFYRITYGETGGNSPVQEFTVYPYMATISGLKPGVDYTITVYAYTSGFSISINYRT

SEQ ID NO: 190 - protein, 8 aa, Artificial Sequence
  Synthetic ATI_1420_F10  BC loop
PAPPSYVF

SEQ ID NO: 191 - protein, 4 aa, Artificial Sequence
  Synthetic ATI_1420_F10 DE loop
YPYM

SEQ ID NO: 192 - protein, 6 aa, Artificial Sequence
  Synthetic ATI_1420_F10 FG loop
YTSGFS

SEQ ID NO: 193 - protein, 85 aa, Artificial Sequence
  Synthetic ATI_1420_F10 w/ N leader
GVSDVPRDLEVVAATPTSLLISWPAPPSYVFYRITYGETGGNSPVQEFTVYPYMATISGLKPGVDYTITVYAYTSGFSISINYRT

SEQ ID NO: 194 - protein, 91 aa, Artificial Sequence
  Synthetic ATI_1420_F10  w/ N leader + his tag
GVSDVPRDLEVVAATPTSLLISWPAPPSYVFYRITYGETGGNSPVQEFTVYPYMATISGLKPGVDYTITVYAYTSGFSISINYRTHHHHHH

SEQ ID NO: 195 - protein, 92 aa, Artificial Sequence
  Synthetic ATI_1420_F10 w/ N leader and C tail
GVSDVPRDLEVVAATPTSLLISWPAPPSYVFYRITYGETGGNSPVQEFTVYPYMATISGLKPGVDYTITVYAYTSGFSISINYRTEIDKPSQ

SEQ ID NO: 196 - protein, 98 aa, Artificial Sequence
  Synthetic ATI_1420_F10  w/ N leader and C tail + his tag
GVSDVPRDLEVVAATPTSLLISWPAPPSYVFYRITYGETGGNSPVQEFTVYPYMATISGLKPGVDYTITVYAYTSGFSISINYRTEIDKPSQHHHHHH

SEQ ID NO: 197 - protein, 87 aa, Artificial Sequence
  Synthetic ATI_1420_F10  w/ N leader and modified C-terminus including PC
GVSDVPRDLEVVAATPTSLLISWPAPPSYVFYRITYGETGGNSPVQEFTVYPYMATISGLKPGVDYTITVYAYTSGFSISINYRTPC

SEQ ID NO: 198 - protein, 93 aa, Artificial Sequence
  Synthetic ATI_1420_F10  w/ N leader and modified C-terminus including PC + his tag
GVSDVPRDLEVVAATPTSLLISWPAPPSYVFYRITYGETGGNSPVQEFTVYPYMATISGLKPGVDYTITVYAYTSGFSISINYRTPCHHHHHH

SEQ ID NO: 199 - protein, 99 aa, Artificial Sequence
  Synthetic ATI_1420_F10 full length
MGVSDVPRDLEVVAATPTSLLISWPAPPSYVFYRITYGETGGNSPVQEFTVYPYMATISGLKPGVDYTITVYAYTSGFSISINYRTEIDKPSQHHHHHH

SEQ ID NO: 200 - protein, 79 aa, Artificial Sequence
  Synthetic ATI_1421_C05_core
EVVAATPTSLLISWYMDHKSKYRITYGETGGNSPVQEFTVPDQRATISGLKPGVDYTITVYALSEAHYLRDKISINYRT

SEQ ID NO: 201 - protein, 7 aa, Artificial Sequence
  Synthetic ATI_1421_C05  BC loop
YMDHKSK

SEQ ID NO: 202 - protein, 4 aa, Artificial Sequence
  Synthetic ATI_1421_C05 DE loop
PDQR

SEQ ID NO: 203 - protein, 10 aa, Artificial Sequence
  Synthetic ATI_1421_C05 FG loop
LSEAHYLRDK

SEQ ID NO: 204 - protein, 88 aa, Artificial Sequence
  Synthetic ATI_1421_C05 w/ N leader
GVSDVPRDLEVVAATPTSLLISWYMDHKSKYRITYGETGGNSPVQEFTVPDQRATISGLKPGVDYTITVYALSEAHYLRDKISINYRT

SEQ ID NO: 205 - protein, 94 aa, Artificial Sequence
  Synthetic ATI_1421_C05  w/ N leader + his tag
GVSDVPRDLEVVAATPTSLLISWYMDHKSKYRITYGETGGNSPVQEFTVPDQRATISGLKPGVDYTITVYALSEAHYLRDKISINYRTHHHHHH

SEQ ID NO: 206 - protein, 95 aa, Artificial Sequence
  Synthetic ATI_1421_C05 w/ N leader and C tail
GVSDVPRDLEVVAATPTSLLISWYMDHKSKYRITYGETGGNSPVQEFTVPDQRATISGLKPGVDYTITVYALSEAHYLRDKISINYRTEIDKPSQ

SEQ ID NO: 207 - protein, 101 aa, Artificial Sequence
  Synthetic ATI_1421_C05  w/ N leader and C tail + his tag
GVSDVPRDLEVVAATPTSLLISWYMDHKSKYRITYGETGGNSPVQEFTVPDQRATISGLKPGVDYTITVYALSEAHYLRDKISINYRTEIDKPSQHHHHHH

SEQ ID NO: 208 - protein, 90 aa, Artificial Sequence
  Synthetic ATI_1421_C05  w/ N leader and modified C-terminus including PC
GVSDVPRDLEVVAATPTSLLISWYMDHKSKYRITYGETGGNSPVQEFTVPDQRATISGLKPGVDYTITVYALSEAHYLRDKISINYRTPC

SEQ ID NO: 209 - protein, 96 aa, Artificial Sequence
  Synthetic ATI_1421_C05 w/ N leader and modified C-terminus including PC + his tag
GVSDVPRDLEVVAATPTSLLISWYMDHKSKYRITYGETGGNSPVQEFTVPDQRATISGLKPGVDYTITVYALSEAHYLRDKISINYRTPCHHHHHH

SEQ ID NO: 210 - protein, 102 aa, Artificial Sequence
  Synthetic ATI_1421_C05 full length
MGVSDVPRDLEVVAATPTSLLISWYMDHKSKYRITYGETGGNSPVQEFTVPDQRATISGLKPGVDYTITVYALSEAHYLRDKISINYRTEIDKPSQHHHHHH

SEQ ID NO: 211 - protein, 79 aa, Artificial Sequence
  Synthetic ATI_1421_C06 core
EVVAATPTSLLISWENLASYQYRITYGETGGNSPVQEFTVPDQAATISGLKPGVDYTITVYALQTAHYYRQHISINYRT

SEQ ID NO: 212 - protein, 7 aa, Artificial Sequence
  Synthetic ATI_1421_C06 BC loop
ENLASYQ

SEQ ID NO: 213 - protein, 4 aa, Artificial Sequence
  Synthetic ATI_1421_C06DE loop
PDQA

SEQ ID NO: 214 - protein, 10 aa, Artificial Sequence
  Synthetic ATI_1421_C06FG loop
LQTAHYYRQH

SEQ ID NO: 215 - protein, 88 aa, Artificial Sequence
  Synthetic ATI_1421_C06 w/ N leader
GVSDVPRDLEVVAATPTSLLISWENLASYQYRITYGETGGNSPVQEFTVPDQAATISGLKPGVDYTITVYALQTAHYYRQHISINYRT

SEQ ID NO: 216 - protein, 94 aa, Artificial Sequence
  Synthetic ATI_1421_C06 w/ N leader + his tag
GVSDVPRDLEVVAATPTSLLISWENLASYQYRITYGETGGNSPVQEFTVPDQAATISGLKPGVDYTITVYALQTAHYYRQHISINYRTHHHHHH

SEQ ID NO: 217 - protein, 95 aa, Artificial Sequence
  Synthetic ATI_1421_C06w/ N leader and C tail
GVSDVPRDLEVVAATPTSLLISWENLASYQYRITYGETGGNSPVQEFTVPDQAATISGLKPGVDYTITVYALQTAHYYRQHISINYRTEIDKPSQ

SEQ ID NO: 218 - protein, 101 aa, Artificial Sequence
  Synthetic ATI_1421_C06 w/ N leader and C tail + his tag
GVSDVPRDLEVVAATPTSLLISWENLASYQYRITYGETGGNSPVQEFTVPDQAATISGLKPGVDYTITVYALQTAHYYRQHISINYRTEIDKPSQHHHHHH

SEQ ID NO: 219 - protein, 90 aa, Artificial Sequence
  Synthetic ATI_1421_C06 w/ N leader and modified C-terminus including PC
GVSDVPRDLEVVAATPTSLLISWENLASYQYRITYGETGGNSPVQEFTVPDQAATISGLKPGVDYTITVYALQTAHYYRQHISINYRTPC

SEQ ID NO: 220 - protein, 96 aa, Artificial Sequence
  Synthetic ATI_1421_C06 w/ N leader and modified C-terminus including PC + his tag
GVSDVPRDLEVVAATPTSLLISWENLASYQYRITYGETGGNSPVQEFTVPDQAATISGLKPGVDYTITVYALQTAHYYRQHISINYRTPCHHHHHH

SEQ ID NO: 221 - protein, 102 aa, Artificial Sequence
  Synthetic ATI_1421_C06 full length
MGVSDVPRDLEVVAATPTSLLISWENLASYQYRITYGETGGNSPVQEFTVPDQAATISGLKPGVDYTITVYALQTAHYYRQHISINYRTEIDKPSQHHHHHH

SEQ ID NO: 222 - protein, 79 aa, Artificial Sequence
  Synthetic ATI 1421_D05 core
EVVAATPTSLLISWYYVQYNDYRITYGETGGNSPVQEFTVPDQSATISGLKPGVDYTITVYALEKAHYYRQNISINYRT

SEQ ID NO: 223 - protein, 7 aa, Artificial Sequence
  Synthetic ATI 1421_D05 BC loop
YYVQYND

SEQ ID NO: 224 - protein, 4 aa, Artificial Sequence
  Synthetic ATI 1421_D05 DE loop
PDQS

SEQ ID NO: 225 - protein, 10 aa, Artificial Sequence
  Synthetic ATI 1421_D05 FG loop
LEKAHYYRQN

SEQ ID NO: 226 - protein, 88 aa, Artificial Sequence
  Synthetic ATI 1421_D05 w/ N leader
GVSDVPRDLEVVAATPTSLLISWYYVQYNDYRITYGETGGNSPVQEFTVPDQSATISGLKPGVDYTITVYALEKAHYYRQNISINYRT

SEQ ID NO: 227 - protein, 94 aa, Artificial Sequence
  Synthetic ATI 1421_D05 w/ N leader + his tag
GVSDVPRDLEVVAATPTSLLISWYYVQYNDYRITYGETGGNSPVQEFTVPDQSATISGLKPGVDYTITVYALEKAHYYRQNISINYRTHHHHHH

SEQ ID NO: 228 - protein, 95 aa, Artificial Sequence
  Synthetic ATI 1421_D05 w/ N leader and C tail
GVSDVPRDLEVVAATPTSLLISWYYVQYNDYRITYGETGGNSPVQEFTVPDQSATISGLKPGVDYTITVYALEKAHYYRQNISINYRTEIDKPSQ

SEQ ID NO: 229 - protein, 101 aa, Artificial Sequence
  Synthetic ATI 1421_D05 w/ N leader and C tail + his tag
GVSDVPRDLEVVAATPTSLLISWYYVQYNDYRITYGETGGNSPVQEFTVPDQSATISGLKPGVDYTITVYALEKAHYYRQNISINYRTEIDKPSQHHHHHH

SEQ ID NO: 230 - protein, 90 aa, Artificial Sequence
  Synthetic ATI 1421_D05 w/ N leader and modified C-terminus including PC
GVSDVPRDLEVVAATPTSLLISWYYVQYNDYRITYGETGGNSPVQEFTVPDQSATISGLKPGVDYTITVYALEKAHYYRQNISINYRTPC

SEQ ID NO: 231 - protein, 96 aa, Artificial Sequence
  Synthetic ATI 1421_D05 w/ N leader and modified C-terminus including PC + his tag
GVSDVPRDLEVVAATPTSLLISWYYVQYNDYRITYGETGGNSPVQEFTVPDQSATISGLKPGVDYTITVYALEKAHYYRQNISINYRTPCHHHHHH

SEQ ID NO: 232 - protein, 102 aa, Artificial Sequence
  Synthetic ATI 1421_D05  full length
MGVSDVPRDLEVVAATPTSLLISWYYVQYNDYRITYGETGGNSPVQEFTVPDQSATISGLKPGVDYTITVYALEKAHYYRQNISINYRTEIDKPSQHHHHHH

SEQ ID NO: 233 - protein, 79 aa, Artificial Sequence
  Synthetic ATI_1421_D06 core
EVVAATPTSLLISWGHNYDDEYRITYGETGGNSPVQEFTVPDQYATISGLKPGVDYTITVYALAEAHVRKNHISINYRT

SEQ ID NO: 234 - protein, 7 aa, Artificial Sequence
  Synthetic ATI_1421_D06 BC loop
GHNYDDE

SEQ ID NO: 235 - protein, 4 aa, Artificial Sequence
  Synthetic ATI_1421_D06  DE loop
PDQY

SEQ ID NO: 236 - protein, 10 aa, Artificial Sequence
  Synthetic ATI_1421_D06  FG loop
LAEAHVRKNH

SEQ ID NO: 237 - protein, 88 aa, Artificial Sequence
  Synthetic ATI_1421_D06 w/ N leader
GVSDVPRDLEVVAATPTSLLISWGHNYDDEYRITYGETGGNSPVQEFTVPDQYATISGLKPGVDYTITVYALAEAHVRKNHISINYRT

SEQ ID NO: 238 - protein, 94 aa, Artificial Sequence
  Synthetic ATI_1421_D06 w/ N leader + his tag
GVSDVPRDLEVVAATPTSLLISWGHNYDDEYRITYGETGGNSPVQEFTVPDQYATISGLKPGVDYTITVYALAEAHVRKNHISINYRTHHHHHH

SEQ ID NO: 239 - protein, 95 aa, Artificial Sequence
  Synthetic ATI_1421_D06 w/ N leader and C tail
GVSDVPRDLEVVAATPTSLLISWGHNYDDEYRITYGETGGNSPVQEFTVPDQYATISGLKPGVDYTITVYALAEAHVRKNHISINYRTEIDKPSQ

SEQ ID NO: 240 - protein, 101 aa, Artificial Sequence
  Synthetic ATI_1421_D06 w/ N leader and C tail + his tag
GVSDVPRDLEVVAATPTSLLISWGHNYDDEYRITYGETGGNSPVQEFTVPDQYATISGLKPGVDYTITVYALAEAHVRKNHISINYRTEIDKPSQHHHHHH

SEQ ID NO: 241 - protein, 90 aa, Artificial Sequence
  Synthetic ATI_1421_D06 w/ N leader and modified C-terminus including PC
GVSDVPRDLEVVAATPTSLLISWGHNYDDEYRITYGETGGNSPVQEFTVPDQYATISGLKPGVDYTITVYALAEAHVRKNHISINYRTPC

SEQ ID NO: 242 - protein, 96 aa, Artificial Sequence
  Synthetic ATI_1421_D06 w/ N leader and modified C-terminus including PC + his tag
GVSDVPRDLEVVAATPTSLLISWGHNYDDEYRITYGETGGNSPVQEFTVPDQYATISGLKPGVDYTITVYALAEAHVRKNHISINYRTPCHHHHHH

SEQ ID NO: 243 - protein, 102 aa, Artificial Sequence
  Synthetic ATI_1421_D06  full length
MGVSDVPRDLEVVAATPTSLLISWGHNYDDEYRITYGETGGNSPVQEFTVPDQYATISGLKPGVDYTITVYALAEAHVRKNHISINYRTEIDKPSQHHHHHH

SEQ ID NO: 244 - protein, 79 aa, Artificial Sequence
  Synthetic ATI_1421_E03 core
EVVAATPTSLLISWVYHYDAQYRITYGETGGNSPVQEFTVPDQKATISGLKPGVDYTITVYALSEAHHKRDSISINYRT

SEQ ID NO: 245 - protein, 7 aa, Artificial Sequence
  Synthetic ATI_1421_E03 BC loop
VYHYDAQ

SEQ ID NO: 246 - protein, 4 aa, Artificial Sequence
  Synthetic ATI_1421_E03 DE loop
PDQK

SEQ ID NO: 247 - protein, 10 aa, Artificial Sequence
  Synthetic ATI_1421_E03 FG loop
LSEAHHKRDS

SEQ ID NO: 248 - protein, 94 aa, Artificial Sequence
  Synthetic ATI_1421_E03 w/ N leader
GVSDVPRDLEVVAATPTSLLISWVYHYDAQYRITYGETGGNSPVQEFTVPDQKATISGLKPGVDYTITVYALSEAHHKRDSISINYRTHHHHHH

SEQ ID NO: 249 - protein, 95 aa, Artificial Sequence
  Synthetic ATI_1421_E03w/ N leader + his tag
GVSDVPRDLEVVAATPTSLLISWVYHYDAQYRITYGETGGNSPVQEFTVPDQKATISGLKPGVDYTITVYALSEAHHKRDSISINYRTEIDKPSQ

SEQ ID NO: 250 - protein, 101 aa, Artificial Sequence
  Synthetic ATI_1421_E03w/ N leader and C tail
GVSDVPRDLEVVAATPTSLLISWVYHYDAQYRITYGETGGNSPVQEFTVPDQKATISGLKPGVDYTITVYALSEAHHKRDSISINYRTEIDKPSQHHHHHH

SEQ ID NO: 251 - protein, 90 aa, Artificial Sequence
  Synthetic ATI_1421_E03 w/ N leader and C tail + his tag
GVSDVPRDLEVVAATPTSLLISWVYHYDAQYRITYGETGGNSPVQEFTVPDQKATISGLKPGVDYTITVYALSEAHHKRDSISINYRTPC

SEQ ID NO: 252 - protein, 96 aa, Artificial Sequence
  Synthetic ATI_1421_E03 w/ N leader and modified C-terminus including PC
GVSDVPRDLEVVAATPTSLLISWVYHYDAQYRITYGETGGNSPVQEFTVPDQKATISGLKPGVDYTITVYALSEAHHKRDSISINYRTPCHHHHHH

SEQ ID NO: 253 - protein, 88 aa, Artificial Sequence
  Synthetic ATI_1421_E03 w/ N leader and modified C-terminus including PC + his tag
GVSDVPRDLEVVAATPTSLLISWVYHYDAQYRITYGETGGNSPVQEFTVPDQKATISGLKPGVDYTITVYALSEAHHKRDSISINYRT

SEQ ID NO: 254 - protein, 102 aa, Artificial Sequence
  Synthetic ATI_1421_E03  full length
MGVSDVPRDLEVVAATPTSLLISWVYHYDAQYRITYGETGGNSPVQEFTVPDQKATISGLKPGVDYTITVYALSEAHHKRDSISINYRTEIDKPSQHHHHHH

SEQ ID NO: 255 - protein, 79 aa, Artificial Sequence
  Synthetic ATI_1421_E04 core
EVVAATPTSLLISWSYNGPIEYRITYGETGGNSPVQEFTVPDQQATISGLKPGVDYTITVYALEEAHYSRQSISINYRT

SEQ ID NO: 256 - protein, 7 aa, Artificial Sequence
  Synthetic ATI_1421_E04 BC loop
SYNGPIE

SEQ ID NO: 257 - protein, 4 aa, Artificial Sequence
  Synthetic ATI_1421_E04 DE loop
PDQQ

SEQ ID NO: 258 - protein, 10 aa, Artificial Sequence
  Synthetic ATI_1421_E04 FG loop
LEEAHYSRQS

SEQ ID NO: 259 - protein, 88 aa, Artificial Sequence
  Synthetic ATI_1421_E04 w/ N leader
GVSDVPRDLEVVAATPTSLLISWSYNGPIEYRITYGETGGNSPVQEFTVPDQQATISGLKPGVDYTITVYALEEAHYSRQSISINYRT

SEQ ID NO: 260 - protein, 94 aa, Artificial Sequence
  Synthetic ATI_1421_E04 w/ N leader + his tag
GVSDVPRDLEVVAATPTSLLISWSYNGPIEYRITYGETGGNSPVQEFTVPDQQATISGLKPGVDYTITVYALEEAHYSRQSISINYRTHHHHHH

SEQ ID NO: 261 - protein, 95 aa, Artificial Sequence
  Synthetic ATI_1421_E04 w/ N leader and C tail
GVSDVPRDLEVVAATPTSLLISWSYNGPIEYRITYGETGGNSPVQEFTVPDQQATISGLKPGVDYTITVYALEEAHYSRQSISINYRTEIDKPSQ

SEQ ID NO: 262 - protein, 101 aa, Artificial Sequence
  Synthetic ATI_1421_E04 w/ N leader and C tail + his tag
GVSDVPRDLEVVAATPTSLLISWSYNGPIEYRITYGETGGNSPVQEFTVPDQQATISGLKPGVDYTITVYALEEAHYSRQSISINYRTEIDKPSQHHHHHH

SEQ ID NO: 263 - protein, 90 aa, Artificial Sequence
  Synthetic ATI_1421_E04 w/ N leader and modified C-terminus including PC
GVSDVPRDLEVVAATPTSLLISWSYNGPIEYRITYGETGGNSPVQEFTVPDQQATISGLKPGVDYTITVYALEEAHYSRQSISINYRTPC

SEQ ID NO: 264 - protein, 96 aa, Artificial Sequence
  Synthetic ATI_1421_E04 w/ N leader and modified C-terminus including PC + his tag
GVSDVPRDLEVVAATPTSLLISWSYNGPIEYRITYGETGGNSPVQEFTVPDQQATISGLKPGVDYTITVYALEEAHYSRQSISINYRTPCHHHHHH

SEQ ID NO: 265 - protein, 102 aa, Artificial Sequence
  Synthetic ATI_1421_E04 full length
MGVSDVPRDLEVVAATPTSLLISWSYNGPIEYRITYGETGGNSPVQEFTVPDQQATISGLKPGVDYTITVYALEEAHYSRQSISINYRTEIDKPSQHHHHHH

SEQ ID NO: 266 - protein, 79 aa, Artificial Sequence
  Synthetic ATI_1421_F03 core
EVVAATPTSLLISWISVQTYDYRITYGETGGNSPVQEFTVPDQSATISGLKPGVDYTITVYALEKAHYYRQNISINYRT

SEQ ID NO: 267 - protein, 7 aa, Artificial Sequence
  Synthetic ATI_1421_F03 BC loop
ISVQTYD

SEQ ID NO: 268 - protein, 4 aa, Artificial Sequence
  Synthetic ATI_1421_F03 DE loop
PDQS

SEQ ID NO: 269 - protein, 10 aa, Artificial Sequence
  Synthetic ATI_1421_F03 FG loop
LEKAHYYRQN

SEQ ID NO: 270 - protein, 88 aa, Artificial Sequence
  Synthetic ATI_1421_F03 w/ N leader
GVSDVPRDLEVVAATPTSLLISWISVQTYDYRITYGETGGNSPVQEFTVPDQSATISGLKPGVDYTITVYALEKAHYYRQNISINYRT

SEQ ID NO: 271 - protein, 94 aa, Artificial Sequence
  Synthetic ATI_1421_F03 w/ N leader + his tag
GVSDVPRDLEVVAATPTSLLISWISVQTYDYRITYGETGGNSPVQEFTVPDQSATISGLKPGVDYTITVYALEKAHYYRQNISINYRTHHHHHH

SEQ ID NO: 272 - protein, 95 aa, Artificial Sequence
  Synthetic ATI_1421_F03 w/ N leader and C tail
GVSDVPRDLEVVAATPTSLLISWISVQTYDYRITYGETGGNSPVQEFTVPDQSATISGLKPGVDYTITVYALEKAHYYRQNISINYRTEIDKPSQ

SEQ ID NO: 273 - protein, 101 aa, Artificial Sequence
  Synthetic ATI_1421_F03 w/ N leader and C tail + his tag
GVSDVPRDLEVVAATPTSLLISWISVQTYDYRITYGETGGNSPVQEFTVPDQSATISGLKPGVDYTITVYALEKAHYYRQNISINYRTEIDKPSQHHHHHH

SEQ ID NO: 274 - protein, 90 aa, Artificial Sequence
  Synthetic ATI_1421_F03 w/ N leader and modified C-terminus including PC
GVSDVPRDLEVVAATPTSLLISWISVQTYDYRITYGETGGNSPVQEFTVPDQSATISGLKPGVDYTITVYALEKAHYYRQNISINYRTPC

SEQ ID NO: 275 - protein, 96 aa, Artificial Sequence
  Synthetic ATI_1421_F03 w/ N leader and modified C-terminus including PC + his tag
GVSDVPRDLEVVAATPTSLLISWISVQTYDYRITYGETGGNSPVQEFTVPDQSATISGLKPGVDYTITVYALEKAHYYRQNISINYRTPCHHHHHH

SEQ ID NO: 276 - protein, 102 aa, Artificial Sequence
  Synthetic ATI_1421_F03 full length
MGVSDVPRDLEVVAATPTSLLISWISVQTYDYRITYGETGGNSPVQEFTVPDQSATISGLKPGVDYTITVYALEKAHYYRQNISINYRTEIDKPSQHHHHHH

SEQ ID NO: 277 - protein, 79 aa, Artificial Sequence
  Synthetic ATI_1421_F05 core
EVVAATPTSLLISWLARHDARYRITYGETGGNSPVQEFTVPDRMATISGLKPGVDYTITVYALEQAHYYRLYISINYRT

SEQ ID NO: 278 - protein, 7 aa, Artificial Sequence
  Synthetic ATI_1421_F05 BC loop
LARHDAR

SEQ ID NO: 279 - protein, 4 aa, Artificial Sequence
  Synthetic ATI_1421_F05 DE loop
PDRM

SEQ ID NO: 280 - protein, 10 aa, Artificial Sequence
  Synthetic ATI_1421_F05 FG loop
LEQAHYYRLY

SEQ ID NO: 281 - protein, 88 aa, Artificial Sequence
  Synthetic ATI_1421_F05 w/ N leader
GVSDVPRDLEVVAATPTSLLISWLARHDARYRITYGETGGNSPVQEFTVPDRMATISGLKPGVDYTITVYALEQAHYYRLYISINYRT

SEQ ID NO: 282 - protein, 94 aa, Artificial Sequence
  Synthetic ATI_1421_F05 w/ N leader + his tag
GVSDVPRDLEVVAATPTSLLISWLARHDARYRITYGETGGNSPVQEFTVPDRMATISGLKPGVDYTITVYALEQAHYYRLYISINYRTHHHHHH

SEQ ID NO: 283 - protein, 95 aa, Artificial Sequence
  Synthetic ATI_1421_F05 w/ N leader and C tail
GVSDVPRDLEVVAATPTSLLISWLARHDARYRITYGETGGNSPVQEFTVPDRMATISGLKPGVDYTITVYALEQAHYYRLYISINYRTEIDKPSQ

SEQ ID NO: 284 - protein, 101 aa, Artificial Sequence
  Synthetic ATI_1421_F05 w/ N leader and C tail + his tag
GVSDVPRDLEVVAATPTSLLISWLARHDARYRITYGETGGNSPVQEFTVPDRMATISGLKPGVDYTITVYALEQAHYYRLYISINYRTEIDKPSQHHHHHH

SEQ ID NO: 285 - protein, 90 aa, Artificial Sequence
  Synthetic ATI_1421_F05 w/ N leader and modified C-terminus including PC
GVSDVPRDLEVVAATPTSLLISWLARHDARYRITYGETGGNSPVQEFTVPDRMATISGLKPGVDYTITVYALEQAHYYRLYISINYRTPC

SEQ ID NO: 286 - protein, 96 aa, Artificial Sequence
  Synthetic ATI_1421_F05 w/ N leader and modified C-terminus including PC + his tag
GVSDVPRDLEVVAATPTSLLISWLARHDARYRITYGETGGNSPVQEFTVPDRMATISGLKPGVDYTITVYALEQAHYYRLYISINYRTPCHHHHHH

SEQ ID NO: 287 - protein, 102 aa, Artificial Sequence
  Synthetic ATI_1421_F05 full length
MGVSDVPRDLEVVAATPTSLLISWLARHDARYRITYGETGGNSPVQEFTVPDRMATISGLKPGVDYTITVYALEQAHYYRLYISINYRTEIDKPSQHHHHHH

SEQ ID NO: 288 - protein, 82 aa, Artificial Sequence
  Synthetic ATI_1421_G07 core
EVVAATPTSLLISWHSPTSGITYRITYGETGGNSPVQEFTVPYDPSATISGLKPGVDYTITVYAPYGSQYYPGYHISINYRT

SEQ ID NO: 289 - protein, 8 aa, Artificial Sequence
  Synthetic ATI_1421_G07 BC loop
HSPTSGIT

SEQ ID NO: 290 - protein, 5 aa, Artificial Sequence
  Synthetic ATI_1421_G07 DE loop
PYDPS

SEQ ID NO: 291 - protein, 11 aa, Artificial Sequence
  Synthetic ATI_1421_G07 FG loop
PYGSQYYPGYH

SEQ ID NO: 292 - protein, 91 aa, Artificial Sequence
  Synthetic ATI_1421_G07 w/ N leader
GVSDVPRDLEVVAATPTSLLISWHSPTSGITYRITYGETGGNSPVQEFTVPYDPSATISGLKPGVDYTITVYAPYGSQYYPGYHISINYRT

SEQ ID NO: 293 - protein, 97 aa, Artificial Sequence
  Synthetic ATI_1421_G07 w/ N leader + his tag
GVSDVPRDLEVVAATPTSLLISWHSPTSGITYRITYGETGGNSPVQEFTVPYDPSATISGLKPGVDYTITVYAPYGSQYYPGYHISINYRTHHHHHH

SEQ ID NO: 294 - protein, 98 aa, Artificial Sequence
  Synthetic ATI_1421_G07 w/ N leader and C tail
GVSDVPRDLEVVAATPTSLLISWHSPTSGITYRITYGETGGNSPVQEFTVPYDPSATISGLKPGVDYTITVYAPYGSQYYPGYHISINYRTEIDKPSQ

SEQ ID NO: 295 - protein, 104 aa, Artificial Sequence
  Synthetic ATI_1421_G07 w/ N leader and C tail + his tag
GVSDVPRDLEVVAATPTSLLISWHSPTSGITYRITYGETGGNSPVQEFTVPYDPSATISGLKPGVDYTITVYAPYGSQYYPGYHISINYRTEIDKPSQHHHHHH

SEQ ID NO: 296 - protein, 93 aa, Artificial Sequence
  Synthetic ATI_1421_G07 w/ N leader and modified C-terminus including PC
GVSDVPRDLEVVAATPTSLLISWHSPTSGITYRITYGETGGNSPVQEFTVPYDPSATISGLKPGVDYTITVYAPYGSQYYPGYHISINYRTPC

SEQ ID NO: 297 - protein, 99 aa, Artificial Sequence
  Synthetic ATI_1421_G07 w/ N leader and modified C-terminus including PC + his tag
GVSDVPRDLEVVAATPTSLLISWHSPTSGITYRITYGETGGNSPVQEFTVPYDPSATISGLKPGVDYTITVYAPYGSQYYPGYHISINYRTPCHHHHHH

SEQ ID NO: 298 - protein, 105 aa, Artificial Sequence
  Synthetic ATI_1421_G07 full length
MGVSDVPRDLEVVAATPTSLLISWHSPTSGITYRITYGETGGNSPVQEFTVPYDPSATISGLKPGVDYTITVYAPYGSQYYPGYHISINYRTEIDKPSQHHHHHH

SEQ ID NO: 299 - protein, 79 aa, Artificial Sequence
  Synthetic ATI_1421_H03  core
EVVAATPTSLLISWVYHYDAQYRITYGETGGNSPVQEFTVPDSSATISGLKPGVDYTITVYALEQAHIDRTTISINYRT

SEQ ID NO: 300 - protein, 7 aa, Artificial Sequence
  Synthetic ATI_1421_H03  BC loop
VYHYDAQ

SEQ ID NO: 301 - protein, 4 aa, Artificial Sequence
  Synthetic ATI_1421_H03  DE loop
PDSS

SEQ ID NO: 302 - protein, 10 aa, Artificial Sequence
  Synthetic ATI_1421_H03  FG loop
LEQAHIDRTT

SEQ ID NO: 303 - protein, 88 aa, Artificial Sequence
  Synthetic ATI_1421_H03  w/ N leader
GVSDVPRDLEVVAATPTSLLISWVYHYDAQYRITYGETGGNSPVQEFTVPDSSATISGLKPGVDYTITVYALEQAHIDRTTISINYRT

SEQ ID NO: 304 - protein, 94 aa, Artificial Sequence
  Synthetic ATI_1421_H03  w/ N leader + his tag
GVSDVPRDLEVVAATPTSLLISWVYHYDAQYRITYGETGGNSPVQEFTVPDSSATISGLKPGVDYTITVYALEQAHIDRTTISINYRTHHHHHH

SEQ ID NO: 305 - protein, 95 aa, Artificial Sequence
  Synthetic ATI_1421_H03 w/ N leader and C tail
GVSDVPRDLEVVAATPTSLLISWVYHYDAQYRITYGETGGNSPVQEFTVPDSSATISGLKPGVDYTITVYALEQAHIDRTTISINYRTEIDKPSQ

SEQ ID NO: 306 - protein, 101 aa, Artificial Sequence
  Synthetic ATI_1421_H03 w/ N leader and C tail + his tag
GVSDVPRDLEVVAATPTSLLISWVYHYDAQYRITYGETGGNSPVQEFTVPDSSATISGLKPGVDYTITVYALEQAHIDRTTISINYRTEIDKPSQHHHHHH

SEQ ID NO: 307 - protein, 90 aa, Artificial Sequence
  Synthetic ATI_1421_H03 w/ N leader and modified C-terminus including PC
GVSDVPRDLEVVAATPTSLLISWVYHYDAQYRITYGETGGNSPVQEFTVPDSSATISGLKPGVDYTITVYALEQAHIDRTTISINYRTPC

SEQ ID NO: 308 - protein, 96 aa, Artificial Sequence
  Synthetic ATI_1421_H03 w/ N leader and modified C-terminus including PC + his tag
GVSDVPRDLEVVAATPTSLLISWVYHYDAQYRITYGETGGNSPVQEFTVPDSSATISGLKPGVDYTITVYALEQAHIDRTTISINYRTPCHHHHHH

SEQ ID NO: 309 - protein, 102 aa, Artificial Sequence
  Synthetic ATI_1421_H03  full length
MGVSDVPRDLEVVAATPTSLLISWVYHYDAQYRITYGETGGNSPVQEFTVPDSSATISGLKPGVDYTITVYALEQAHIDRTTISINYRTEIDKPSQHHHHHH

SEQ ID NO: 310 - protein, 79 aa, Artificial Sequence
  Synthetic ATI_1421_H05 core
EVVAATPTSLLISWTSVLLKDYRITYGETGGNSPVQEFTVPDQHATISGLKPGVDYTITVYALQNAHHERLYISINYRT

SEQ ID NO: 311 - protein, 7 aa, Artificial Sequence
  Synthetic ATI_1421_H05 BC loop
TSVLLKD

SEQ ID NO: 312 - protein, 4 aa, Artificial Sequence
  Synthetic ATI_1421_H05 DE loop
PDQH

SEQ ID NO: 313 - protein, 10 aa, Artificial Sequence
  Synthetic ATI_1421_H05 FG loop
LQNAHHERLY

SEQ ID NO: 314 - protein, 88 aa, Artificial Sequence
  Synthetic ATI_1421_H05 w/ N leader
GVSDVPRDLEVVAATPTSLLISWTSVLLKDYRITYGETGGNSPVQEFTVPDQHATISGLKPGVDYTITVYALQNAHHERLYISINYRT

SEQ ID NO: 315 - protein, 94 aa, Artificial Sequence
  Synthetic ATI_1421_H05 w/ N leader + his tag
GVSDVPRDLEVVAATPTSLLISWTSVLLKDYRITYGETGGNSPVQEFTVPDQHATISGLKPGVDYTITVYALQNAHHERLYISINYRTHHHHHH

SEQ ID NO: 316 - protein, 95 aa, Artificial Sequence
  Synthetic ATI_1421_H05 w/ N leader and C tail
GVSDVPRDLEVVAATPTSLLISWTSVLLKDYRITYGETGGNSPVQEFTVPDQHATISGLKPGVDYTITVYALQNAHHERLYISINYRTEIDKPSQ

SEQ ID NO: 317 - protein, 101 aa, Artificial Sequence
  Synthetic ATI_1421_H05 w/ N leader and C tail + his tag
GVSDVPRDLEVVAATPTSLLISWTSVLLKDYRITYGETGGNSPVQEFTVPDQHATISGLKPGVDYTITVYALQNAHHERLYISINYRTEIDKPSQHHHHHH

SEQ ID NO: 318 - protein, 90 aa, Artificial Sequence
  Synthetic ATI_1421_H05 w/ N leader and modified C-terminus including PC
GVSDVPRDLEVVAATPTSLLISWTSVLLKDYRITYGETGGNSPVQEFTVPDQHATISGLKPGVDYTITVYALQNAHHERLYISINYRTPC

SEQ ID NO: 319 - protein, 96 aa, Artificial Sequence
  Synthetic ATI_1421_H05 w/ N leader and modified C-terminus including PC + his tag
GVSDVPRDLEVVAATPTSLLISWTSVLLKDYRITYGETGGNSPVQEFTVPDQHATISGLKPGVDYTITVYALQNAHHERLYISINYRTPCHHHHHH

SEQ ID NO: 320 - protein, 102 aa, Artificial Sequence
  Synthetic ATI_1421_H05 full length
MGVSDVPRDLEVVAATPTSLLISWTSVLLKDYRITYGETGGNSPVQEFTVPDQHATISGLKPGVDYTITVYALQNAHHERLYISINYRTEIDKPSQHHHHHH

SEQ ID NO: 321 - protein, 79 aa, Artificial Sequence
  Synthetic ATI_1422_E06 core
EVVAATPTSLLISWLPSYYITYRITYGETGGNSPVQEFTVSKDLATISGLKPGVDYTITVYAFNGSSYYTFGISINYRT

SEQ ID NO: 322 - protein, 7 aa, Artificial Sequence
  Synthetic ATI_1422_E06 BC loop
LPSYYIT

SEQ ID NO: 323 - protein, 4 aa, Artificial Sequence
  Synthetic ATI_1422_E06 DE loop
SKDL

SEQ ID NO: 324 - protein, 10 aa, Artificial Sequence
  Synthetic ATI_1422_E06 FG loop
FNGSSYYTFG

SEQ ID NO: 325 - protein, 88 aa, Artificial Sequence
  Synthetic ATI_1422_E06 w/ N leader
GVSDVPRDLEVVAATPTSLLISWLPSYYITYRITYGETGGNSPVQEFTVSKDLATISGLKPGVDYTITVYAFNGSSYYTFGISINYRT

SEQ ID NO: 326 - protein, 101 aa, Artificial Sequence
  Synthetic ATI_1422_E06 w/ N leader + his tag
GVSDVPRDLEVVAATPTSLLISWLPSYYITYRITYGETGGNSPVQEFTVSKDLATISGLKPGVDYTITVYAFNGSSYYTFGISINYRTEIDKPSQHHHHHH

SEQ ID NO: 327 - protein, 101 aa, Artificial Sequence; identical to SEQ ID NO: 326
  Synthetic ATI_1422_E06 w/ N leader and C tail

SEQ ID NO: 328 - protein, 90 aa, Artificial Sequence
  Synthetic ATI_1422_E06 w/ N leader and C tail + his tag
GVSDVPRDLEVVAATPTSLLISWLPSYYITYRITYGETGGNSPVQEFTVSKDLATISGLKPGVDYTITVYAFNGSSYYTFGISINYRTPC

SEQ ID NO: 329 - protein, 96 aa, Artificial Sequence
  Synthetic ATI_1422_E06 w/ N leader and modified C-terminus including PC
GVSDVPRDLEVVAATPTSLLISWLPSYYITYRITYGETGGNSPVQEFTVSKDLATISGLKPGVDYTITVYAFNGSSYYTFGISINYRTPCHHHHHH

SEQ ID NO: 330 - protein, 101 aa, Artificial Sequence; identical to SEQ ID NO: 326
  Synthetic ATI_1422_E06 w/ N leader and modified C-terminus including PC + his tag

SEQ ID NO: 331 - protein, 89 aa, Artificial Sequence
  Synthetic ATI_1422_E06 full length
MGVSDVPRDLEVVAATPTSLLISWLPSYYITYRITYGETGGNSPVQEFTVSKDLATISGLKPGVDYTITVYAFNGSSYYTFGISINYRT

SEQ ID NO: 332 - protein, 78 aa, Artificial Sequence
  Synthetic ATI_1422_F04 core
EVVAATPTSLLISWSIPSYFISYRITYGETGGNSPVQEFTVYKNYATISGLKPGVDYTITVYASEGIMFYNISINYRT

SEQ ID NO: 333 - protein, 8 aa, Artificial Sequence
  Synthetic ATI_1422_F04 BC loop
SIPSYFIS

SEQ ID NO: 334 - protein, 4 aa, Artificial Sequence
  Synthetic ATI_1422_F04 DE loop
YKNY

SEQ ID NO: 335 - protein, 8 aa, Artificial Sequence
  Synthetic ATI_1422_F04 FG loop
SEGIMFYN

SEQ ID NO: 336 - protein, 87 aa, Artificial Sequence
  Synthetic ATI_1422_F04w/ N leader
GVSDVPRDLEVVAATPTSLLISWSIPSYFISYRITYGETGGNSPVQEFTVYKNYATISGLKPGVDYTITVYASEGIMFYNISINYRT

SEQ ID NO: 337 - protein, 93 aa, Artificial Sequence
  Synthetic ATI_1422_F04w/ N leader + his tag
GVSDVPRDLEVVAATPTSLLISWSIPSYFISYRITYGETGGNSPVQEFTVYKNYATISGLKPGVDYTITVYASEGIMFYNISINYRTHHHHHH

SEQ ID NO: 338 - protein, 94 aa, Artificial Sequence
  Synthetic ATI_1422_F04 w/ N leader and C tail
GVSDVPRDLEVVAATPTSLLISWSIPSYFISYRITYGETGGNSPVQEFTVYKNYATISGLKPGVDYTITVYASEGIMFYNISINYRTEIDKPSQ

SEQ ID NO: 339 - protein, 100 aa, Artificial Sequence
  Synthetic ATI_1422_F04 w/ N leader and C tail + his tag
GVSDVPRDLEVVAATPTSLLISWSIPSYFISYRITYGETGGNSPVQEFTVYKNYATISGLKPGVDYTITVYASEGIMFYNISINYRTEIDKPSQHHHHHH

SEQ ID NO: 340 - protein, 89 aa, Artificial Sequence
  Synthetic ATI_1422_F04 w/ N leader and modified C-terminus including PC
GVSDVPRDLEVVAATPTSLLISWSIPSYFISYRITYGETGGNSPVQEFTVYKNYATISGLKPGVDYTITVYASEGIMFYNISINYRTPC

SEQ ID NO: 341 - protein, 95 aa, Artificial Sequence
  Synthetic ATI_1422_F04 w/ N leader and modified C-terminus including PC + his tag
GVSDVPRDLEVVAATPTSLLISWSIPSYFISYRITYGETGGNSPVQEFTVYKNYATISGLKPGVDYTITVYASEGIMFYNISINYRTPCHHHHHH

SEQ ID NO: 342 - protein, 101 aa, Artificial Sequence
  Synthetic ATI_1422_F04 full length
MGVSDVPRDLEVVAATPTSLLISWSIPSYFISYRITYGETGGNSPVQEFTVYKNYATISGLKPGVDYTITVYASEGIMFYNISINYRTEIDKPSQHHHHHH

SEQ ID NO: 343 - protein, 77 aa, Artificial Sequence
  Synthetic ATI_1422_F05 core
EVVAATPTSLLISWPYPRGPYVFYRITYGETGGNSPVQEFTVYPGQATISGLKPGVDYTITVYAYTSGYVISINYRT

SEQ ID NO: 344 - protein, 9 aa, Artificial Sequence
  Synthetic ATI_1422_F05 BC loop
PYPRGPYVF

SEQ ID NO: 345 - protein, 4 aa, Artificial Sequence
  Synthetic ATI_1422_F05 DE loop
YPGQ

SEQ ID NO: 346 - protein, 6 aa, Artificial Sequence
  Synthetic ATI_1422_F05 FG loop
YTSGYV

SEQ ID NO: 347 - protein, 86 aa, Artificial Sequence
  Synthetic ATI_1422_F05 w/ N leader
GVSDVPRDLEVVAATPTSLLISWPYPRGPYVFYRITYGETGGNSPVQEFTVYPGQATISGLKPGVDYTITVYAYTSGYVISINYRT

SEQ ID NO: 348 - protein, 99 aa, Artificial Sequence
  Synthetic ATI_1422_F05 w/ N leader + his tag
GVSDVPRDLEVVAATPTSLLISWPYPRGPYVFYRITYGETGGNSPVQEFTVYPGQATISGLKPGVDYTITVYAYTSGYVISINYRTEIDKPSQHHHHHH

SEQ ID NO: 349 - protein, 93 aa, Artificial Sequence
  Synthetic ATI_1422_F05 w/ N leader and C tail
GVSDVPRDLEVVAATPTSLLISWPYPRGPYVFYRITYGETGGNSPVQEFTVYPGQATISGLKPGVDYTITVYAYTSGYVISINYRTEIDKPSQ

SEQ ID NO: 350 - protein, 99 aa, Artificial Sequence; identical to SEQ ID NO: 348
  Synthetic ATI_1422_F05 w/ N leader and C tail + his tag

SEQ ID NO: 351 - protein, 88 aa, Artificial Sequence
  Synthetic ATI_1422_F05 w/ N leader and modified C-terminus including PC
GVSDVPRDLEVVAATPTSLLISWPYPRGPYVFYRITYGETGGNSPVQEFTVYPGQATISGLKPGVDYTITVYAYTSGYVISINYRTPC

SEQ ID NO: 352 - protein, 94 aa, Artificial Sequence
  Synthetic ATI_1422_F05 w/ N leader and modified C-terminus including PC + his tag
GVSDVPRDLEVVAATPTSLLISWPYPRGPYVFYRITYGETGGNSPVQEFTVYPGQATISGLKPGVDYTITVYAYTSGYVISINYRTPCHHHHHH

SEQ ID NO: 353 - protein, 100 aa, Artificial Sequence
  Synthetic ATI_1422_F05 full length
MGVSDVPRDLEVVAATPTSLLISWPYPRGPYVFYRITYGETGGNSPVQEFTVYPGQATISGLKPGVDYTITVYAYTSGYVISINYRTEIDKPSQHHHHHH

SEQ ID NO: 354 - protein, 80 aa, Artificial Sequence
  Synthetic ATI_1422_H04 core
EVVAATPTSLLISWYLPSYYVQYRITYGETGGNSPVQEFTVKSYNATISGLKPGVDYTITVYARMGVYYLSYSISINYRT

SEQ ID NO: 355 - protein, 8 aa, Artificial Sequence
  Synthetic ATI_1422_H04 BC loop
YLPSYYVQ

SEQ ID NO: 356 - protein, 4 aa, Artificial Sequence
  Synthetic ATI_1422_H04 DE loop
KSYN

SEQ ID NO: 357 - protein, 10 aa, Artificial Sequence
  Synthetic ATI_1422_H04 FG loop
RMGVYYLSYS

SEQ ID NO: 358 - protein, 89 aa, Artificial Sequence
  Synthetic ATI_1422_H04 w/ N leader
GVSDVPRDLEVVAATPTSLLISWYLPSYYVQYRITYGETGGNSPVQEFTVKSYNATISGLKPGVDYTITVYARMGVYYLSYSISINYRT

SEQ ID NO: 359 - protein, 95 aa, Artificial Sequence
  Synthetic ATI_1422_H04 w/ N leader + his tag
GVSDVPRDLEVVAATPTSLLISWYLPSYYVQYRITYGETGGNSPVQEFTVKSYNATISGLKPGVDYTITVYARMGVYYLSYSISINYRTHHHHHH

SEQ ID NO: 360 - protein, 96 aa, Artificial Sequence
  Synthetic ATI_1422_H04 w/ N leader and C tail
GVSDVPRDLEVVAATPTSLLISWYLPSYYVQYRITYGETGGNSPVQEFTVKSYNATISGLKPGVDYTITVYARMGVYYLSYSISINYRTEIDKPSQ

SEQ ID NO: 361 - protein, 102 aa, Artificial Sequence
  Synthetic ATI_1422_H04 w/ N leader and C tail + his tag
GVSDVPRDLEVVAATPTSLLISWYLPSYYVQYRITYGETGGNSPVQEFTVKSYNATISGLKPGVDYTITVYARMGVYYLSYSISINYRTEIDKPSQHHHHHH

SEQ ID NO: 362 - protein, 91 aa, Artificial Sequence
  Synthetic ATI_1422_H04 w/ N leader and modified C-terminus including PC
GVSDVPRDLEVVAATPTSLLISWYLPSYYVQYRITYGETGGNSPVQEFTVKSYNATISGLKPGVDYTITVYARMGVYYLSYSISINYRTPC

SEQ ID NO: 363 - protein, 97 aa, Artificial Sequence
  Synthetic ATI_1422_H04 w/ N leader and modified C-terminus including PC + his tag
GVSDVPRDLEVVAATPTSLLISWYLPSYYVQYRITYGETGGNSPVQEFTVKSYNATISGLKPGVDYTITVYARMGVYYLSYSISINYRTPCHHHHHH

SEQ ID NO: 364 - protein, 103 aa, Artificial Sequence
  Synthetic ATI_1422_H04  full length
MGVSDVPRDLEVVAATPTSLLISWYLPSYYVQYRITYGETGGNSPVQEFTVKSYNATISGLKPGVDYTITVYARMGVYYLSYSISINYRTEIDKPSQHHHHHH

SEQ ID NO: 365 - protein, 78 aa, Artificial Sequence
  Synthetic ATI_1422_H05 core
EVVAATPTSLLISWQGQLSPSFYRITYGETGGNSPVQEFTVVAGMATISGLKPGVDYTITVYATSDVYFYSISINYRT

SEQ ID NO: 366 - protein, 8 aa, Artificial Sequence
  Synthetic ATI_1422_H05 BC loop
QGQLSPSF

SEQ ID NO: 367 - protein, 4 aa, Artificial Sequence
  Synthetic ATI_1422_H05 DE loop
VAGM

SEQ ID NO: 368 - protein, 8 aa, Artificial Sequence
  Synthetic ATI_1422_H05 FG loop
TSDVYFYS

SEQ ID NO: 369 - protein, 87 aa, Artificial Sequence
  Synthetic ATI_1422_H05 w/ N leader
GVSDVPRDLEVVAATPTSLLISWQGQLSPSFYRITYGETGGNSPVQEFTVVAGMATISGLKPGVDYTITVYATSDVYFYSISINYRT

SEQ ID NO: 370 - protein, 93 aa, Artificial Sequence
  Synthetic ATI_1422_H05 w/ N leader + his tag
GVSDVPRDLEVVAATPTSLLISWQGQLSPSFYRITYGETGGNSPVQEFTVVAGMATISGLKPGVDYTITVYATSDVYFYSISINYRTHHHHHH

SEQ ID NO: 371 - protein, 94 aa, Artificial Sequence
  Synthetic ATI_1422_H05 w/ N leader and C tail
GVSDVPRDLEVVAATPTSLLISWQGQLSPSFYRITYGETGGNSPVQEFTVVAGMATISGLKPGVDYTITVYATSDVYFYSISINYRTEIDKPSQ

SEQ ID NO: 372 - protein, 100 aa, Artificial Sequence
  Synthetic ATI_1422_H05 w/ N leader and C tail + his tag
GVSDVPRDLEVVAATPTSLLISWQGQLSPSFYRITYGETGGNSPVQEFTVVAGMATISGLKPGVDYTITVYATSDVYFYSISINYRTEIDKPSQHHHHHH

SEQ ID NO: 373 - protein, 89 aa, Artificial Sequence
  Synthetic ATI_1422_H05 w/ N leader and modified C-terminus including PC
GVSDVPRDLEVVAATPTSLLISWQGQLSPSFYRITYGETGGNSPVQEFTVVAGMATISGLKPGVDYTITVYATSDVYFYSISINYRTPC

SEQ ID NO: 374 - protein, 95 aa, Artificial Sequence
  Synthetic ATI_1422_H05 w/ N leader and modified C-terminus including PC + his tag
GVSDVPRDLEVVAATPTSLLISWQGQLSPSFYRITYGETGGNSPVQEFTVVAGMATISGLKPGVDYTITVYATSDVYFYSISINYRTPCHHHHHH

SEQ ID NO: 375 - protein, 101 aa, Artificial Sequence
  Synthetic  full length
MGVSDVPRDLEVVAATPTSLLISWQGQLSPSFYRITYGETGGNSPVQEFTVVAGMATISGLKPGVDYTITVYATSDVYFYSISINYRTEIDKPSQHHHHHH

SEQ ID NO: 376 - protein, 82 aa, Artificial Sequence
  Synthetic ATI_1422_G05 core
EVVAATPTSLLISWIAPYYSVIYRITYGETGGNSPVQEFTVTGSGYATISGLKPGVDYTITVYATYCASVASYAFISINYRT

SEQ ID NO: 377 - protein, 8 aa, Artificial Sequence
  Synthetic ATI_1422_G05 BC loop
IAPYYSVI

SEQ ID NO: 378 - protein, 5 aa, Artificial Sequence
  Synthetic ATI_1422_G05 DE loop
TGSGY

SEQ ID NO: 379 - protein, 11 aa, Artificial Sequence
  Synthetic ATI_1422_G05 FG loop
TYCASVASYAF

SEQ ID NO: 380 - protein, 91 aa, Artificial Sequence
  Synthetic ATI_1422_G05 w/ N leader
GVSDVPRDLEVVAATPTSLLISWIAPYYSVIYRITYGETGGNSPVQEFTVTGSGYATISGLKPGVDYTITVYATYCASVASYAFISINYRT

SEQ ID NO: 381 - protein, 97 aa, Artificial Sequence
  Synthetic ATI_1422_G05 w/ N leader + his tag
GVSDVPRDLEVVAATPTSLLISWIAPYYSVIYRITYGETGGNSPVQEFTVTGSGYATISGLKPGVDYTITVYATYCASVASYAFISINYRTHHHHHH

SEQ ID NO: 382 - protein, 98 aa, Artificial Sequence
  Synthetic ATI_1422_G05 w/ N leader and C tail
GVSDVPRDLEVVAATPTSLLISWIAPYYSVIYRITYGETGGNSPVQEFTVTGSGYATISGLKPGVDYTITVYATYCASVASYAFISINYRTEIDKPSQ

SEQ ID NO: 383 - protein, 104 aa, Artificial Sequence
  Synthetic ATI_1422_G05 w/ N leader and C tail + his tag
GVSDVPRDLEVVAATPTSLLISWIAPYYSVIYRITYGETGGNSPVQEFTVTGSGYATISGLKPGVDYTITVYATYCASVASYAFISINYRTEIDKPSQHHHHHH

SEQ ID NO: 384 - protein, 93 aa, Artificial Sequence
  Synthetic ATI_1422_G05 w/ N leader and modified C-terminus including PC
GVSDVPRDLEVVAATPTSLLISWIAPYYSVIYRITYGETGGNSPVQEFTVTGSGYATISGLKPGVDYTITVYATYCASVASYAFISINYRTPC

SEQ ID NO: 385 - protein, 99 aa, Artificial Sequence
  Synthetic ATI_1422_G05 w/ N leader and modified C-terminus including PC + his tag
GVSDVPRDLEVVAATPTSLLISWIAPYYSVIYRITYGETGGNSPVQEFTVTGSGYATISGLKPGVDYTITVYATYCASVASYAFISINYRTPCHHHHHH

SEQ ID NO: 386 - protein, 105 aa, Artificial Sequence
  Synthetic ATI_1422_G05 full length
MGVSDVPRDLEVVAATPTSLLISWIAPYYSVIYRITYGETGGNSPVQEFTVTGSGYATISGLKPGVDYTITVYATYCASVASYAFISINYRTEIDKPSQHHHHHH

SEQ ID NO: 387 - protein, 82 aa, Artificial Sequence
  Synthetic ATI_1760_C02 core
EVVAATPTSLLISWIAPYYSVIYRITYGETGGNSPVQEFTVPGSAYATISGLKPGVDYTITVYASSGASIAAYAFISINYRT

SEQ ID NO: 388 - protein, 8 aa, Artificial Sequence
  Synthetic ATI_1760_C02 BC loop
IAPYYSVI

SEQ ID NO: 389 - protein, 5 aa, Artificial Sequence
  Synthetic ATI_1760_C02 DE loop
PGSAY

SEQ ID NO: 390 - protein, 11 aa, Artificial Sequence
  Synthetic ATI_1760_C02 FG loop
SSGASIAAYAF

SEQ ID NO: 391 - protein, 91 aa, Artificial Sequence
  Synthetic ATI_1760_C02 w/ N leader
GVSDVPRDLEVVAATPTSLLISWIAPYYSVIYRITYGETGGNSPVQEFTVPGSAYATISGLKPGVDYTITVYASSGASIAAYAFISINYRT

SEQ ID NO: 392 - protein, 97 aa, Artificial Sequence
  Synthetic ATI_1760_C02 w/ N leader + his tag
GVSDVPRDLEVVAATPTSLLISWIAPYYSVIYRITYGETGGNSPVQEFTVPGSAYATISGLKPGVDYTITVYASSGASIAAYAFISINYRTHHHHHH

SEQ ID NO: 393 - protein, 98 aa, Artificial Sequence
  Synthetic ATI_1760_C02 w/ N leader and C tail
GVSDVPRDLEVVAATPTSLLISWIAPYYSVIYRITYGETGGNSPVQEFTVPGSAYATISGLKPGVDYTITVYASSGASIAAYAFISINYRTEIDKPSQ

SEQ ID NO: 394 - protein, 104 aa, Artificial Sequence
  Synthetic ATI_1760_C02 w/ N leader and C tail + his tag
GVSDVPRDLEVVAATPTSLLISWIAPYYSVIYRITYGETGGNSPVQEFTVPGSAYATISGLKPGVDYTITVYASSGASIAAYAFISINYRTEIDKPSQHHHHHH

SEQ ID NO: 395 - protein, 93 aa, Artificial Sequence
  Synthetic ATI_1760_C02 w/ N leader and modified C-terminus including PC
GVSDVPRDLEVVAATPTSLLISWIAPYYSVIYRITYGETGGNSPVQEFTVPGSAYATISGLKPGVDYTITVYASSGASIAAYAFISINYRTPC

SEQ ID NO: 396 - protein, 99 aa, Artificial Sequence
  Synthetic ATI_1760_C02 w/ N leader and modified C-terminus including PC + his tag
GVSDVPRDLEVVAATPTSLLISWIAPYYSVIYRITYGETGGNSPVQEFTVPGSAYATISGLKPGVDYTITVYASSGASIAAYAFISINYRTPCHHHHHH

SEQ ID NO: 397 - protein, 105 aa, Artificial Sequence
  Synthetic ATI_1760_C02 full length
MGVSDVPRDLEVVAATPTSLLISWIAPYYSVIYRITYGETGGNSPVQEFTVPGSAYATISGLKPGVDYTITVYASSGASIAAYAFISINYRTEIDKPSQHHHHHH

SEQ ID NO: 398 - protein, 82 aa, Artificial Sequence
  Synthetic ATI_1760_E01 core
EVVAATPTSLLISWIAPYYSVKYRITYGETGGNSPVQEFTVAGADYATISGLKPGVDYTITVYATYGASIASYAFISINYRT

SEQ ID NO: 399 - protein, 8 aa, Artificial Sequence
  Synthetic ATI_1760_E01 BC loop
IAPYYSVK

SEQ ID NO: 400 - protein, 5 aa, Artificial Sequence
  Synthetic ATI_1760_E01 DE loop
AGADY

SEQ ID NO: 401 - protein, 11 aa, Artificial Sequence
  Synthetic ATI_1760_E01 FG loop
TYGASIASYAF

SEQ ID NO: 402 - protein, 91 aa, Artificial Sequence
  Synthetic ATI_1760_E01 w/ N leader
GVSDVPRDLEVVAATPTSLLISWIAPYYSVKYRITYGETGGNSPVQEFTVAGADYATISGLKPGVDYTITVYATYGASIASYAFISINYRT

SEQ ID NO: 403 - protein, 97 aa, Artificial Sequence
  Synthetic ATI_1760_E01 w/ N leader + his tag
GVSDVPRDLEVVAATPTSLLISWIAPYYSVKYRITYGETGGNSPVQEFTVAGADYATISGLKPGVDYTITVYATYGASIASYAFISINYRTHHHHHH

SEQ ID NO: 404 - protein, 98 aa, Artificial Sequence
  Synthetic ATI_1760_E01 w/ N leader and C tail
GVSDVPRDLEVVAATPTSLLISWIAPYYSVKYRITYGETGGNSPVQEFTVAGADYATISGLKPGVDYTITVYATYGASIASYAFISINYRTEIDKPSQ

SEQ ID NO: 405 - protein, 104 aa, Artificial Sequence
  Synthetic ATI_1760_E01 w/ N leader and C tail + his tag
GVSDVPRDLEVVAATPTSLLISWIAPYYSVKYRITYGETGGNSPVQEFTVAGADYATISGLKPGVDYTITVYATYGASIASYAFISINYRTEIDKPSQHHHHHH

SEQ ID NO: 406 - protein, 93 aa, Artificial Sequence
  Synthetic ATI_1760_E01 w/ N leader and modified C-terminus including PC
GVSDVPRDLEVVAATPTSLLISWIAPYYSVKYRITYGETGGNSPVQEFTVAGADYATISGLKPGVDYTITVYATYGASIASYAFISINYRTPC

SEQ ID NO: 407 - protein, 99 aa, Artificial Sequence
  Synthetic ATI_1760_E01 w/ N leader and modified C-terminus including PC + his tag
GVSDVPRDLEVVAATPTSLLISWIAPYYSVKYRITYGETGGNSPVQEFTVAGADYATISGLKPGVDYTITVYATYGASIASYAFISINYRTPCHHHHHH

SEQ ID NO: 408 - protein, 105 aa, Artificial Sequence
  Synthetic ATI_1760_E01 full length
MGVSDVPRDLEVVAATPTSLLISWIAPYYSVKYRITYGETGGNSPVQEFTVAGADYATISGLKPGVDYTITVYATYGASIASYAFISINYRTEIDKPSQHHHHHH

SEQ ID NO: 409 - protein, 82 aa, Artificial Sequence
  Synthetic ATI_1760_F01 core
EVVAATPTSLLISWIAPYYAVMYRITYGETGGNSPVQEFTVPGGGYATISGLKPGVDYTITVYATGGASIAAYAFISINYRT

SEQ ID NO: 410 - protein, 8 aa, Artificial Sequence
  Synthetic ATI_1760_F01 BC loop
IAPYYAVM

SEQ ID NO: 411 - protein, 5 aa, Artificial Sequence
  Synthetic ATI_1760_F01 DE loop
PGGGY

SEQ ID NO: 412 - protein, 11 aa, Artificial Sequence
  Synthetic ATI_1760_F01 FG loop
TGGASIAAYAF

SEQ ID NO: 413 - protein, 91 aa, Artificial Sequence
  Synthetic ATI_1760_F01 w/ N leader
GVSDVPRDLEVVAATPTSLLISWIAPYYAVMYRITYGETGGNSPVQEFTVPGGGYATISGLKPGVDYTITVYATGGASIAAYAFISINYRT

SEQ ID NO: 414 - protein, 97 aa, Artificial Sequence
  Synthetic ATI_1760_F01 w/ N leader + his tag
GVSDVPRDLEVVAATPTSLLISWIAPYYAVMYRITYGETGGNSPVQEFTVPGGGYATISGLKPGVDYTITVYATGGASIAAYAFISINYRTHHHHHH

SEQ ID NO: 415 - protein, 98 aa, Artificial Sequence
  Synthetic ATI_1760_F01 w/ N leader and C tail
GVSDVPRDLEVVAATPTSLLISWIAPYYAVMYRITYGETGGNSPVQEFTVPGGGYATISGLKPGVDYTITVYATGGASIAAYAFISINYRTEIDKPSQ

SEQ ID NO: 416 - protein, 104 aa, Artificial Sequence
  Synthetic ATI_1760_F01 w/ N leader and C tail + his tag
GVSDVPRDLEVVAATPTSLLISWIAPYYAVMYRITYGETGGNSPVQEFTVPGGGYATISGLKPGVDYTITVYATGGASIAAYAFISINYRTEIDKPSQHHHHHH

SEQ ID NO: 417 - protein, 93 aa, Artificial Sequence
  Synthetic ATI_1760_F01 w/ N leader and modified C-terminus including PC
GVSDVPRDLEVVAATPTSLLISWIAPYYAVMYRITYGETGGNSPVQEFTVPGGGYATISGLKPGVDYTITVYATGGASIAAYAFISINYRTPC

SEQ ID NO: 418 - protein, 99 aa, Artificial Sequence
  Synthetic ATI_1760_F01 w/ N leader and modified C-terminus including PC + his tag
GVSDVPRDLEVVAATPTSLLISWIAPYYAVMYRITYGETGGNSPVQEFTVPGGGYATISGLKPGVDYTITVYATGGASIAAYAFISINYRTPCHHHHHH

SEQ ID NO: 419 - protein, 105 aa, Artificial Sequence
  Synthetic ATI_1760_F01 full length
MGVSDVPRDLEVVAATPTSLLISWIAPYYAVMYRITYGETGGNSPVQEFTVPGGGYATISGLKPGVDYTITVYATGGASIAAYAFISINYRTEIDKPSQHHHHHH

SEQ ID NO: 420 - protein, 79 aa, Artificial Sequence
  Synthetic ATI_1494_D03 core
EVVAATPTSLLISWSYPSYHLYRITYGETGGNSPVQEFTVHIDYATISGLKPGVDYTITVYAQSPPYDIYYEISINYRT

SEQ ID NO: 421 - protein, 7 aa, Artificial Sequence
  Synthetic ATI_1494_D03 BC loop
SYPSYHL

SEQ ID NO: 422 - protein, 4 aa, Artificial Sequence
  Synthetic ATI_1494_D03 DE loop
HIDY

SEQ ID NO: 423 - protein, 10 aa, Artificial Sequence
  Synthetic ATI_1494_D03 FG loop
QSPPYDIYYE

SEQ ID NO: 424 - protein, 88 aa, Artificial Sequence
  Synthetic ATI_1494_D03 w/ N leader
GVSDVPRDLEVVAATPTSLLISWSYPSYHLYRITYGETGGNSPVQEFTVHIDYATISGLKPGVDYTITVYAQSPPYDIYYEISINYRT

SEQ ID NO: 425 - protein, 94 aa, Artificial Sequence
  Synthetic ATI_1494_D03 w/ N leader + his tag
GVSDVPRDLEVVAATPTSLLISWSYPSYHLYRITYGETGGNSPVQEFTVHIDYATISGLKPGVDYTITVYAQSPPYDIYYEISINYRTHHHHHH

SEQ ID NO: 426 - protein, 95 aa, Artificial Sequence
  Synthetic ATI_1494_D03 w/ N leader and C tail
GVSDVPRDLEVVAATPTSLLISWSYPSYHLYRITYGETGGNSPVQEFTVHIDYATISGLKPGVDYTITVYAQSPPYDIYYEISINYRTEIDKPSQ

SEQ ID NO: 427 - protein, 101 aa, Artificial Sequence
  Synthetic ATI_1494_D03 w/ N leader and C tail + his tag
GVSDVPRDLEVVAATPTSLLISWSYPSYHLYRITYGETGGNSPVQEFTVHIDYATISGLKPGVDYTITVYAQSPPYDIYYEISINYRTEIDKPSQHHHHHH

SEQ ID NO: 428 - protein, 90 aa, Artificial Sequence
  Synthetic ATI_1494_D03 w/ N leader and modified C-terminus including PC
GVSDVPRDLEVVAATPTSLLISWSYPSYHLYRITYGETGGNSPVQEFTVHIDYATISGLKPGVDYTITVYAQSPPYDIYYEISINYRTPC

SEQ ID NO: 429 - protein, 96 aa, Artificial Sequence
  Synthetic ATI_1494_D03 w/ N leader and modified C-terminus including PC + his tag
GVSDVPRDLEVVAATPTSLLISWSYPSYHLYRITYGETGGNSPVQEFTVHIDYATISGLKPGVDYTITVYAQSPPYDIYYEISINYRTPCHHHHHH

SEQ ID NO: 430 - protein, 102 aa, Artificial Sequence
  Synthetic ATI_1494_D03 full length
MGVSDVPRDLEVVAATPTSLLISWSYPSYHLYRITYGETGGNSPVQEFTVHIDYATISGLKPGVDYTITVYAQSPPYDIYYEISINYRTEIDKPSQHHHHHH

SEQ ID NO: 431 - protein, 79 aa, Artificial Sequence
  Synthetic ATI_1494_D04 core
EVVAATPTSLLISWMESSSNSYRITYGETGGNSPVQEFTVPDQLATISGLKPGVDYTITVYALANAHYMRVGISINYRT

SEQ ID NO: 432 - protein, 7 aa, Artificial Sequence
  Synthetic ATI_1494_D04 BC loop
MESSSNS

SEQ ID NO: 433 - protein, 4 aa, Artificial Sequence
  Synthetic ATI_1494_D04 DE loop
PDQL

SEQ ID NO: 434 - protein, 10 aa, Artificial Sequence
  Synthetic ATI_1494_D04 FG loop
LANAHYMRVG

SEQ ID NO: 435 - protein, 88 aa, Artificial Sequence
  Synthetic ATI_1494_D04 w/ N leader
GVSDVPRDLEVVAATPTSLLISWMESSSNSYRITYGETGGNSPVQEFTVPDQLATISGLKPGVDYTITVYALANAHYMRVGISINYRT

SEQ ID NO: 436 - protein, 94 aa, Artificial Sequence
  Synthetic ATI_1494_D04 w/ N leader + his tag
GVSDVPRDLEVVAATPTSLLISWMESSSNSYRITYGETGGNSPVQEFTVPDQLATISGLKPGVDYTITVYALANAHYMRVGISINYRTHHHHHH

SEQ ID NO: 437 - protein, 95 aa, Artificial Sequence
  Synthetic ATI_1494_D04 w/ N leader and C tail
GVSDVPRDLEVVAATPTSLLISWMESSSNSYRITYGETGGNSPVQEFTVPDQLATISGLKPGVDYTITVYALANAHYMRVGISINYRTEIDKPSQ

SEQ ID NO: 438 - protein, 101 aa, Artificial Sequence
  Synthetic ATI_1494_D04 w/ N leader and C tail + his tag
GVSDVPRDLEVVAATPTSLLISWMESSSNSYRITYGETGGNSPVQEFTVPDQLATISGLKPGVDYTITVYALANAHYMRVGISINYRTEIDKPSQHHHHHH

SEQ ID NO: 439 - protein, 90 aa, Artificial Sequence
  Synthetic ATI_1494_D04 w/ N leader and modified C-terminus including PC
GVSDVPRDLEVVAATPTSLLISWMESSSNSYRITYGETGGNSPVQEFTVPDQLATISGLKPGVDYTITVYALANAHYMRVGISINYRTPC

SEQ ID NO: 440 - protein, 96 aa, Artificial Sequence
  Synthetic ATI_1494_D04 w/ N leader and modified C-terminus including PC + his tag
GVSDVPRDLEVVAATPTSLLISWMESSSNSYRITYGETGGNSPVQEFTVPDQLATISGLKPGVDYTITVYALANAHYMRVGISINYRTPCHHHHHH

SEQ ID NO: 441 - protein, 102 aa, Artificial Sequence
  Synthetic ATI_1494_D04 full length
MGVSDVPRDLEVVAATPTSLLISWMESSSNSYRITYGETGGNSPVQEFTVPDQLATISGLKPGVDYTITVYALANAHYMRVGISINYRTEIDKPSQHHHHHH

SEQ ID NO: 442 - protein, 79 aa, Artificial Sequence
  Synthetic ATI_1523_A08 core
  misc_feature (21)..(21): Xaa can be any naturally occurring amino acid
EVVAATPTSLLISWISVQTYXYRITYGETGGNSPVQEFTVPDQSATISGLKPGVDYTITVYALEKAHYYRQNISINYRT

SEQ ID NO: 443 - protein, 7 aa, Artificial Sequence
  Synthetic ATI_1523_A08 BC loop
  misc_feature (7)..(7): Xaa can be any naturally occurring amino acid
ISVQTYX

SEQ ID NO: 444 - protein, 4 aa, Artificial Sequence
  Synthetic ATI_1523_A08 DE loop
PDQS

SEQ ID NO: 445 - protein, 10 aa, Artificial Sequence
  Synthetic ATI_1523_A08 FG loop
LEKAHYYRQN

SEQ ID NO: 446 - protein, 88 aa, Artificial Sequence
  Synthetic ATI_1523_A08 w/ N leader
  misc_feature (30)..(30): Xaa can be any naturally occurring amino acid
GVSDVPRDLEVVAATPTSLLISWISVQTYXYRITYGETGGNSPVQEFTVPDQSATISGLKPGVDYTITVYALEKAHYYRQNISINYRT

SEQ ID NO: 447 - protein, 94 aa, Artificial Sequence
  Synthetic ATI_1523_A08 w/ N leader + his tag
  misc_feature (30)..(30): Xaa can be any naturally occurring amino acid
GVSDVPRDLEVVAATPTSLLISWISVQTYXYRITYGETGGNSPVQEFTVPDQSATISGLKPGVDYTITVYALEKAHYYRQNISINYRTHHHHHH

SEQ ID NO: 448 - protein, 95 aa, Artificial Sequence
  Synthetic ATI_1523_A08 w/ N leader and C tail
  misc_feature (30)..(30): Xaa can be any naturally occurring amino acid
GVSDVPRDLEVVAATPTSLLISWISVQTYXYRITYGETGGNSPVQEFTVPDQSATISGLKPGVDYTITVYALEKAHYYRQNISINYRTEIDKPSQ

SEQ ID NO: 449 - protein, 101 aa, Artificial Sequence
  Synthetic ATI_1523_A08 w/ N leader and C tail + his tag
  misc_feature (30)..(30): Xaa can be any naturally occurring amino acid
GVSDVPRDLEVVAATPTSLLISWISVQTYXYRITYGETGGNSPVQEFTVPDQSATISGLKPGVDYTITVYALEKAHYYRQNISINYRTEIDKPSQHHHHHH

SEQ ID NO: 450 - protein, 90 aa, Artificial Sequence
  Synthetic ATI_1523_A08 w/ N leader and modified C-terminus including PC
  misc_feature (30)..(30): Xaa can be any naturally occurring amino acid
GVSDVPRDLEVVAATPTSLLISWISVQTYXYRITYGETGGNSPVQEFTVPDQSATISGLKPGVDYTITVYALEKAHYYRQNISINYRTPC

SEQ ID NO: 451 - protein, 96 aa, Artificial Sequence
  Synthetic ATI_1523_A08 w/ N leader and modified C-terminus including PC + his tag
  misc_feature (30)..(30): Xaa can be any naturally occurring amino acid
GVSDVPRDLEVVAATPTSLLISWISVQTYXYRITYGETGGNSPVQEFTVPDQSATISGLKPGVDYTITVYALEKAHYYRQNISINYRTPCHHHHHH

SEQ ID NO: 452 - protein, 102 aa, Artificial Sequence
  Synthetic ATI_1523_A08 full length
  misc_feature (31)..(31): Xaa can be any naturally occurring amino acid
MGVSDVPRDLEVVAATPTSLLISWISVQTYXYRITYGETGGNSPVQEFTVPDQSATISGLKPGVDYTITVYALEKAHYYRQNISINYRTEIDKPSQHHHHHH

SEQ ID NO: 453 - protein, 79 aa, Artificial Sequence
  Synthetic ATI_1523_B10 core
  misc_feature (20)..(20): Xaa can be any naturally occurring amino acid
EVVAATPTSLLISWVYHYDXQYRITYGETGGNSPVQEFTVPDQKATISGLKPGVDYTITVYALSEAHHKRDSISINYRT

SEQ ID NO: 454 - protein, 7 aa, Artificial Sequence
  Synthetic ATI_1523_B10  BC loop
  misc_feature (6)..(6): Xaa can be any naturally occurring amino acid
VYHYDXQ

SEQ ID NO: 455 - protein, 4 aa, Artificial Sequence
  Synthetic ATI_1523_B10  DE loop
PDQK

SEQ ID NO: 456 - protein, 10 aa, Artificial Sequence
  Synthetic ATI_1523_B10  FG loop
LSEAHHKRDS

SEQ ID NO: 457 - protein, 88 aa, Artificial Sequence
  Synthetic ATI_1523_B10 w/ N leader
  misc_feature (29)..(29): Xaa can be any naturally occurring amino acid
GVSDVPRDLEVVAATPTSLLISWVYHYDXQYRITYGETGGNSPVQEFTVPDQKATISGLKPGVDYTITVYALSEAHHKRDSISINYRT

SEQ ID NO: 458 - protein, 94 aa, Artificial Sequence
  Synthetic ATI_1523_B10 w/ N leader + his tag
  misc_feature (29)..(29): Xaa can be any naturally occurring amino acid
GVSDVPRDLEVVAATPTSLLISWVYHYDXQYRITYGETGGNSPVQEFTVPDQKATISGLKPGVDYTITVYALSEAHHKRDSISINYRTHHHHHH

SEQ ID NO: 459 - protein, 95 aa, Artificial Sequence
  Synthetic ATI_1523_B10 w/ N leader and C tail
  misc_feature (29)..(29): Xaa can be any naturally occurring amino acid
GVSDVPRDLEVVAATPTSLLISWVYHYDXQYRITYGETGGNSPVQEFTVPDQKATISGLKPGVDYTITVYALSEAHHKRDSISINYRTEIDKPSQ

SEQ ID NO: 460 - protein, 101 aa, Artificial Sequence
  Synthetic ATI_1523_B10 w/ N leader and C tail + his tag
  misc_feature (29)..(29): Xaa can be any naturally occurring amino acid
GVSDVPRDLEVVAATPTSLLISWVYHYDXQYRITYGETGGNSPVQEFTVPDQKATISGLKPGVDYTITVYALSEAHHKRDSISINYRTEIDKPSQHHHHHH

SEQ ID NO: 461 - protein, 90 aa, Artificial Sequence
  Synthetic ATI_1523_B10 w/ N leader and modified C-terminus including PC
  misc_feature (29)..(29): Xaa can be any naturally occurring amino acid
GVSDVPRDLEVVAATPTSLLISWVYHYDXQYRITYGETGGNSPVQEFTVPDQKATISGLKPGVDYTITVYALSEAHHKRDSISINYRTPC

SEQ ID NO: 462 - protein, 96 aa, Artificial Sequence
  Synthetic ATI_1523_B10 w/ N leader and modified C-terminus including PC + his tag
  misc_feature (29)..(29): Xaa can be any naturally occurring amino acid
GVSDVPRDLEVVAATPTSLLISWVYHYDXQYRITYGETGGNSPVQEFTVPDQKATISGLKPGVDYTITVYALSEAHHKRDSISINYRTPCHHHHHH

SEQ ID NO: 463 - protein, 102 aa, Artificial Sequence
  Synthetic ATI_1523_B10  full length
  misc_feature (30)..(30): Xaa can be any naturally occurring amino acid
MGVSDVPRDLEVVAATPTSLLISWVYHYDXQYRITYGETGGNSPVQEFTVPDQKATISGLKPGVDYTITVYALSEAHHKRDSISINYRTEIDKPSQHHHHHH

SEQ ID NO: 464 - protein, 79 aa, Artificial Sequence
  Synthetic ATI_1523_C07 core
EVVAATPTSLLISWRMHTDPDYRITYGETGGNSPVQEFTVPDQEATISGLKPGVDYTITVYAIQTAHYYRINISINYRT

SEQ ID NO: 465 - protein, 7 aa, Artificial Sequence
  Synthetic ATI_1523_C07 BC loop
RMHTDPD

SEQ ID NO: 466 - protein, 4 aa, Artificial Sequence
  Synthetic ATI_1523_C07 DE loop
PDQE

SEQ ID NO: 467 - protein, 10 aa, Artificial Sequence
  Synthetic ATI_1523_C07 FG loop
IQTAHYYRIN

SEQ ID NO: 468 - protein, 88 aa, Artificial Sequence
  Synthetic ATI_1523_C07 w/ N leader
GVSDVPRDLEVVAATPTSLLISWRMHTDPDYRITYGETGGNSPVQEFTVPDQEATISGLKPGVDYTITVYAIQTAHYYRINISINYRT

SEQ ID NO: 469 - protein, 94 aa, Artificial Sequence
  Synthetic ATI_1523_C07 w/ N leader + his tag
GVSDVPRDLEVVAATPTSLLISWRMHTDPDYRITYGETGGNSPVQEFTVPDQEATISGLKPGVDYTITVYAIQTAHYYRINISINYRTHHHHHH

SEQ ID NO: 470 - protein, 95 aa, Artificial Sequence
  Synthetic ATI_1523_C07 w/ N leader and C tail
GVSDVPRDLEVVAATPTSLLISWRMHTDPDYRITYGETGGNSPVQEFTVPDQEATISGLKPGVDYTITVYAIQTAHYYRINISINYRTEIDKPSQ

SEQ ID NO: 471 - protein, 101 aa, Artificial Sequence
  Synthetic ATI_1523_C07 w/ N leader and C tail + his tag
GVSDVPRDLEVVAATPTSLLISWRMHTDPDYRITYGETGGNSPVQEFTVPDQEATISGLKPGVDYTITVYAIQTAHYYRINISINYRTEIDKPSQHHHHHH

SEQ ID NO: 472 - protein, 90 aa, Artificial Sequence
  Synthetic ATI_1523_C07 w/ N leader and modified C-terminus including PC
GVSDVPRDLEVVAATPTSLLISWRMHTDPDYRITYGETGGNSPVQEFTVPDQEATISGLKPGVDYTITVYAIQTAHYYRINISINYRTPC

SEQ ID NO: 473 - protein, 96 aa, Artificial Sequence
  Synthetic ATI_1523_C07 w/ N leader and modified C-terminus including PC + his tag
GVSDVPRDLEVVAATPTSLLISWRMHTDPDYRITYGETGGNSPVQEFTVPDQEATISGLKPGVDYTITVYAIQTAHYYRINISINYRTPCHHHHHH

SEQ ID NO: 474 - protein, 102 aa, Artificial Sequence
  Synthetic ATI_1523_C07 full length
MGVSDVPRDLEVVAATPTSLLISWRMHTDPDYRITYGETGGNSPVQEFTVPDQEATISGLKPGVDYTITVYAIQTAHYYRINISINYRTEIDKPSQHHHHHH

SEQ ID NO: 475 - protein, 79 aa, Artificial Sequence
  Synthetic ATI_1523_D07 core
EVVAATPTSLLISWENLASYQYRITYGETGGNSPVQEFTVPDVQATISGLKPGVDYTITVYALPYIHMKQRVISINYRT

SEQ ID NO: 476 - protein, 7 aa, Artificial Sequence
  Synthetic ATI_1523_D07 BC loop
ENLASYQ

SEQ ID NO: 477 - protein, 4 aa, Artificial Sequence
  Synthetic ATI_1523_D07 DE loop
PDVQ

SEQ ID NO: 478 - protein, 10 aa, Artificial Sequence
  Synthetic ATI_1523_D07 FG loop
LPYIHMKQRV

SEQ ID NO: 479 - protein, 88 aa, Artificial Sequence
  Synthetic ATI_1523_D07 w/ N leader
GVSDVPRDLEVVAATPTSLLISWENLASYQYRITYGETGGNSPVQEFTVPDVQATISGLKPGVDYTITVYALPYIHMKQRVISINYRT

SEQ ID NO: 480 - protein, 94 aa, Artificial Sequence
  Synthetic ATI_1523_D07 w/ N leader + his tag
GVSDVPRDLEVVAATPTSLLISWENLASYQYRITYGETGGNSPVQEFTVPDVQATISGLKPGVDYTITVYALPYIHMKQRVISINYRTHHHHHH

SEQ ID NO: 481 - protein, 95 aa, Artificial Sequence
  Synthetic ATI_1523_D07 w/ N leader and C tail
GVSDVPRDLEVVAATPTSLLISWENLASYQYRITYGETGGNSPVQEFTVPDVQATISGLKPGVDYTITVYALPYIHMKQRVISINYRTEIDKPSQ

SEQ ID NO: 482 - protein, 101 aa, Artificial Sequence
  Synthetic ATI_1523_D07 w/ N leader and C tail + his tag
GVSDVPRDLEVVAATPTSLLISWENLASYQYRITYGETGGNSPVQEFTVPDVQATISGLKPGVDYTITVYALPYIHMKQRVISINYRTEIDKPSQHHHHHH

SEQ ID NO: 483 - protein, 90 aa, Artificial Sequence
  Synthetic ATI_1523_D07 w/ N leader and modified C-terminus including PC
GVSDVPRDLEVVAATPTSLLISWENLASYQYRITYGETGGNSPVQEFTVPDVQATISGLKPGVDYTITVYALPYIHMKQRVISINYRTPC

SEQ ID NO: 484 - protein, 96 aa, Artificial Sequence
  Synthetic ATI_1523_D07 w/ N leader and modified C-terminus including PC + his tag
GVSDVPRDLEVVAATPTSLLISWENLASYQYRITYGETGGNSPVQEFTVPDVQATISGLKPGVDYTITVYALPYIHMKQRVISINYRTPCHHHHHH

SEQ ID NO: 485 - protein, 102 aa, Artificial Sequence
  Synthetic ATI_1523_D07  full length
MGVSDVPRDLEVVAATPTSLLISWENLASYQYRITYGETGGNSPVQEFTVPDVQATISGLKPGVDYTITVYALPYIHMKQRVISINYRTEIDKPSQHHHHHH

SEQ ID NO: 486 - protein, 79 aa, Artificial Sequence
  Synthetic ATI_1523_D08 core
EVVAATPTSLLISWMRYYDAYYRITYGETGGNSPVQEFTVPDQSATISGLKPGVDYTITVYALEKAHYYRQNISINYRT

SEQ ID NO: 487 - protein, 7 aa, Artificial Sequence
  Synthetic ATI_1523_D08 BC loop
MRYYDAY

SEQ ID NO: 488 - protein, 4 aa, Artificial Sequence
  Synthetic ATI_1523_D08 DE loop
PDQS

SEQ ID NO: 489 - protein, 10 aa, Artificial Sequence
  Synthetic ATI_1523_D08 FG loop
LEKAHYYRQN

SEQ ID NO: 490 - protein, 88 aa, Artificial Sequence
  Synthetic ATI_1523_D08 w/ N leader
GVSDVPRDLEVVAATPTSLLISWMRYYDAYYRITYGETGGNSPVQEFTVPDQSATISGLKPGVDYTITVYALEKAHYYRQNISINYRT

SEQ ID NO: 491 - protein, 94 aa, Artificial Sequence
  Synthetic ATI_1523_D08 w/ N leader + his tag
GVSDVPRDLEVVAATPTSLLISWMRYYDAYYRITYGETGGNSPVQEFTVPDQSATISGLKPGVDYTITVYALEKAHYYRQNISINYRTHHHHHH

SEQ ID NO: 492 - protein, 95 aa, Artificial Sequence
  Synthetic ATI_1523_D08 w/ N leader and C tail
GVSDVPRDLEVVAATPTSLLISWMRYYDAYYRITYGETGGNSPVQEFTVPDQSATISGLKPGVDYTITVYALEKAHYYRQNISINYRTEIDKPSQ

SEQ ID NO: 493 - protein, 101 aa, Artificial Sequence
  Synthetic ATI_1523_D08 w/ N leader and C tail + his tag
GVSDVPRDLEVVAATPTSLLISWMRYYDAYYRITYGETGGNSPVQEFTVPDQSATISGLKPGVDYTITVYALEKAHYYRQNISINYRTEIDKPSQHHHHHH

SEQ ID NO: 494 - protein, 90 aa, Artificial Sequence
  Synthetic ATI_1523_D08 w/ N leader and modified C-terminus including PC
GVSDVPRDLEVVAATPTSLLISWMRYYDAYYRITYGETGGNSPVQEFTVPDQSATISGLKPGVDYTITVYALEKAHYYRQNISINYRTPC

SEQ ID NO: 495 - protein, 96 aa, Artificial Sequence
  Synthetic ATI_1523_D08 w/ N leader and modified C-terminus including PC + his tag
GVSDVPRDLEVVAATPTSLLISWMRYYDAYYRITYGETGGNSPVQEFTVPDQSATISGLKPGVDYTITVYALEKAHYYRQNISINYRTPCHHHHHH

SEQ ID NO: 496 - protein, 102 aa, Artificial Sequence
  Synthetic ATI_1523_D08 full length
MGVSDVPRDLEVVAATPTSLLISWMRYYDAYYRITYGETGGNSPVQEFTVPDQSATISGLKPGVDYTITVYALEKAHYYRQNISINYRTEIDKPSQHHHHHH

SEQ ID NO: 497 - protein, 79 aa, Artificial Sequence
  Synthetic ATI_1523_E08 core
EVVAATPTSLLISWHHYQHYEYRITYGETGGNSPVQEFTVPDMGATISGLKPGVDYTITVYALEEAHSDRSSISINYRT

SEQ ID NO: 498 - protein, 7 aa, Artificial Sequence
  Synthetic ATI_1523_E08 BC loop
HHYQHYE

SEQ ID NO: 499 - protein, 4 aa, Artificial Sequence
  Synthetic ATI_1523_E08 DE loop
PDMG

SEQ ID NO: 500 - protein, 10 aa, Artificial Sequence
  Synthetic ATI_1523_E08  FG loop
LEEAHSDRSS

SEQ ID NO: 501 - protein, 88 aa, Artificial Sequence
  Synthetic ATI_1523_E08 w/ N leader
GVSDVPRDLEVVAATPTSLLISWHHYQHYEYRITYGETGGNSPVQEFTVPDMGATISGLKPGVDYTITVYALEEAHSDRSSISINYRT

SEQ ID NO: 502 - protein, 94 aa, Artificial Sequence
  Synthetic ATI_1523_E08 w/ N leader + his tag
GVSDVPRDLEVVAATPTSLLISWHHYQHYEYRITYGETGGNSPVQEFTVPDMGATISGLKPGVDYTITVYALEEAHSDRSSISINYRTHHHHHH

SEQ ID NO: 503 - protein, 95 aa, Artificial Sequence
  Synthetic ATI_1523_E08 w/ N leader and C tail
GVSDVPRDLEVVAATPTSLLISWHHYQHYEYRITYGETGGNSPVQEFTVPDMGATISGLKPGVDYTITVYALEEAHSDRSSISINYRTEIDKPSQ

SEQ ID NO: 504 - protein, 101 aa, Artificial Sequence
  Synthetic ATI_1523_E08 w/ N leader and C tail + his tag
GVSDVPRDLEVVAATPTSLLISWHHYQHYEYRITYGETGGNSPVQEFTVPDMGATISGLKPGVDYTITVYALEEAHSDRSSISINYRTEIDKPSQHHHHHH

SEQ ID NO: 505 - protein, 90 aa, Artificial Sequence
  Synthetic ATI_1523_E08 w/ N leader and modified C-terminus including PC
GVSDVPRDLEVVAATPTSLLISWHHYQHYEYRITYGETGGNSPVQEFTVPDMGATISGLKPGVDYTITVYALEEAHSDRSSISINYRTPC

SEQ ID NO: 506 - protein, 96 aa, Artificial Sequence
  Synthetic ATI_1523_E08 w/ N leader and modified C-terminus including PC + his tag
GVSDVPRDLEVVAATPTSLLISWHHYQHYEYRITYGETGGNSPVQEFTVPDMGATISGLKPGVDYTITVYALEEAHSDRSSISINYRTPCHHHHHH

SEQ ID NO: 507 - protein, 102 aa, Artificial Sequence
  Synthetic ATI_1523_E08 full length
MGVSDVPRDLEVVAATPTSLLISWHHYQHYEYRITYGETGGNSPVQEFTVPDMGATISGLKPGVDYTITVYALEEAHSDRSSISINYRTEIDKPSQHHHHHH

SEQ ID NO: 508 - protein, 76 aa, Artificial Sequence
  Synthetic ATI_1523_F01 core
EVVAATPTSLLISWYKPSTIVTYRITYGETGGNSPVQEFTVYGYNATISGLKPGVDYTITVYAVHGVRFISINYRT

SEQ ID NO: 509 - protein, 8 aa, Artificial Sequence
  Synthetic ATI_1523_F01 BC loop
YKPSTIVT

SEQ ID NO: 510 - protein, 4 aa, Artificial Sequence
  Synthetic ATI_1523_F01 DE loop
YGYN

SEQ ID NO: 511 - protein, 6 aa, Artificial Sequence
  Synthetic ATI_1523_F01 FG loop
VHGVRF

SEQ ID NO: 512 - protein, 85 aa, Artificial Sequence
  Synthetic ATI_1523_F01 w/ N leader
GVSDVPRDLEVVAATPTSLLISWYKPSTIVTYRITYGETGGNSPVQEFTVYGYNATISGLKPGVDYTITVYAVHGVRFISINYRT

SEQ ID NO: 513 - protein, 91 aa, Artificial Sequence
  Synthetic ATI_1523_F01 w/ N leader + his tag
GVSDVPRDLEVVAATPTSLLISWYKPSTIVTYRITYGETGGNSPVQEFTVYGYNATISGLKPGVDYTITVYAVHGVRFISINYRTHHHHHH

SEQ ID NO: 514 - protein, 92 aa, Artificial Sequence
  Synthetic ATI_1523_F01 w/ N leader and C tail
GVSDVPRDLEVVAATPTSLLISWYKPSTIVTYRITYGETGGNSPVQEFTVYGYNATISGLKPGVDYTITVYAVHGVRFISINYRTEIDKPSQ

SEQ ID NO: 515 - protein, 98 aa, Artificial Sequence
  Synthetic ATI_1523_F01 w/ N leader and C tail + his tag
GVSDVPRDLEVVAATPTSLLISWYKPSTIVTYRITYGETGGNSPVQEFTVYGYNATISGLKPGVDYTITVYAVHGVRFISINYRTEIDKPSQHHHHHH

SEQ ID NO: 516 - protein, 87 aa, Artificial Sequence
  Synthetic ATI_1523_F01 w/ N leader and modified C-terminus including PC
GVSDVPRDLEVVAATPTSLLISWYKPSTIVTYRITYGETGGNSPVQEFTVYGYNATISGLKPGVDYTITVYAVHGVRFISINYRTPC

SEQ ID NO: 517 - protein, 93 aa, Artificial Sequence
  Synthetic ATI_1523_F01 w/ N leader and modified C-terminus including PC + his tag
GVSDVPRDLEVVAATPTSLLISWYKPSTIVTYRITYGETGGNSPVQEFTVYGYNATISGLKPGVDYTITVYAVHGVRFISINYRTPCHHHHHH

SEQ ID NO: 518 - protein, 99 aa, Artificial Sequence
  Synthetic ATI_1523_F01 full length
MGVSDVPRDLEVVAATPTSLLISWYKPSTIVTYRITYGETGGNSPVQEFTVYGYNATISGLKPGVDYTITVYAVHGVRFISINYRTEIDKPSQHHHHHH

SEQ ID NO: 519 - protein, 78 aa, Artificial Sequence
  Synthetic ATI_1523_F04 core
EVVAATPTSLLISWGGSLSPTFYRITYGETGGNSPVQEFTVTYQGATISGLKPGVDYTITVYATEGIVYYQISINYRT

SEQ ID NO: 520 - protein, 8 aa, Artificial Sequence
  Synthetic ATI_1523_F04 BC loop
GGSLSPTF

SEQ ID NO: 521 - protein, 4 aa, Artificial Sequence
  Synthetic ATI_1523_F04 DE loop
TYQG

SEQ ID NO: 522 - protein, 8 aa, Artificial Sequence
  Synthetic ATI_1523_F04 FG loop
TEGIVYYQ

SEQ ID NO: 523 - protein, 87 aa, Artificial Sequence
  Synthetic ATI_1523_F04 w/ N leader
GVSDVPRDLEVVAATPTSLLISWGGSLSPTFYRITYGETGGNSPVQEFTVTYQGATISGLKPGVDYTITVYATEGIVYYQISINYRT

SEQ ID NO: 524 - protein, 93 aa, Artificial Sequence
  Synthetic ATI_1523_F04 w/ N leader + his tag
GVSDVPRDLEVVAATPTSLLISWGGSLSPTFYRITYGETGGNSPVQEFTVTYQGATISGLKPGVDYTITVYATEGIVYYQISINYRTHHHHHH

SEQ ID NO: 525 - protein, 94 aa, Artificial Sequence
  Synthetic ATI_1523_F04 w/ N leader and C tail
GVSDVPRDLEVVAATPTSLLISWGGSLSPTFYRITYGETGGNSPVQEFTVTYQGATISGLKPGVDYTITVYATEGIVYYQISINYRTEIDKPSQ

SEQ ID NO: 526 - protein, 100 aa, Artificial Sequence
  Synthetic ATI_1523_F04 w/ N leader and C tail + his tag
GVSDVPRDLEVVAATPTSLLISWGGSLSPTFYRITYGETGGNSPVQEFTVTYQGATISGLKPGVDYTITVYATEGIVYYQISINYRTEIDKPSQHHHHHH

SEQ ID NO: 527 - protein, 89 aa, Artificial Sequence
  Synthetic ATI_1523_F04 w/ N leader and modified C-terminus including PC
GVSDVPRDLEVVAATPTSLLISWGGSLSPTFYRITYGETGGNSPVQEFTVTYQGATISGLKPGVDYTITVYATEGIVYYQISINYRTPC

SEQ ID NO: 528 - protein, 95 aa, Artificial Sequence
  Synthetic ATI_1523_F04 w/ N leader and modified C-terminus including PC + his tag
GVSDVPRDLEVVAATPTSLLISWGGSLSPTFYRITYGETGGNSPVQEFTVTYQGATISGLKPGVDYTITVYATEGIVYYQISINYRTPCHHHHHH

SEQ ID NO: 529 - protein, 101 aa, Artificial Sequence
  Synthetic ATI_1523_F04 full length
MGVSDVPRDLEVVAATPTSLLISWGGSLSPTFYRITYGETGGNSPVQEFTVTYQGATISGLKPGVDYTITVYATEGIVYYQISINYRTEIDKPSQHHHHHH

SEQ ID NO: 530 - protein, 79 aa, Artificial Sequence
  Synthetic ATI_1523_F08 core
EVVAATPTSLLISWVYHYDAQYRITYGETGGNSPVQEFTVPDQKATISGLKPGVDYTITVYALPRAHMDRSHISINYRT

SEQ ID NO: 531 - protein, 7 aa, Artificial Sequence
  Synthetic ATI_1523_F08 BC loop
VYHYDAQ

SEQ ID NO: 532 - protein, 4 aa, Artificial Sequence
  Synthetic ATI_1523_F08 DE loop
PDQK

SEQ ID NO: 533 - protein, 10 aa, Artificial Sequence
  Synthetic ATI_1523_F08 FG loop
LPRAHMDRSH

SEQ ID NO: 534 - protein, 88 aa, Artificial Sequence
  Synthetic ATI_1523_F08 w/ N leader
GVSDVPRDLEVVAATPTSLLISWVYHYDAQYRITYGETGGNSPVQEFTVPDQKATISGLKPGVDYTITVYALPRAHMDRSHISINYRT

SEQ ID NO: 535 - protein, 94 aa, Artificial Sequence
  Synthetic ATI_1523_F08 w/ N leader + his tag
GVSDVPRDLEVVAATPTSLLISWVYHYDAQYRITYGETGGNSPVQEFTVPDQKATISGLKPGVDYTITVYALPRAHMDRSHISINYRTHHHHHH

SEQ ID NO: 536 - protein, 95 aa, Artificial Sequence
  Synthetic ATI_1523_F08 w/ N leader and C tail
GVSDVPRDLEVVAATPTSLLISWVYHYDAQYRITYGETGGNSPVQEFTVPDQKATISGLKPGVDYTITVYALPRAHMDRSHISINYRTEIDKPSQ

SEQ ID NO: 537 - protein, 101 aa, Artificial Sequence
  Synthetic ATI_1523_F08 w/ N leader and C tail + his tag
GVSDVPRDLEVVAATPTSLLISWVYHYDAQYRITYGETGGNSPVQEFTVPDQKATISGLKPGVDYTITVYALPRAHMDRSHISINYRTEIDKPSQHHHHHH

SEQ ID NO: 538 - protein, 90 aa, Artificial Sequence
  Synthetic ATI_1523_F08 w/ N leader and modified C-terminus including PC
GVSDVPRDLEVVAATPTSLLISWVYHYDAQYRITYGETGGNSPVQEFTVPDQKATISGLKPGVDYTITVYALPRAHMDRSHISINYRTPC

SEQ ID NO: 539 - protein, 96 aa, Artificial Sequence
  Synthetic ATI_1523_F08 w/ N leader and modified C-terminus including PC + his tag
GVSDVPRDLEVVAATPTSLLISWVYHYDAQYRITYGETGGNSPVQEFTVPDQKATISGLKPGVDYTITVYALPRAHMDRSHISINYRTPCHHHHHH

SEQ ID NO: 540 - protein, 102 aa, Artificial Sequence
  Synthetic ATI_1523_F08 full length
MGVSDVPRDLEVVAATPTSLLISWVYHYDAQYRITYGETGGNSPVQEFTVPDQKATISGLKPGVDYTITVYALPRAHMDRSHISINYRTEIDKPSQHHHHHH

SEQ ID NO: 541 - protein, 79 aa, Artificial Sequence
  Synthetic ATI_1523_G06 core
EVVAATPTSLLISWRIKSYHKYRITYGETGGNSPVQEFTVRSYAATISGLKPGVDYTITVYAIMEETHLAYAISINYRT

SEQ ID NO: 542 - protein, 7 aa, Artificial Sequence
  Synthetic ATI_1523_G06 BC loop
RIKSYHK

SEQ ID NO: 543 - protein, 4 aa, Artificial Sequence
  Synthetic ATI_1523_G06 DE loop
RSYA

SEQ ID NO: 544 - protein, 10 aa, Artificial Sequence
  Synthetic ATI_1523_G06 FG loop
IMEETHLAYA

SEQ ID NO: 545 - protein, 88 aa, Artificial Sequence
  Synthetic ATI_1523_G06 w/ N leader
GVSDVPRDLEVVAATPTSLLISWRIKSYHKYRITYGETGGNSPVQEFTVRSYAATISGLKPGVDYTITVYAIMEETHLAYAISINYRT

SEQ ID NO: 546 - protein, 94 aa, Artificial Sequence
  Synthetic ATI_1523_G06 w/ N leader + his tag
GVSDVPRDLEVVAATPTSLLISWRIKSYHKYRITYGETGGNSPVQEFTVRSYAATISGLKPGVDYTITVYAIMEETHLAYAISINYRTHHHHHH

SEQ ID NO: 547 - protein, 95 aa, Artificial Sequence
  Synthetic ATI_1523_G06 w/ N leader and C tail
GVSDVPRDLEVVAATPTSLLISWRIKSYHKYRITYGETGGNSPVQEFTVRSYAATISGLKPGVDYTITVYAIMEETHLAYAISINYRTEIDKPSQ

SEQ ID NO: 548 - protein, 101 aa, Artificial Sequence
  Synthetic ATI_1523_G06 w/ N leader and C tail + his tag
GVSDVPRDLEVVAATPTSLLISWRIKSYHKYRITYGETGGNSPVQEFTVRSYAATISGLKPGVDYTITVYAIMEETHLAYAISINYRTEIDKPSQHHHHHH

SEQ ID NO: 549 - protein, 90 aa, Artificial Sequence
  Synthetic ATI_1523_G06 w/ N leader and modified C-terminus including PC
GVSDVPRDLEVVAATPTSLLISWRIKSYHKYRITYGETGGNSPVQEFTVRSYAATISGLKPGVDYTITVYAIMEETHLAYAISINYRTPC

SEQ ID NO: 550 - protein, 96 aa, Artificial Sequence
  Synthetic ATI_1523_G06 w/ N leader and modified C-terminus including PC + his tag
GVSDVPRDLEVVAATPTSLLISWRIKSYHKYRITYGETGGNSPVQEFTVRSYAATISGLKPGVDYTITVYAIMEETHLAYAISINYRTPCHHHHHH

SEQ ID NO: 551 - protein, 102 aa, Artificial Sequence
  Synthetic ATI_1523_G06 full length
MGVSDVPRDLEVVAATPTSLLISWRIKSYHKYRITYGETGGNSPVQEFTVRSYAATISGLKPGVDYTITVYAIMEETHLAYAISINYRTEIDKPSQHHHHHH

SEQ ID NO: 552 - protein, 79 aa, Artificial Sequence
  Synthetic ATI_1523_G07 core
EVVAATPTSLLISWVYPQADDYRITYGETGGNSPVQEFTVPDQNATISGLKPGVDYTITVYALAEAHLVRIYISINYRT

SEQ ID NO: 553 - protein, 7 aa, Artificial Sequence
  Synthetic ATI_1523_G07 BC loop
VYPQADD

SEQ ID NO: 554 - protein, 4 aa, Artificial Sequence
  Synthetic ATI_1523_G07 DE loop
PDQN

SEQ ID NO: 555 - protein, 10 aa, Artificial Sequence
  Synthetic ATI_1523_G07 FG loop
LAEAHLVRIY

SEQ ID NO: 556 - protein, 88 aa, Artificial Sequence
  Synthetic ATI_1523_G07 w/ N leader
GVSDVPRDLEVVAATPTSLLISWVYPQADDYRITYGETGGNSPVQEFTVPDQNATISGLKPGVDYTITVYALAEAHLVRIYISINYRT

SEQ ID NO: 557 - protein, 94 aa, Artificial Sequence
  Synthetic ATI_1523_G07 w/ N leader + his tag
GVSDVPRDLEVVAATPTSLLISWVYPQADDYRITYGETGGNSPVQEFTVPDQNATISGLKPGVDYTITVYALAEAHLVRIYISINYRTHHHHHH

SEQ ID NO: 558 - protein, 95 aa, Artificial Sequence
  Synthetic ATI_1523_G07 w/ N leader and C tail
GVSDVPRDLEVVAATPTSLLISWVYPQADDYRITYGETGGNSPVQEFTVPDQNATISGLKPGVDYTITVYALAEAHLVRIYISINYRTEIDKPSQ

SEQ ID NO: 559 - protein, 101 aa, Artificial Sequence
  Synthetic ATI_1523_G07 w/ N leader and C tail + his tag
GVSDVPRDLEVVAATPTSLLISWVYPQADDYRITYGETGGNSPVQEFTVPDQNATISGLKPGVDYTITVYALAEAHLVRIYISINYRTEIDKPSQHHHHHH

SEQ ID NO: 560 - protein, 90 aa, Artificial Sequence
  Synthetic ATI_1523_G07 w/ N leader and modified C-terminus including PC
GVSDVPRDLEVVAATPTSLLISWVYPQADDYRITYGETGGNSPVQEFTVPDQNATISGLKPGVDYTITVYALAEAHLVRIYISINYRTPC

SEQ ID NO: 561 - protein, 96 aa, Artificial Sequence
  Synthetic ATI_1523_G07 w/ N leader and modified C-terminus including PC + his tag
GVSDVPRDLEVVAATPTSLLISWVYPQADDYRITYGETGGNSPVQEFTVPDQNATISGLKPGVDYTITVYALAEAHLVRIYISINYRTPCHHHHHH

SEQ ID NO: 562 - protein, 102 aa, Artificial Sequence
  Synthetic ATI_1523_G07 full length
MGVSDVPRDLEVVAATPTSLLISWVYPQADDYRITYGETGGNSPVQEFTVPDQNATISGLKPGVDYTITVYALAEAHLVRIYISINYRTEIDKPSQHHHHHH

SEQ ID NO: 563 - protein, 79 aa, Artificial Sequence
  Synthetic ATI_1523_H07 core
  misc_feature (21)..(21): Xaa can be any naturally occurring amino acid
EVVAATPTSLLISWVYHYDAXYRITYGETGGNSPVQEFTVPDQKATISGLKPGVDYTITVYALSEAHHKRDSISINYRT

SEQ ID NO: 564 - protein, 7 aa, Artificial Sequence
  Synthetic ATI_1523_H07 BC loop
  misc_feature (7)..(7): Xaa can be any naturally occurring amino acid
VYHYDAX

SEQ ID NO: 565 - protein, 4 aa, Artificial Sequence
  Synthetic ATI_1523_H07 DE loop
PDQK

SEQ ID NO: 566 - protein, 10 aa, Artificial Sequence
  Synthetic ATI_1523_H07 FG loop
LSEAHHKRDS

SEQ ID NO: 567 - protein, 88 aa, Artificial Sequence
  Synthetic ATI_1523_H07 w/ N leader
  misc_feature (30)..(30): Xaa can be any naturally occurring amino acid
GVSDVPRDLEVVAATPTSLLISWVYHYDAXYRITYGETGGNSPVQEFTVPDQKATISGLKPGVDYTITVYALSEAHHKRDSISINYRT

SEQ ID NO: 568 - protein, 94 aa, Artificial Sequence
  Synthetic ATI_1523_H07 w/ N leader + his tag
  misc_feature (30)..(30): Xaa can be any naturally occurring amino acid
GVSDVPRDLEVVAATPTSLLISWVYHYDAXYRITYGETGGNSPVQEFTVPDQKATISGLKPGVDYTITVYALSEAHHKRDSISINYRTHHHHHH

SEQ ID NO: 569 - protein, 95 aa, Artificial Sequence
  Synthetic ATI_1523_H07 w/ N leader and C tail
  misc_feature (30)..(30): Xaa can be any naturally occurring amino acid
GVSDVPRDLEVVAATPTSLLISWVYHYDAXYRITYGETGGNSPVQEFTVPDQKATISGLKPGVDYTITVYALSEAHHKRDSISINYRTEIDKPSQ

SEQ ID NO: 570 - protein, 101 aa, Artificial Sequence
  Synthetic ATI_1523_H07 w/ N leader and C tail + his tag
  misc_feature (30)..(30): Xaa can be any naturally occurring amino acid
GVSDVPRDLEVVAATPTSLLISWVYHYDAXYRITYGETGGNSPVQEFTVPDQKATISGLKPGVDYTITVYALSEAHHKRDSISINYRTEIDKPSQHHHHHH

SEQ ID NO: 571 - protein, 90 aa, Artificial Sequence
  Synthetic ATI_1523_H07 w/ N leader and modified C-terminus including PC
  misc_feature (30)..(30): Xaa can be any naturally occurring amino acid
GVSDVPRDLEVVAATPTSLLISWVYHYDAXYRITYGETGGNSPVQEFTVPDQKATISGLKPGVDYTITVYALSEAHHKRDSISINYRTPC

SEQ ID NO: 572 - protein, 96 aa, Artificial Sequence
  Synthetic ATI_1523_H07 w/ N leader and modified C-terminus including PC + his tag
  misc_feature (30)..(30): Xaa can be any naturally occurring amino acid
GVSDVPRDLEVVAATPTSLLISWVYHYDAXYRITYGETGGNSPVQEFTVPDQKATISGLKPGVDYTITVYALSEAHHKRDSISINYRTPCHHHHHH

SEQ ID NO: 573 - protein, 101 aa, Artificial Sequence; identical to SEQ ID NO: 570
  Synthetic ATI_1523_H07 full length
  misc_feature (30)..(30): Xaa can be any naturally occurring amino acid

SEQ ID NO: 574 - protein, 10 aa, Artificial Sequence
  Synthetic N-terminal leader
MGVSDVPRDL

SEQ ID NO: 575 - protein, 9 aa, Artificial Sequence
  Synthetic N-terminal leader
GVSDVPRDL

SEQ ID NO: 576 - protein, 9 aa, Artificial Sequence
  Synthetic N-terminal leader
  misc_feature (1)..(2): Xaa may or may not be present; if one present, Xaa is Met OR Gly; if both present, Xaa Xaa is Met-Gly
XXSDVPRDL

SEQ ID NO: 577 - protein, 8 aa, Artificial Sequence
  Synthetic N-terminal leader
  misc_feature (1)..(2): Xaa may or may not be present; if one present, Xaa is Met OR Gly; if both present, Xaa Xaa is Met-Gly
XXDVPRDL

SEQ ID NO: 578 - protein, 7 aa, Artificial Sequence
  Synthetic N-terminal leader
  misc_feature (1)..(2): Xaa may or may not be present; if one present, Xaa is Met OR Gly; if both present, Xaa Xaa is Met-Gly
XXVPRDL

SEQ ID NO: 579 - protein, 6 aa, Artificial Sequence
  Synthetic N-terminal leader
  misc_feature (1)..(2): Xaa may or may not be present; if one present, Xaa is Met OR Gly; if both present, Xaa Xaa is Met-Gly
XXPRDL

SEQ ID NO: 580 - protein, 5 aa, Artificial Sequence
  Synthetic N-terminal leader
  misc_feature (1)..(2): Xaa may or may not be present; if one present, Xaa is Met OR Gly; if both present, Xaa Xaa is Met-Gly
XXRDL

SEQ ID NO: 581 - protein, 4 aa, Artificial Sequence
  Synthetic N-terminal leader
  misc_feature (1)..(2): Xaa may or may not be present; if one present, Xaa is Met OR Gly; if both present, Xaa Xaa is Met-Gly
XXDL

SEQ ID NO: 582 - protein, 6 aa, Artificial Sequence
  Synthetic N-terminal leader
MASTSG

SEQ ID NO: 583 - protein, 20 aa, Artificial Sequence
  Synthetic N-terminal leader
METDTLLLWVLLLWVPGSTG

SEQ ID NO: 584 - protein, 4 aa, Artificial Sequence
  Synthetic C-terminal tail
EIEK

SEQ ID NO: 585 - protein, 5 aa, Artificial Sequence
  Synthetic C-terminal tail
EGSGC

SEQ ID NO: 586 - protein, 7 aa, Artificial Sequence
  Synthetic C-terminal tail
EIEKPCQ

SEQ ID NO: 587 - protein, 7 aa, Artificial Sequence
  Synthetic C-terminal tail
EIEKPSQ

SEQ ID NO: 588 - protein, 5 aa, Artificial Sequence
  Synthetic C-terminal tail
EIEKP

SEQ ID NO: 589 - protein, 6 aa, Artificial Sequence
  Synthetic C-terminal tail
EIEKPS

SEQ ID NO: 590 - protein, 6 aa, Artificial Sequence
  Synthetic C-terminal tail
EIEKPC

SEQ ID NO: 591 - protein, 4 aa, Artificial Sequence
  Synthetic C-terminal tail
EIDK

SEQ ID NO: 592 - protein, 7 aa, Artificial Sequence
  Synthetic C-terminal tail
EIDKPCQ

SEQ ID NO: 593 - protein, 7 aa, Artificial Sequence
  Synthetic C-terminal tail
EIDKPSQ

SEQ ID NO: 594 - protein, 7 aa, Artificial Sequence
  Synthetic C-terminal tail
EIEPKSS

SEQ ID NO: 595 - protein, 6 aa, Artificial Sequence
  Synthetic C-terminal tail
EIDKPC

SEQ ID NO: 596 - protein, 5 aa, Artificial Sequence
  Synthetic C-terminal tail
EIDKP

SEQ ID NO: 597 - protein, 6 aa, Artificial Sequence
  Synthetic C-terminal tail
EIDKPS

SEQ ID NO: 598 - protein, 9 aa, Artificial Sequence
  Synthetic C-terminal tail
EIDKPSQLE

SEQ ID NO: 599 - protein, 12 aa, Artificial Sequence
  Synthetic C-terminal tail
EIEDEDEDEDED

SEQ ID NO: 600 - protein, 5 aa, Artificial Sequence
  Synthetic C-terminal tail
EGSGS

SEQ ID NO: 601 - protein, 9 aa, Artificial Sequence
  Synthetic C-terminal tail
EIDKPCQLE

SEQ ID NO: 602 - protein, 13 aa, Artificial Sequence
  Synthetic C-terminal tail
EIDKPSQHHHHHH

SEQ ID NO: 603 - protein, 10 aa, Artificial Sequence
  Synthetic C-terminal tail
GSGCHHHHHH

SEQ ID NO: 604 - protein, 11 aa, Artificial Sequence
  Synthetic C-terminal tail
EGSGCHHHHHH

SEQ ID NO: 605 - protein, 4 aa, Artificial Sequence
  Synthetic C-terminal tail
PIDK

SEQ ID NO: 606 - protein, 4 aa, Artificial Sequence
  Synthetic C-terminal tail
PIEK

SEQ ID NO: 607 - protein, 5 aa, Artificial Sequence
  Synthetic C-terminal tail
PIDKP

SEQ ID NO: 608 - protein, 5 aa, Artificial Sequence
  Synthetic C-terminal tail
PIEKP

SEQ ID NO: 609 - protein, 6 aa, Artificial Sequence
  Synthetic C-terminal tail
PIDKPS

SEQ ID NO: 610 - protein, 6 aa, Artificial Sequence
  Synthetic C-terminal tail
PIEKPS

SEQ ID NO: 611 - protein, 6 aa, Artificial Sequence
  Synthetic C-terminal tail
PIDKPC

SEQ ID NO: 612 - protein, 6 aa, Artificial Sequence
  Synthetic C-terminal tail
PIEKPC

SEQ ID NO: 613 - protein, 7 aa, Artificial Sequence
  Synthetic C-terminal tail
PIDKPSQ

SEQ ID NO: 614 - protein, 7 aa, Artificial Sequence
  Synthetic C-terminal tail
PIEKPSQ

SEQ ID NO: 615 - protein, 7 aa, Artificial Sequence
  Synthetic C-terminal tail
PIDKPCQ

SEQ ID NO: 616 - protein, 7 aa, Artificial Sequence
  Synthetic C-terminal tail
PIEKPCQ

SEQ ID NO: 617 - protein, 7 aa, Artificial Sequence
  Synthetic C-terminal tail
PHHHHHH

SEQ ID NO: 618 - protein, 8 aa, Artificial Sequence
  Synthetic C-terminal tail
PCHHHHHH

SEQ ID NO: 619 - protein, 6 aa, Artificial Sequence
  Synthetic 6X-His tag
HHHHHH

SEQ ID NO: 620 - protein, 227 aa, Homo sapiens
  misc_feature: Human IgG1 Fc domain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 621 - protein, 16 aa, Artificial Sequence
  Synthetic Core hinge region of Fc
DKTHTCPPCPAPELLG

SEQ ID NO: 622 - protein, 24 aa, Artificial Sequence
  Synthetic Exemplary hinge sequence
EPKSSDKTHTCPPCPAPELLGGPS

SEQ ID NO: 623 - protein, 24 aa, Artificial Sequence
  Synthetic Exemplary hinge sequence
EPKSSDKTHTCPPCPAPELLGGSS

SEQ ID NO: 624 - protein, 24 aa, Artificial Sequence
  Synthetic Exemplary hinge sequence
EPKSSGSTHTCPPCPAPELLGGSS

SEQ ID NO: 625 - protein, 19 aa, Artificial Sequence
  Synthetic Exemplary hinge sequence
DKTHTCPPCPAPELLGGPS

SEQ ID NO: 626 - protein, 19 aa, Artificial Sequence
  Synthetic Exemplary hinge sequence
DKTHTCPPCPAPELLGGSS

SEQ ID NO: 627 - protein, 208 aa, Artificial Sequence
  Synthetic Fc with CH2 and CH3 regions of IgG1 for Adnectin-hinge-Fc construct
VFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 628 - protein, 206 aa, Artificial Sequence
  Synthetic Fc with CH2 and CH3 regions of IgG1 for Fc-hinge-Adnectin construct
VFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 629 - protein, 8 aa, Artificial Sequence
  Synthetic Linker 1
GAGGGGSG

SEQ ID NO: 630 - protein, 6 aa, Artificial Sequence
  Synthetic Linker 2
EPKSSD

SEQ ID NO: 631 - protein, 5 aa, Artificial Sequence
  Synthetic Linker 3
PVGVV

SEQ ID NO: 632 - protein, 21 aa, Artificial Sequence
  Synthetic Linker 4
ESPKAQASSVPTAQPQAEGLA

SEQ ID NO: 633 - protein, 17 aa, Artificial Sequence
  Synthetic Linker 5
ELQLEESAAEAQDGELD

SEQ ID NO: 634 - protein, 9 aa, Artificial Sequence
  Synthetic Linker 6
GQPDEPGGS

SEQ ID NO: 635 - protein, 13 aa, Artificial Sequence
  Synthetic Linker 7
GGSGSGSGSGSGS

SEQ ID NO: 636 - protein, 17 aa, Artificial Sequence
  Synthetic Linker 8
ELQLEESAAEAQEGELE

SEQ ID NO: 637 - protein, 5 aa, Artificial Sequence
  Synthetic Linker 9
GSGSG

SEQ ID NO: 638 - protein, 4 aa, Artificial Sequence
  Synthetic Linker 10
GSGC

SEQ ID NO: 639 - protein, 7 aa, Artificial Sequence
  Synthetic Linker 11
AGGGGSG

SEQ ID NO: 640 - protein, 4 aa, Artificial Sequence
  Synthetic Linker 12
GSGS

SEQ ID NO: 641 - protein, 8 aa, Artificial Sequence
  Synthetic Linker 13
QPDEPGGS

SEQ ID NO: 642 - protein, 6 aa, Artificial Sequence
  Synthetic Linker 14
GSGSGS

SEQ ID NO: 643 - protein, 6 aa, Artificial Sequence
  Synthetic Linker 15
TVAAPS

SEQ ID NO: 644 - protein, 8 aa, Artificial Sequence
  Synthetic Linker 16
KAGGGGSG

SEQ ID NO: 645 - protein, 13 aa, Artificial Sequence
  Synthetic Linker 17
KGSGSGSGSGSGS

SEQ ID NO: 646 - protein, 9 aa, Artificial Sequence
  Synthetic Linker 18
KQPDEPGGS

SEQ ID NO: 647 - protein, 18 aa, Artificial Sequence
  Synthetic Linker 19
KELQLEESAAEAQDGELD

SEQ ID NO: 648 - protein, 7 aa, Artificial Sequence
  Synthetic Linker 20
KTVAAPS

SEQ ID NO: 649 - protein, 9 aa, Artificial Sequence
  Synthetic Linker 21
KAGGGGSGG

SEQ ID NO: 650 - protein, 14 aa, Artificial Sequence
  Synthetic Linker 22
KGSGSGSGSGSGSG

SEQ ID NO: 651 - protein, 10 aa, Artificial Sequence
  Synthetic Linker 23
KQPDEPGGSG

SEQ ID NO: 652 - protein, 19 aa, Artificial Sequence
  Synthetic Linker 24
KELQLEESAAEAQDGELDG

SEQ ID NO: 653 - protein, 8 aa, Artificial Sequence
  Synthetic Linker 25
KTVAAPSG

SEQ ID NO: 654 - protein, 8 aa, Artificial Sequence
  Synthetic Linker 26
AGGGGSGG

SEQ ID NO: 655 - protein, 7 aa, Artificial Sequence
  Synthetic Linker 27
AGGGGSG

SEQ ID NO: 656 - protein, 13 aa, Artificial Sequence
  Synthetic Linker 28
GSGSGSGSGSGSG

SEQ ID NO: 657 - protein, 9 aa, Artificial Sequence
  Synthetic Linker 29
QPDEPGGSG

SEQ ID NO: 658 - protein, 7 aa, Artificial Sequence
  Synthetic Linker 30
TVAAPSG

SEQ ID NO: 659 - protein, 7 aa, Artificial Sequence
  Synthetic Linker 31
PSTSTST

SEQ ID NO: 660 - protein, 7 aa, Artificial Sequence
  Synthetic Linker 32
EIDKPSQ

SEQ ID NO: 661 - protein, 8 aa, Artificial Sequence
  Synthetic Linker 33
GSGSGSGS

SEQ ID NO: 662 - protein, 10 aa, Artificial Sequence
  Synthetic Linker 34
GSGSGSGSGS

SEQ ID NO: 663 - protein, 12 aa, Artificial Sequence
  Synthetic Linker 35
GSGSGSGSGSGS

SEQ ID NO: 664 - protein, 14 aa, Artificial Sequence
  Synthetic Linker 36
GSGSGSGSGSGSGS

SEQ ID NO: 665 - protein, 13 aa, Artificial Sequence
  Synthetic Linker 37
GGSGSGSGSGSGS

SEQ ID NO: 666 - protein, 16 aa, Artificial Sequence
  Synthetic Linker 38
GGSGSGSGSGSGSGSG

SEQ ID NO: 667 - protein, 15 aa, Artificial Sequence
  Synthetic Linker 39
GSEGSEGSEGSEGSE

SEQ ID NO: 668 - protein, 8 aa, Artificial Sequence
  Synthetic Linker 40
GGSEGGSE

SEQ ID NO: 669 - protein, 35 aa, Artificial Sequence
  Synthetic Linker 41
GGGGSGGGGSGGGGSGGGGSGGGGSGGGGSGGGGS

SEQ ID NO: 670 - protein, 25 aa, Artificial Sequence
  Synthetic Linker 42
GGGGSGGGGSGGGGSGGGGSGGGGS

SEQ ID NO: 671 - protein, 16 aa, Artificial Sequence
  Synthetic Linker 43
GGGGSGGGGSGGGGSG

SEQ ID NO: 672 - protein, 7 aa, Artificial Sequence
  Synthetic Linker 44
GPGPGPG

SEQ ID NO: 673 - protein, 11 aa, Artificial Sequence
  Synthetic Linker 45
GPGPGPGPGPG

SEQ ID NO: 674 - protein, 6 aa, Artificial Sequence
  Synthetic Linker 46
PAPAPA

SEQ ID NO: 675 - protein, 12 aa, Artificial Sequence
  Synthetic Linker 47
PAPAPAPAPAPA

SEQ ID NO: 676 - protein, 18 aa, Artificial Sequence
  Synthetic Linker 48
PAPAPAPAPAPAPAPAPA

SEQ ID NO: 677 - protein, 20 aa, Artificial Sequence
  Synthetic Linker 49
GSGSGSGSGSGSGSGSGSGS

SEQ ID NO: 678 - protein, 20 aa, Artificial Sequence
  Synthetic Linker 50
GGGGSGGGGSGGGGSGGGGS

SEQ ID NO: 679 - protein, 123 aa, Artificial Sequence
  Synthetic 12A4 VH
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 680 - protein, 106 aa, Artificial Sequence
  Synthetic 12A4 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 681 - protein, 5 aa, Artificial Sequence
  Synthetic 12A4 VH CDR1
TYAIS

SEQ ID NO: 682 - protein, 16 aa, Artificial Sequence
  Synthetic 12A4 VH CDR2
GIIPIFGKAHYAQKFQ

SEQ ID NO: 683 - protein, 14 aa, Artificial Sequence
  Synthetic 12A4 VH CDR3
KFHFVSGSPFGMDV

SEQ ID NO: 684 - protein, 11 aa, Artificial Sequence
  Synthetic 12A4 VL CDR1
RASQSVSSYLA

SEQ ID NO: 685 - protein, 7 aa, Artificial Sequence
  Synthetic 12A4 VL CDR2
DASNRAT

SEQ ID NO: 686 - protein, 8 aa, Artificial Sequence
  Synthetic 12A4 VL CDR3
QQRSNWPT